(19) United States
(12) Patent Application Publication
(10) Pub. No.: US 2026/0191998 A1
(43) Pub. Date: Jul. 9, 2026

(54) INDOLE PHTHALOCYANINE COMPOUND, PREPARATION METHOD THEREFOR, AND USE THEREOF IN TUMOR DIAGNOSIS IMAGING

(71) Applicants: JIANGSU YAHONG MEDITECH CO., LTD., Taizhou Jiangsu (CN); ASIERIS PHARMACEUTICLALS (SHANGHAI) CO., LTD., Shanghai (CN)

(72) Inventors: Maojiang WU, Shanghai (CN); Tielin WANG, Shanghai (CN); Peipei ZHENG, Shanghai (CN)

(21) Appl. No.: 19/120,368

(22) PCT Filed: Oct. 12, 2023
(86) PCT No.: PCT/CN2023/124246

§ 371 Date: Apr. 11, 2025

(30) Foreign Application Priority Data
Oct. 12, 2022 (CN) 202211250126.5
Sep. 28, 2023 (CN) 202311284553.X

(51) Int. Cl. A61K 49/00; A61K 47/42; B82Y 5/00; C07D 401/14; C07D 403/08; C07D 471/04; C07F 9/6558

(52) U.S. Cl. CPC A61K 49/0032; A61K 47/42; C07D 401/14; C07D 403/08; C07D 471/04; C07F 9/65583; A61K 2123/00; B82Y 5/00

(57) ABSTRACT

The present invention relates to an indole phthalocyanine compound, a preparation method therefor, and a use thereof in tumor diagnosis imaging. Specifically, the present invention relates to a compound having a structure represented by general formula (I) or a pharmaceutically acceptable salt thereof, and a use thereof as a contrast agent. By using the contrast agent of the present invention, near-infrared real-time imaging having high contrast and a clear and distinguishable imaging result can be implemented.

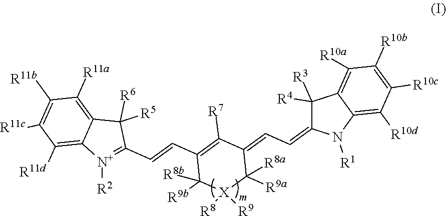

### FIELD OF THE INVENTION

The present invention relates to indole phthalocyanine compounds, methods for preparing the same, and their uses in tumor diagnostic imaging.

### BACKGROUND OF THE INVENTION

Molecular imaging is an interdisciplinary field formed by the fusion of molecular biology and medical imaging. The molecular imaging technology has advantages such as high sensitivity and quantifiability, compared with traditional imaging examinations. The molecular imaging technology or molecular image technique mainly includes magnetic resonance (MR) molecular imaging, nuclear medicine molecular imaging, and optical molecular imaging. Among them, the Near Infrared Fluorescence (NIRF) imaging technology in optical molecular imaging has been widely used in clinical surgical procedures, and the basic principle thereof is to use near-infrared light emitted by a fluorescent tracer after excitation to identify sites such as blood vessels, lymph nodes and tumor lesions, thereby assisting clinical doctors in performing surgical procedures.

Currently, indocyanine green (ICG) has been widely used as a near-infrared fluorescent tracer in the clinical field. However, since ICG does not specifically recognize tumor tissue, the use of ICG in surgical tumor resection only enables to indirectly label tumor lesions according to vascular and tissular differences. The imaging result is poor, and specifically manifested as insufficient sensitivity, poor tumor specificity and short imaging time. There is no specific contrast diagnostic reagent for bladder cancer in clinic.

A problem to be solved is to find a contrast agent that has strong targeting to tumors, enables to clearly distinguish them from surrounding normal tissues, enables to shorten imaging time especially for bladder perfusion, has sufficient duration in tumors, and has good biocompatibility without obvious toxicity and accumulation.

### SUMMARY OF THE INVENTION

The present invention provides a novel near-infrared fluorescent labeling technology for tumors.

Therefore, the object of the present invention is to provide a compound with a structure of general formula (I) or a pharmaceutically acceptable salt thereof,

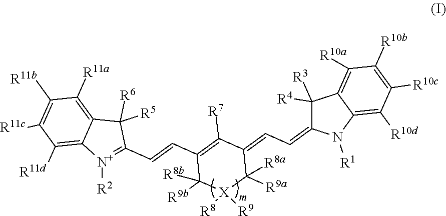

wherein:
X is C, O, S or N⁺;
R¹ and R² are each independently selected from the group consisting of L₁-L₂-L₃-Q;
L₁ is selected from the group consisting of bond, —(CH₂)ₚ—, —(CH₂O)_{q}—, —(CH₂CH₂O)ᵥ—, —(CH₂O)_{q}—(CH₂)ₚ—, —(CH₂)ₚ—(CH₂O)_{q}—, —(CH₂)ₚ—(CH₂O)_{q}—(CH₂)ₚ—, —(CH₂CH₂O)ᵥ—(CH₂)ₚ— and —(CH₂CH₂O)ᵥ—(CH₂O)_{q}—;
L₂ is selected from the group consisting of bond, aryl, heteroaryl, cycloalkyl and heterocyclyl;
L₃ is selected from the group consisting of bond, —(CH₂)ₚ—, —(CH₂O)_{q}—, —(CH₂CH₂O)ᵥ—, —(CH₂O)_{q}—(CH₂)ₚ—, —(CH₂)ₚ—(CH₂O)_{q}—, —(CH₂)ₚ—(CH₂O)_{q}—(CH₂)ₚ—, —(CH₂CH₂O)ᵥ—(CH₂)ₚ— and —(CH₂CH₂O)ᵥ—(CH₂O)_{q}—;
Q is selected from the group consisting of R¹², —OR¹³, —C(O)R¹², —C(O)OR¹³, —C(O)NHR¹³, —SO₃H and

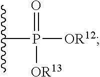

R¹² is selected from the group consisting of hydrogen, halogen, amino, hydroxy, thiol, alkyl, alkylamino, alkenyl, alkynyl, cycloalkyl, heterocyclyl, aryl and heteroaryl; the alkyl, alkenyl, alkynyl, cycloalkyl, heterocyclyl, aryl or heteroaryl is optionally further substituted by one or more groups selected from the group consisting of halogen, amino, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R¹³ is selected from the group consisting of hydrogen, alkyl, alkenyl, alkynyl, cycloalkyl, heterocyclyl, aryl and heteroaryl; the alkyl, alkenyl, alkynyl, cycloalkyl, heterocyclyl, aryl or heteroaryl is optionally further substituted by one or more groups selected from the group consisting of halogen, amino, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R³ and R⁴ are each independently selected from the group consisting of hydrogen, halogen, amino, nitro, cyano, hydroxy, thiol, oxo, alkyl, alkoxy, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R⁵ and R⁶ are each independently selected from the group consisting of hydrogen, halogen, amino, nitro, cyano, hydroxy, thiol, oxo, alkyl, alkoxy, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R⁷ is selected from the group consisting of hydrogen, halogen, nitro, cyano, hydroxy, thiol, oxo, alkyl, OR^{a}, NR^{b}R^{c}, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R⁸ and R⁹ are each independently selected from the group consisting of hydrogen, halogen, nitro, cyano, hydroxy, thiol, oxo, alkyl, OR^{a}, NR^{b}R^{c}, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
R^{8a} and R^{9a} are each independently selected from the group consisting of hydrogen, halogen, amino, nitro, cyano, hydroxy, thiol, oxo, alkyl, alkoxy, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R^{8b} and R^{9b} are each independently selected from the group consisting of hydrogen, halogen, amino, nitro, cyano, hydroxy, thiol, oxo, alkyl, alkoxy, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R^{10a}, R^{10b}, R^{10c} and R^{10d} are each independently selected from the group consisting of hydrogen, halogen, nitro, cyano, hydroxy, thiol, oxo, alkyl, OR^{a}, NR^{b}R^{c}, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
or any two adjacent ones of R^{10a}, R^{10b}, R^{10c} and R^{10d}, together with the atoms to which they are attached, form an aryl or heteroaryl, and the aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
R^{11a}, R^{11b}, R^{11c} and R^{11d} are each independently selected from the group consisting of hydrogen, halogen, nitro, cyano, hydroxy, thiol, oxo, alkyl, OR^{a}, NR^{b}R^{c}, —SO₃H, —S(O)₂R^{a}, —C(O)OR^{a}, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
or any two adjacent ones of R^{11a}, R^{11b}, R^{11c} and R^{11d}, together with the atoms to which they are attached, form an aryl or heteroaryl, and the aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
R^{a} is selected from the group consisting of hydrogen, alkyl, alkenyl, alkynyl, cycloalkyl, heterocyclyl, aryl and heteroaryl, wherein the alkyl, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, sulfonic group, —NHC(O)-L₄-NR^{b}R^{c}, —OC(O)-L₄-NR^{b}R^{c}, alkyl, haloalkyl, alkoxy, haloalkoxy, hydroxyalkyl, alkenyl, alkynyl, cycloalkyl, heterocyclyl, aryl and heteroaryl; wherein -L₄- is alkenylene;
R^{b} and R^{c} are each independently selected from the group consisting of hydrogen, alkyl, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
m is 0 or 1;
p is an integer from 1 to 10;
q is an integer from 1 to 10;
v is an integer from 1 to 10.

In another aspect of the present invention, provided is a compound represented by general formula (IA) or a pharmaceutically acceptable salt thereof,

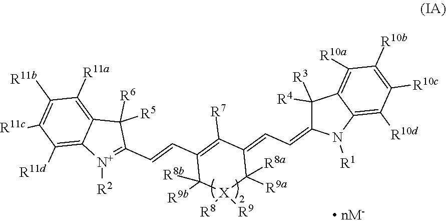

wherein:
X is C, O, S or N⁺;
M⁻ is an acid ion, and preferably chloride ion, bromide ion, iodide ion, formate ion, acetate ion or trifluoroacetate ion;
n is 1 or 2, when X is one N⁺, n is 2, and when X is C, O or S, n is 1;
R¹ and R² are each independently selected from the group consisting of L₁-L₂-L₃-Q;
L₁ is selected from the group consisting of bond, —(CH₂)ₚ—, —(CH₂O)_{q}—, —(CH₂CH₂O)ᵥ—, —(CH₂O)_{q}—(CH₂)ₚ—, —(CH₂)ₚ—(CH₂O)_{q}—, —(CH₂)ₚ—(CH₂O)_{q}—(CH₂)ₚ—, —(CH₂CH₂O)ᵥ—(CH₂)ₚ— and —(CH₂CH₂O)ᵥ—(CH₂O)_{q}—;
L₂ is selected from the group consisting of bond, aryl, heteroaryl, cycloalkyl and heterocyclyl;
L₃ is selected from the group consisting of bond, —(CH₂)ₚ—, —(CH₂O)_{q}—, —(CH₂CH₂O)ᵥ—, —(CH₂O)_{q}—(CH₂)ₚ—, —(CH₂)ₚ—(CH₂O)_{q}—, —(CH₂)ₚ—(CH₂O)_{q}—(CH₂)ₚ—, —(CH₂CH₂O)ᵥ—(CH₂)ₚ— and —(CH₂CH₂O)ᵥ—(CH₂O)_{q}—;
Q is selected from the group consisting of R¹², —OR¹³, —C(O)R¹², —C(O)OR¹³, —C(O)NHR¹³, —SO₃H and

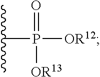

R¹² is selected from the group consisting of hydrogen, halogen, amino, hydroxy, thiol, alkyl, alkylamino, alkenyl, alkynyl, cycloalkyl, heterocyclyl, aryl and heteroaryl; the alkyl, alkenyl, alkynyl, cycloalkyl, heterocyclyl, aryl or heteroaryl is optionally further substituted by one or more groups selected from the group consisting of halogen, amino, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R¹³ is selected from the group consisting of hydrogen, alkyl, alkenyl, alkynyl, cycloalkyl, heterocyclyl, aryl and heteroaryl; the alkyl, alkenyl, alkynyl, cycloalkyl, heterocyclyl, aryl or heteroaryl is optionally further substituted by one or more groups selected from the group consisting of halogen, amino, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R³ and R⁴ are each independently selected from the group consisting of hydrogen, halogen, amino, nitro, cyano, hydroxy, thiol, oxo, alkyl, alkoxy, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R⁵ and R⁶ are each independently selected from the group consisting of hydrogen, halogen, amino, nitro, cyano, hydroxy, thiol, oxo, alkyl, alkoxy, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R⁷ is selected from the group consisting of hydrogen, halogen, nitro, cyano, hydroxy, thiol, oxo, alkyl, OR^{a}, SR^{a}, NR^{b}R^{c}, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R⁸ and R⁹ are each independently selected from the group consisting of hydrogen, halogen, nitro, cyano, hydroxy, thiol, oxo, alkyl, OR^{a}, NR^{b}R^{c}, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
R^{8a} and R^{9a} are each independently selected from the group consisting of hydrogen, halogen, amino, nitro, cyano, hydroxy, thiol, oxo, alkyl, alkoxy, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R^{8b} and R^{9b} are each independently selected from the group consisting of hydrogen, halogen, amino, nitro, cyano, hydroxy, thiol, oxo, alkyl, alkoxy, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R^{10a}, R^{10b}, R^{10c} and R^{10d} are each independently selected from the group consisting of hydrogen, halogen, nitro, cyano, hydroxy, thiol, oxo, alkyl, OR^{a}, NR^{b}R^{c}, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
or any two adjacent ones of R^{10a}, R^{10b}, R^{10c} and R^{10d}, together with the atoms to which they are attached, form an aryl or heteroaryl, and the aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
R^{11a}, R^{11b}, R^{11c} and R^{11d} are each independently selected from the group consisting of hydrogen, halogen, nitro, cyano, hydroxy, thiol, oxo, alkyl, OR^{a}, NR^{b}R^{c}, —SO₃H, —S(O)₂R^{a}, —C(O)OR^{a}, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
or any two adjacent ones of R^{11a}, R^{11b}, R^{11c} and R^{11d}, together with the atoms to which they are attached, form an aryl or heteroaryl, and the aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
R^{a} is selected from the group consisting of hydrogen, alkyl, alkenyl, alkynyl, cycloalkyl, heterocyclyl, aryl and heteroaryl, wherein the alkyl, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, sulfonic group, —NHC(O)-L₄-NR^{b}R^{c}, —OC(O)-L₄-NR^{b}R^{c}, alkyl, haloalkyl, alkoxy, haloalkoxy, hydroxyalkyl, alkenyl, alkynyl, cycloalkyl, heterocyclyl, aryl and heteroaryl; wherein -L₄- is alkenylene;
R^{b} and R^{c} are each independently selected from the group consisting of hydrogen, alkyl, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
m is 0 or 1;
p is an integer from 1 to 10;
q is an integer from 1 to 10;
v is an integer from 1 to 10.

In an embodiment, the compound with the structure of general formula (I) or a pharmaceutically acceptable salt thereof is a compound with a structure of general formula (II) or a pharmaceutically acceptable salt thereof,

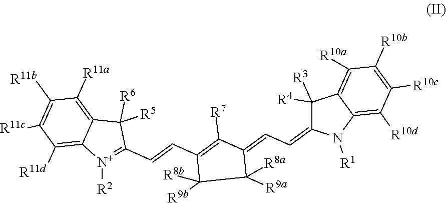

wherein, R¹ to R⁷, R^{8a}, R^{8b}, R^{9a}, R^{9b}, R^{10a}, R^{10b}, R^{10c}, R^{10d}, R^{11a}, R^{11b}, R^{11c} and R^{11d} are as defined in general formula (I).

In another embodiment, the compound with the structure of general formula (I) or a pharmaceutically acceptable salt thereof is a compound with a structure of general formula (III) or a pharmaceutically acceptable salt thereof,

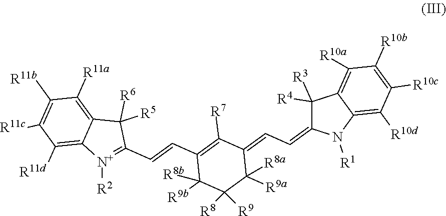

wherein, R¹ to R⁹, R^{8a}, R^{8b}, R^{9a}, R^{9b}, R^{10a}, R^{10b}, R^{10c}, R^{10d}, R^{11a}, R^{11b}, R^{11c} and R^{11d} are as defined in general formula (I).

In another embodiment, the compound represented by general formula (IA) or a pharmaceutically acceptable salt thereof is a compound represented by general formula (IIA) or (IIIA) or a pharmaceutically acceptable salt thereof,

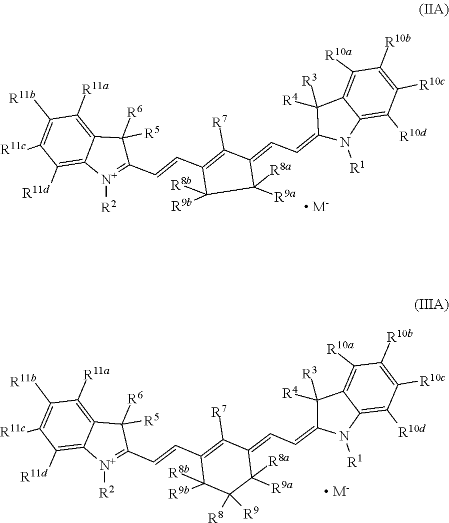

wherein, M⁻, R¹ to R⁹, R^{8a}, R^{8b}, R^{9a}, R^{9b}, R^{10a}, R^{10b}, R^{10c}, R^{10d}, R^{11a}, R^{11b}, R^{11c} and R^{11d} are as defined in general formula (IA).

In another embodiment, the compound with the structure of general formula (I) or a pharmaceutically acceptable salt thereof is a compound with a structure of general formula (IV) or a pharmaceutically acceptable salt thereof,

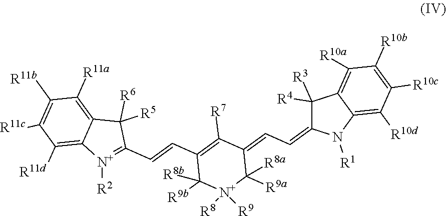

wherein, R¹ to R⁹, R^{8a}, R^{8b}, R^{9a}, R^{9b}, R^{10a}, R^{10b}, R^{10c}, R^{10d}, R^{11a}, R^{11b}, R^{11c} and R^{11d} are as defined in general formula (I).

In another embodiment, the compound represented by general formula (IA) or a pharmaceutically acceptable salt thereof is a compound represented by general formula (IVA) or a pharmaceutically acceptable salt thereof,

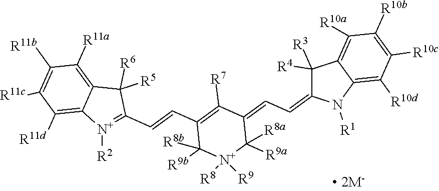

wherein, M⁻, R¹ to R⁹, R^{8a}, R^{8b}, R^{9a}, R^{9b}, R^{10a}, R^{10b}, R^{10c}, R^{10d}, R^{11a}, R^{11b}, R^{11c} and R^{11d} are as defined in general formula (IA).

In another embodiment, in the compound with the structure of general formula (I), (II), (III), (IV), (IA), (IIA), (IIIA) or (IVA) or a pharmaceutically acceptable salt thereof, R¹ is L₁-L₂-Q;
L₁ is selected from the group consisting of —(CH₂)ₚ— and —(CH₂O)_{q}—(CH₂)ₚ—;
L₂ is selected from the group consisting of bond, C₆₋₁₀ aryl and 5 to 10 membered heteroaryl; and preferably selected from the group consisting of bond and phenyl;
Q is selected from the group consisting of R¹², —C(O)OR¹³, —C(O)NHR¹³, —SO₃H and

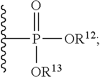

R¹² is selected from the group consisting of hydrogen, hydroxy and C₁₋₆ alkyl;
R¹³ is selected from the group consisting of hydrogen, C₆₋₁₀ aryl and 5 to 10 membered heteroaryl; and preferably selected from the group consisting of hydrogen and phenyl.
p is an integer from 1 to 10, and preferably an integer from 1 to 6;
q is an integer from 1 to 10, and preferably an integer from 1 to 6.

In another embodiment, in the compound with the structure of general formula (I), (II), (III), (IV), (IA), (IIA), (IIIA) or (IVA) or a pharmaceutically acceptable salt thereof,
R² is L₁-Q;
L₁ is —(CH₂)ₚ—;
Q is selected from the group consisting of R¹², —C(O)OR¹³, —C(O)NHR¹³, —SO₃H and

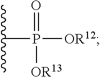

R¹² is selected from the group consisting of hydrogen, hydroxy and C₁₋₆ alkyl;
R¹³ is selected from the group consisting of hydrogen, C₆₋₁₀ aryl and 5 to 10 membered heteroaryl; and preferably selected from the group consisting of hydrogen and phenyl.
p is an integer from 1 to 10, and preferably an integer from 1 to 6.

In another embodiment, in the compound with the structure of general formula (I), (II), (III), (IV), (IA), (IIA), (IIIA) or (IVA) or a pharmaceutically acceptable salt thereof, R^{10a}, R^{10b}, R^{10c} and R^{10d} are each independently selected from the group consisting of hydrogen and OR^{a}, and R^{a} is C₁₋₆ alkyl.

In another embodiment, in the compound with the structure of general formula (I), (II), (III), (IV), (IA), (IIA), (IIIA) or (IVA) or a pharmaceutically acceptable salt thereof, R^{11a}, R^{11b}, R^{11c} and R^{11d} are each independently selected from the group consisting of hydrogen, carboxy, OR^{a}, NR^{b}R^{c}, —SO₃H, —S(O)₂R^{a} and —C(O)OR^{a},
R^{a} is C₁₋₆ alkyl;
R^{b} and R^{c} are each independently C₁₋₆ alkyl.

In another embodiment, in the compound with the structure of general formula (I), (II), (III), (IV), (IA), (IIA), (IIIA) or (IVA) or a pharmaceutically acceptable salt thereof, any two adjacent ones of R^{10a}, R^{10b}, R^{10c} and R^{10d}, together with the atoms to which they are attached, form a C₆₋₁₀ aryl or 5 to 10 membered heteroaryl, and preferably phenyl; the aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl.

In another embodiment, in the compound with the structure of general formula (I), (II), (III), (IV), (IA), (IIA), (IIIA) or (IVA) or a pharmaceutically acceptable salt thereof, any two adjacent ones of R^{11a}, R^{11b}, R^{11c} and R^{11d}, together with the atoms to which they are attached, form a C₆₋₁₀ aryl or 5 to 10 membered heteroaryl, and preferably phenyl; the aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl.

In another embodiment, the compound with the structure of general formula (I) or a pharmaceutically acceptable salt thereof is a compound with a structure of general formula (V) or a pharmaceutically acceptable salt thereof,

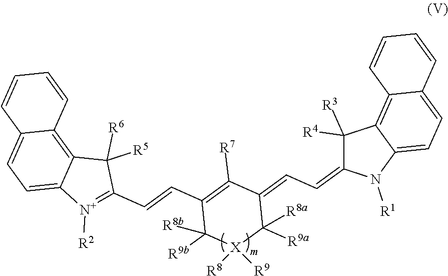

wherein, R¹ to R⁹, R^{8a}, R^{8b}, R^{9a}, R^{9b}, X and m are as defined in general formula (I).

In another embodiment, in the compounds with the structure of general formula (I), (II), (III), (IV), (V), (IA), (IIA), (IIIA) or (IVA) or a pharmaceutically acceptable salt thereof,
R¹ is L₁-Q;
L₁ is —(CH₂)ₚ—;
Q is selected from the group consisting of —C(O)OR¹³, —C(O)NHR¹³, —SO₃H and

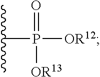

R¹³ is selected from the group consisting of hydrogen, C₆₋₁₀ aryl and 5 to 10 membered heteroaryl; and preferably selected from the group consisting of hydrogen and phenyl;
p is an integer from 1 to 10, and preferably an integer from 1 to 6;
R² is C₁₋₆ alkyl.

In another embodiment, in the compound with the structure of general formula (I), (II), (III), (IV), (V), (IA), (IIA), (IIIA) or (IVA) or a pharmaceutically acceptable salt thereof,
R¹ is L₁-Q;
L₁ is —(CH₂O)_{q}—(CH₂)ₚ—;
Q is selected from the group consisting of —C(O)OR¹³ and —C(O)NHR¹³;
R¹³ is selected from the group consisting of hydrogen, C₆₋₁₀ aryl and 5 to 10 membered heteroaryl; and preferably phenyl;
p is an integer from 1 to 6, preferably an integer from 1 to 4, and more preferably 1 or 2;
q is an integer from 1 to 6, preferably an integer from 1 to 4, and more preferably 1 or 2;
R² is C₁₋₆ alkyl.

In another embodiment, in the compound with the structure of general formula (I), (II), (III), (IV), (V), (IA), (IIA), (IIIA) or (IVA) or a pharmaceutically acceptable salt thereof,
R¹ is L₁-L₂-Q;
L₁ is —(CH₂)ₚ—;
L₂ is selected from the group consisting of C₆₋₁₀ aryl and 5 to 10 membered heteroaryl, and preferably phenyl;
Q is —C(O)OR¹³;
R¹³ is selected from the group consisting of hydrogen, C₆₋₁₀ aryl and 5 to 10 membered heteroaryl; and preferably phenyl;
p is an integer from 1 to 4, and preferably an integer from 1 to 2;
R² is C₁₋₆ alkyl.

In another embodiment, in the compound with the structure of general formula (I), (II), (III), (IV), (V), (IA), (IIA), (IIIA) or (IVA) or a pharmaceutically acceptable salt thereof,
R¹ is C₁₋₆ alkyl;
R² is C₁₋₆ alkyl.

In another embodiment, in the compound with the structure of general formula (I), (II), (III), (IV), (V), (IA), (IIA), (IIIA) or (IVA) or a pharmaceutically acceptable salt thereof,
R¹ and R² are each independently selected from L₁-Q;
L₁ is —(CH₂)ₚ—;
Q is selected from the group consisting of R¹², —C(O)OR¹³, —C(O)NHR¹³, —SO₃H and

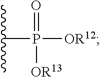

R¹² is selected from the group consisting of hydrogen, hydroxy and C₁₋₆ alkyl;
R¹³ is selected from the group consisting of hydrogen, C₆₋₁₀ aryl and 5 to 10 membered heteroaryl; and preferably selected from the group consisting of hydrogen and phenyl;
p is an integer from 1 to 10, and preferably an integer from 1 to 6.

In another embodiment, in the compound with the structure of general formula (I), (II), (III), (IV), (V), (IA), (IIA), (IIIA) or (IVA) or a pharmaceutically acceptable salt thereof, R⁷ is selected from the group consisting of hydrogen, halogen, hydroxy, OR^{a}, SR^{a} and NR^{b}R^{c};
R^{a} is selected from the group consisting of C₆₋₁₀ aryl and 5 to 10 membered heteroaryl, and preferably phenyl; wherein, the aryl or heteroaryl is optionally further substituted by one or more substituents selected from the group consisting of halogen, amino, oxo, thioxo, nitro, cyano, hydroxy, thiol, carboxy, ester, sulfonic group, —NHC(O)-L₄-NR^{b}R^{c}, —OC(O)-L₄-NR^{b}R^{c}, alkyl, haloalkyl, alkoxy, haloalkoxy, hydroxyalkyl, alkenyl, alkynyl, cycloalkyl, heterocyclyl, aryl and heteroaryl; wherein -L₄- is alkenylene;
R^{b} and R^{c} are each independently selected from the group consisting of hydrogen and C₁₋₁₀ alkyl, wherein the C₁₋₁₀ alkyl is optionally further substituted by carboxy.

In another embodiment, in the compound with the structure of general formula (I), (II), (III), (IV), (V), (IA), (IIA), (IIIA) or (IVA) or a pharmaceutically acceptable salt thereof, R⁷ is SR^{a}; R^{a} is C₁₋₆ alkyl, and the C₁₋₆ alkyl is optionally substituted by carboxy.

In another embodiment, in the compound with the structure of general formula (I), (II), (III), (IV), (V), (IA), (IIA), (IIIA) or (IVA) or a pharmaceutically acceptable salt thereof, R⁸ and R⁹ are each independently selected from the group consisting of hydrogen and C₁₋₆ alkyl.

In another embodiment, in the compound with the structure of general formula (I), (II), (III), (IV), (V), (IA), (IIA), (IIIA) or (IVA) or a pharmaceutically acceptable salt thereof, R^{8a} and R^{9a} are each independently hydrogen.

In another embodiment, in the compound with the structure of general formula (I), (II), (III), (IV), (V), (IA), (IIA), (IIIA) or (IVA) or a pharmaceutically acceptable salt thereof, R^{8b} and R^{9b} are each independently hydrogen.

In another embodiment, in the compound with the structure of general formula (I), (II), (III), (IV), (V), (IA), (IIA), (IIIA) or (IVA) or a pharmaceutically acceptable salt thereof, R³ and R⁴ are each independently selected from the group consisting of hydrogen and C₁₋₆ alkyl.

In another embodiment, in the compound with the structure of general formula (I), (II), (III), (IV), (V), (IA), (IIA), (IIIA) or (IVA) or a pharmaceutically acceptable salt thereof, R⁵ and R⁶ are each independently selected from the group consisting of hydrogen and C₁₋₆ alkyl.

In another embodiment, in the compound with the structure of general formula (I), (II), (III), (IV), (V), (IA), (IIA), (IIIA) or (IVA) or a pharmaceutically acceptable salt thereof, the pharmaceutically acceptable salt is halide salt or carboxylate salt, and preferably hydrochloride salt, formate salt, acetate salt or trifluoroacetate salt.

Typical compounds of the present invention include, but are not limited to:

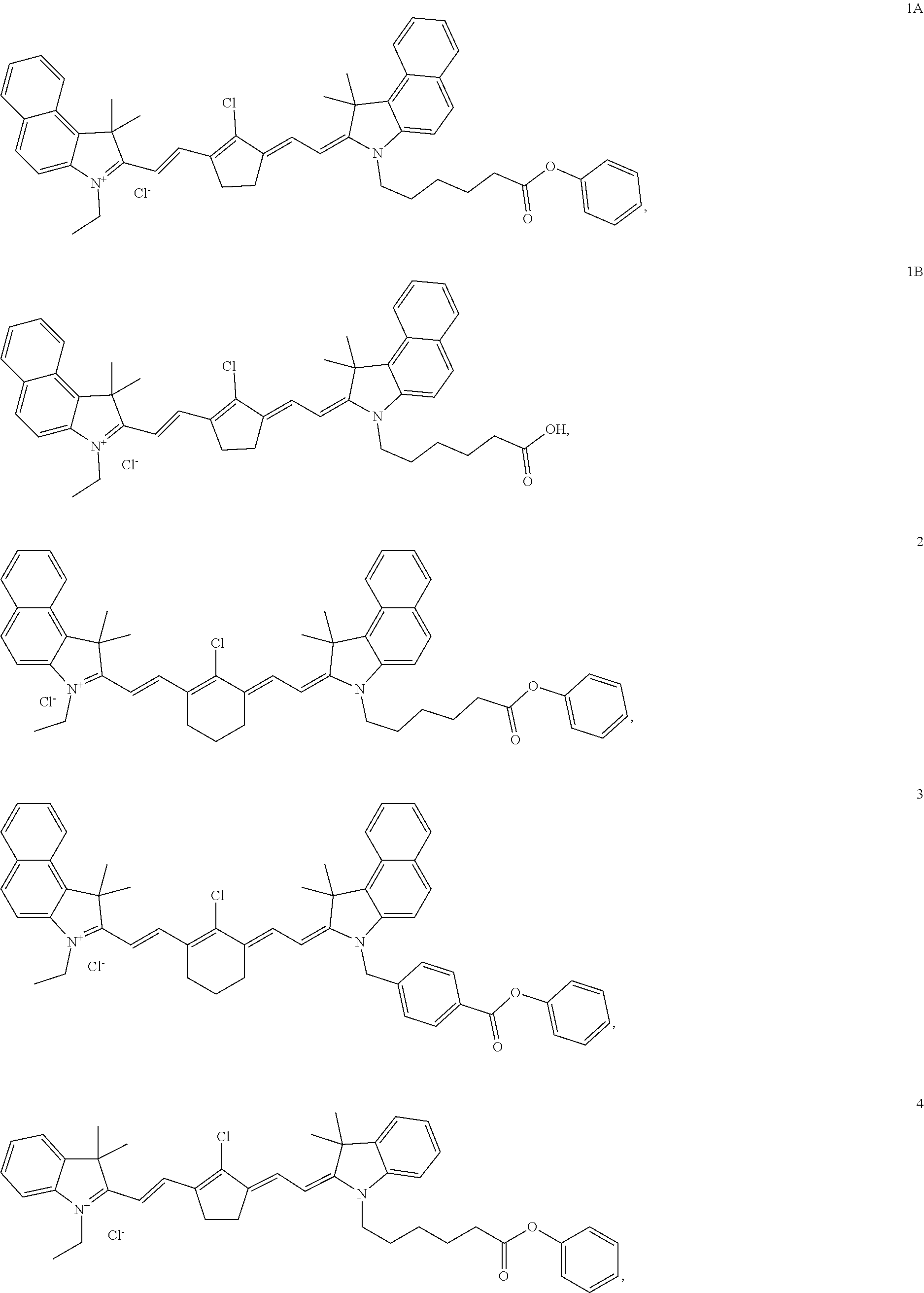

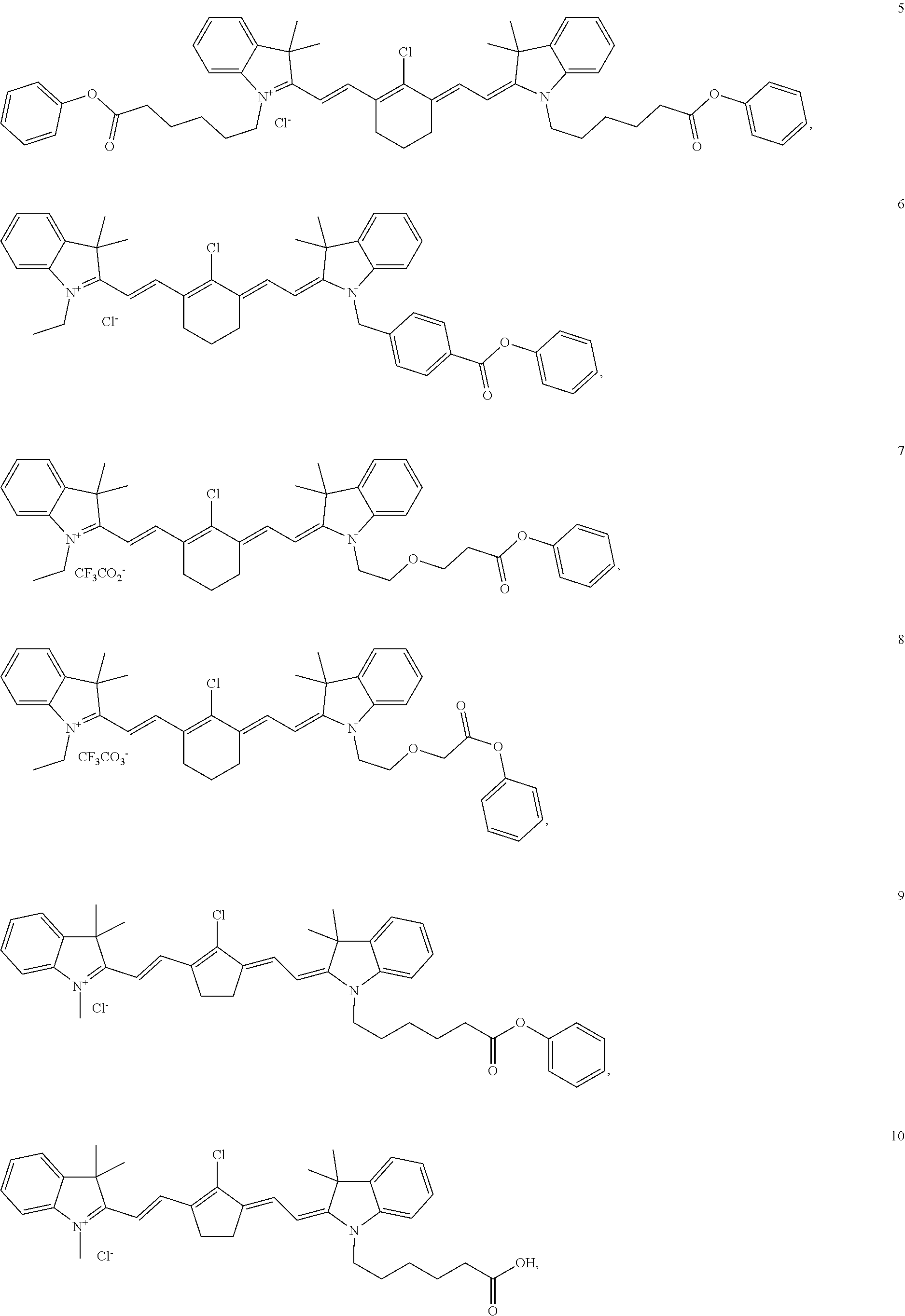

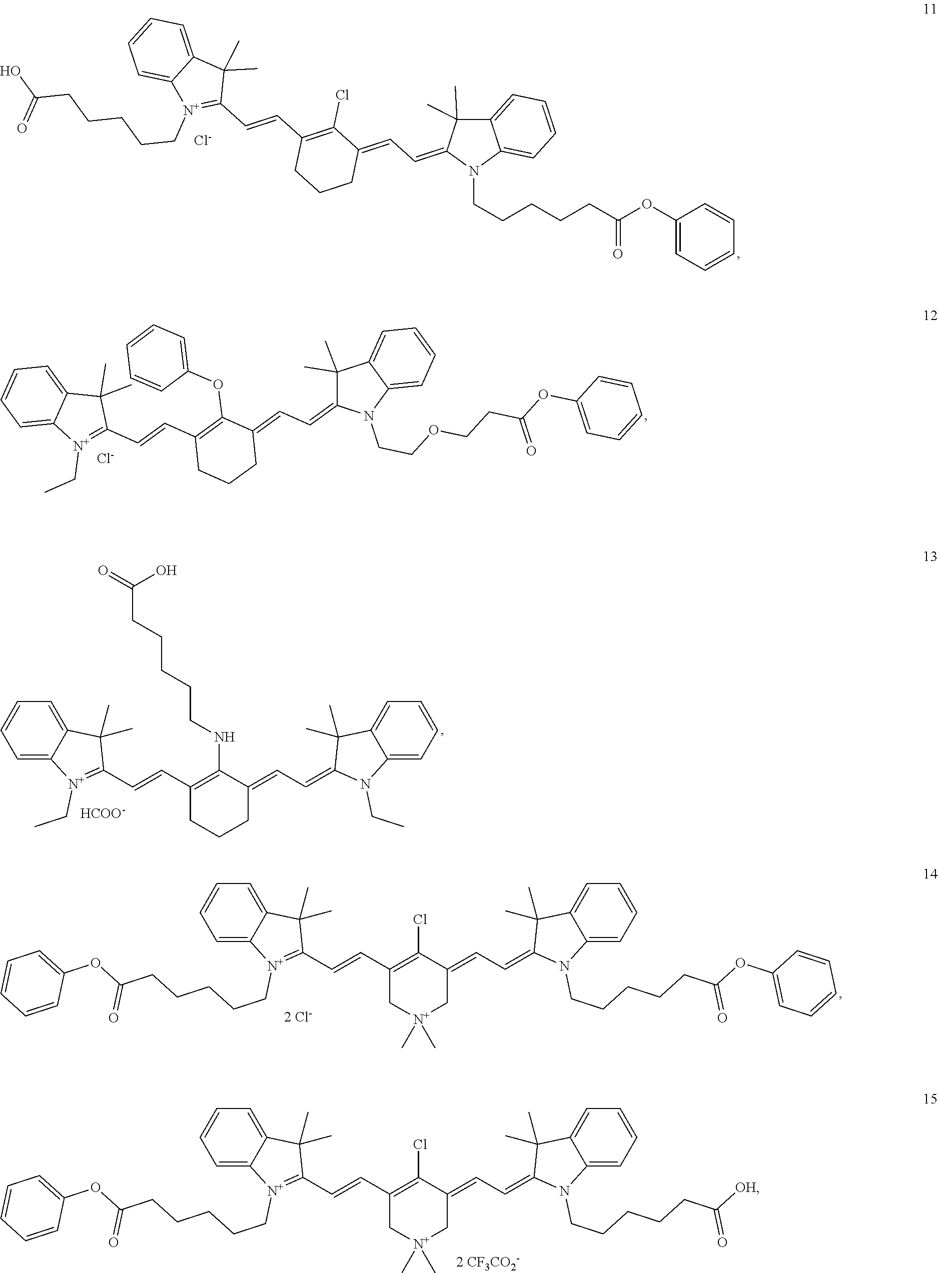

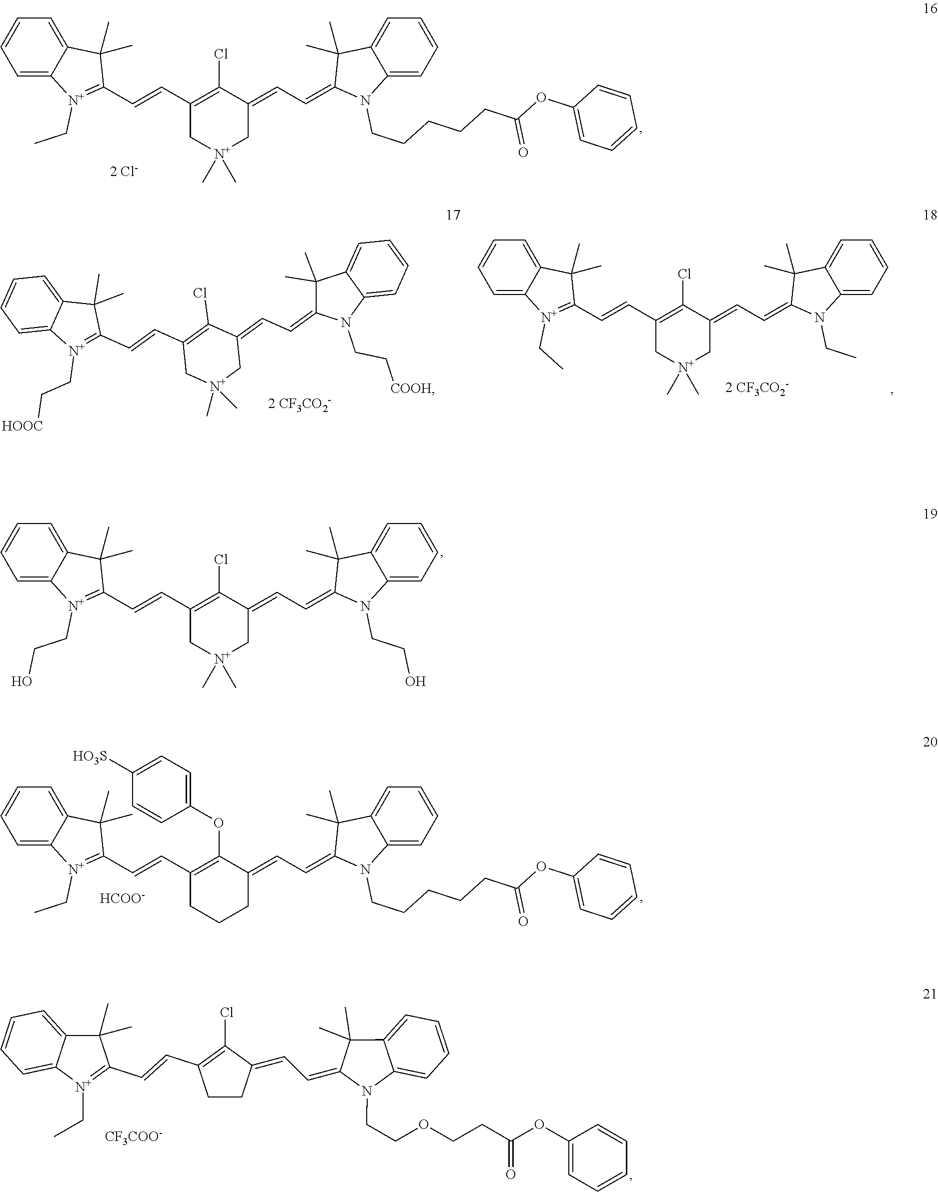

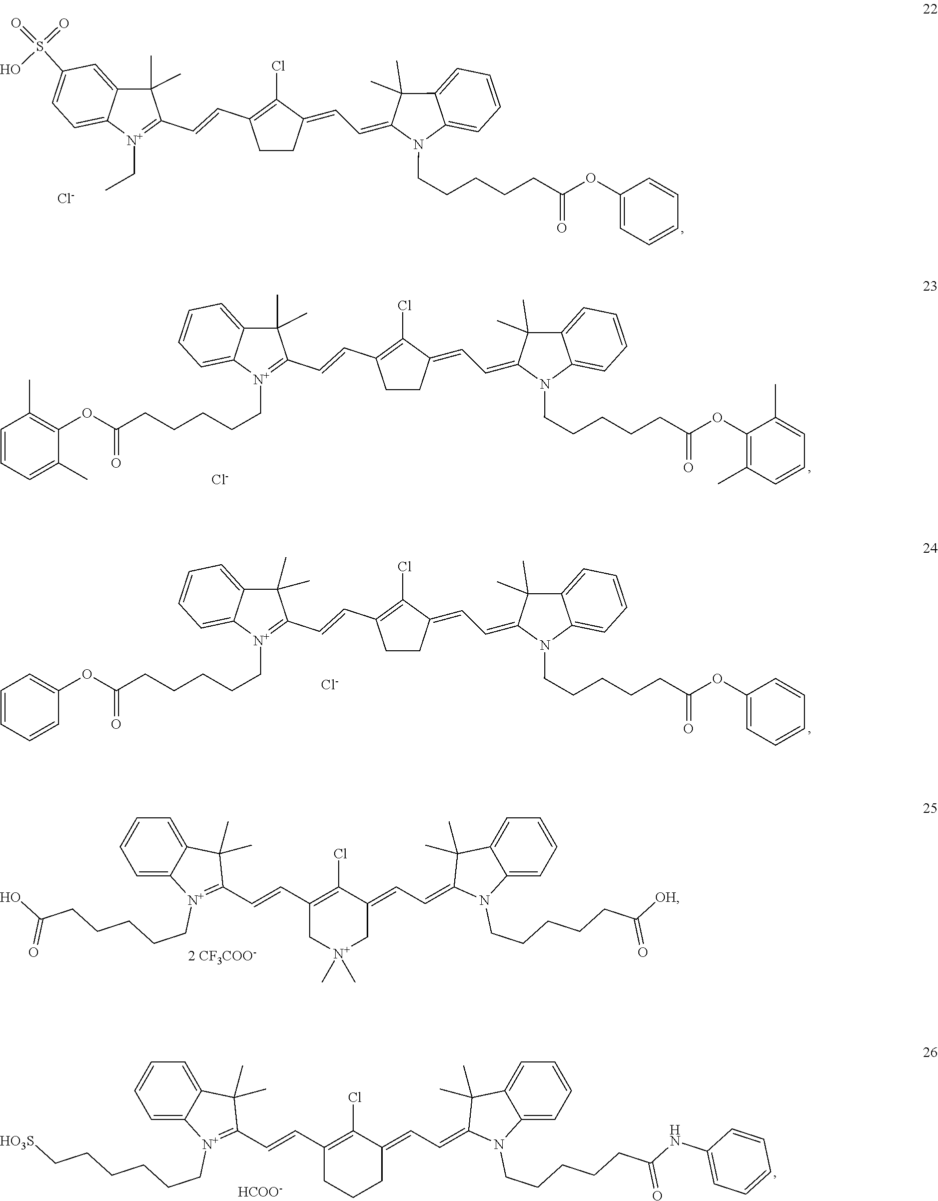

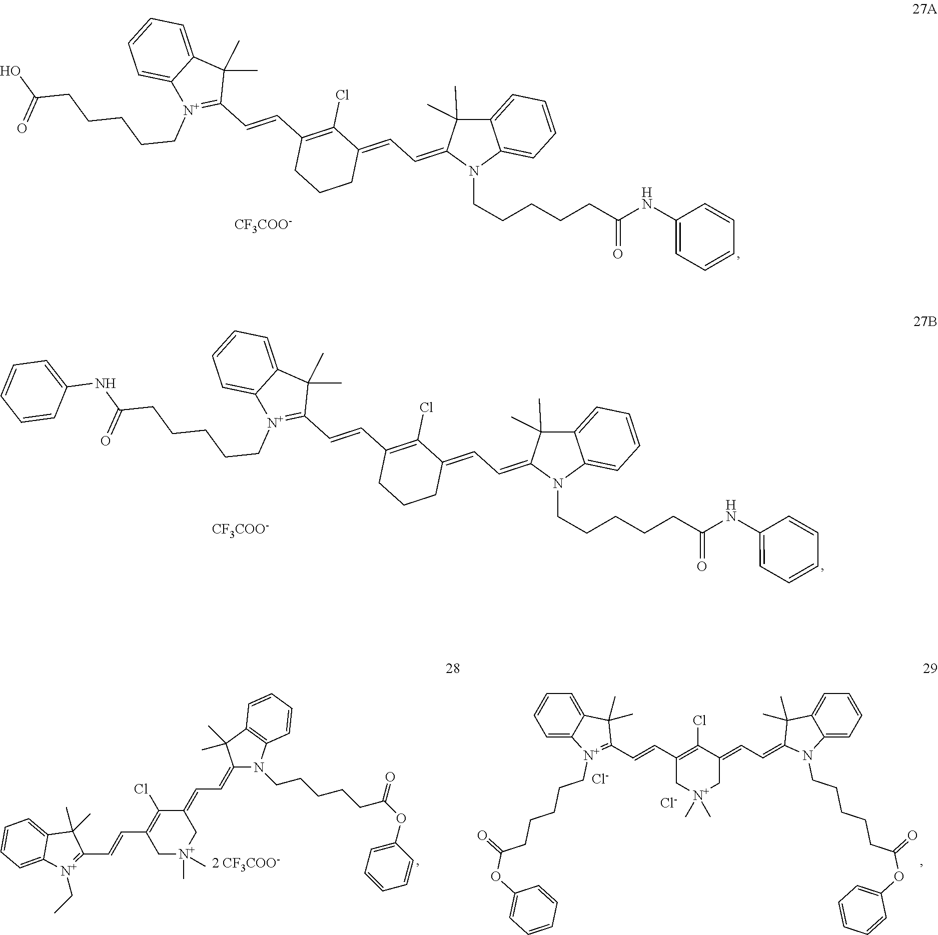

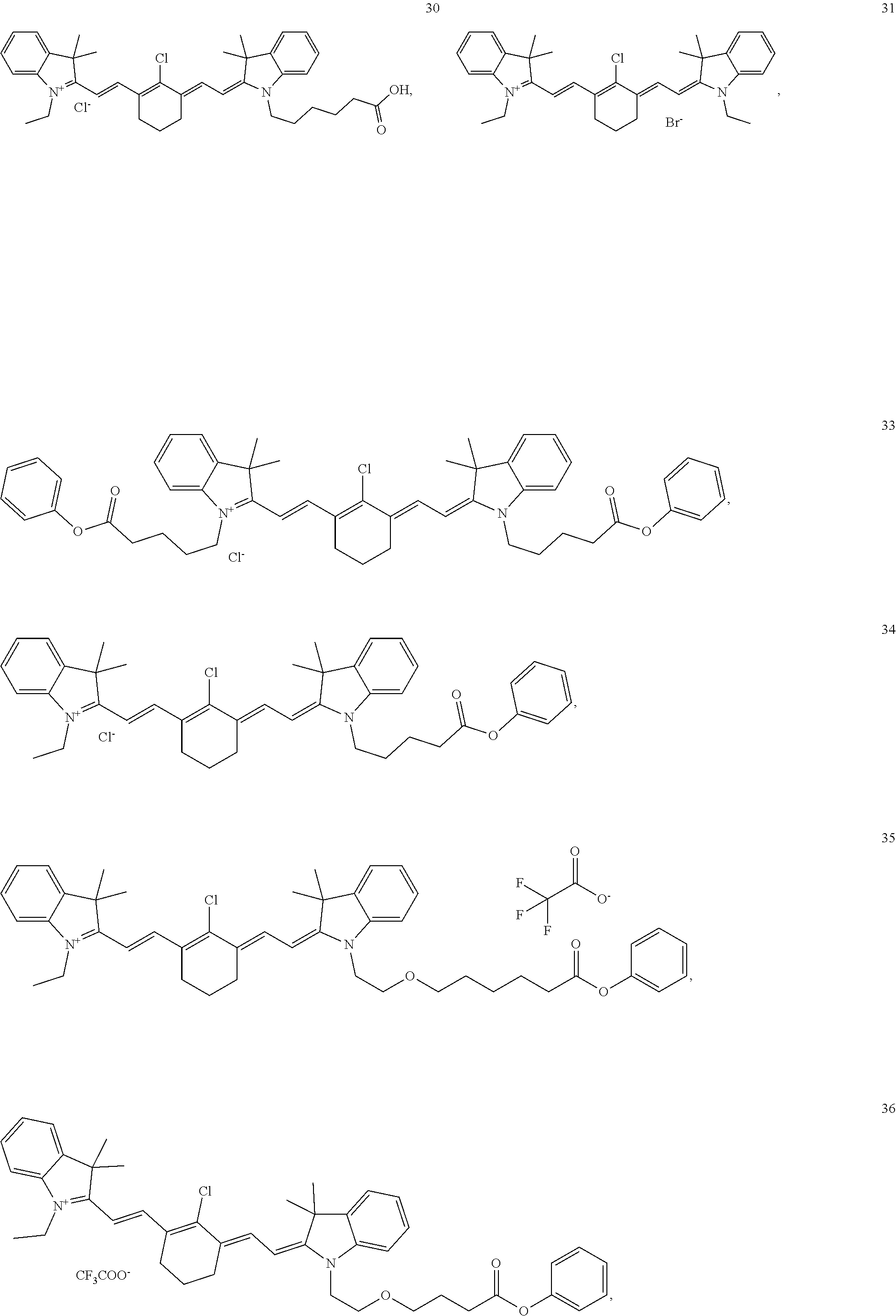

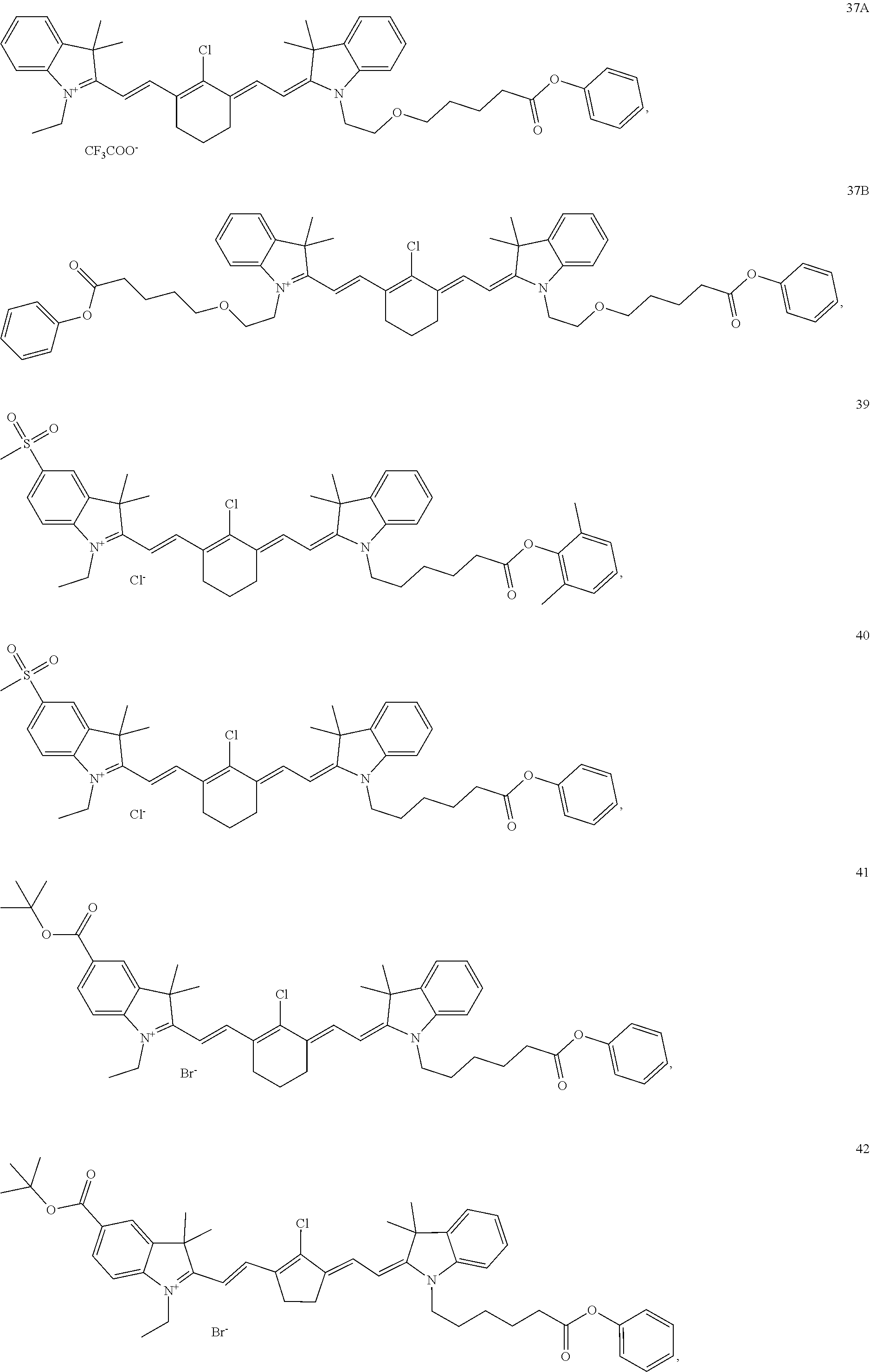

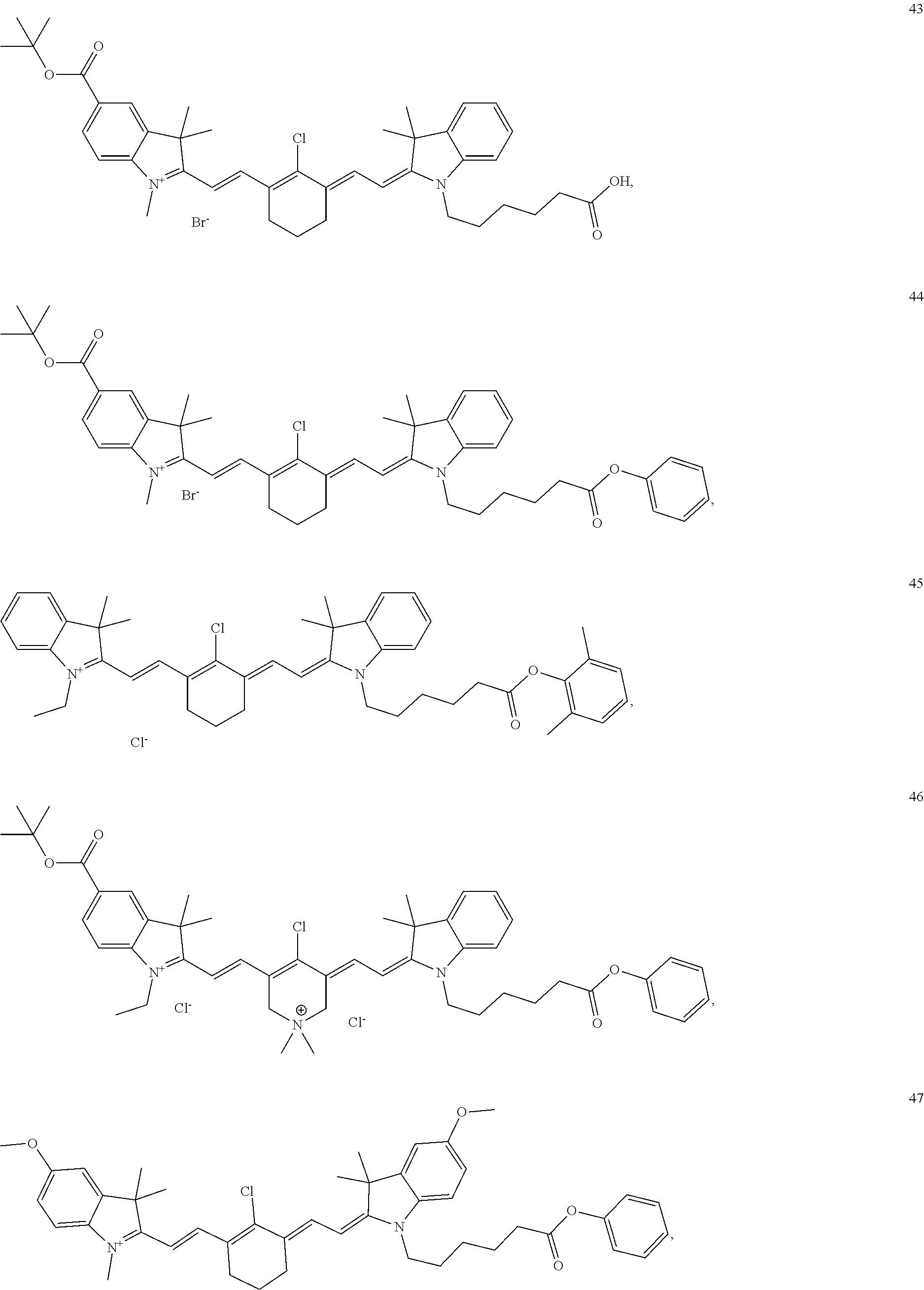

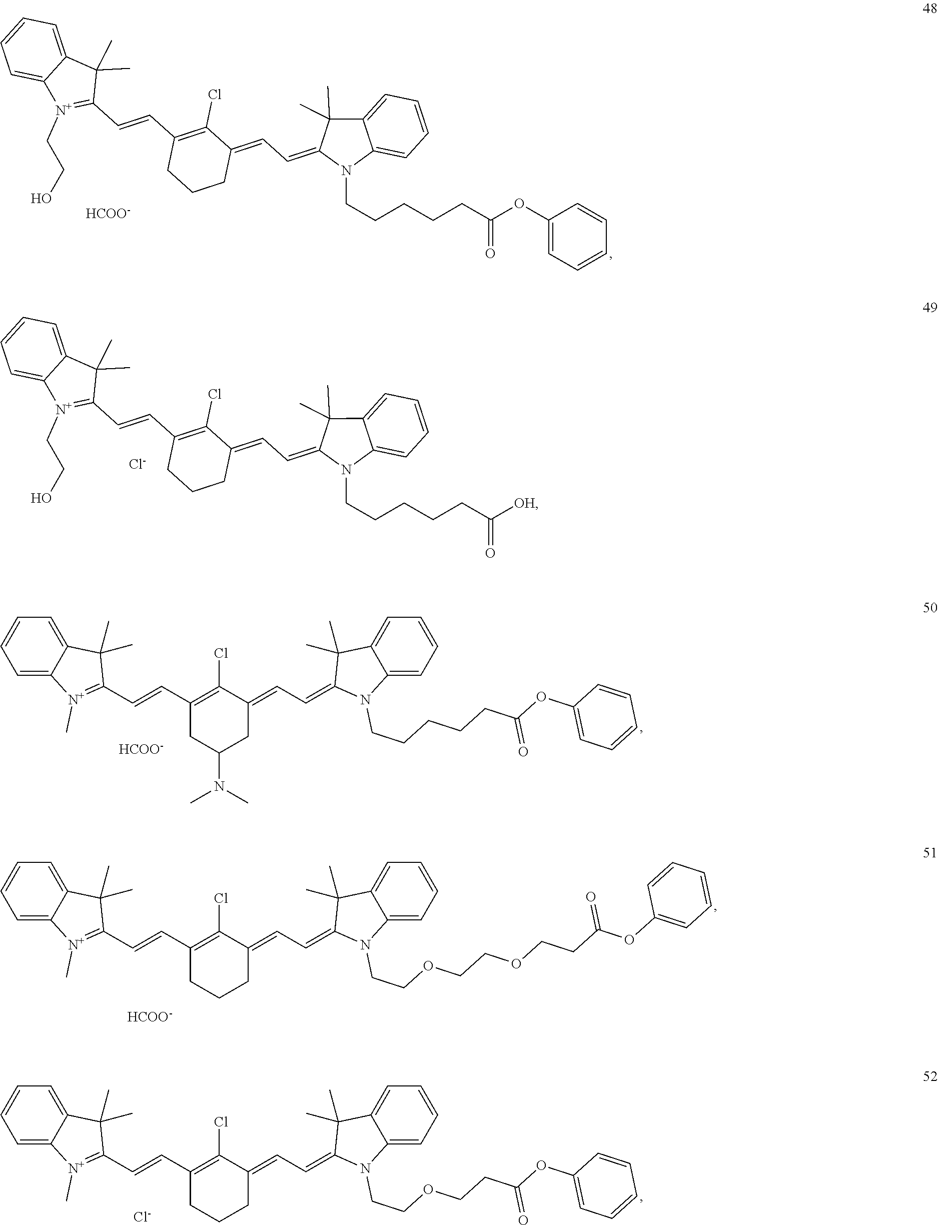

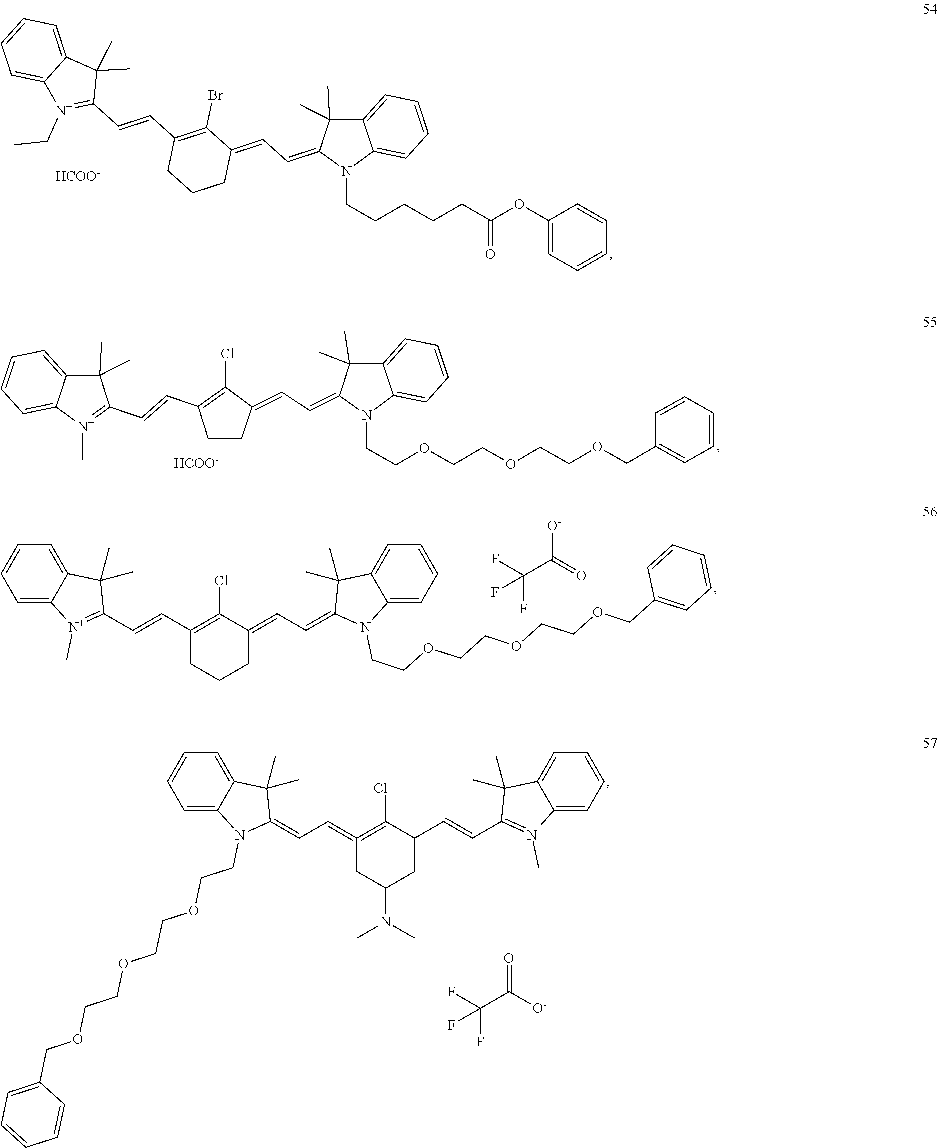

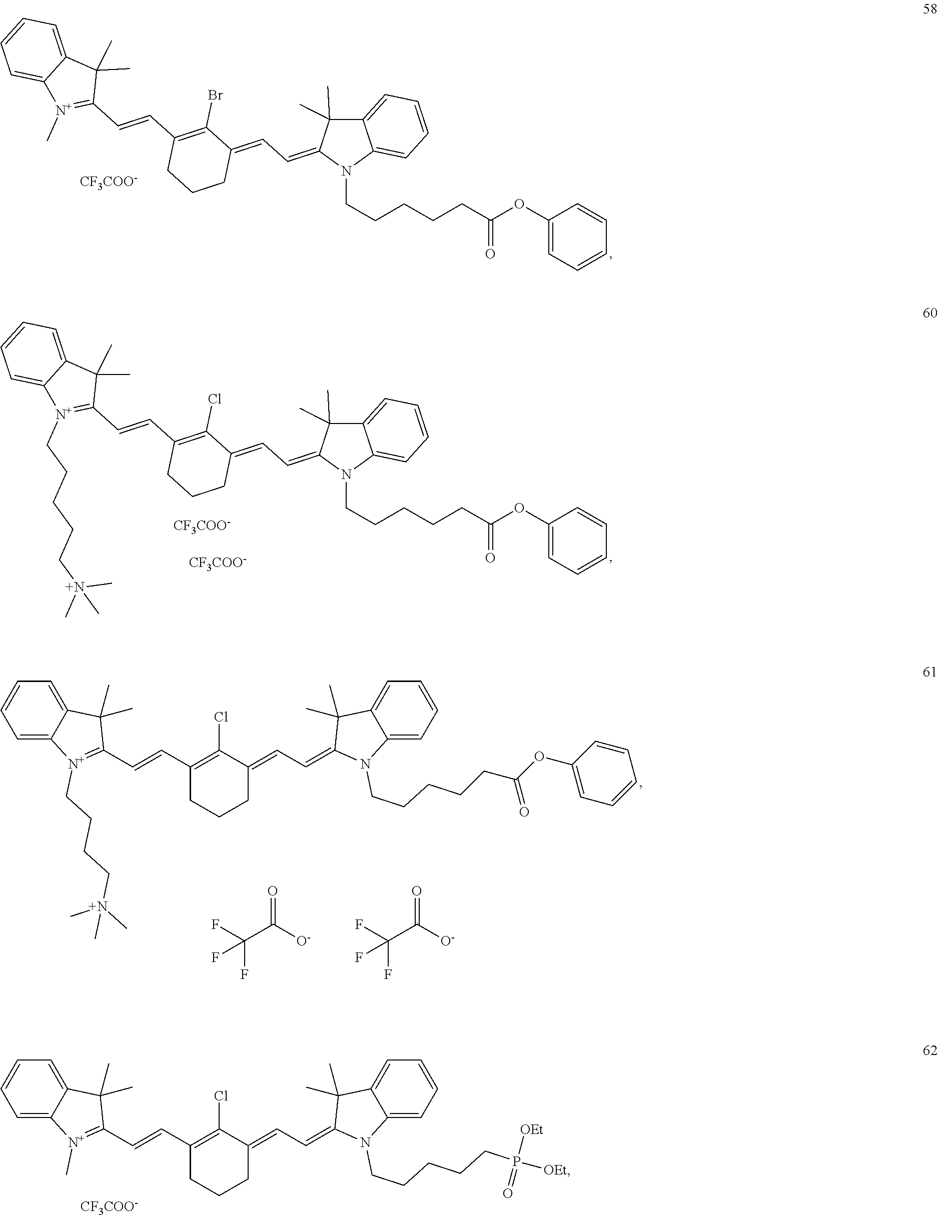

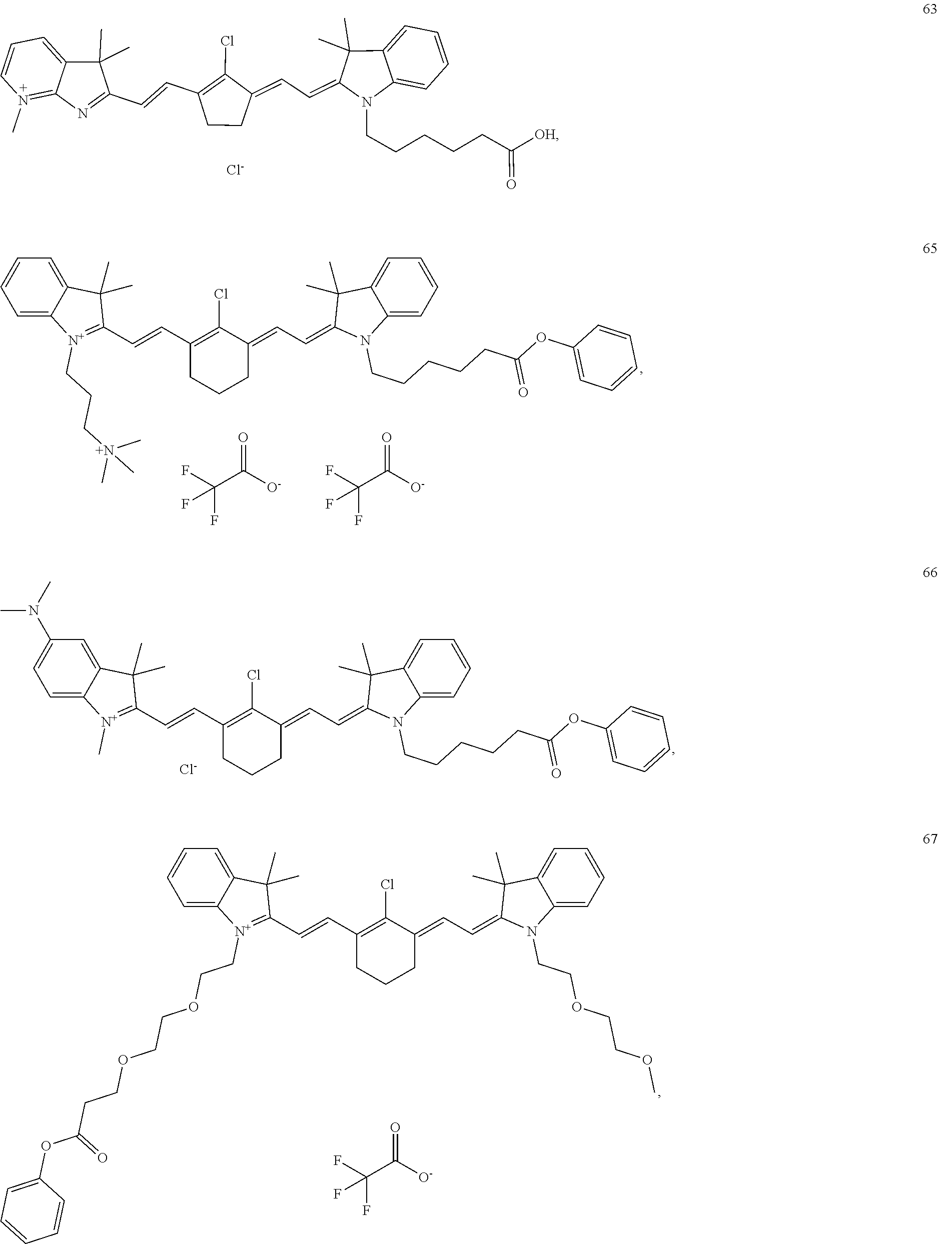

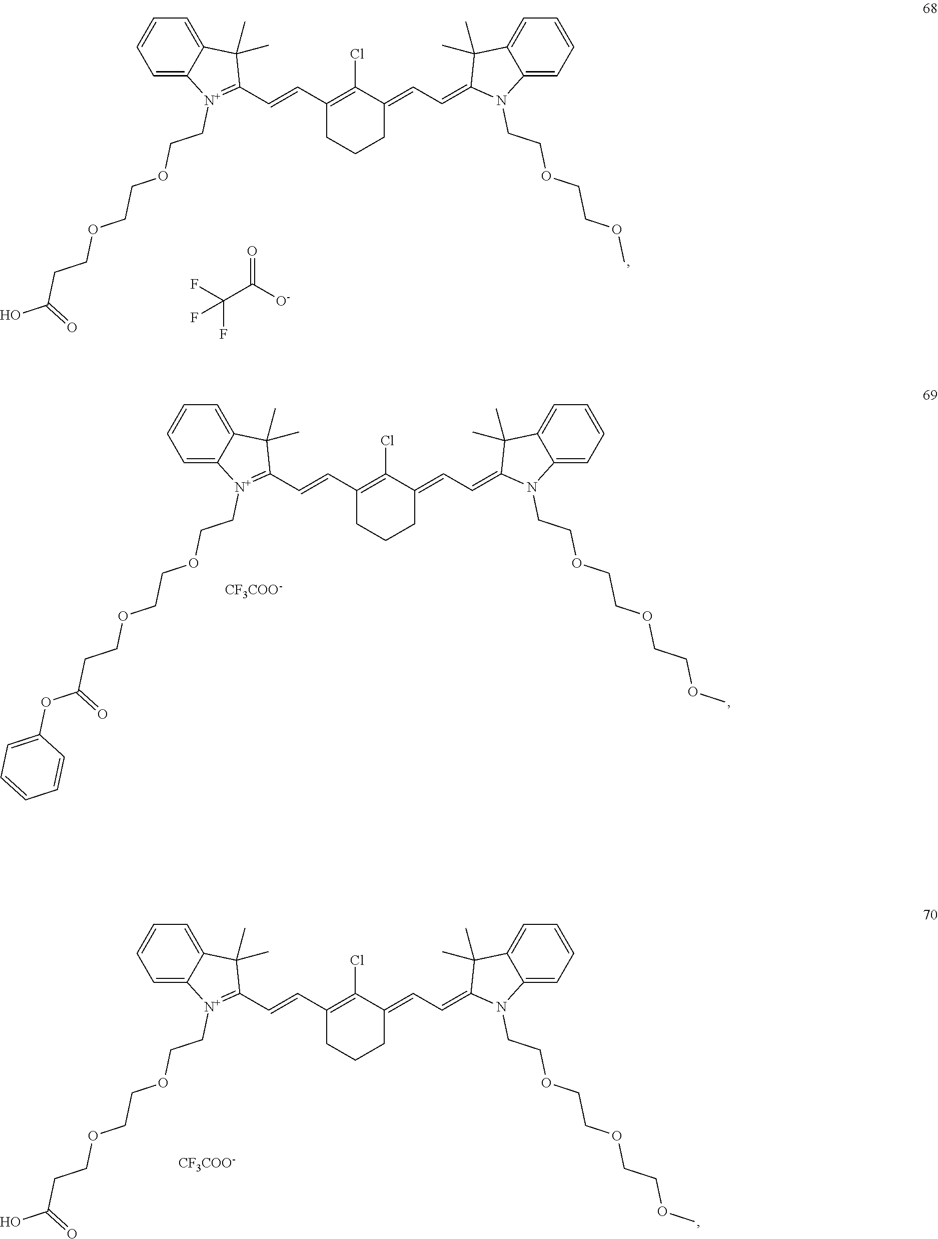

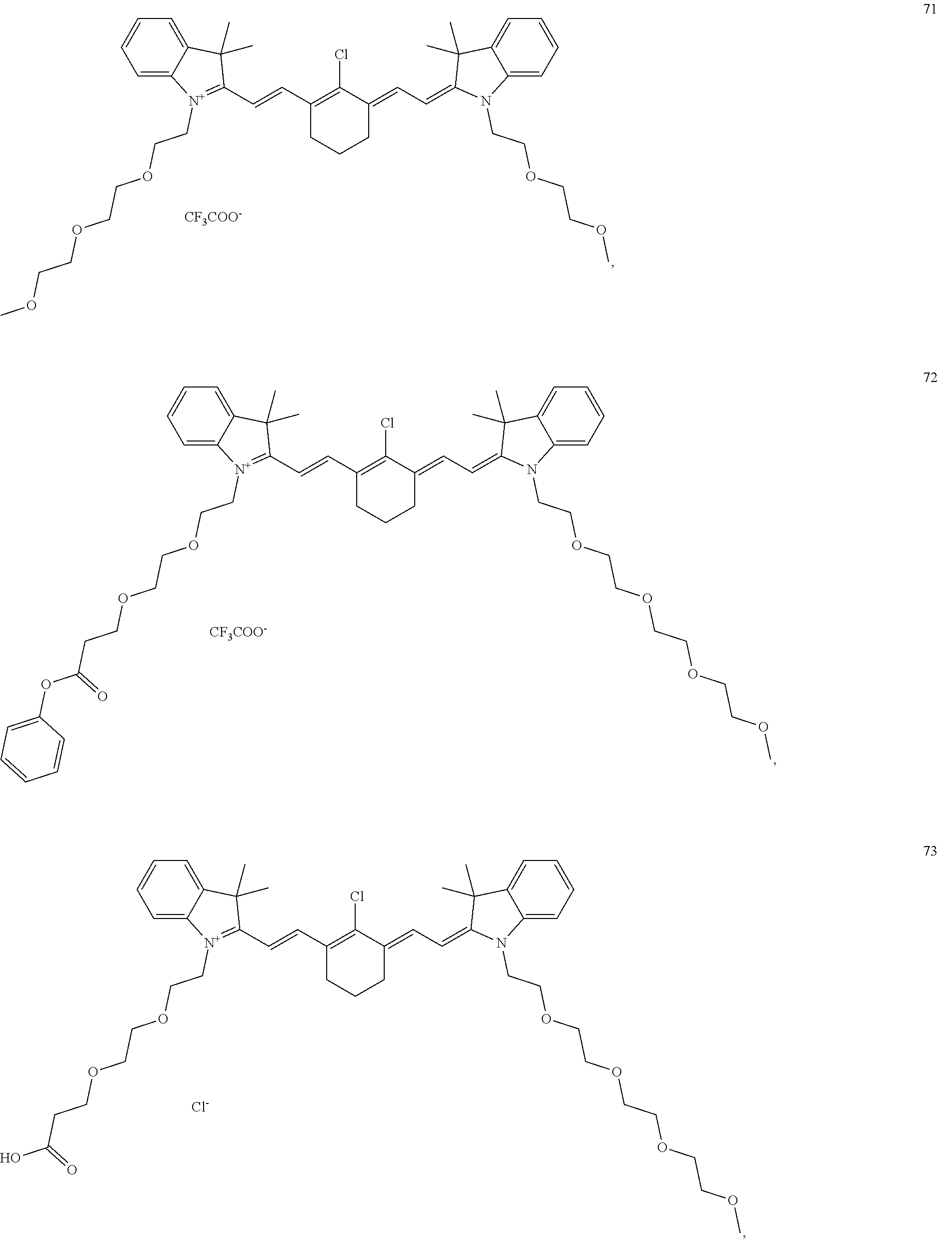

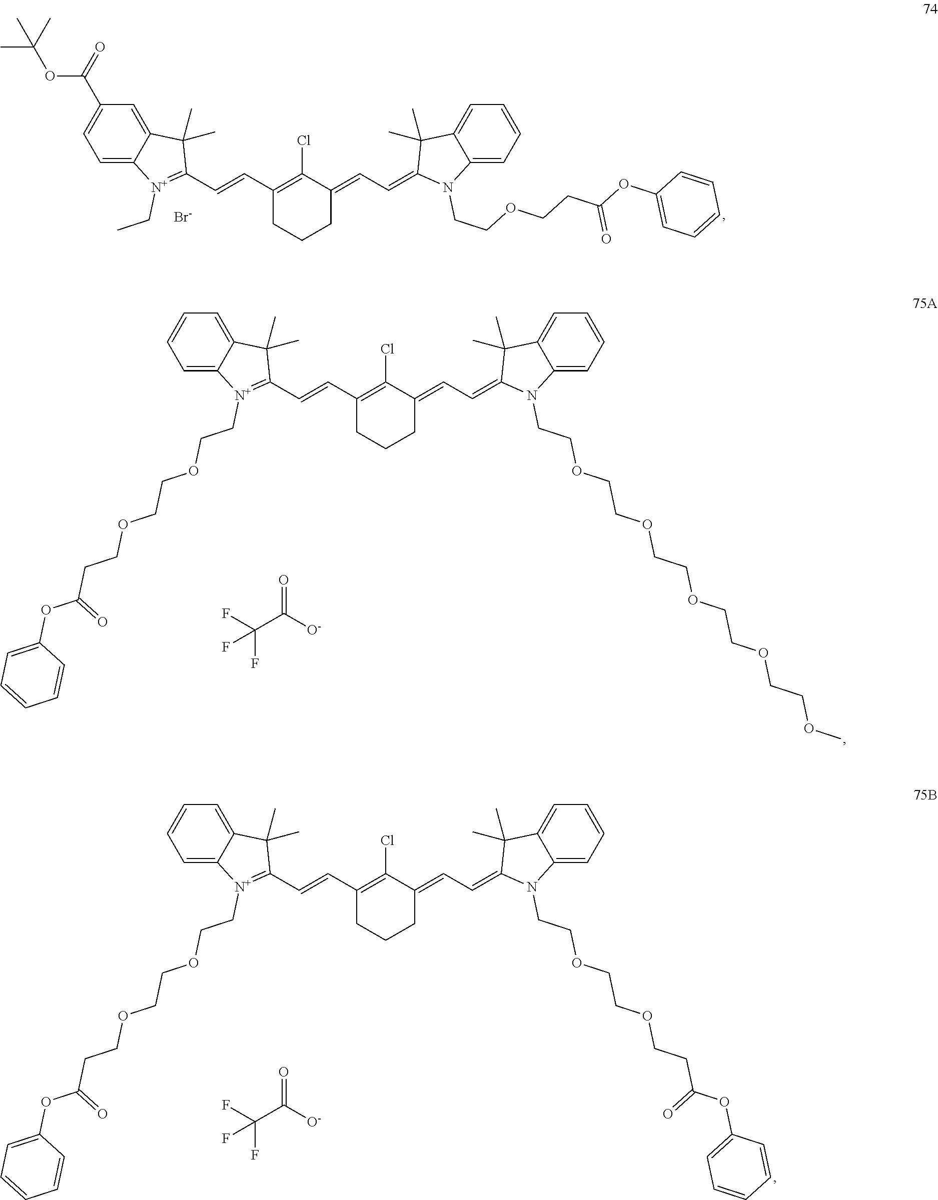 [SMILES: CC[N+]1=C(/C=C/C2=C(Cl)C(=C/C=C3/N(CCOCCC(=O)Oc4ccccc4)c4ccccc4C3(C)C)/CCC2)C(C)(C)c2cc(C(=O)OC(C)(C)C)ccc21 ; [Br-] ; COCCOCCOCCOCCOCCN1/C(=C/C=C2\CCCC(/C=C/C3=[N+](CCOCCOCCC(=O)Oc4ccccc4)c4ccccc4C3(C)C)=C2Cl)C(C)(C)c2ccccc21 ; O=C([O-])C(F)(F)F ; CC1(C)C(/C=C/C2=C(Cl)C(=C/C=C3/N(CCOCCOCCC(=O)Oc4ccccc4)c4ccccc4C3(C)C)/CCC2)=[N+](CCOCCOCCC(=O)Oc2ccccc2)c2ccccc21 ; O=C([O-])C(F)(F)F]

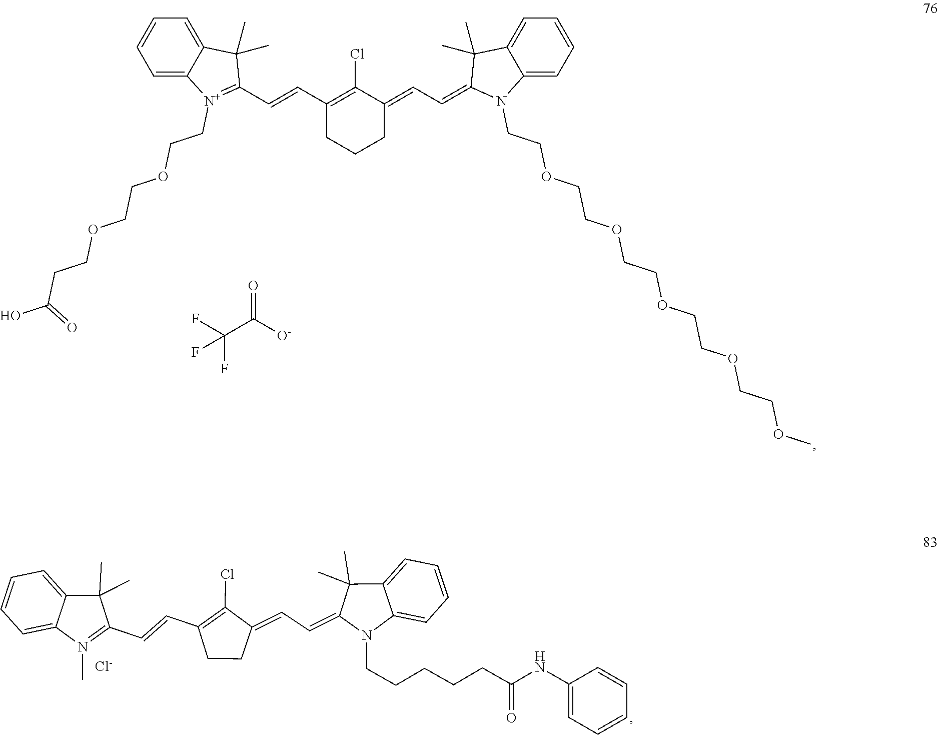

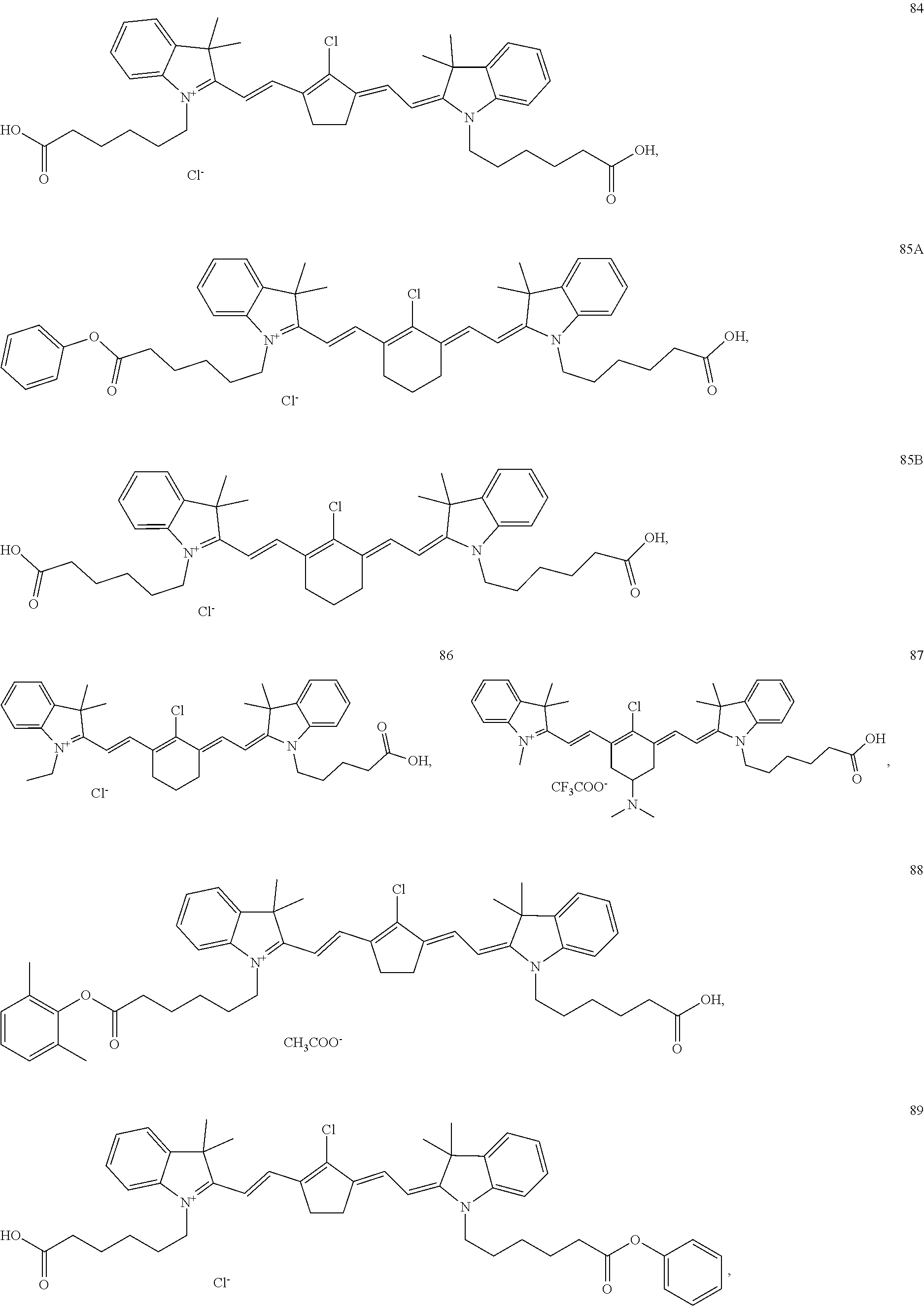

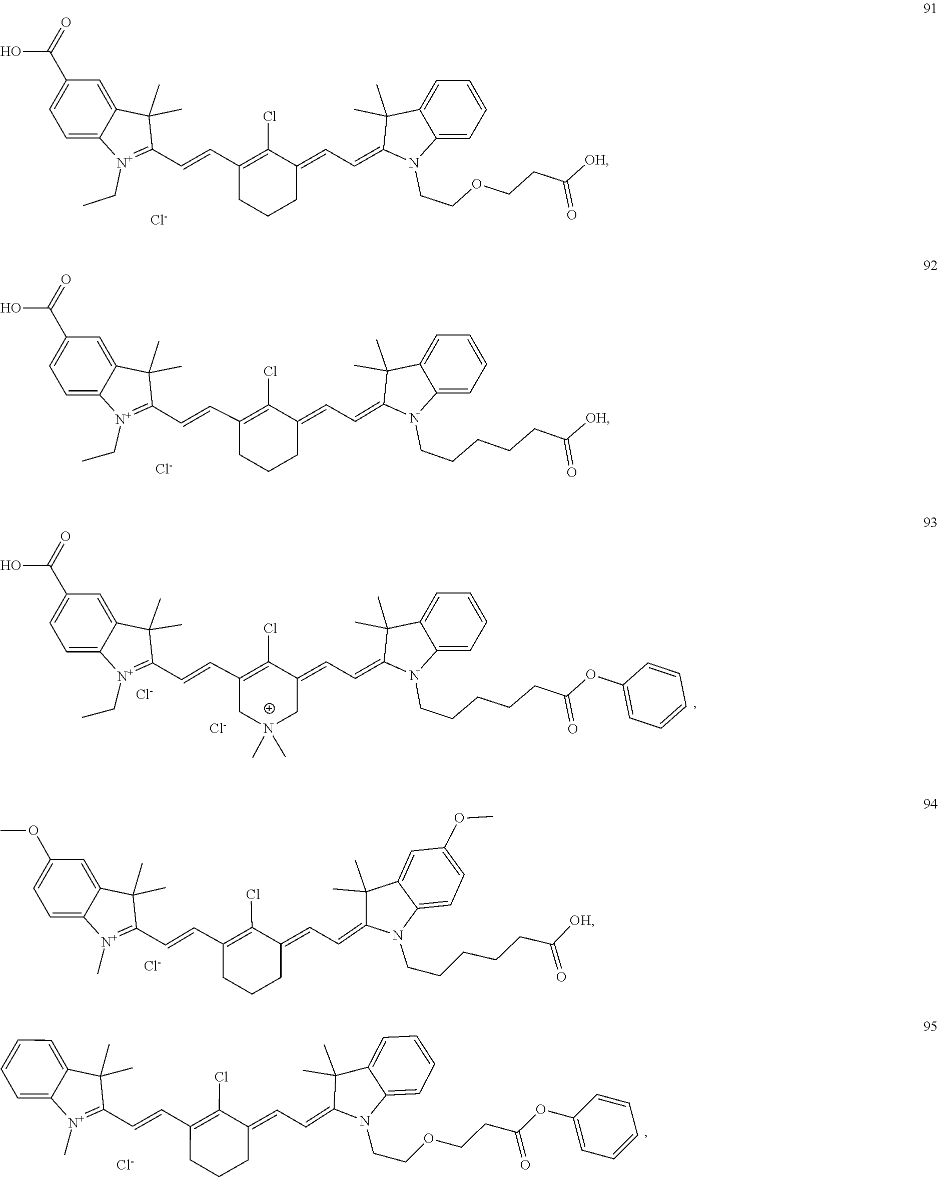

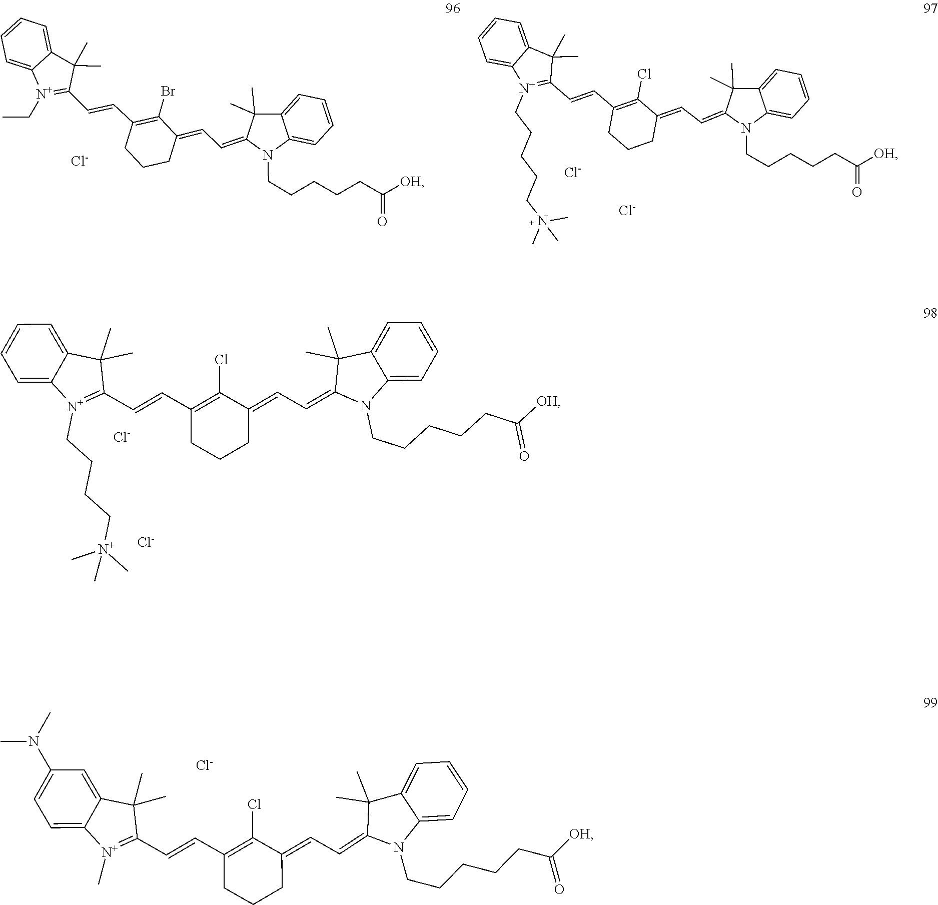

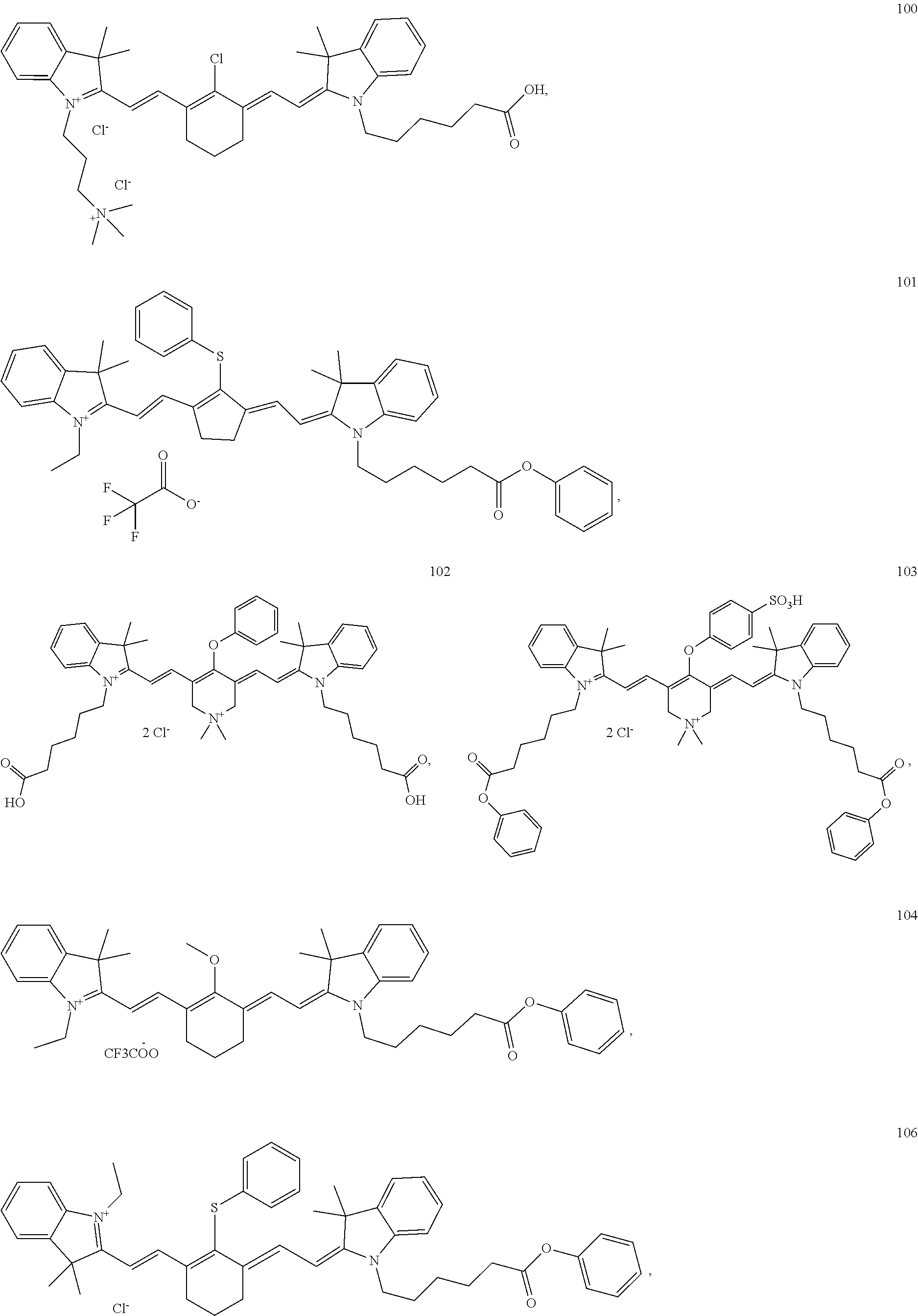

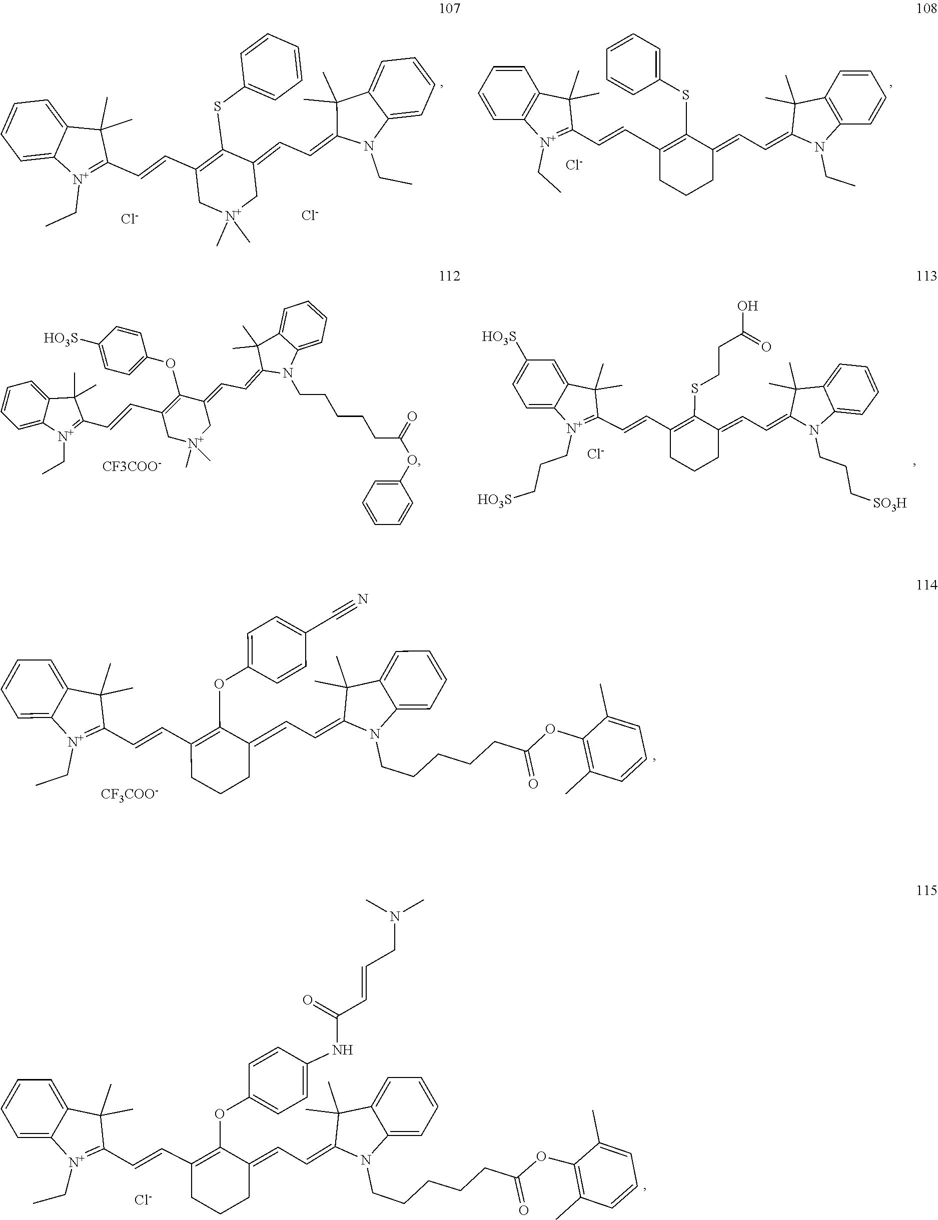

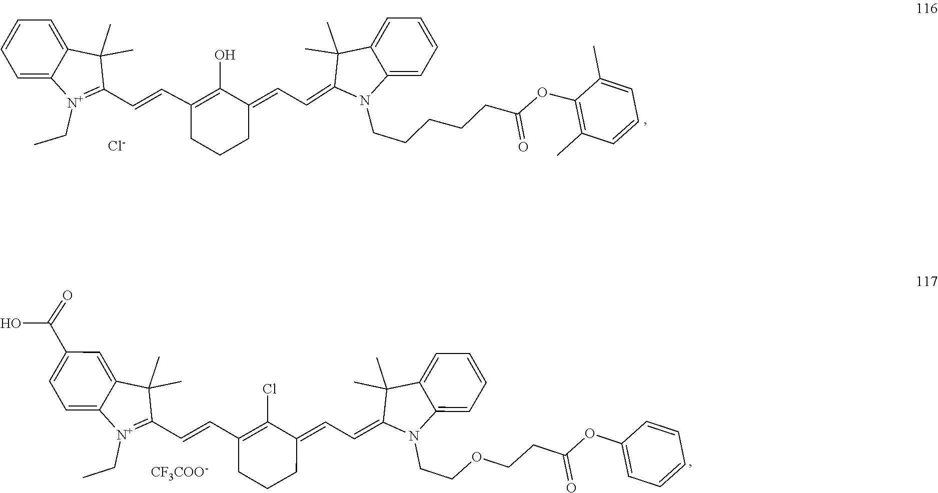

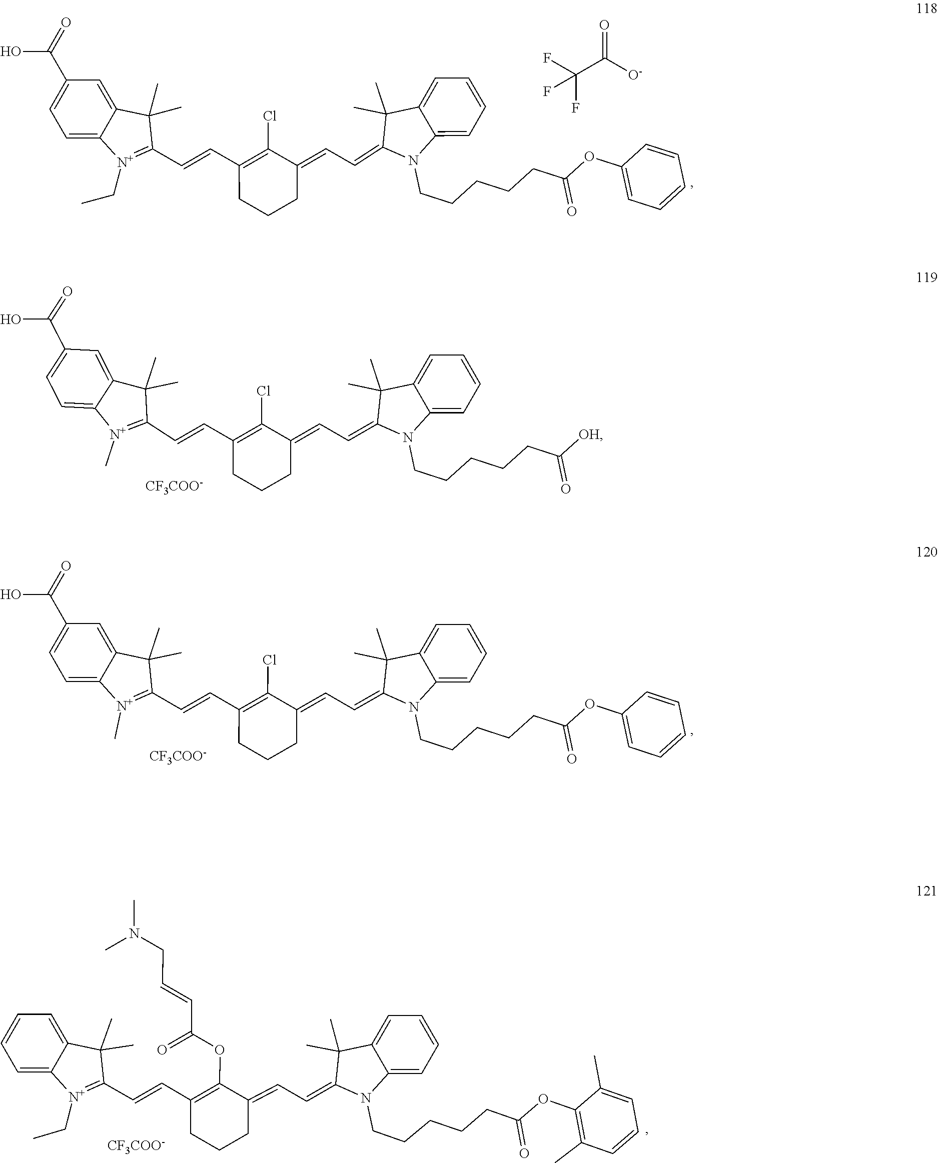

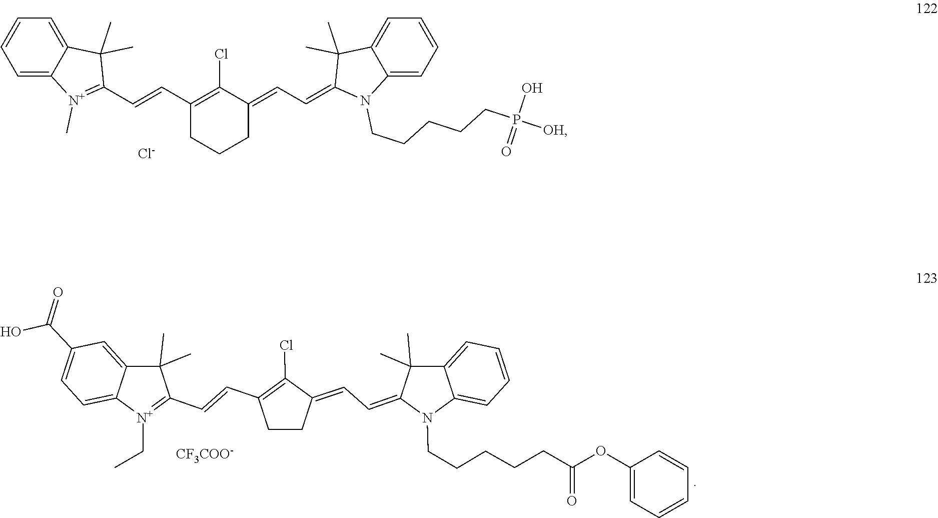

In another aspect of the present invention, provided is a compound with a structure of general formula (VI) or a pharmaceutically acceptable salt thereof,

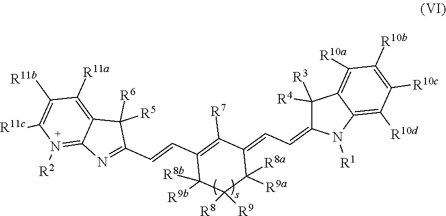

wherein, R¹ to R⁹, R^{8a}, R^{8b}, R^{9a}, R^{9b}, R^{10a}, R^{10b}, R^{10c}, R^{10d}, R^{11a}, R^{11b} and R^{11c} are as defined in general formula (I); s is 0 or 1;
preferably,
R¹ is L₁-L₂-Q;
L₁ is —(CH₂)ₚ—;
L₂ is a bond;
Q is —C(O)OR¹³;
R¹³ is selected from the group consisting of hydrogen and phenyl;
R² is C₁₋₆ alkyl;
R³ and R⁴ are each independently selected from the group consisting of hydrogen and C₁₋₆ alkyl, and preferably C₁₋₆ alkyl;
R⁵ and R⁶ are each independently selected from the group consisting of hydrogen and C₁₋₆ alkyl, and preferably C₁₋₆ alkyl;
R⁷ is a halogen;
R⁸ and R⁹ are each independently hydrogen;
R^{8a} and R^{9a} are each independently hydrogen;
R^{8b} and R^{9b} are each independently hydrogen;
R^{10a}, R^{10b}, R^{10c} and R^{10d} are each independently hydrogen;
R^{11a}, R^{11b} and R^{11c} are each independently hydrogen.

In a specific embodiment, the compound with the structure of general formula (VI) or a pharmaceutically acceptable salt thereof is selected from the group consisting of:

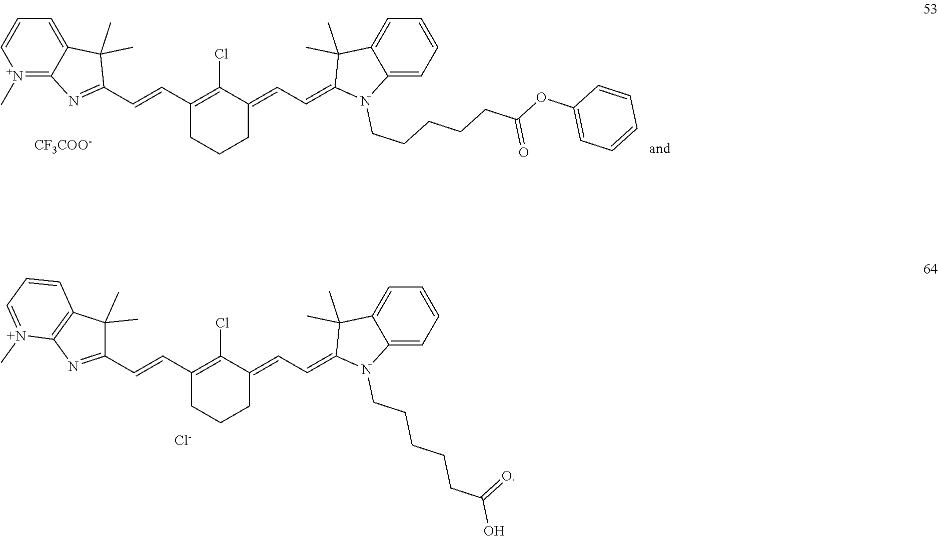

In another aspect of the present invention, provided is a compound with a structure of general formula (VII) or a pharmaceutically acceptable salt thereof,

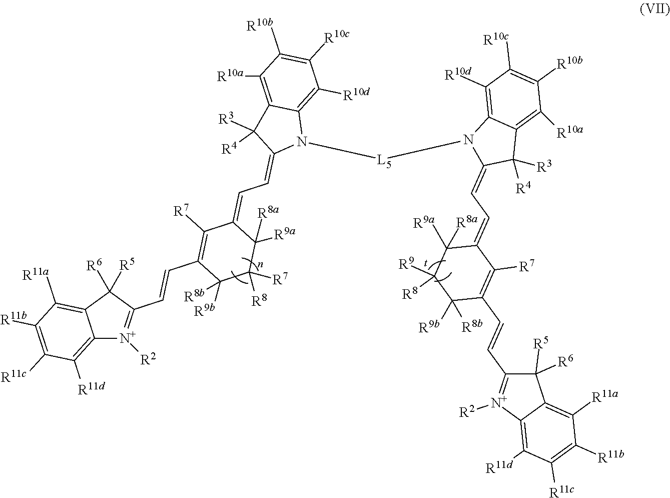

wherein, R² to R⁹, R^{8a}, R^{8b}, R^{9a}, R^{9b}, R^{10a}, R^{10b}, R^{10c}, R^{10d}, R^{11a}, R^{11b}, R^{11c} are as defined in general formula (I);
t is 0 or 1;
L₅ is —(CH₂)ₜ₁—C(O)NH—(CH₂)ₜ₂—NHC(O)—(CH₂)ₜ₃—;
t₁, t₂ and t₃ are each independently selected from the group consisting of integers from 1 to 10; and preferably selected from the group consisting of integers from 2 to 6;
preferably,
R² is C₁₋₆ alkyl;
R³ and R⁴ are each independently selected from the group consisting of hydrogen and C₁₋₆ alkyl, and preferably C₁₋₆ alkyl;
R⁵ and R⁶ are each independently selected from the group consisting of hydrogen and C₁₋₆ alkyl, and preferably C₁₋₆ alkyl;
R⁷ is a halogen;
R⁸ and R⁹ are each independently hydrogen;
R^{8a} and R^{9a} are each independently hydrogen;
R^{8b} and R^{9b} are each independently hydrogen;
R^{10a}, R^{10b}, R^{10c} and R^{10d} are each independently hydrogen;
R^{11a}, R^{11b} and R^{11c} are each independently hydrogen.

In a specific embodiment, the compound with the structure of general formula (VII) or a pharmaceutically acceptable salt thereof is selected from the group consisting of:

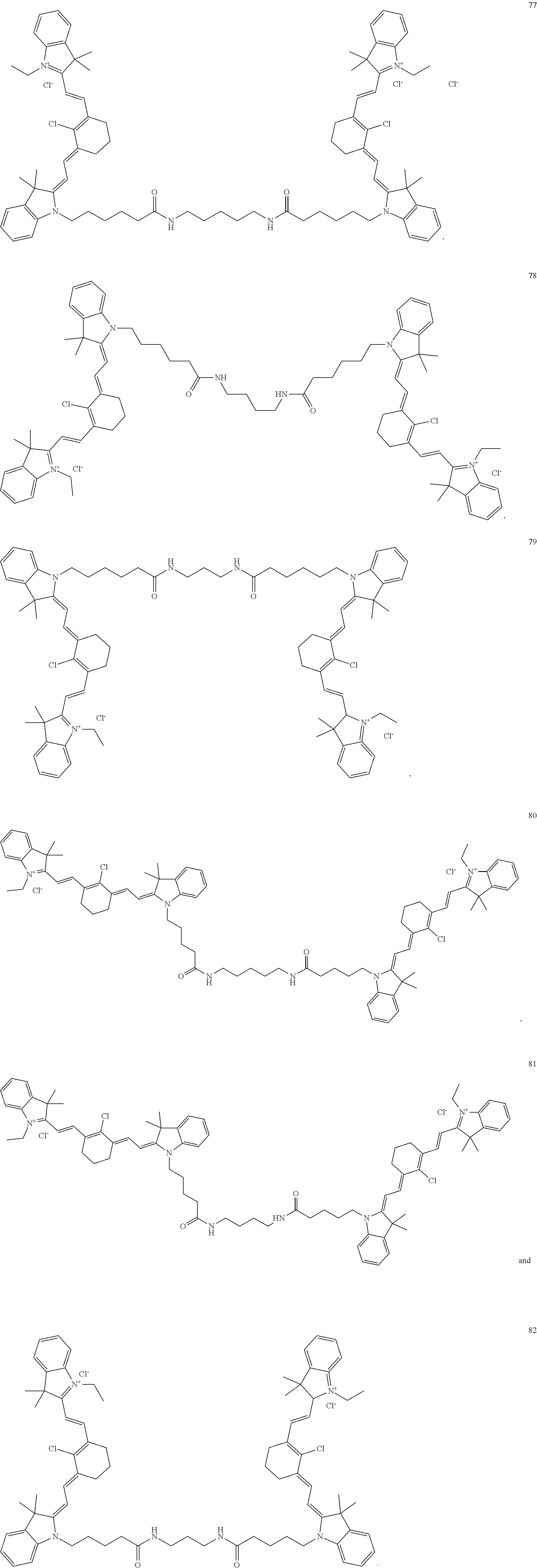

In another aspect of the present invention, provided is a compound with a structure of general formula (VIII) or a pharmaceutically acceptable salt thereof,

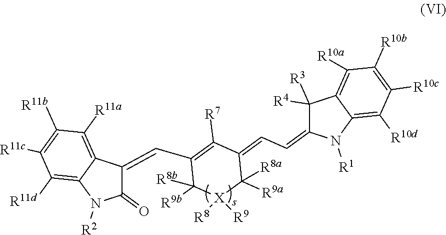

wherein, X, R¹ to R⁴, R⁷ to R⁹, R^{8a}, R^{8b}, R^{9a}, R^{9b}, R^{10a}, R^{10b}, R^{10c}, R^{10d}, R^{11a}, R^{11b}, R^{11c} and R^{11d} are as defined in general formula (I); s is 0 or 1;
preferably,
X is N⁺;
s is 1;
R¹ is L₁-L₂-Q;
L₁ is —(CH₂)ₚ—;
L₂ is a bond;
Q is —C(O)OR¹³;
R¹³ is selected from the group consisting of hydrogen and phenyl;
R² is C₁₋₆ alkyl;
R³ and R⁴ are each independently selected from the group consisting of hydrogen and C₁₋₆ alkyl, and preferably C₁₋₆ alkyl;
R⁷ is a halogen;
R⁸ and R⁹ are each independently C₁₋₆ alkyl;
R^{8a} and R^{9a} are each independently hydrogen;
R^{8b} and R^{9b} are each independently hydrogen;
R^{10a}, R^{10b}, R^{10c} and R^{10d} are each independently hydrogen;
R^{11a}, R^{11b} and R^{11c} are each independently hydrogen.

In a specific embodiment, the compound with the structure of general formula (VIII) or a pharmaceutically acceptable salt thereof is selected from:

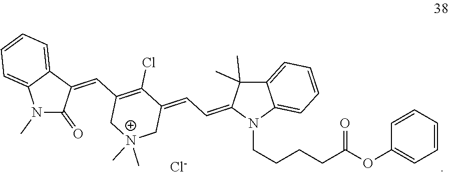

In another aspect of the present invention, provided is a contrast agent comprising the compound or a pharmaceutically acceptable salt thereof according to the present invention and an anionic surfactant.

In an embodiment, in the contrast agent, the anionic surfactant is selected from the group consisting of carboxylate salt, sulfonate salt, sulfate salt, phosphate salt and lipopeptide anionic surfactants, preferably is sulfonate salt or lipopeptide anionic surfactant, and more preferably is surfactin, sodium dodecyl sulfonate and/or sodium dodecyl benzene sulfonate.

In another embodiment, in the contrast agent, the molar ratio of the compound or a pharmaceutically acceptable salt thereof to the anionic surfactant ranges from 1:20 to 10:1, preferably from 1:10 to 10:1, and further preferably from 1:5 to 5:1.

In another embodiment, in the contrast agent, the compound or a pharmaceutically acceptable salt thereof has a final concentration ranging from 1 to 100 μM, and preferably from 5 to 50 μM or from 10 to 30 μM.

In another embodiment, a solution of the contrast agent has a particle size ranging from 10 to 200 nm, preferably from 30 to 150 nm, and more preferably from 50 to 100 nm.

In another embodiment, the contrast agent is in a form of solution or lyophilized powder.

The present invention also provides an assay kit comprising the compound or a pharmaceutically acceptable salt thereof according to the present invention and an anionic surfactant.

In an embodiment, in the kit, the anionic surfactant is selected from the group consisting of carboxylate salt, sulfonate salt, sulfate salt, phosphate salt and lipopeptide anionic surfactants, preferably is sulfonate salt or lipopeptide anionic surfactant, and more preferably is surfactin, sodium dodecyl sulfonate and/or sodium dodecyl benzene sulfonate.

In another embodiment, in the kit, the molar ratio of the compound or a pharmaceutically acceptable salt thereof to the anionic surfactant ranges from 1:20 to 10:1, preferably from 1:10 to 10:1, and further preferably from 1:5 to 5:1.

In another embodiment, in the kit, the compound or a pharmaceutically acceptable salt thereof in the kit has a final concentration ranging from 1 to 100 μM, and preferably from 5 to 50 μM or from 10 to 30 μM.

In another embodiment, in the kit, a solution of the contrast agent in the kit has a particle size ranging from 10 to 200 nm, preferably from 30 to 150 nm, and more preferably from 50 to 100 nm.

In another embodiment, in the kit, the contrast agent is in a form of solution or lyophilized powder.

The present invention also relates to a pharmaceutical composition comprising the compound or a pharmaceutically acceptable salt thereof according to the present invention, and one or more pharmaceutically acceptable carriers.

The present invention also relates to a use of the compound or a pharmaceutically acceptable salt thereof according to the present invention or a pharmaceutical composition comprising the same in the preparation of a contrast agent for tumor, wherein the tumor is preferably bladder cancer.

The present invention also relates to the compound or a pharmaceutically acceptable salt thereof according to the present invention or a pharmaceutical composition comprising the same for use as a contrast agent.

The present invention also relates to a method for tumor imaging comprising administering to a subject in need thereof the compound or a pharmaceutically acceptable salt thereof according to the present invention or a pharmaceutical composition comprising the same.

The molecules of the present invention exhibit a significant difference in uptake ability between normal cells and tumor cells in a cell model, such that tumor cells can improve their absorption exponentially, forming an obvious contrast to other cells.

The molecules of the present invention exhibit excellent tumor-specific targeting effect in an animal subcutaneous tumor model, enabling to form an obvious contrast to surrounding normal tissues, thereby facilitating tumor diagnosis.

### Explanation of Terms

Unless stated to the contrary, the terms used in the specification and claims have the meanings described below.

The carbon, hydrogen, oxygen, sulfur, nitrogen or halogen involved in the groups and compounds described in the present invention all include their isotopes, i.e., the carbon, hydrogen, oxygen, sulfur, nitrogen or halogen involved in the groups and compounds described in the present invention is optionally further replaced by one or more corresponding isotopes, in which the isotope of carbon includes ¹²C, ¹³C and ¹⁴C, the isotope of hydrogen includes protium (H), deuterium (D, also known as heavy hydrogen) and tritium (T, also known as superheavy hydrogen), the isotope of oxygen includes ¹⁶O, ¹⁷O and ¹⁸O, the isotope of sulfur includes ³²S, ³³S, ³⁴S and ³⁶S, the isotope of nitrogen includes ¹⁴N and ¹⁵N, the isotope of fluorine includes ¹⁹F, the isotope of chlorine includes ³⁵Cl and ³⁷Cl, and the isotope of bromine includes ⁷⁹Br and ⁸¹Br.

The term “alkyl” refers to a saturated aliphatic hydrocarbon group, which is a straight or branched chain group comprising 1 to 20 carbon atoms, preferably an alkyl having 1 to 12 carbon atoms, and more preferably an alkyl having 1 to 6 carbon atoms, an alkyl having 1 to 4 carbon atoms or an alkyl having 1 to 3 carbon atoms. Non-limiting examples include methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and various branched isomers thereof. More preferably, the alkyl is a lower alkyl having 1 to 6 carbon atoms, and non-limiting examples include methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl and the like. The alkyl may be substituted or unsubstituted. When substituted, the substituent(s) may be substituted at any available connection point. The substituent(s) may be one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, oxo, carboxy and carboxylic ester group.

The term “alkylene” refers to a divalent alkyl group with the alkyl as defined above, which has 1 to 20 (for example 1, 2, 3, 4, 5, 6, 7, 8, 9, 10, 11, 12, 13, 14, 15, 16, 17, 18, 19 or 20) carbon atoms (i.e., C₁₋₂₀ alkylene). The alkylene is preferably an alkylene having 1 to 12 carbon atoms (i.e., C₁₋₁₂ alkylene), more preferably an alkylene having 1 to 6 carbon atoms (i.e., C₁₋₆ alkylene), and further preferably an alkylene having 1 to 4 carbon atoms (i.e., C₁₋₄ alkylene). Non-limiting examples of the alkylene include, but are not limited to, methylene (—CH₂—), 1,1-ethylidene (—CH(CH₃)—), 1,2-ethylidene (—CH₂CH₂)—, 1,1-propylidene (—CH(CH₂CH₃)—), 1,2-propylidene (—CH₂CH(CH₃)—), 1,3-propylidene (—CH₂CH₂CH₂—), 1,4-butylidene (—CH₂CH₂CH₂CH₂—) and the like. The alkylene may be substituted or unsubstituted, and when substituted, it may be substituted at any available connection point. The substituent(s) may be one or more selected from the group consisting of alkyl, alkenyl, alkynyl, alkoxy, haloalkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio and oxo.

The term “alkenyl” refers to a monovalent hydrocarbon group consisting of at least two carbon atoms and at least one carbon-carbon double bond, and preferably an alkenyl having 2 to 4 carbon atoms, such as ethenyl, 1-propenyl, 2-propenyl, 1-, 2- or 3-butenyl and the like. The alkenyl may be substituted or unsubstituted. When it is substituted, the substituent(s) may be one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio and heterocyclylthio.

The term “alkynyl” refers to a monovalent hydrocarbon group consisting of at least two carbon atoms and at least one carbon-carbon triple bond, and preferably an alkynyl having 2 to 4 carbon atoms or preferably an alkynyl having 3 to 4 carbon atoms, such as ethynyl, propynyl, butynyl and the like. The alkynyl may be substituted or unsubstituted. When it is substituted, the substituent(s) may be one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio and heterocyclylthio.

The term “cycloalkyl” refers to a saturated or partially unsaturated monocyclic or polycyclic hydrocarbon group, in which the cycloalkyl ring has 3 to 20 carbon atoms, preferably 3 to 12 carbon atoms, and more preferably 3 to 6 carbon atoms. Non-limiting examples of the monocyclic cycloalkyl include cyclopropyl, cyclobutyl, cyclopentyl, cyclopentenyl, cyclohexyl, cyclohexenyl, cyclohexadienyl, cycloheptyl, cycloheptatrienyl, cyclooctyl and the like. The polycyclic cycloalkyl includes spiro cycloalkyl, fused cycloalkyl and bridged cycloalkyl.

The term “spiro cycloalkyl” refers to a 5 to 20 membered polycyclic group with individual rings sharing one carbon atom (called a spiro atom), which may contain one or more double bonds, but none of the rings of which has a completely conjugated π-electron system. The spiro cycloalkyl is preferably a 6 to 14 membered spiro cycloalkyl, and more preferably a 7 to 10 membered spiro cycloalkyl. According to the number of the spiro atoms shared between the rings, the spiro cycloalkyl is divided into a mono-spiro cycloalkyl, di-spiro cycloalkyl or poly-spiro cycloalkyl, and is preferably a mono-spiro cycloalkyl and di-spiro cycloalkyl, and more preferably a 4-membered/4-membered, 4-membered/5-membered, 4-membered/6-membered, 5-membered/5-membered or 5-membered/6-membered mono-spiro cycloalkyl. Non-limiting examples of the spiro cycloalkyl include:

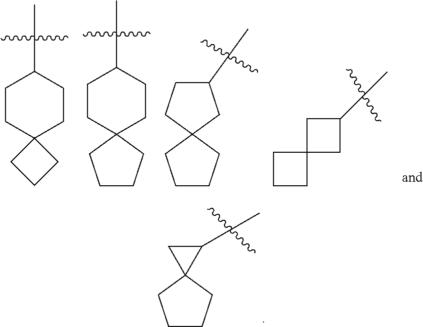

The term “fused cycloalkyl” refers to a 5 to 20 membered full-carbon polycyclic group, in which each ring shares a pair of adjacent carbon atoms with another ring in the system, and one or more rings may contain one or more double bonds, but none of the rings has a completely conjugated π-electron system. The fused cycloalkyl is preferably a 6 to 14 membered fused cycloalkyl, and more preferably a 7 to 10 membered fused cycloalkyl. According to the number of membered rings, the fused cycloalkyl may be divided into a bicyclic, tricyclic, tetracyclic or polycyclic fused cycloalkyl, and is preferably a bicyclic or tricyclic fused cycloalkyl, and more preferably a 5-membered/5-membered or 5-membered/6-membered bicyclic cycloalkyl. Non-limiting examples of the fused cycloalkyl include:

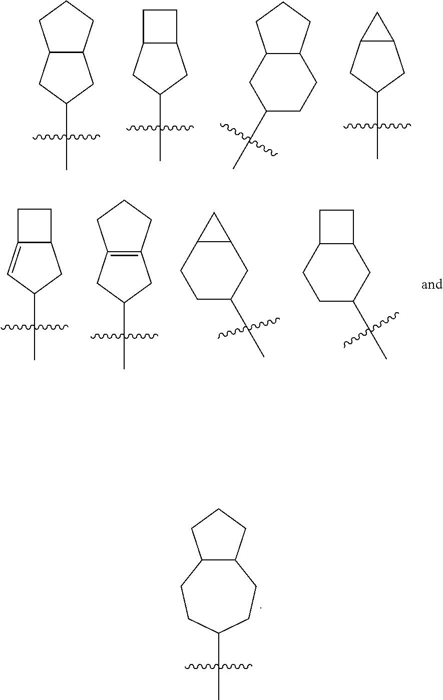

The term “bridged cycloalkyl” refers to a 5 to 20 membered full-carbon polycyclic group with any two of rings thereof sharing two carbon atoms that are not directly connected to each other, which may have one or more double bonds, but none of the rings of which has a completely conjugated π-electron system. The bridged cycloalkyl is preferably a 6 to 14 membered bridged cycloalkyl, and more preferably a 7 to 10 membered bridged cycloalkyl. According to the number of membered rings, the bridged cycloalkyl may be divided into a bicyclic, tricyclic, tetracyclic or polycyclic bridged cycloalkyl, and is preferably a bicyclic, tricyclic or tetracyclic bridged cycloalkyl, and more preferably a bicyclic or tricyclic bridged cycloalkyl. Non-limiting examples of the bridged cycloalkyl include:

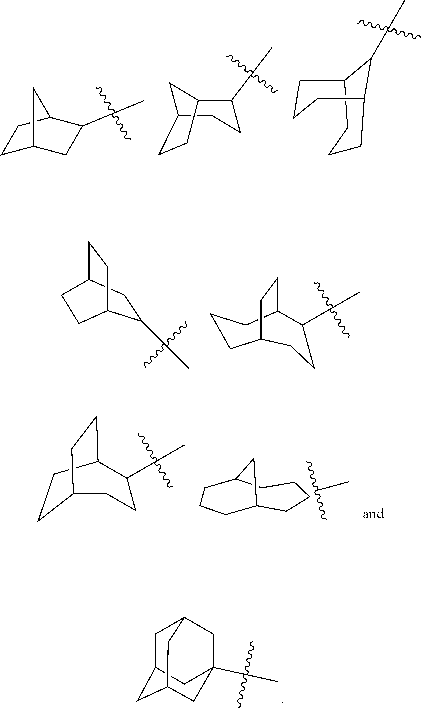

The cycloalkyl ring may be fused to an aryl, heteroaryl or heterocyclyl ring, in which the ring linking to the parent structure is cycloalkyl. Non-limiting examples include indanyl, tetrahydronaphthyl, benzocycloheptyl and the like. The cycloalkyl may be optionally substituted or unsubstituted. When it is substituted, the substituent(s) may be one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, oxo, carboxy or carboxylic ester group.

The term “heterocyclyl” refers to a saturated or partially unsaturated monocyclic or polycyclic hydrocarbon group comprising 3 to 20 ring atoms, in which one or more ring atoms are heteroatoms selected from the group consisting of N, O and S(O)ₘ (wherein m is an integer from 0 to 2), but excluding —O—O—, —O—S— or —S—S— in the ring, the remaining ring atoms being carbon atoms. Preferably, the heterocyclyl comprises 4 to 12 ring atoms, 1 to 4 atoms of which are heteroatoms; and more preferably 7 to 12 ring atoms, 1 to 4 atoms of which are heteroatoms. Non-limiting examples of the monocyclic heterocyclyl include pyrrolidinyl, imidazolyl, tetrahydrofuranyl, tetrahydrothienyl, dihydroimidazolyl, dihydrofuranyl, dihydropyrazolyl, dihydropyrrolyl, piperidinyl, piperazinyl, morpholinyl, thiomorpholinyl, homopiperazinyl, pyranyl and the like, and preferably, 1,2,5-oxadiazolyl, pyranyl or morpholinyl. The polycyclic heterocyclyl includes spiro heterocyclyl, fused heterocyclyl and bridged heterocyclyl.

The term “spiro heterocyclyl” refers to a 5 to 20 membered polycyclic heterocyclyl group with individual rings sharing one atom (called a spiro atom), in which one or more ring atoms are heteroatoms selected from the group consisting of N, O and S(O)ₘ (wherein m is an integer from 0 to 2), and the remaining ring atoms are carbon atoms. The spiro heterocyclyl may contain one or more double bonds, but none of the rings thereof has a completely conjugated π-electron system. The spiro heterocyclyl is preferably a 6 to 14 membered spiro heterocyclyl, and more preferably a 7 to 12 membered spiro heterocyclyl. According to the number of the spiro atoms shared between the rings, the spiro heterocyclyl is divided into mono-spiro heterocyclyl, di-spiro heterocyclyl or poly-spiro heterocyclyl, and is preferably a mono-spiro heterocyclyl and di-spiro heterocyclyl, and more preferably a 4-membered/4-membered, 4-membered/5-membered, 4-membered/6-membered, 5-membered/5-membered or 5-membered/6-membered mono-spiro heterocyclyl. Non-limiting examples of the spiro heterocyclyl include:

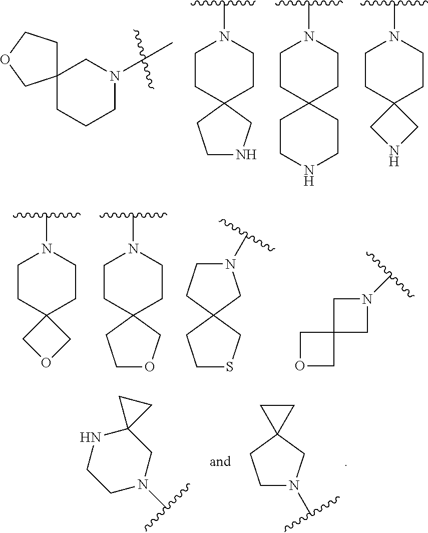

The term “fused heterocyclyl” refers to a 5 to 20 membered polycyclic heterocyclyl group, in which each ring shares a pair of adjacent atoms with another ring in the system, one or more rings may contain one or more double bonds, but none of the rings has a completely conjugated π-electron system, and one or more ring atoms are heteroatoms selected from the group consisting of N, O and S(O)ₘ (wherein m is an integer from 0 to 2), with the remaining ring atoms being carbon atoms. The fused heterocyclyl is preferably a 6 to 14 membered fused heterocyclyl, and more preferably a 7 to 12 membered fused heterocyclyl. According to the number of membered rings, the fused heterocyclyl may be divided into a bicyclic, tricyclic, tetracyclic or polycyclic fused heterocyclyl, and is preferably a bicyclic or tricyclic fused heterocyclyl, and more preferably a 5-membered/5-membered or 5-membered/6-membered bicyclic fused heterocyclyl. Non-limiting examples of the fused heterocyclyl include:

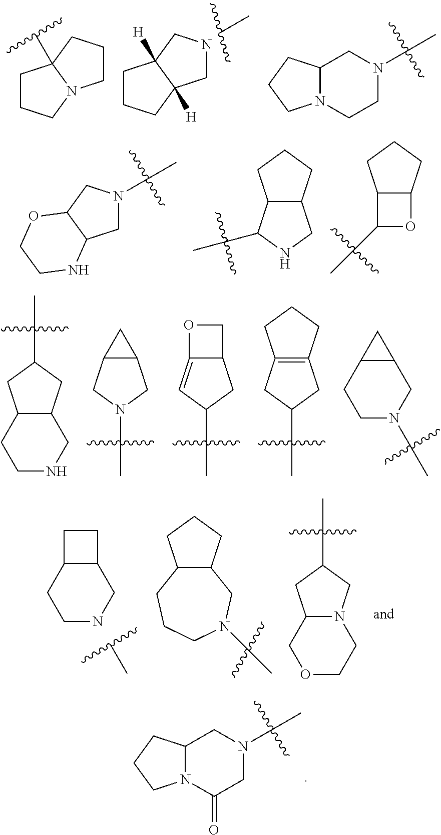

The term “bridged heterocyclyl” refers to a 5 to 14 membered polycyclic heterocyclyl group with any two of rings thereof sharing two atoms that are not directly connected to each other, which may have one or more double bonds, but none of the rings of which has a completely conjugated π-electron system, and in which one or more ring atoms are heteroatoms selected from the group consisting of N, O and S(O)ₘ (wherein m is an integer from 0 to 2), with the remaining ring atoms being carbon atoms. The bridged heterocyclyl is preferably a 6 to 14 membered bridged heterocyclyl, and more preferably a 7 to 12 membered bridged heterocyclyl. According to the number of membered rings, the bridged heterocyclyl may be divided into a bicyclic, tricyclic, tetracyclic or polycyclic bridged heterocyclyl, and is preferably a bicyclic, tricyclic or tetracyclic bridged heterocyclyl, and more preferably a bicyclic or tricyclic bridged heterocyclyl. Non-limiting examples of the bridged heterocyclyl include:

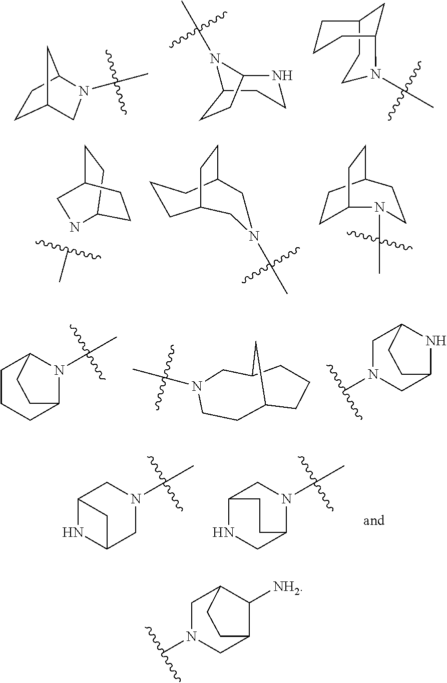

The heterocyclyl ring may be fused to an aryl, heteroaryl or cycloalkyl ring, in which the ring linking to the parent structure is heterocyclyl. Non-limiting examples thereof include:

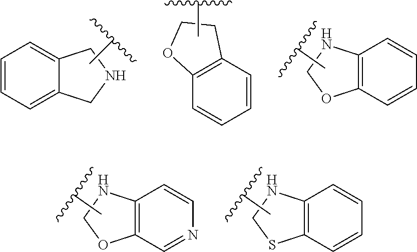

and the like.

The heterocyclyl may be optionally substituted or unsubstituted. When it is substituted, the substituent(s) may be one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, oxo, carboxy or carboxylic ester group.

The term “aryl” refers to a 6 to 14 membered full-carbon monocyclic or fused polycyclic (i.e., rings sharing a pair of adjacent carbon atoms) group having a conjugated π-electron system, and preferably a 6 to 10 membered aryl, for example, phenyl and naphthyl. The aryl is more preferably phenyl. The aryl ring may be fused to a heteroaryl, heterocyclyl or cycloalkyl ring, in which the ring linking to the parent structure is an aryl ring. Non-limiting examples thereof include:

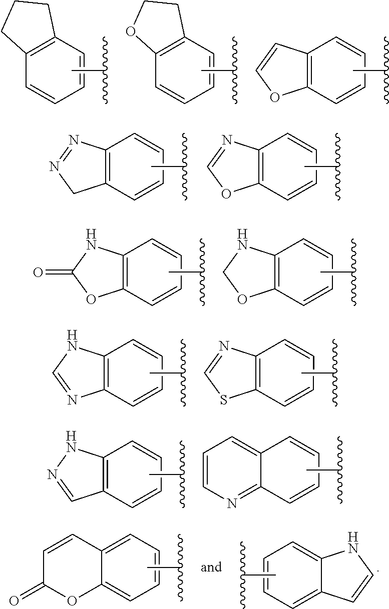

The aryl may be substituted or unsubstituted. When it is substituted, the substituent(s) may be one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, carboxy or carboxylic ester group.

The term “heteroaryl” refers to a heteroaromatic system having 5 to 14 ring atoms, which comprises 1 to 4 heteroatoms selected from the group consisting of O, S and N. The heteroaryl is preferably a 5 to 10 membered heteroaryl having 1 to 3 heteroatoms, and more preferably a 5 or 6 membered heteroaryl having 1 to 2 heteroatoms, for example imidazolyl, furyl, thienyl, thiazolyl, pyrazolyl, oxazolyl, pyrrolyl, tetrazolyl, pyridyl, pyrimidinyl, thiadiazolyl, pyrazinyl and the like, preferably imidazolyl, thiazolyl, pyrazolyl, pyrimidinyl or thiazolyl, and more preferably pyrazolyl or thiazolyl. The heteroaryl ring may be fused to a aryl, heterocyclyl or cycloalkyl ring, in which the ring linking to the parent structure is a heteroaryl ring. Non-limiting examples thereof include:

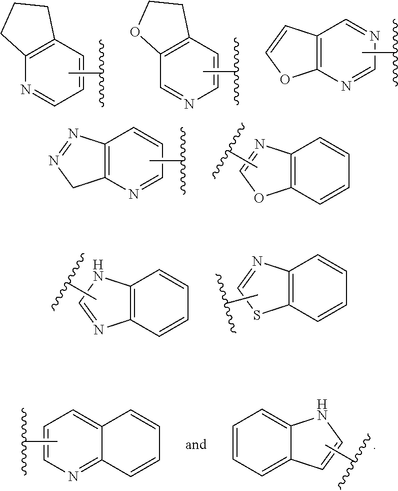

The heteroaryl may be optionally substituted or unsubstituted. When it is substituted, the substituent(s) may be one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, carboxy or carboxylic ester group.

The term “alkoxy” refers to an —O-(alkyl) group, in which the alkyl is as described above. Non-limiting examples of the alkoxy include methoxy, ethoxy, propoxy, butoxy, cyclopropyloxy, cyclobutyloxy, cyclopentyloxy and cyclohexyloxy. The alkoxy may be optionally substituted or unsubstituted. When it is substituted, the substituent(s) may be one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, carboxy or carboxylic ester group.

The above alkenyl, alkynyl, cycloalkyl, heterocyclyl, aryl and heteroaryl comprise residues resulting from removing one hydrogen atom from the parent rings, or residues resulting from removing two hydrogen atoms from the same ring atom or two different ring atoms of the parent rings, i.e., “alkenylene”, “alkynylene”, “cycloalkylene”, “heterocyclylene”, “arylene” and “heteroarylene”.

The term “cycloalkoxy” refers to an —O-(cycloalkyl) group, in which the cycloalkyl is as defined above.

The term “heterocycloalkoxy” refers to an —O-(heterocyclyl) group, in which the heterocyclyl is as defined above.

The term “haloalkyl” refers to an alkyl group substituted by one or more halogens, in which the alkyl is as defined above.

The term “haloalkoxy” refers to an alkoxy group substituted by one or more halogens, in which the alkoxy is as defined above.

The term “hydroxyalkyl” refers to an alkyl group substituted by a hydroxy group, in which the alkyl is as defined above.

The term “hydroxy” refers to an —OH group.

The term “halogen” refers to fluorine, chlorine, bromine or iodine.

The term “amino” refers to a —NH₂ group.

The term “cyano” refers to a —CN group.

The term “nitro” refers to a —NO₂ group.

The term “oxo” refers to an ═O group.

The term “thioxo” refers to a ═S group.

The term “carboxy” refers to a —C(O)OH group.

The term “thiol” refers to a —SH group.

The term “ester” refers to a —C(O)O(alkyl) or a —C(O)O(cycloalkyl) group, in which the alkyl and cycloalkyl are as defined above.

The term “acyl” refers to a compound with a —C(O)R group, wherein R is alkyl, cycloalkyl, heterocyclyl, aryl and heteroaryl.

The term “sulfonic group” refers to a —SO₃H group.

The term “optional” or “optionally” means that the event or circumstance described subsequently can, but need not, occur, and such a description includes the situation in which the event or circumstance does or does not occur. For example, the expression “heterocyclyl optionally substituted by an alkyl group” means that an alkyl group can be, but need not be, present, and such a description includes the situation of the heterocyclyl being substituted by an alkyl group and the situation of the heterocyclyl being not substituted by an alkyl group.

The term “substituted” means that one or more hydrogen atoms in a group, preferably up to 5, and more preferably 1 to 3 hydrogen atoms in a group, are independently substituted by a corresponding number of substituents. It goes without saying that the substituents only exist in their possible chemical position. Those skilled in the art are able to determine whether the substitution is possible or impossible by experiments or theory without excessive efforts. For example, the combination of amino or hydroxy having free hydrogen and carbon atoms having unsaturated bond(s) (such as olefinic) may be unstable.

The term “pharmaceutical composition” represents a mixture containing one or more compounds described herein or physiologically/pharmaceutically acceptable salts or prodrugs thereof and other components such as physiologically/pharmaceutically acceptable carriers and excipients. The purpose of the pharmaceutical composition is to facilitate administration of a drug to an organism, which is conducive to absorption of an active ingredient(s) and thus exhibit biological activity.

The term “pharmaceutically acceptable salt” refers to a salt of the compound of the present invention, which is safe and effective in mammals and has desired biological activity.

The term “carrier” refers to a carrier or diluent which does not cause significant irritation to an organism and does not eliminate the biological activity and property of the compound administrated.

### DESCRIPTION OF THE DRAWINGS

FIG. 1 shows the fluorescence microscopic imaging of the compounds of the invention in human bladder cancer cell line RT112.

FIG. 2 shows the fluorescence microscopic imaging of the compounds of the invention in human bladder cancer cell line UM-UC-3.

FIG. 3 shows the fluorescence microscopic imaging of the compounds of the invention in human bladder cancer cell line RT112, with or without addition of BSP.

FIG. 4 shows the fluorescence microscopic imaging of the compounds of the invention in human bladder cancer cell line UM-UC-3, with or without addition of BSP.

FIG. 5 shows the fluorescence imaging of the compounds of the invention in the bladder cancer tumor model mice.

### DETAILED DESCRIPTION OF THE INVENTION

The compounds of the present invention and the preparation thereof are further understood through examples, which illustrate some methods of preparing or using the compounds. However, it is understood that the present invention is not limited to these examples. All currently known or further developed variations of the present invention are considered as falling within the scope of the present invention described and claimed herein.

The compounds of the present invention are prepared by using convenient starting materials and general preparation procedures. The present invention provides typical or preferential reaction conditions, such as reaction temperature, duration, solvent, pressure and molar ratio of reactants. However, unless specially specified, other reaction conditions can also be adopted. Optimal conditions may vary with the use of specific reactants or solvents, but under normal circumstances, the steps and conditions optimized for reaction can be determined.

In addition, some protecting groups may be used in the present invention to protect certain functional groups from unnecessary reactions. The protecting groups suitable for various functional groups and their protection or deprotection conditions are already well known to those skilled in the art. For example, “Protective Groups in Organic Synthesis” by T. W. Greene and G. M. Wuts (3rd edition, Wiley, New York, 1999) and the citations in the book describe the protection or deprotection of a large number of protecting groups in detail.

The isolation and purification of compounds and intermediates are carried out by appropriate methods and steps according to specific needs, such as filtration, extraction, distillation, crystallization, column chromatography, preparative thin-layer chromatography, preparative high performance liquid chromatography or a combination of the above methods. The specific methods used may be referred to the examples described in the present invention. Of course, other similar isolation and purification means can also be used. They can be characterized using conventional methods (including physical constants and spectral data).

The structures of the compounds are identified by nuclear magnetic resonance (NMR) and/or mass spectrometry (MS). NMR shift is given in units of 10⁻⁶ (ppm). NMR is determined on a Brukerdps 300 nuclear magnetic spectrometer. The solvents for determination are deuterated dimethyl sulfoxide (DMSO-d₆), deuterated chloroform (CDCl₃), and deuterated methanol (CD₃OD), and the internal standard is tetramethylsilane (TMS).

MS is determined on a LC (Agilent 1260 Infinity)/MS (G6125B) mass spectrometer (manufacturer: Agilent).

Preparative liquid chromatography is determined using a lc6000 high performance liquid chromatograph (manufacturer: Chuangxintongheng Science & Technology CO., LTD.). The chromatographic column is Daisogel C18 10 μm 100 A (30 mm×250 mm), and the mobile phase is acetonitrile/water.

GF254 silica gel plates from Qingdao Haiyang Chemical are used for thin-layer chromatography (TLC). For the silica gel plates used, the specification is 0.20 mm to 0.25 mm for the thin-layer chromatography for monitoring of reactions, and 0.5 mm for the thin-layer chromatography for separation and purification.

Silica gels of 100 to 200 mesh, 200 to 300 mesh and 300 to 400 mesh from Qingdao Haiyang Chemical are used as a carrier for silica gel column chromatography.

The known starting materials of the present invention can be prepared by the known methods in the art, or can be purchased from Wanghua Mall, Beijing Ouhe Technology, Sigma, J&K Scientific, Yishiming, Shanghai Shuya Chemical, Shanghai Innochem Science & Technology, Energy Chemical, Shanghai Bide Pharmatech, Nanjing PharmaBlock and the like.

Unless specifically stated in the examples, all reactions can be carried out under nitrogen atmosphere.

Argon or nitrogen atmosphere means that a reaction flask is connected to an argon or nitrogen balloon with a volume of about 1 L.

The reaction solvent, organic solvent or inert solvent is each expressed as the solvent used that does not participate in the reaction under the described reaction conditions, which includes for example, benzene, toluene, acetonitrile, tetrahydrofuran (THF), dimethylformamide (DMF), chloroform, dichloromethane, ether, methanol, N-methylpyrrolidone (NMP), pyridine and the like. Unless specifically stated in the examples, a solution refers to an aqueous solution.

The chemical reactions described in the present invention are generally carried out under normal pressure. The reaction time and conditions are, for example, between −78° C. and 200° C. at one atmosphere, and completed within about 1 to 24 hours. If the reaction is carried out overnight, the reaction time is generally 16 hours. Unless specifically stated in the examples, the reaction temperature is room temperature, which is 20° C. to 30° C.

Reaction processes in the examples are monitored by thin-layer chromatography (TLC). The developing systems used for the reactions are: A: dichloromethane-methanol system, B: petroleum ether-ethyl acetate system, and C: acetone, and the volume ratios of the solvents are adjusted depending on the polarity of the compounds.

The eluent systems of the column chromatography and the developing systems of the thin-layer chromatography used for purifying the compounds include: A: dichloromethane-methanol system, and B: petroleum ether-ethyl acetate system, and the volume ratios of the solvents are adjusted depending on the polarity of the compounds. A small amount of alkaline or acidic reagents such as triethylamine and trifluoroacetic acid can also be added for adjustment.

Unless otherwise defined, all professional and scientific terms used herein have the same meaning as those familiar to those skilled in the art. In addition, any methods and materials similar or equivalent to the content described herein can be applied to the methods of the present invention.

### Preparation Example 1: Preparation of 2,3,3-trimethylindole-5-carboxylic Acid (A8A)

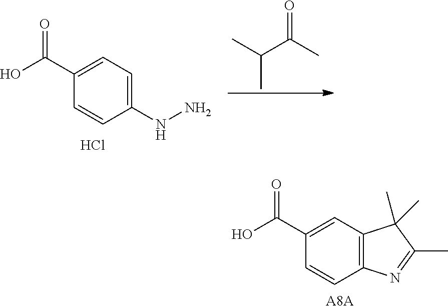

3-Methylbutan-2-one (25.1 g, 292 mmol, 31.2 mL, 1.10 eq) was added to a solution of 4-hydrazinobenzoic acid hydrochloride (50.0 g, 265 mmol, 1.00 eq) in AcOH (250 mL). The resulting mixture was kept at 120° C. and stirred for 12 hours. The reaction solution was concentrated under reduced pressure, and the residues were diluted with DCM (200 mL) and adjusted to pH=7 with saturated NaHCO₃ solution. The organic phase was dried over anhydrous sodium sulfate, filtered, and concentrated under reduced pressure. The residues were purified by silica gel column chromatography (SiO₂, petroleum ether/ethyl acetate=100/1 to 1/1) to obtain Compound A8A (25.0 g, 123 mmol, yield 46.4%) as a yellow solid.

¹H NMR (400 MHz, DMSO) δ 12.8 (s, 1H), 7.99 (s, 1H), 7.90-7.93 (m, 1H), 7.50 (d, J=7.6 Hz, 1H), 2.25 (s, 3H), 1.27 (s, 6H).

LCMS: m/z=204.0 (M+H)⁺.

The following compounds were prepared according to the above preparation method using the corresponding substituted hydrazine:

| No. | Structure | Hydrazine | NMR or MS data, and appearance |
|---|---|---|---|
| A7A | 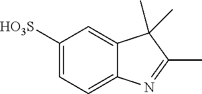 [SMILES: CC1=Nc2ccc(S(=O)(=O)O)cc2C1(C)C] | 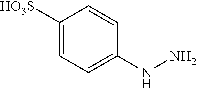 [SMILES: NNc1ccc(S(=O)(=O)O)cc1] | ¹H NMR (400 MHz, DMSO) δ 7.74 (d, J = 1.2 Hz, 1H), 7.63 (dd, J1 = 8.0 Hz, J2 = 1.6 Hz, 1H), 7.43 (d, J = 8.0 Hz, 1H), 2.36 (s, 3H), 1.33 (s, 6H). Pinkish purple solid. |
| A10A | 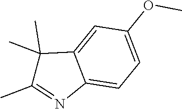 | 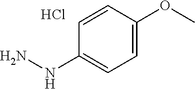 | ¹H NMR (400 MHz, CDCl₃) δ 7.43 (d, J = 9.2 Hz, 1H), 6.80-6.84 (m, 2H), 3.82 (s, 3H), 2.24 (s, 3H), 1.28 (s, 6H). LCMS: m/z = 190.2 (M + H)⁺, as a yellow oil. |
| A13B | 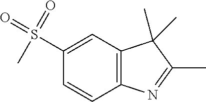 [SMILES: CC1=Nc2ccc(S(C)(=O)=O)cc2C1(C)C] | 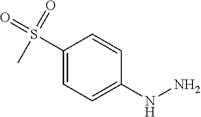 | LCMS: m/z = 238.2 (M + H)⁺. |

### Preparation Example 2: Preparation of tert-butyl 1-ethyl-2,3,3 trimethyl-indol-1-ium-5-carboxylate Iodide (A8)

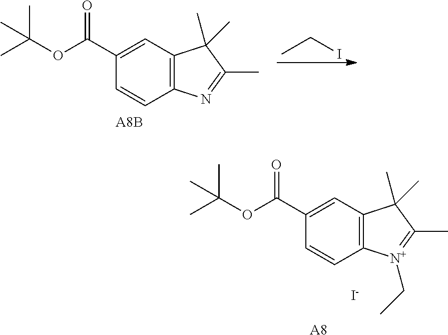

A mixture of Compound A8B (see Preparation Example 3 for the preparation method) (9.00 g, 34.7 mmol, 1.00 eq), iodoethane (10.8 g, 69.4 mmol, 5.55 mL, 2.00 eq) and MeCN (90.0 mL) was stirred at 100° C. for 32 hours. The reaction mixture was concentrated under reduced pressure, and the residues were pulped with MTBE (30.0 mL) for 30 minutes at 25° C. Then, the resulting mixture was purified by reversed-phase chromatography (Phenomenex luna C18 250×50 mm×15 μm; mobile phase: [water (FA)-ACN]; B %: 50%-80%, 10 min) to obtain Compound A8 (10.0 g) as a red solid.

LCMS: m/z=288.3 (M+H)⁺.

The following compounds were prepared according to the above preparation method using the corresponding raw material:

| No. | Structure | Raw material | NMR or MS data, and appearance |
|---|---|---|---|
| A12 | 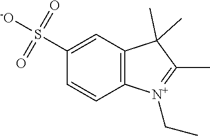 | 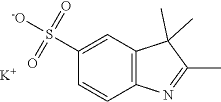 | LCMS: m/z = 268.2 C₁₃H₁₇NO₃S, as a yellow solid |
| A11A | 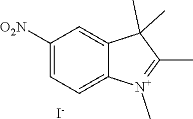 [SMILES: CC1=[N+](C)c2ccc([N+](=O)[O-])cc2C1(C)C ; [I-]] | 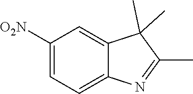 | ¹H NMR: (400 MHz, MeOD) δ 8.70 (s, 1H), 8.55 (d, J = 8. Hz, 1H), 8.11 (s, 1H), 4.14 (s, 3H), 1.70 (s, 9H). LCMS: m/z = 219.2 C₁₂H₁₅N₂O₂⁺. Yellow solid. |
| A10 | 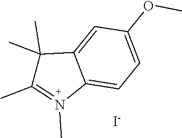 | 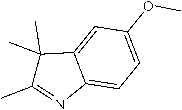 | LCMS: m/z = 204.2 C₁₃H₁₈NO⁺. Gray solid. |
| A13 | 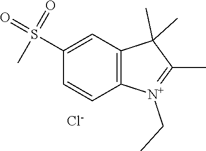 [SMILES: CC[N+]1=C(C)C(C)(C)c2cc(S(C)(=O)=O)ccc21 ; [Cl-]] | 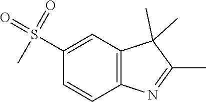 | ¹H NMR: (400 MHz, CD₃CN) δ 8.29 (s, 1H), 8.19 (dd, J1 = 1.6 Hz, J2 = 6.8 Hz, 1H), 8.04 (d, J = 8.8 Hz, 1H), 4.52 (dd, J1 = 7.6 Hz, J2 = 7.2 Hz, 1H), 3.17 (s, 3H), 2.84 (s, 3H), 1.62 (s, 6H), 1.52 (t, J = 7.6 Hz). LCMS: m/z = 266.1 C₁₄H₂₀NO₂S⁺. Yellow solid. |
| A9 | 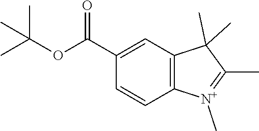 [SMILES: CC1=[N+](C)c2ccc(C(=O)OC(C)(C)C)cc2C1(C)C] | 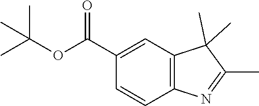 | LCMS: m/z = 274.1 C₁₇H₂₄NO₂⁺. Red solid. |

### Preparation Example 3: Preparation of tert-butyl 2,3,3-trimethylindole-5-carboxylate (A8B)

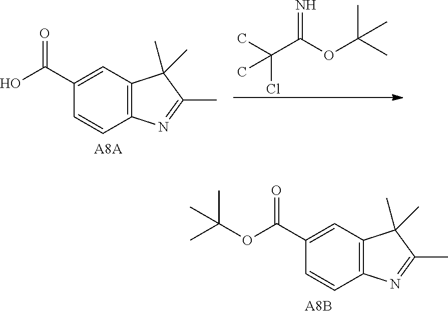

BF₃·Et₂O (4.75 g, 15.8 mmol, 4.12 mL, 47.0% purity, 0.400 eq) was added to a solution of 2,3,3-trimethylindole-5-carboxylic acid (8.00 g, 39.4 mmol, 1.00 eq) and tert-butyl trichloroacetimidate (17.2 g, 78.7 mmol, 14.1 mL, 2.00 eq) in DCM (80.0 mL) and THF (40.0 mL). The mixture was stirred for 12 hours at 25° C. The resulting mixture was kept around 0° C., and NaHCO₃ solution (50.0 mL) was added dropwise thereto. The resulting mixture was diluted with H₂O (100 mL), extracted with EA (200 mL×2), washed with brine (50.0 mL×2), dried over anhydrous Na₂SO₄, filtered and concentrated. The residues were pulped with PE (50.0 mL) for 30 minutes at 25° C. to obtain Compound A8B (9.00 g, crude) as a yellow oil.

LCMS: m/z=260.4 (M+H)⁺.

### Preparation Example 4: Preparation of 5-amino-1,2,3,3-tetramethyl-3H-indol-1-ium Iodide (A11B)

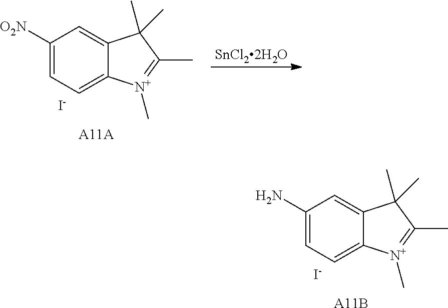

SnCl₂·2H₂O (57.0 g, 253 mmol, 7.00 eq) was added to a solution of Compound A11A (12.5 g, 36.1 mmol, 1.00 eq) in HCl (60.0 mL). The mixture was stirred for 16 hours at 20° C. The resulting mixture was diluted with 300 mL of water, adjusted to pH=10 with 5 M NaOH, and filtered. The parent solution was extracted with DCM (600 mL). The organic phase was washed with 300 mL of brine, dried over anhydrous Na₂SO₄ and filtered. The aqueous phase was lyophilized before addition of DCM (200 mL), stirred and filtered. The organic phases were combined, dried over anhydrous Na₂SO₄, filtered, and concentrated to obtain Compound A11B (7.05 g) as a green solid.

¹H NMR (400 MHz, CDCl₃) δ 6.57 (s, 1H), 6.52 (d, J=7.2 Hz, 1H), 6.37 (s, 1H), 3.82 (s, 2H), 3.17 (s, 3H), 1.33 (s, 9H).

LCMS: m/z=189.3, C₁₂H₁₇N₂⁺.

### Preparation Example 5: Preparation of 5-(dimethylamino)-1,2,3,3-tetramethyl-3H-indol-1-ium Iodide (A11)

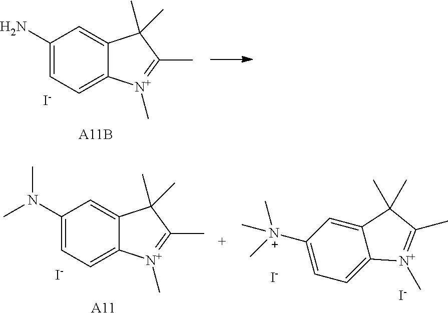

A mixture of Compound A11B (4.00 g, 12.7 mmol, 1.00 eq), MeI (8.98 g, 63.3 mmol, 3.94 mL, 5.00 eq) and ACN (20.0 mL) in a sealing tube was kept at 80° C. under 15 Psi for 12 hours. The resulting mixture was concentrated under reduced pressure, and the residues were purified by reversed-phase chromatography (column: Phenomenex luna C18 (250 mm×70 mm, 10 μm); mobile phase: [water (HCl)-ACN]; gradient: 1%-35% B, 22 min) to obtain Compound A11 (1.40 g, 3.97 mmol, 15.7% yield, 97.7% purity) as a yellow solid.

¹H NMR (400 MHz, DMSO-_{d6}) δ 7.68 (d, J=8.8 Hz, 1H), 7.18 (s, 1H), 6.88 (d, J=8.4 Hz, 1H), 3.90 (s, 3H), 2.65 (s, 3H), 1.47 (s, 6H).

LCMS: m/z=217.1, C₁₄H₂₁N₂⁺.

In addition, trimethyl-(1,2,3,3-tetramethylindol-1-ium-5-yl)amine diiodide (720 mg, 1.38 mmol, 10.93% yield, 93.4% purity) was obtained as a yellow solid. LCMS: m/z=231.0, C₁₅H₂₄N₂²⁺.

### Preparation Example 6: Preparation of (4-methylsulfonylphenyl)hydrazine (A13A)

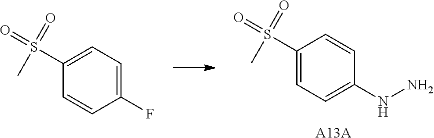

A solution of 1-fluoro-4-methylsulfonylbenzene (20.0 g, 115 mmol, 1.00 eq), hydrazine hydrate (13.0 g, 221 mmol, 12.6 mL, 85.0% purity, 1.92 eq) and EtOH (200 mL) was stirred for 16 hours at 80° C. The resulting mixture was concentrated under reduced pressure, and the residues were purified by column chromatography (SiO₂, PE:EA=1:0 to 1:1) to obtain Compound A13A (7.80 g, 41.9 mmol, 36.5 yield) as a yellow solid.

¹H NMR (400 MHz, CDCl₃) δ 7.74 (s, 1H), 7.71 (s, 1H), 6.90 (s, 1H), 6.87 (s, 1H), 5.74 (s, 1H), 3.70 (s, 2H), 3.01 (s, 3H).

### Preparation Example 7: Preparation of (E)-2-chloro-5-(dimethylamino)-3-(hydroxymethylene)cyclohex-1-ene-1-carbaldehyde (B4)

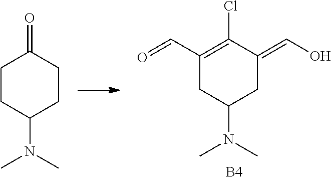

POCl₃ (16.3 g, 106 mmol, 9.90 mL, 3.00 eq) was added to DMF (10.0 mL) at 0° C. The mixture was kept at 0 to 5° C. and stirred for 30 minutes. 4-(Dimethylamino)cyclohexanone (5.00 g, 35.4 mmol, 1.00 eq) was added to the solution, heated to 80° C. and stirred for 3 hours. The reaction mixture was added to HCl (1.00 M, 100 mL), stirred for 12 hours at 20° C. The mixture was filtered, and the filter cake was dried to obtain Compound B4 (3.90 g, crude) as a yellow solid.

¹H NMR (400 MHz, DMSO) δ 8.89 (s, 2H), 3.40-3.44 (m, 1H), 3.02-3.07 (m, 2H), 2.76 (s, 6H), 2.36-2.40 (m, 2H).

LCMS: m/z=216.3 (M+H)⁺.

### Preparation Example 8: Preparation of phenyl 3-[2-(2,3,3-trimethylindolin-1-ium-1-yl)ethoxy]propionate Bromide (C4)

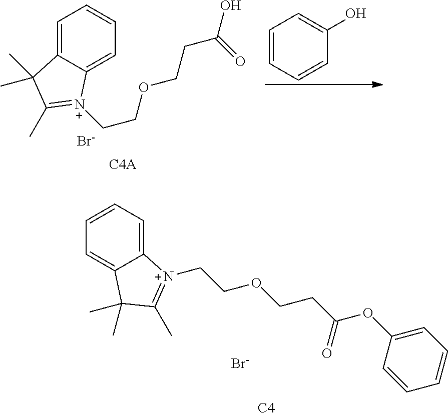 [SMILES: CC1=[N+](CCOCCC(=O)O)c2ccccc2C1(C)C ; Oc1ccccc1 ; [Br-] ; CC1=[N+](CCOCCC(=O)Oc2ccccc2)c2ccccc2C1(C)C ; [Br-] ; "C4"]

HBTU (7.41 g, 19.5 mmol, 1.20 eq) and DIEA (5.26 g, 40.7 mmol, 7.09 mL, 2.50 eq) was added to a solution of 3-[2-(2,3,3-trimethylindolin-1-ium-1-yl)ethoxy]propionic acid bromide (C4A) (see Preparation Example 13 for the preparation method) (5.80 g, 16.3 mmol, 1.00 eq) in DMF (50.0 mL). The mixture was stirred for 30 minutes at 25° C. Then, phenol (1.69 g, 17.9 mmol, 1.57 mL, 1.10 eq) was added, and stirred for 12 hours at 25° C. H₂O (300 mL) was added to the reaction mixture, and extracted with EA (200 mL×3). The organic phase was dried over Na₂SO₄ and concentrated. The residues were purified by reversed-phase HPLC (0.1% HCl condition; column: Phenomenex luna C18 250 mm×50 mm×15 μm; mobile phase: [water (FA)-ACN]; B %: 50%-80%, 10 min), and then lyophilized to obtain Compound C4 (3.10 g) as a brown oil.

LCMS: m/z=352.2, C₂₂H₂₆NO₃⁺.

### Preparation Example 9: Preparation of 1-(6-(2,6-dimethylphenoxy)-6-oxohexyl)-2,3,3-trimethyl-3H-indol-1-ium Chloride (C11)

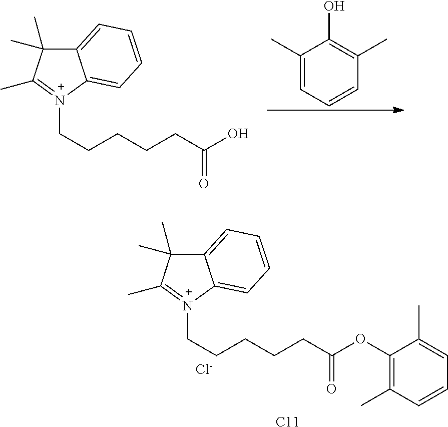

Compound C11 was prepared according to the method of Preparation Example 8, except that 1-(5-carboxypentyl)-2,3,3-trimethyl-3H-indol-1-ium was used instead of C4A, and 2,6-dimethylphenol was used instead of phenol.

### Preparation Example 10: Preparation of 2,3,3-trimethyl-1-(5-(trimethylammonium)pentyl)-3H-indol-1-ium Bromide (C14)

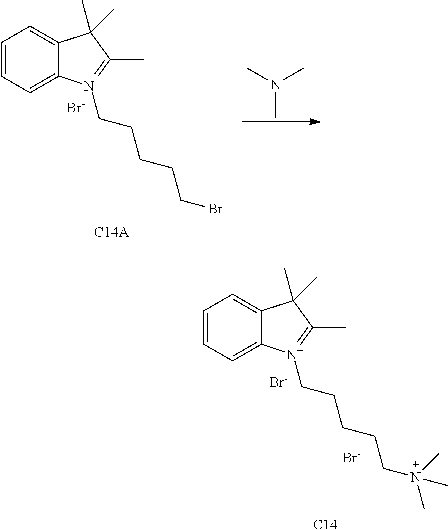

A mixture of 1-(5-bromopentyl)-2,3,3-trimethyl-indol-1-ium bromide (C14A) (see Preparation Example 22 for the preparation method in) (8.00 g, 20.6 mmol, 1.00 eq), N,N-dimethylcarbinamine (14.7 g, 82.2 mmol, 17.2 mL, 4.00 eq) and MeCN (80.0 mL) was stirred for 12 hours at 25° C. The resulting mixture was concentrated under reduced pressure, and purified by reversed-phase HPLC (column: Phenomenex luna, C18 150 mm×40 mm×15 μm; mobile phase: [water (HCl)-ACN]; B %: 30%-60%, 10 min) to obtain Compound C14 (2.30 g, 5.13 mmol, 24.7% yield) as a pink oil.

¹H NMR: (400 MHz, DMSO) δ 8.01-8.03 (m, 1H), 7.85-7.87 (m, 1H), 7.62-7.65 (m, 2H), 4.51 (t, J=7.6 Hz, 2H), 3.30-3.34 (m, 2H), 3.07 (s, 9H), 2.88 (s, 3H), 1.86-1.94 (m, 2H), 1.72-1.80 (m, 2H), 1.55 (s, 6H), 1.39-1.47 (m, 2H).

LCMS: m/z=144.3, C₁₉H₃₂N₂²⁺/2.

### Preparation Example 11: Preparation of 2,3,3-trimethyl-1-(4-(trimethylammonium)butyl)-3H-indol-1-ium Chloride (C15)

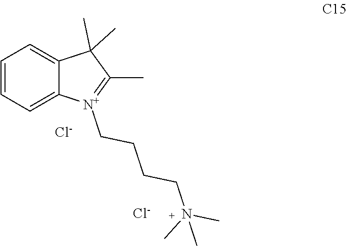

Compound C15 was prepared by the same method as Preparation Example 10, except that Compound C15A was used instead of C14A.

LCMS: m/z=273.3, C₁₈H₃₀N₂₂⁺.

### Preparation Example 12: Preparation of 5-methoxy-2,3,3-trimethyl-1-(6-oxo-6-phenoxyhexyl)-3H-indol-1-ium Bromide (C21B)

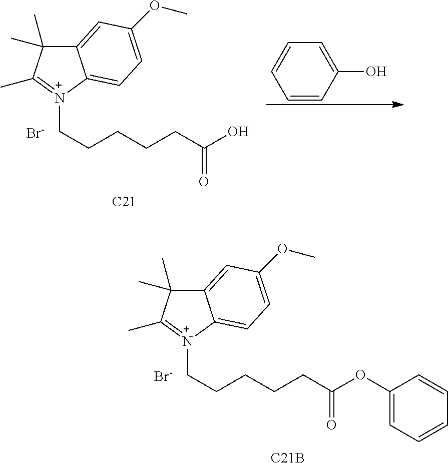

Compound C21B was prepared according to the method of Preparation Example 8, except that Compound C21 was used instead of C4A.

LCMS: m/z=380.3, C₂₄H₃₀NO₃⁺.

### Preparation Example 13: Preparation of Compound C4A

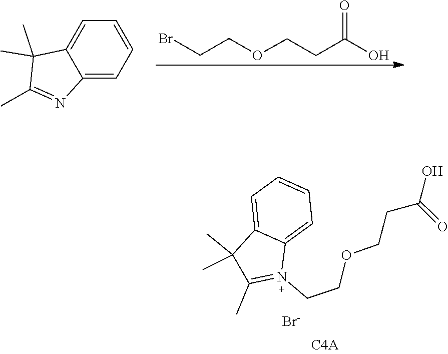

LCMS: m/z=276.2, C₁₆H₂₂NO₃⁺. Yellow oil.

### Preparation Example 14: Preparation of Compound A7

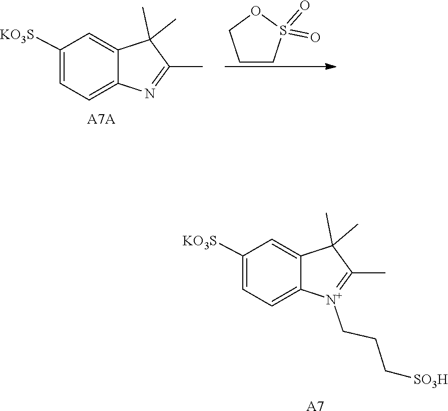

¹H NMR: (400 MHz, D₂O) δ 8.11 (s, 1H), 8.02 (d, J=8.4 Hz, 1H), 7.92 (d, J=8.4 Hz, 1H), 4.65 (d, J=16.4 Hz, 2H), 3.31 (s, 3H), 2.38-2.34 (m, 2H), 2.04 (s, 1H), 1.59 (s, 6H).

LCMS: m/z=362. Purple solid.

### Preparation Example 15: Preparation of Compound C10

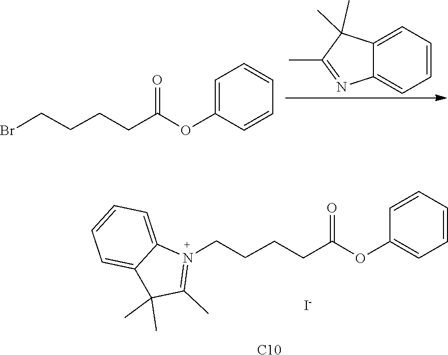

LCMS: m/z=336.2, C₂₂H₂₆NO₂⁺. Purple solid.

### Preparation Example 16: Preparation of Compound C7

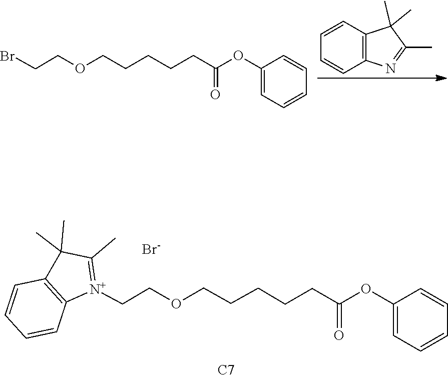

LCMS: m/z=394.3, C₂₅H₃₂NO₃⁺. Red oil.

### Preparation Example 17: Preparation of Compound C8

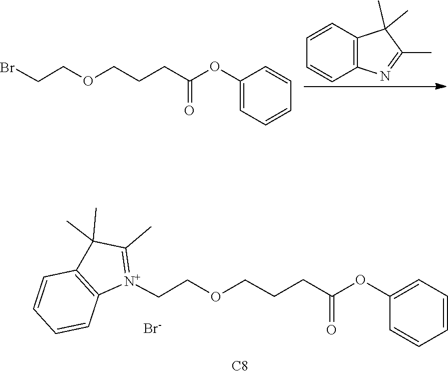

LCMS: m/z=366.2, C₂₃H₂₈NO₃⁺. Yellow oil.

### Preparation Example 18: Preparation of Compound C9

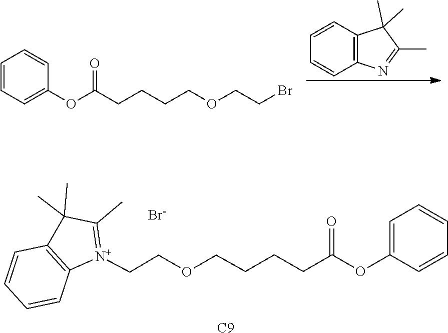

LCMS: m/z=380.2, C₂₄H₃₀NO₃⁺. Yellow oil.

### Preparation Example 19: Preparation of Compound C21

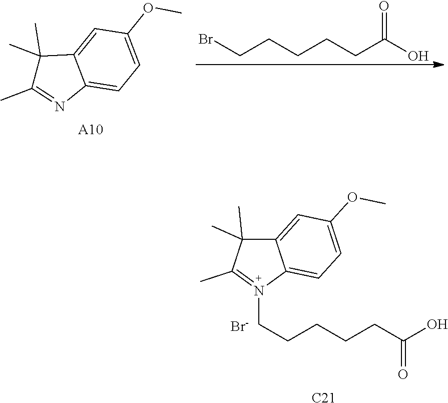

LCMS: m/z=304.2, C₁₈H₂₆NO₃⁺. Green solid.

### Preparation Example 20: Preparation of Compound C13

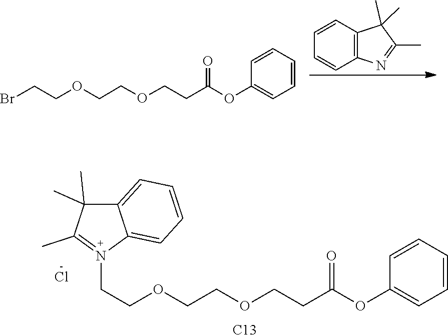

LCMS: m/z=396.3, C₂₄H₃₀NO₄⁺. Yellow oil.

### Preparation Example 21: Preparation of Compound C12

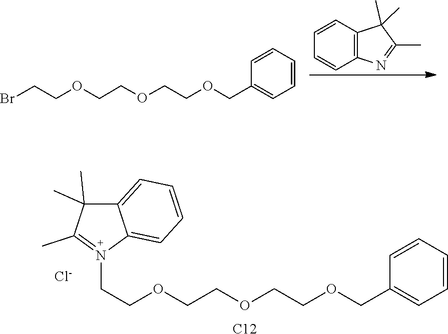

¹H NMR (400 MHz, CDCl₃) δ 7.66-7.58 (m, 1H), 7.57-7.56 (m, 2H), 7.55-7.54 (m, 1H), 7.37-7.32 (m, 5H), 5.83-5.57 (m, 4H), 4.80 (t, J=4.80 Hz, 2H), 4.51 (s, 2H), 3.97 (d, J=4.40 Hz, 2H), 3.56-3.54 (m, 2H), 3.51-3.50 (m, 2H), 2.74 (s, 3H), 1.47 (s, 6H).

LCMS: m/z=382.2, C₂₄H₃₂NO₃⁺. Brownish black solid.

### Preparation Example 22: Preparation of Compound C14A

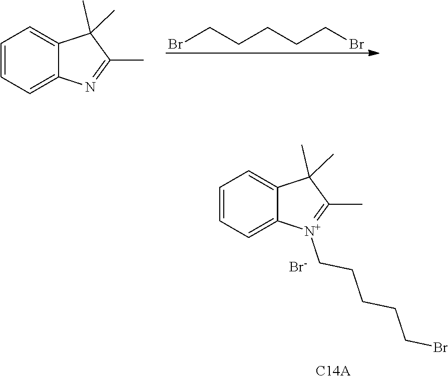

LCMS: m/z=310.2, C₁₆H₂₃BrN⁺. Red solid.

### Preparation Example 23: Preparation of Compound C15A

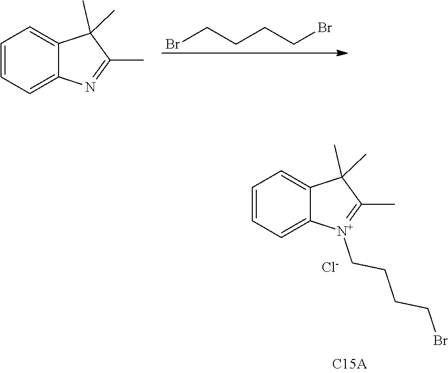

LCMS: m/z=296.1, C₁₅H₂₁BrN⁺. Brown oil.

### Preparation Example 24: Preparation of Compound C16

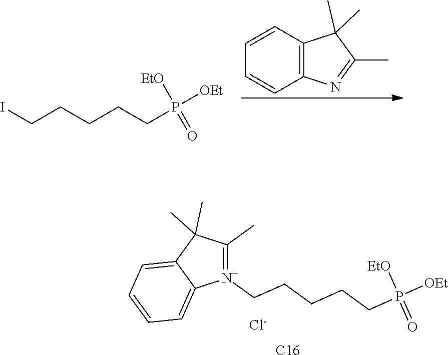

¹H NMR (400 MHz, CDCl₃) δ 7.55-7.68 (m, 4H), 4.85 (t, J=6.8 Hz, 2H), 4.04-4.10 (m, 4H), 3.18 (s, 3H), 1.98-2.00 (m, 3H), 1.64-1.78 (m, 11H), 1.30 (t, J=7.2 Hz, 6H).

LCMS: m/z=366.3, C₂₀H₃₃NO₃P⁺. Brown oil.

### Preparation Example 25: Preparation of Compound C17

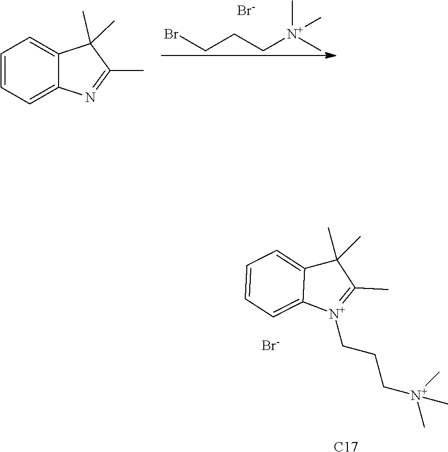

LCMS: m/z=259.3, C₁₇H₂₈N₂₂⁺. Yellow solid.

### Preparation Example 26: Preparation of Compound C18

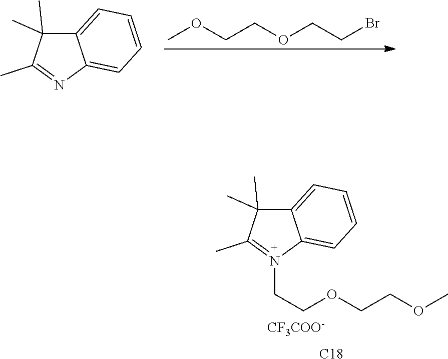

LCMS: m/z=262.2, C₁₆H₂₄NO₂⁺. Green solid.

### Preparation Example 27: Preparation of Compound C19

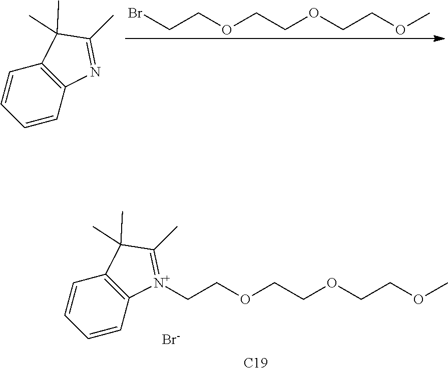

¹H NMR (400 MHz, CDCl₃) δ 7.62-7.64 (m, 1H), 7.49-7.53 (m, 3H), 4.76 (s, 2H), 3.95 (t, J=5.2 Hz, 2H), 3.55-3.57 (m, 2H), 3.48-3.52 (m, 4H), 3.42-3.44 (m, 2H), 3.32 (s, 3H), 2.00 (s, 6H), 1.55 (s, 3H).

LCMS: m/z=306.3, C₁₈H₂₈NO₃⁺. Red oil.

### Preparation Example 28: Preparation of Compound C20

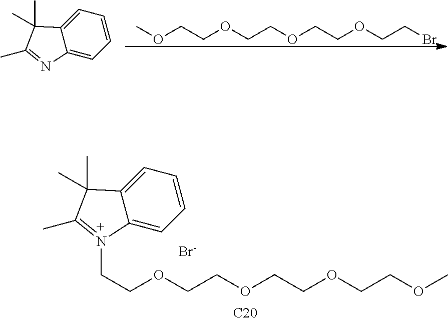

LCMS: m/z=350.3, C₂₀H₃₂NO₄⁺. Yellow oil.

### Preparation Example 29: Preparation of Compound C23

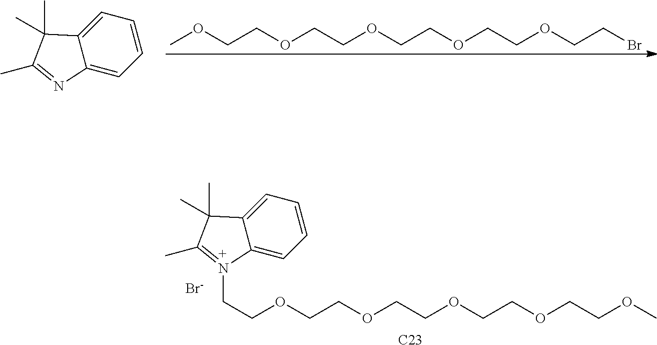

LCMS: m/z=394.2, C₂₂H₃₆NO₅⁺. Brown oil.

### Example 1: Preparation of 2-((E)-2-((E)-2-chloro-3-((E)-2-(1,1-dimethyl-3-(6-oxo-6-phenoxyhexyl)-1,3-dihydro-2H-benzo[e]indol-2-ylidene)ethenyl)cyclopent-1-en-1-yl)ethenyl)-3-ethyl-1,1-dimethyl-1H-benzo[e]indol-3-ium Chloride (1A) and 2-((E)-2-((E)-3-((E)-2-(3-(5-carboxypentyl)-1,1-dimethyl-1,3-dihydro-2H-benzo[e]indol-2-ylidene)ethenyl)-2-chlorocyclopent-1-en-1-yl)ethenyl)-3-ethyl-1,1-dimethyl-1H-benzo[e]indol-3-ium Chloride (1B)

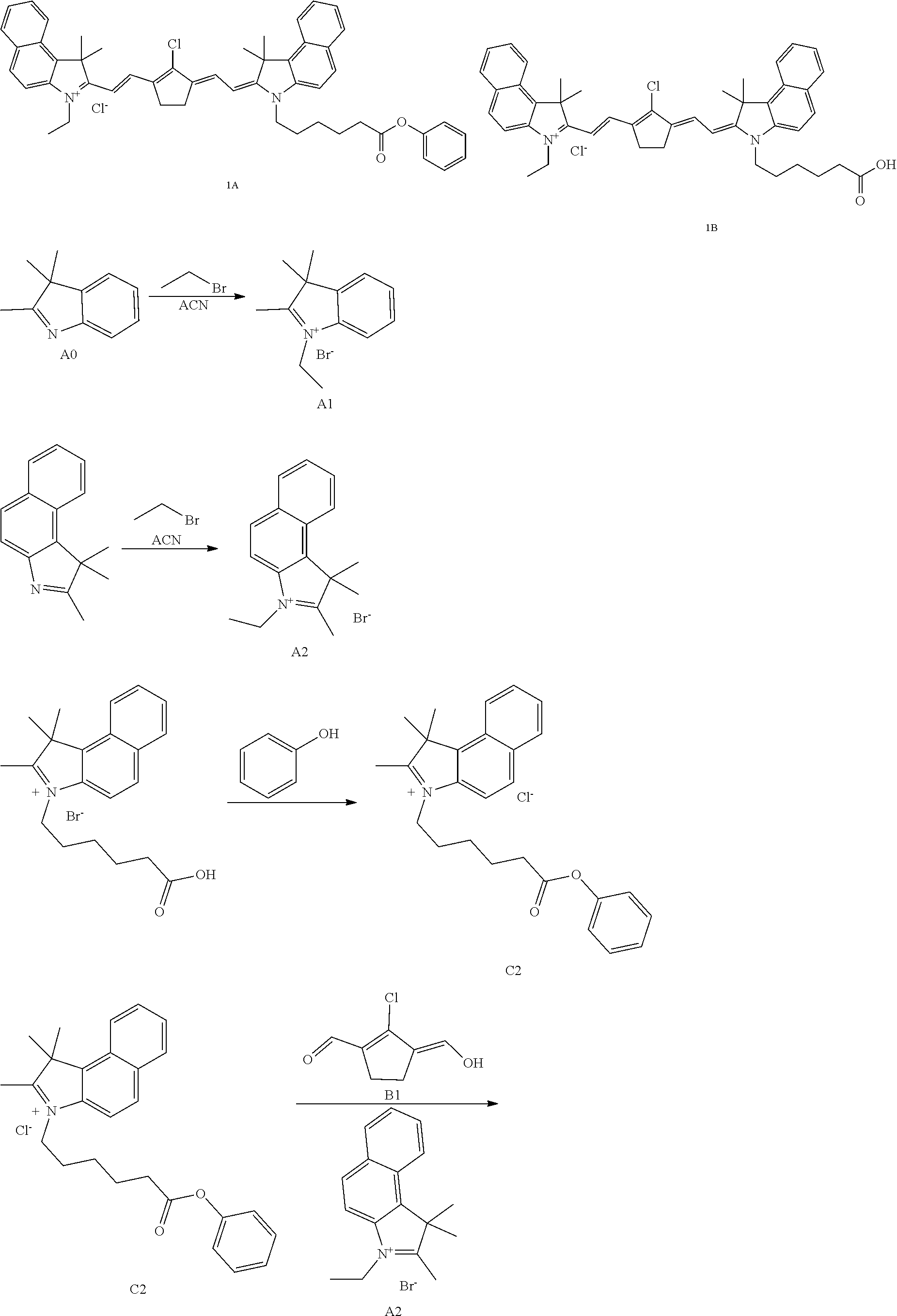

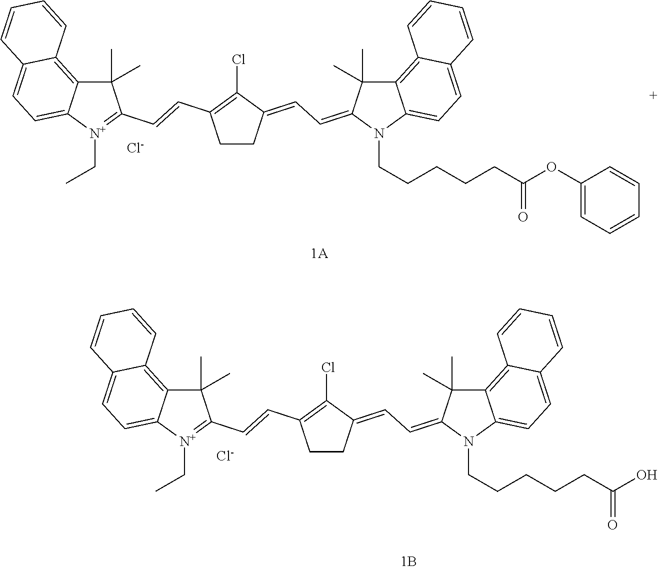 [SMILES: CC[N+]1=C(/C=C/C2=C(Cl)C(=C/C=C3/N(CCCCCC(=O)Oc4ccccc4)c4ccc5ccccc5c4C3(C)C)/CC2)C(C)(C)c2c1ccc1ccccc21 ; [Cl-] ; CC[N+]1=C(/C=C/C2=C(Cl)C(=C/C=C3/N(CCCCCC(=O)O)c4ccc5ccccc5c4C3(C)C)/CC2)C(C)(C)c2c1ccc1ccccc21 ; [Cl-]]

### Step 1: Preparation of 1-ethyl-2,3,3-trimethyl-3H-indol-1-ium Bromide (A1)

A solution of 2,3,3-trimethylindole (A0) (10.0 g, 62.8 mmol, 1.00 eq), bromoethane (10.3 g, 94.2 mmol, 7.03 mL, 1.50 eq) and acetonitrile (80.0 mL) was heated to 85° C. and stirred for 24 hours. The reaction mixture was concentrated, and pulped with MTBE (60.0 mL) to obtain Compound A1 (12.7 g, 47.4 mmol, yield 75.4%) as a brown solid.

¹H NMR (400 MHz, CDCl₃) δ 7.99-8.01 (m, 1H), 7.85-7.87 (m, 1H), 7.61-7.64 (m, 2H), 4.49-4.55 (m, 2H), 2.86 (s, 3H), 1.54 (s, 6H), 1.45 (t, J=7.4 Hz, 3H).

LCMS: m/z=188.1 (positive ion).

### Step 2: Preparation of 3-ethyl-1,1,2-trimethyl-1H-benzo[e]indol-3-ium Bromide (A2)

1,1,2-Trimethylbenzo[e]indole (20.0 g, 95.5 mmol, 1.00 eq) and bromoethane (15.6 g, 143 mmol, 10.7 mL, 1.50 eq) were mixed in ACN (200 mL), evacuated and purged with N₂ three times. The mixture was then heated for 36 hours at 85° C. The resulting mixture was detected by LCMS, showing a target product generated with a yield of about 56%. The reaction mixture was concentrated, pulped with MTBE/DCM=6:1 (300 mL/50.0 mL) for 12 hours at 25° C., and filtered to obtain a filter cake, i.e., Compound A2 (16.0 g, 47.9 mmol, 50.2% yield, 95.4% purity) as a product.

### Step 3: Preparation of phenyl 6-(1,1,2-trimethylbenzo[e]indol-3-ium-3-yl)n-hexanoate Chloride (C2)

6-(1,1,2-Trimethylbenzo[e]indol-3-ium-3-yl)n-hexanoic acid bromide (5.00 g, 15.4 mmol, 1.00 eq) was dissolved in a mixed solvent of DMF (15.0 mL) and DCM (20.0 mL), and EDCI (7.01 g, 18.4 mmol, 1.20 eq) and DIEA (4.98 g, 38.5 mmol, 6.71 mL, 2.50 eq) were added. The resulting mixture was stirred for 0.5 hour at 25° C., then phenol (1.60 g, 16.9 mmol, 1.49 mL, 1.10 eq) was added, and the reaction was continued for 12 hours at this temperature. The detection showed 6-(1,1,2-trimethylbenzo[e]indol-3-ium-3-yl)n-hexanoic acid bromide was consumed completely. The reaction solution was concentrated, separated and purified by reversed-phase HPLC (0.1% HCl). Compound C2 (2.40 g, 5.99 mmol, 38.9% yield) was obtained as a brown solid after lyophilization.

LCMS: m/z=400.2, C₂₇H₃₀NO₂⁺.

### Step 4: Preparation of Compound 1B

A solution of Compound A2 (1.00 g, 3.15 mmol, 1.00 eq), Compound C2 (1.89 g, 4.72 mmol, 1.50 eq) and acetic anhydride (30.0 mL) was stirred for 1 hour at 70° C., and NaOAc (776 mg, 9.45 mmol, 3.00 eq) was added. Then (E)-2-chloro-3-(hydroxymethylene)cyclopent-1-en-1-carbaldehyde (Compound B1) (500 mg, 3.15 mmol, 1.00 eq) was added in portions. After stirring for 1 hour at 80° C., the solvent was removed through concentration. The residues were separated and purified by reversed-phase chromatography (0.1% HCl condition) to obtain Compound 1A (53.7 mg, 68.4 μmol, 2.17% yield, 96.8% purity) as a brown solid, and Compound 1B (4.90 mg, 6.80 μmol, 0.22% yield, 95.0% purity) as a white solid.

### Compound 1A:

¹H NMR (400 MHz, CDCl₃) δ 8.11-8.15 (m, 2H), 7.93-7.98 (m, 5H), 7.60-7.62 (m, 2H), 7.44-7.47 (m, 4H), 7.28-7.32 (m, 3H), 7.19-7.20 (m, 1H), 6.97-6.99 (m, 2H), 6.25-6.30 (m, 2H), 4.41-4.49 (m, 4H), 3.11-3.16 (m, 4H), 2.61-2.65 (m, 2H), 2.01 (d, J=4.8 Hz, 12H), 1.85-1.87 (m, 2H), 1.67-1.69 (m, 3H), 1.51-1.55 (m, 4H).

LCMS: m/z=759.6 (M+H)⁺.

### Compound 1B:

LCMS: m/z=683.5 (M+H)⁺.

### Example 2: Preparation of phenyl 6-[(2E)-2-[(2E)-2-[2-chloro-3-[(E)-2-(3-ethyl-1,1-dimethyl-benzo[e]indol-3-ium-2-yl)ethenyl]cyclohex-2-en-1-yl]ethylidene]-1,1-dimethyl-benzo[e]indol-3-yl]n-hexanoate (2)

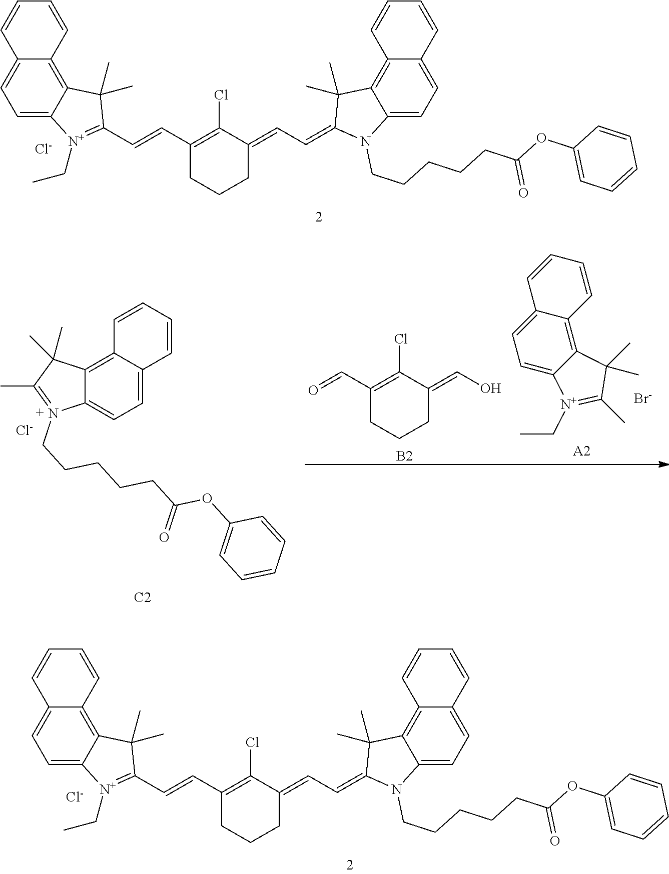

To a solution of Compound A2 (1.01 g, 3.16 mmol, 1.00 eq) in Ac₂O (12.0 mL) was added a solution of Compound C2 (1.90 g, 4.74 mmol, 1.50 eq) in Ac₂O (12.0 mL). After keeping the temperature at 70° C. for 3 hours, the reaction solution was concentrated. The residues were dissolved in EtOH (40.0 mL) and heated to 80° C. Compound B2 (purchased from WuXi AppTec, 545 mg, 3.16 mmol, 1 eq) and NaOAc (778 mg, 9.49 mmol, 3.00 eq) were added, and stirring was continued for 2 hours at 80° C. After the reaction solution was concentrated, the residues were purified by preparative chromatography (chromatographic column: YMC Triart C18 150 mm×25 mm×5 μm; mobile phase[water (HCl)-ACN]; B %: 76%-100%, 10 min), and lyophilized to obtain Compound 2 (52.47 mg, 65.9 μmol, 34.0% yield, 97.3% purity) as a green solid.

¹H NMR: (400 MHz, CDCl₃) δ 8.47-8.41 (m, 1H), 8.13 (d, J=8.0 Hz, 2H), 7.98-7.94 (m, 4H), 7.62 (d, J=7.2 Hz, 2H), 7.53-7.46 (m, 3H), 7.31-7.27 (m, 2H), 7.20-7.15 (m, 1H), 6.98-6.95 (m, 2H), 6.41-6.22 (m, 1H), 4.41 (s, 3H), 2.95-2.57 (m, 5H), 2.02-1.96 (m, 20H), 1.89-1.80 (m, 3H), 1.70-1.62 (m, 2H), 1.53 (t, J=5.1 Hz, 3H).

### Example 3: Preparation of 2-((E)-2-((E)-2-chloro-3-((E)-2-(1,1-dimethyl-3-(4-(phenoxycarbonyl)phenyl)-1,3-dihydro-2H-benzo[e]indol-2-ylidene)ethenyl)cyclohex-1-en-1-yl)ethenyl)-3-ethyl-1,1-dimethyl-1H-benzo[e]indol-3-ium Chloride (3)

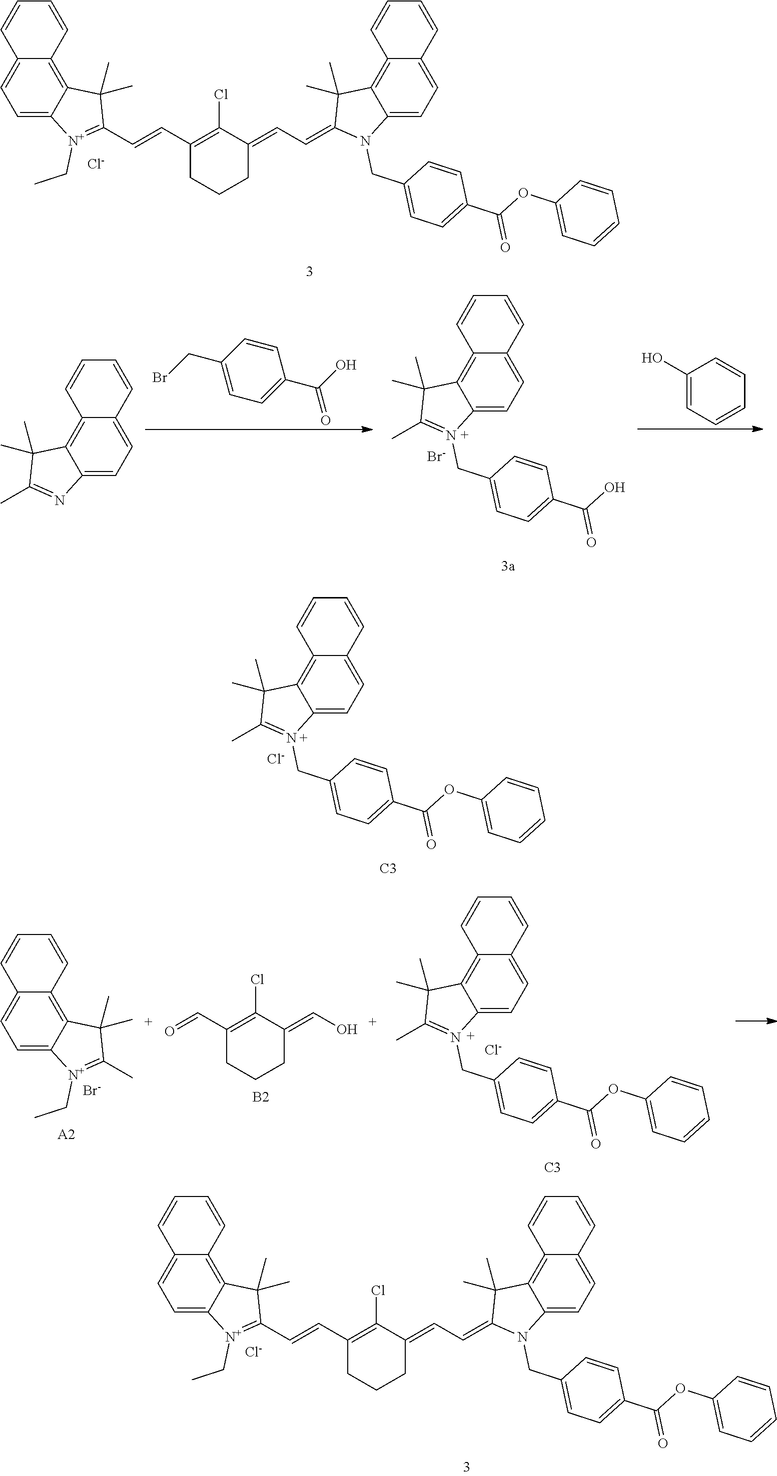

### Step 1: Preparation of 3-(4-carboxybromo)-1,1,2-trimethyl-1H-benzo[e]indol-3-ium Bromide (3a)

To a solution of 1,1,2-trimethylbenzo[e]indole (3.75 g, 17.90 mmol, 1.00 eq) in ACN (50.0 mL) was added 4-(bromomethyl)benzoic acid (3.85 g, 17.9 mmol, 1.00 eq). The mixture was stirred for 12 hours at 85° C. The reaction solution was concentrated under reduced pressure, and the residues were pulped with MTBE/DCM=1:2 (210 mL/30 mL) for 30 minutes at 25° C., and filtered to obtain a filter cake, i.e., Compound 3a (6.50 g, 15.3 mmol, 85.5% yield) as a product, which was a brown solid.

¹H NMR: (400 MHz, CDCl₃) δ 8.41 (d, J=8.4 Hz, 1H), 8.22-8.16 (m, 2H), 7.97-7.91 (m, 3H), 7.82-7.73 (m, 2H), 4.22-4.16 (m, 4H), 7.57-7.52 (m, 2H), 6.06 (s, 2H), 3.07 (s, 3H), 1.85 (s, 6H).

### Step 2: Preparation of 1,1,2-trimethyl-3-(4-(phenoxycarbonyl)bromo)-1H-benzo[e]indol-3-ium Chloride (C3)

Compound 3a (4.50 g, 10.6 mmol, 1.00 eq) was dissolved in DMF (10.0 mL) and DCM (60.0 mL), and HBTU (4.83 g, 12.7 mmol, 1.20 eq) and DIEA (3.43 g, 26.5 mmol, 4.62 mL, 2.50 eq) were added. The mixed solution was stirred for 0.5 hour at 25° C., and then phenol (1.10 g, 11.6 mmol, 1.03 mL, 1.10 eq) was added. The mixture was then stirred for 12 hours at 25° C. The reaction solution was concentrated, and the residues were purified by reversed-phase chromatography (0.1% HCl condition) to obtain Compound C3 (1.7 g, 4.04 mmol, 38.12% yield) as a brown solid.

LCMS: m/z=420.2 C₂₉H₂₆ClNO₂⁻.

### Step 3: Preparation of Compound 3

A mixture of Compound A2 (353 mg, 1.11 mmol, 1.00 eq), 1,1,2-trimethyl-3-(4-(phenoxycarbonyl)bromo)-1H-benzo[e]indol-3-ium chloride (C3) (0.700 g, 1.66 mmol, 1.50 eq) and Ac₂O (14.0 mL) was stirred for 1 hour at 70° C., and NaOAc (273 mg, 3.33 mmol, 3.00 eq) and Compound B2 (192 mg, 1.11 mmol, 1.00 eq) were added. The reaction solution was heated to 80° C. and stirred for 1 hour. The reaction mixture was concentrated, and the residues were purified by preparative chromatography (HCl condition) to obtain Compound 3 (104.57 mg, 126 μmol, 11.4% yield, 95.7% purity) as a green solid.

¹H NMR (400 MHz, CDCl₃) δ 8.51-8.55 (m, 1H), 8.29-8.32 (m, 1H), 8.14-8.22 (m, 4H), 7.89-8.02 (m, 2H), 7.40-7.67 (m, 2H), 7.40-7.58 (m, 9H), 7.31-7.34 (m, 1H), 7.19-7.21 (m, 2H), 6.50-6.57 (m, 1H), 6.15-6.18 (m, 1H), 5.65 (s, 2H), 4.58-4.60 (m, 2H), 2.80 (s, 2H), 2.63 (s, 2H), 2.10 (s, 6H), 2.03 (s, 6H), 2.00-2.01 (m, 2H), 1.50-1.60 (m, 3H).

LCMS: m/z=793.3.

### Example 4: Preparation of 2-((E)-2-((E)-2-chloro-3-(2-((E)-3,3-dimethyl-1-(6-oxo-6-phenoxyhexyl)indolin-2-ylidene)ethenyl)cyclopent-1-en-1-yl)ethenyl)-1-ethyl-3,3-dimethyl-3H-indol-1-ium Chloride (4)

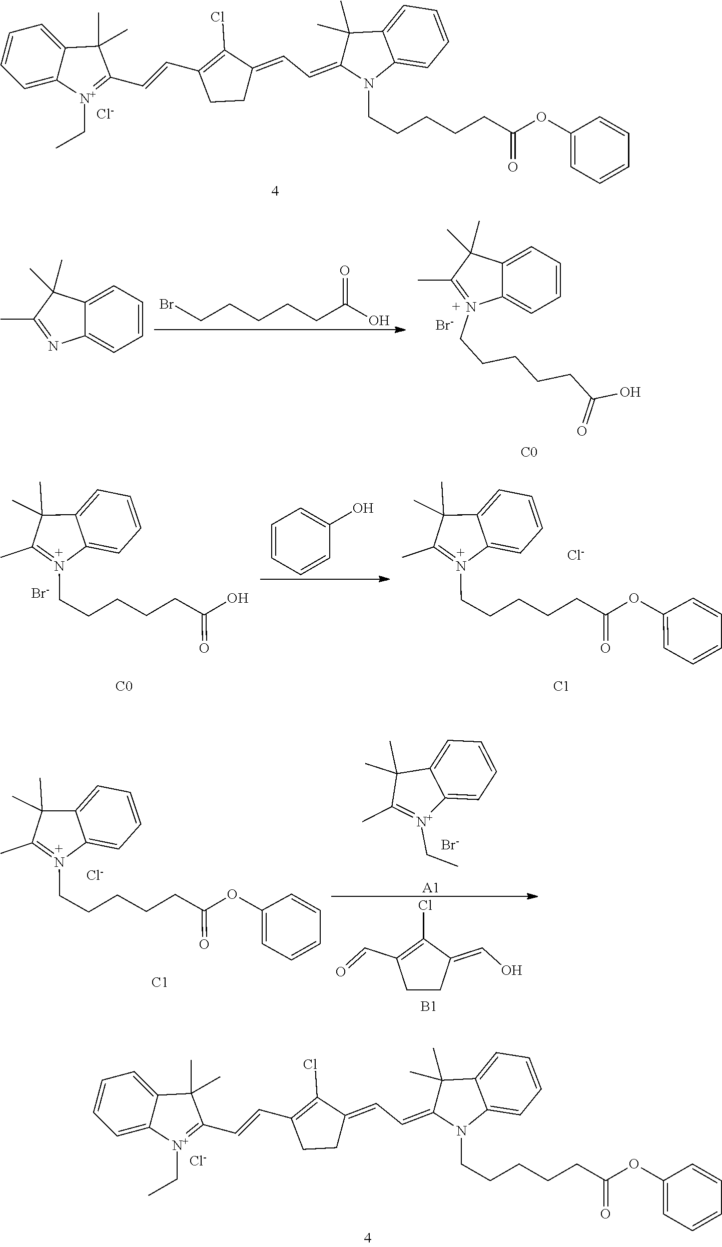

### Step 1: Preparation of 1-(5-carboxypentyl)-2,3,3-trimethyl-3H-indol-1-ium Bromide (C0)

A solution of 2,3,3-trimethylindole (5 g, 31.40 mmol, 1 eq) and 6-bromohexanoic acid (12.25 g, 62.80 mmol, 2 eq) in ACN (40 mL) was stirred for 24 hours at 85° C. The reaction solution was concentrated under reduced pressure to remove the solvent to give a crude product. Then, the crude product was pulped with DCM:MTBE=1:1 (60 mL) for 30 minutes at 0° C. to obtain Compound C0 (6.9 g, 19.48 mmol, 62.02% yield) as a pink solid.

¹H NMR: (400 MHz, CDCl₃) δ 7.99-7.97 (m, 1H), 7.86-7.83 (m, 1H), 7.63-7.61 (m, 2H), 4.48-4.44 (m, 2H), 2.85 (s, 3H), 2.24-2.20 (t, J=7.2 Hz, 2H), 1.86-1.80 (m, 2H), 1.57-1.54 (m, 8H), 1.44-1.41 (m, 2H).

### Step 2: Preparation of 2,3,3-trimethyl-1-(6-oxo-6-phenoxyhexyl)-3H-indol-1-ium Chloride (C1)

Compound C0 (4.00 g, 11.3 mmol, 1.00 eq) was mixed in DCM (50.0 mL), and then HBTU (5.14 g, 13.5 mmol, 1.20 eq) and DIEA (3.65 g, 28.2 mmol, 4.92 mL, 2.50 eq) were added. The resulting mixture was stirred for 0.5 hour at 25° C., then phenol (1.06 g, 11.3 mmol, 993 μL, 1.00 eq) was added, and the reaction was continued for 12 hours at 25° C. The resulting mixture was purified by reversed-phase HPLC (0.1% HCl condition), and lyophilized to obtain Compound C1 (2.00 g, 4.53 mmol, 40.1% yield, 79.3% purity) as a product, which was a brown solid.

LCMS: m/z=350.1, C₂₃H₂₈NO₂⁺.

### Step 3: Preparation of Compound 4

After a solution of Compound C1 (1.32 g, 3.78 mmol, 1.50 eq), Compound A1 (676 mg, 2.52 mmol, 1.00 eq) and Ac₂O (20.0 mL) was stirred for 1 hour at 70° C., NaOAc (621 mg, 7.56 mmol, 3.00 eq) and Compound B1 (400 mg, 2.52 mmol, 1.00 eq) were added. The resulting mixture was heated to 80° C. and stirred for 1 hour. The reaction mixture was then concentrated under reduced pressure. The residues were purified by preparative chromatography (HCl condition: column: YMC Triart C18 150 mm×25 mm×5 μm; mobile phase: [water (HCl)-ACN]; B %: 70%-100%, 10 min), and lyophilized to obtain Compound 4 (68.07 mg, 89.0 μmol, 3.53% yield, 91% purity) as a green solid.

¹H NMR (400 MHz, DMSO) δ 7.83 (t, J=14 Hz, 2H), 7.34-7.43 (m, 6H), 7.22-7.23 (m, 3H), 7.11-7.16 (m, 2H), 7.02-7.06 (m, 2H), 6.05-6.11 (m, 2H), 4.16-4.21 (m, 4H), 2.63 (t, J=7.2 Hz, 2H), 1.78-1.93 (m, 8H), 1.71 (d, J=4.4 Hz, 12H), 1.60-1.64 (m, 2H), 1.46 (t, J=7.2 Hz, 3H).

LCMS: m/z=659.2, C₄₃H₄₈ClN₂O₂⁺.

### Example 5: Preparation of 2-((E)-2-((E)-2-chloro-3-(2-((E)-3,3-dimethyl-1-(6-oxo-6-phenoxyhexyl)indolin-2-ylidene)ethenyl)cyclohex-1-en-1-yl)ethenyl)-3,3-dimethyl-1-(6-oxo-6-phenoxyhexyl)-3H-indol-1-ium Chloride (5)

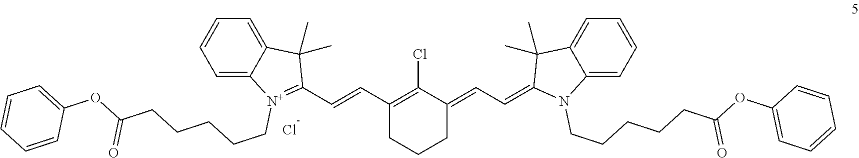 [SMILES: CC1(C)C(/C=C/C2=C(Cl)C(=C/C=C3/N(CCCCCC(=O)Oc4ccccc4)c4ccccc4C3(C)C)/CCC2)=[N+](CCCCCC(=O)Oc2ccccc2)c2ccccc21 ; Cl]

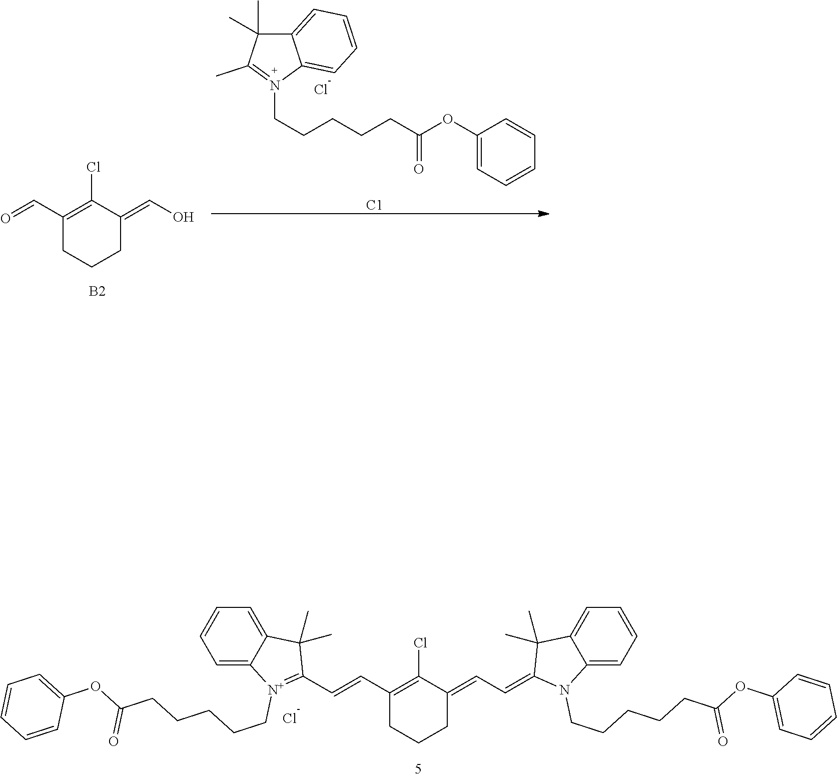

Compound C1 (3.05 g, 8.69 mmol, 2.00 eq) was added to a solution of Compound B2 (750 mg, 4.35 mmol, 1.00 eq), NaOAc (1.07 g, 13.0 mmol, 3.00 eq) and Ac₂O (30.0 mL). The resulting mixture was stirred for 40 minutes at 70° C. The solvent was concentrated, and the residues were purified by preparative chromatography (HCl condition: column: Phenomenex luna C18 150 mm×40 mm×15 μm; mobile phase: [water (HCl)-ACN]; B %: 70%-100%, 10 min) to obtain Compound 5 (263 mg, 288 μmol, 6.62% yield, 95.4% purity) as a green solid.

¹H NMR (400 MHz, CDCl₃) δ 8.36 (d, J=14 Hz, 2H), 7.35-7.42 (m, 8H), 7.27-7.28 (m, 1H), 7.24-7.26 (m, 5H), 7.03 (d, J=7.6 Hz, 4H), 6.30 (d, J=13.6 Hz, 2H), 4.25 (s, 4H), 2.72 (s, 4H), 2.60-2.64 (m, 4H), 1.93-1.95 (m, 6H), 1.83-1.89 (m, 4H), 1.72 (s, 12H), 1.63-1.64 (m, 4H).

LCMS: m/z=835.5 (M+H)⁺.

### Example 6: Preparation of 2-((E)-2-((E)-2-chloro-3-(2-((E)-3,3-dimethyl-1-(4-(phenoxycarbonyl)bromo)indolin-2-ylidene)ethenyl)cyclohex-1-en-1-yl)ethenyl)-1-ethyl-3,3-dimethyl-3H-indol-1-ium Chloride (6)

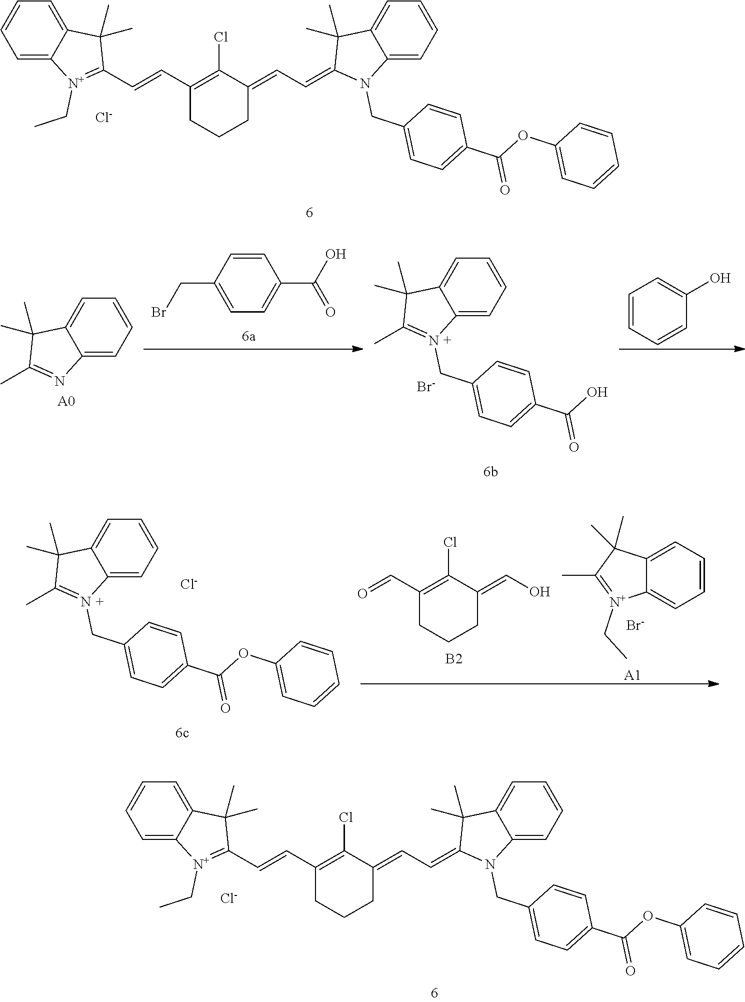

### Step 1: Preparation of 1-(4-carboxybromo-2,3,3-trimethyl-3H-indol-1-ium Bromide (6b)

Acetonitrile (50.0 mL) was added to compound A0 (6.00 g, 37.6 mmol, 1.00 eq), and then 4-(bromomethyl)benzoic acid (6a, purchased from Shanghai Bide Pharmatech Co., Ltd.) (12.1 g, 56.5 mmol, 1.50 eq) was added. The mixture was heated for 12 hours at 85° C. The reaction solution was concentrated under reduced pressure, and then pulped with MTBE/DCM=1:2 (60.0 mL/120 mL) for 30 minutes at 25° C. The mixture was filtered to obtain Compound 6b (11.0 g, 28.1 mmol, 74.6% yield, 95.7% purity) as a yellow solid.

¹H NMR (400 MHz, DMSO) δ 7.97 (d, J=8.0 Hz, 2H), 7.88 (d, J=7.2 Hz, 1H), 7.77 (d, J=7.2 Hz, 1H), 7.63-7.51 (m, 4H), 5.92 (s, 2H), 2.97 (s, 3H), 1.62 (s, 6H).

### Step 2: Preparation of 2,3,3-trimethyl-1-(4-(phenoxycarbonyl)bromo)-3H-indol-1-ium Chloride (6c)

Compound 6b (6.00 g, 16.0 mmol, 1.00 eq) was dissolved in DMF (10.0 mL) and DCM (40.0 mL), and then HBTU (7.30 g, 19.2 mmol, 1.20 eq) and DIEA (5.18 g, 40.0 mmol, 6.98 mL, 2.50 eq) were added. The reaction solution was stirred for 0.5 hour at 25° C. Phenol (1.66 g, 17.6 mmol, 1.55 mL, 1.10 eq) was added to the reaction solution, and stirring was continued for 12 hours. The reaction solution was concentrated, and the residues were purified by reversed-phase preparative chromatography (0.1% HCl condition). Compound 6c (3.6 g, 8.85 mmol, 55.22% yield, 91.1% purity) was obtained as a red solid after lyophilization.

LCMS: m/z=370.1, C₂₅H₂₄NO₂⁺.

### Step 3: Preparation of Compound 6

Compound 6c (965 mg, 2.61 mmol, 1.50 eq) and 1-ethyl-2,3,3-methyl-3H-indol-1-ium bromide (A1) (466 mg, 1.74 mmol, 1.00 eq) were dissolved in Ac₂O (20.0 mL), and stirred for 1 hour at 70° C. Compound B2 (300 mg, 1.74 mmol, 1.00 eq) was added, followed by the addition of NaOAc (427 mg, 5.21 mmol, 16.3 μL, 3.00 eq). The mixture was stirred for 1 hour at 80° C. The reaction solution was concentrated, and the resulting residues were separated by reversed-phase preparative chromatography to obtain green Compound 6 (200 mg, 187 μmol, 10.7% yield, 65.0% purity). Further purification provided a green solid (83.47 mg, 113 μmol, 39.2% yield, 94.1% purity).

¹H NMR (400 MHz, CDCl₃) δ 8.45 (d, J=14.4 Hz, 2H), 8.26-8.19 (m, 3H), 7.47-7.38 (m, 7H), 7.35-7.28 (m, 3H), 7.26-7.22 (m, 2H), 7.20-7.18 (m, 2H), 7.01 (d, J=8.0 Hz, 2H), 6.44 (d, J=14.4 Hz, 1H), 6.03 (d, J=13.2 Hz, 1H), 5.79-5.52 (m, 1H), 5.41 (s, 2H), 4.43-4.35 (m, 2H), 2.72 (s, 2H), 2.55 (s, 2H), 1.91 (s, 2H), 1.77 (s, 6H), 1.73 (s, 6H), 1.48 (t, J=5.1 Hz, 3H).

### Example 7: Preparation of 2-((E)-2-((E)-2-chloro-3-(2-((E)-3,3-dimethyl-1-(2-(3-oxo-3-phenoxypropoxy)ethyl)indolin-2-ylidene)ethenyl)cyclohex-1-en-1-yl)ethenyl)-1-ethyl-3,3-dimethyl-3H-indol-1-ium Trifluoroacetate (7)

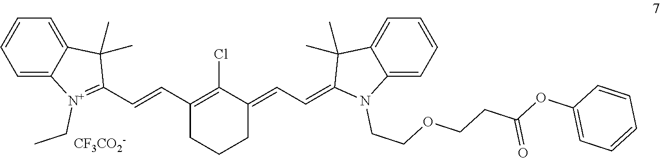

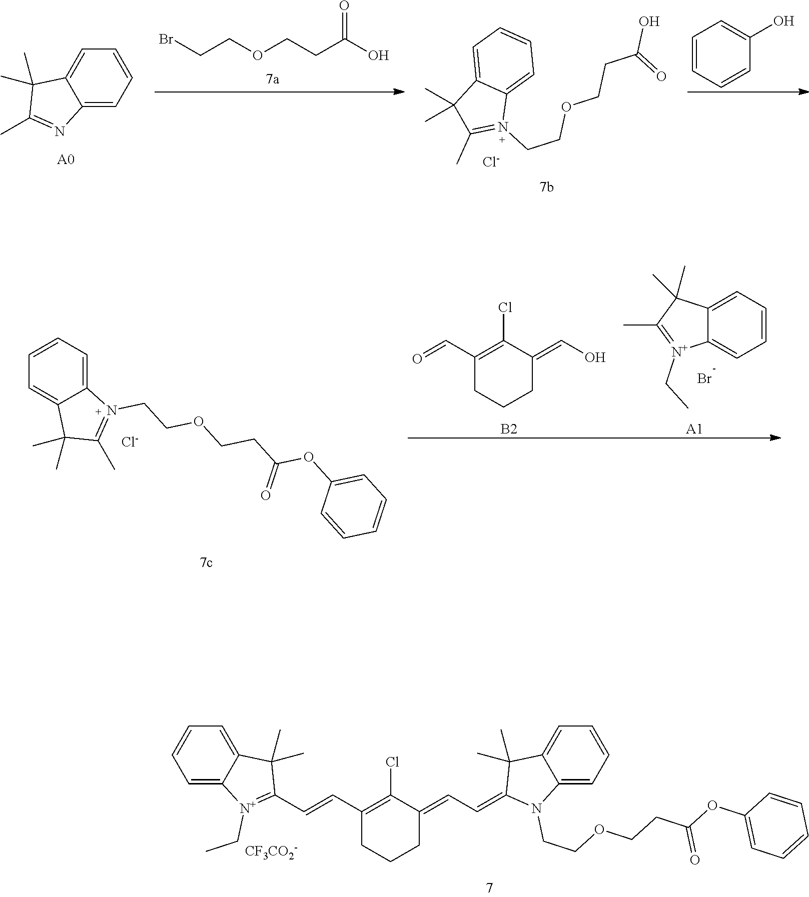

### Step 1: Preparation of 1-(2-(2-carbonylethoxy)ethyl)-2,3,3-trimethyl-3H-indol-1-ium Chloride (7b)

3-(2-Bromoethoxy)propionic acid (7a) (2.97 g, 15.1 mmol, 2.00 eq) was added to a solution of Compound A0 (1.20 g, 7.54 mmol, 1.00 eq) and acetonitrile (15.0 mL). The resulting mixture was stirred at 90° C. for 36 hours. After the solvent was concentrated, the residues were purified by preparative chromatography (HCl condition) to obtain Compound 7b (1.60 g, 4.49 mmol, 59.6 yield) as a colourless paste.

LCMS: m/z=276.1 (M+H)⁺.

### Step 2: Preparation of 2,3,3-trimethyl-1-(2-(3-oxo-3-phenoxypropoxy)ethyl)-3H-indol-1-ium Chloride (7c)

HBTU (2.04 g, 5.39 mmol, 1.20 eq) and DIEA (1.45 g, 11.2 mmol, 1.96 mL, 2.50 eq) were added to a solution of Compound 7b (1.60 g, 4.49 mmol, 1.00 eq), DMF (3.00 mL) and DCM (22.0 mL), and stirred at 25° C. for 30 minutes. Phenol (465 mg, 4.94 mmol, 435 μL, 1.10 eq) was added and stirred at 25° C. for 12 hours. The resulting mixture was concentrated under reduced pressure, and the residues were purified by preparative chromatography (0.1% HCl condition) to obtain Compound 7c (1.40 g, 3.97 mmol, 88.5% yield) as a yellow oil.

LCMS: m/z=352.2 (M+H)⁺.

### Step 3: Preparation of Compound 7

A solution of Compound 7c (306 mg, 869 μmol, 1.00 eq), Compound A1 (350 mg, 1.30 mmol, 1.50 eq) and acetic anhydride (8.00 mL) was stirred at 80° C. for 1.5 hours. Then NaOAc (214 mg, 2.61 mmol, 3.00 eq) and Compound B2 (150 mg, 869 μmol, 1.00 eq) were added at 80° C., and stirring was continued for 1.5 hours. The reaction solution was concentrated, and the residues were purified by preparative chromatography (TFA condition) to obtain Compound 7 (60.0 mg, 80.3 μmol, 9.24% yield, 90.5% purity) as a brown solid.

¹H NMR (400 MHz, CDCl₃) δ 8.34 (t, J=14 Hz, 2H), 7.36-7.40 (m, 4H), 7.29-7.35 (m, 3H), 7.26-7.27 (m, 2H), 7.25-7.26 (m, 1H), 7.13-7.25 (m, 1H), 6.85 (d, J=7.6 Hz, 2H), 6.37 (d, J=14 Hz, 1H), 6.13 (d, J=14 Hz, 1H), 4.41 (s, 2H), 4.12-4.17 (m, 2H), 3.97 (s, 2H), 3.82-3.85 (m, 2H), 2.70-2.74 (m, 2H), 2.65-2.68 (m, 4H), 1.91-1.93 (m, 2H), 1.74 (s, 6H), 1.72 (s, 6H), 1.44 (t, J=7.2 Hz, 3H).

LCMS: m/z=675.4 (M+H)⁺.

### Example 8: Preparation of 2-((E)-2-((E)-2-chloro-3-(2-((E)-3,3-dimethyl-1-(2-(2-oxo-2-phenoxyethoxy)ethyl)indolin-2-ylidene)ethenyl)cyclohex-1-en-1-yl)ethenyl)-1-ethyl-3,3-dimethyl-3H-indol-1-ium Trifluoroacetate (8)

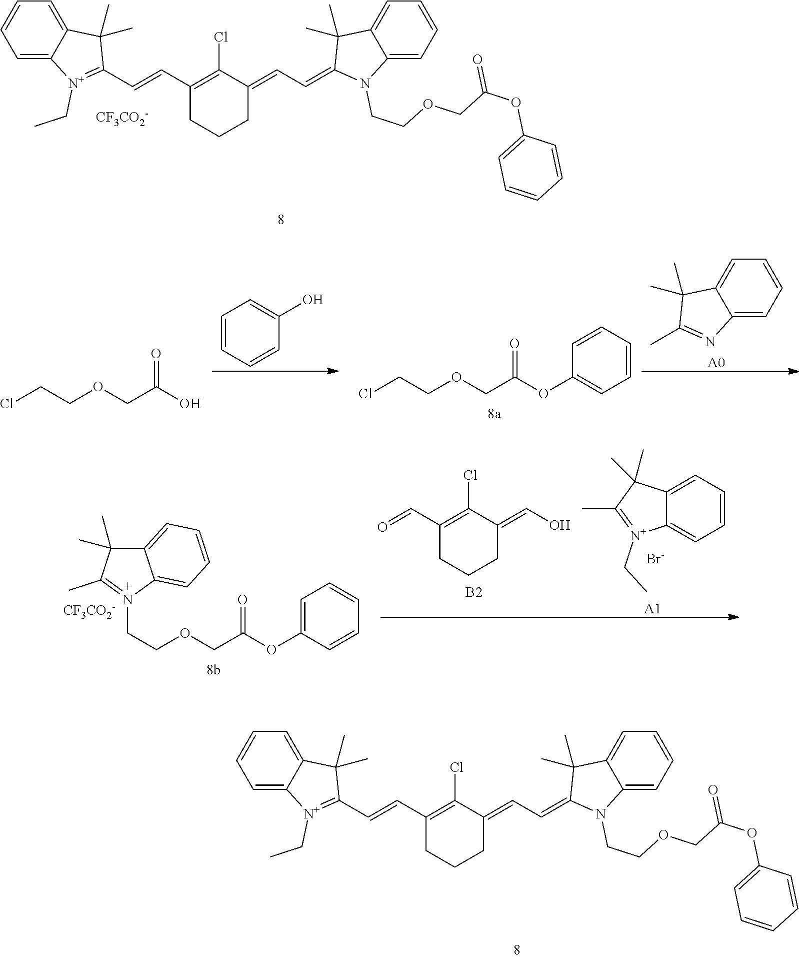

### Step 1: Preparation of phenyl 2-(2-chloroethoxy)acetate (8a)

HBTU (16.4 g, 43.3 mmol, 1.20 eq) and DIEA (11.7 g, 90.2 mmol, 15.7 mL, 2.50 eq) were added to a solution of 2-(2-chloroethoxy)acetic acid (5.00 g, 36.1 mmol, 1.00 eq) in DCM (70.0 mL). After stirring at 25° C. for 30 minutes, phenol (3.74 g, 39.7 mmol, 3.49 mL, 1.10 eq) was added and stirred for 12 hours at 25° C. The solvent was removed through concentration. Water (100 mL) was added, and the resulting mixture was extracted with ethyl acetate (100 mL×3). The organic phases were combined, dried over Na₂SO₄, and filtered. The filtrate was concentrated under reduced pressure. The residues were purified by silica gel column chromatography (petroleum ether/ethyl acetate=100/1 to 1/1) and thin-layer chromatography (petroleum ether/ethyl acetate=3/1, R_{f}=0.5, KMnO₄) to obtain Compound 8a (6.40 g, 29.8 mmol, yield 82.6%) as a yellow solid.

¹H NMR (400 MHz, CDCl₃) δ 7.39-7.43 (m, 2H), 7.27-7.29 (m, 1H), 7.13-7.15 (m, 2H), 4.45 (s, 2H), 3.95 (t, J=5.8 Hz, 2H), 3.73 (t, J=5.6 Hz, 2H).

### Step 2: Preparation of 2,3,3-trimethyl-1-(2-(2-oxo-2-phenoxyethoxy)ethyl)-3H-indol-1-ium Trifluoroacetate (8b)

A mixture of Compound 8a (4.44 g, 20.7 mmol, 2.00 eq), Compound A0 (1.65 g, 10.4 mmol, 1.00 eq) and NaI (7.77 g, 51.8 mmol, 5.00 eq) was heated to 180° C., and stirred for 1 hour. After cooling, the parent solution was diluted with ACN (30.0 mL), filtered and concentrated. The concentrate was purified by preparative chromatography (TFA condition) to obtain Compound 8b (1.20 g, 3.55 mmol) as a brown oil.

LCMS: m/z=338.1 (M+H)⁺.

### Step 3: Preparation of Compound 8

A solution of Compound 8b (588 mg, 1.74 mmol, 1.50 eq) and Compound A1 (366 mg, 1.16 mmol, 1.00 eq) in acetic anhydride (2.00 mL) was stirred at 70° C. for 1.5 hours, then NaOAc (285 mg, 3.48 mmol, 3.00 eq) and Compound B2 (200 mg, 1.16 mmol, 1.00 eq) were added, and stirred at 80° C. for another 1.5 hours. The mixture was concentrated under reduced pressure, and the residues were purified by preparative chromatography (0.1% TFA condition) to obtain Compound 8 (92.0 mg, 106 μmol, 9.17% yield, 89.8% purity, TFA) as a green solid.

¹H NMR (400 MHz, CDCl₃) δ 8.30-8.39 (m, 2H), 7.35-7.42 (m, 7H), 7.24-7.26 (m, 3H), 7.12 (d, 8.0 Hz, 1H), 7.00-7.02 (m, 2H), 6.44 (d, J=14.4 Hz, 1H), 6.13 (d, J=14 Hz, 1H), 4.49-4.51 (m, 2H), 4.35 (s, 2H), 4.10-4.17 (m, 4H), 2.63-2.69 (m, 4H), 1.92 (s, 1H), 1.90-1.91 (m, 2H), 1.72 (s, 12H), 1.42-1.46 (m, 3H).

LCMS: m/z=661.5 (M+H)⁺.

### Example 9: Preparation of 2-((E)-2-((E)-2-chloro-3-(2-((E)-3,3-dimethyl-1-(6-oxo-6-phenoxyhexyl)indolin-2-ylidene)ethenyl)cyclopent-1-en-1-yl)ethenyl)-1,3,3-trimethyl-3H-indol-1-ium Chloride (9)

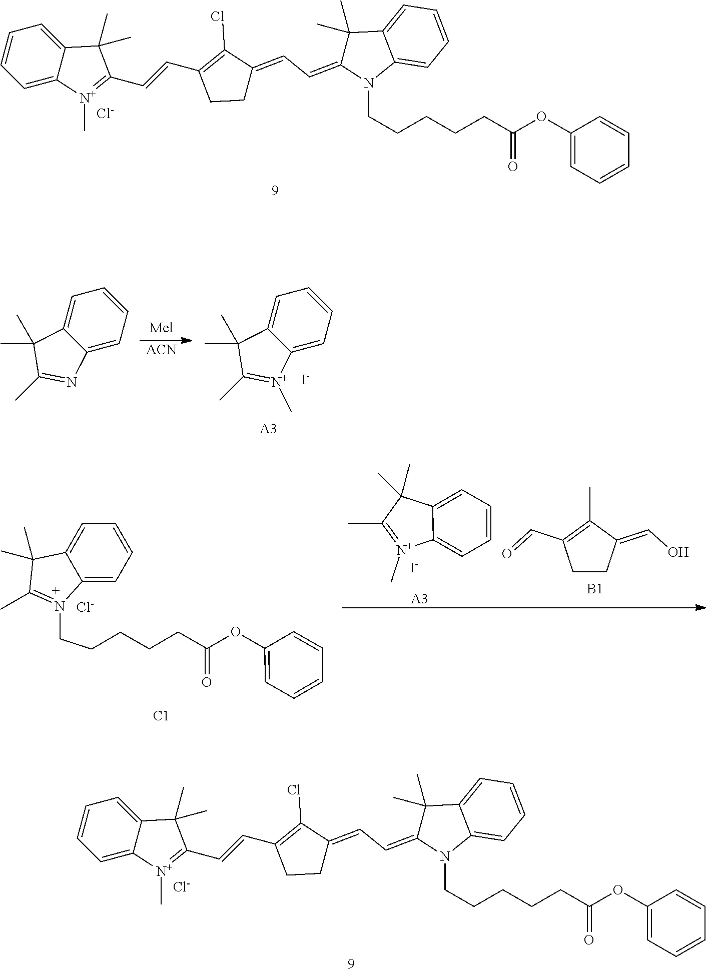

### Step 1: Preparation of 1,2,3,3-tetramethyl-3H-indol-1-ium Iodide (A3)

A solution of 2,3,3-trimethylindole (3.00 g, 18.8 mmol, 1.00 eq) and MeI (4.01 g, 28.2 mmol, 1.76 mL, 1.50 eq) in ACN (30.0 mL) was evacuated and purged with nitrogen 3 times. The mixture was stirred at 90° C. under nitrogen atmosphere for 12 hours. The reaction solution was concentrated under reduced pressure to remove the solvent, thereby obtaining a crude produce. Then, the crude product was pulped with MTBE/DCM=5:1 (20 mL) at 25° C. for 30 minutes, and filtered to obtain a filter cake, i.e., a product of Compound A3 (3.50 g, 11.4 mmol, 60.4% yield, 98% purity), which was a purple solid.

### Step 2: Preparation of Compound 9

A mixture of Compound C1 (2.00 g, 5.71 mmol, 1.50 eq), Compound A3 (1.15 g, 3.80 mmol, 1.00 eq) and Ac₂O (30.0 mL) was stirred at 70° C. for 1 hour, and then NaOAc (936 mg, 11.4 mmol, 3.00 eq) and Compound B1 (603 mg, 3.80 mmol, 1.00 eq) were added successively. The reaction mixture was stirred at 80° C. for 1 hour and concentrated under reduced pressure. The residues were purified by preparative chromatography (0.1% HCl condition) to obtain Compound 9 (900 mg, 1.23 mmol, 32.21% yield, 88% purity) as a green solid.

LCMS: m/z=645.2, C₄₂H₄₆ClN₂O₂⁺.

### Example 10: Preparation of 2-((E)-2-((E)-3-(2-((E)-1-(5-carboxypentyl)-3,3-dimethylindolin-2-ylidene)ethenyl)-2-chlorocyclopent-1-en-1-yl)ethenyl)-1,3,3-trimethyl-3H-indol-1-ium Chloride (10)

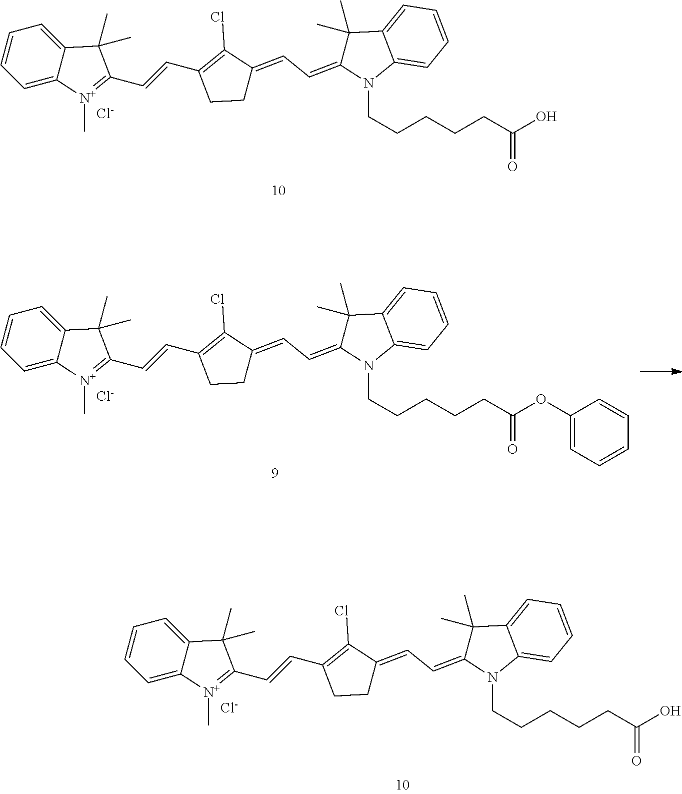 [SMILES: C[N+]1=C(/C=C/C2=C(Cl)C(=C/C=C3/N(CCCCCC(=O)O)c4ccccc4C3(C)C)/CC2)C(C)(C)c2ccccc21 ; [Cl-] ; C[N+]1=C(/C=C/C2=C(Cl)C(=C/C=C3/N(CCCCCC(=O)Oc4ccccc4)c4ccccc4C3(C)C)/CC2)C(C)(C)c2ccccc21 ; [Cl-] ; C[N+]1=C(/C=C/C2=C(Cl)C(=C/C=C3/N(CCCCCC(=O)O)c4ccccc4C3(C)C)/CC2)C(C)(C)c2ccccc21 ; [Cl-]]

HCl/dioxane solution (1 M, 1.86 mL, 6.00 eq) was added to a solution of Compound 9 (200 mg, 309 μmol, 1.00 eq) in dioxane (2.00 mL). The solution was kept at 50° C. and stirred for 2 hours. The reaction solution was concentrated, and the residues were purified by preparative chromatography (column: YMC Triart C18 150 mm×25 mm×5 μm; mobile phase: [water (HCl)-ACN]; B %: 58%-88%, 10 min) to obtain Compound 10 (41.0 mg, 71.0 μmol, 23.0% yield, 98.7% purity) as a green solid.

¹H NMR (400 MHz, CDCl₃) δ 7.85-7.81 (m, 2H), 7.41-7.36 (m, 4H), 7.25-7.22 (m, 2H), 7.15-7.13 (m, 2H), 6.08-6.03 (m, 2H), 4.08-4.06 (m, 2H), 3.67 (s, 3H), 3.03 (s, 4H), 2.54-2.49 (m, 2H), 1.87-1.76 (m, 4H), 1.71 (s, 12H), 1.60-1.54 (m, 2H).

LCMS: m/z=569.4, C₃₆H₄₂ClN₂O₂⁺.

### Example 11: Preparation of 1-(5-carboxyphenyl)-2-((E)-2-((E)-2-chloro-3-(2-((E)-3,3-dimethyl-1-(6-oxo-6-phenoxyhexyl)indolin-2-ylidene)ethenyl)cyclohex-1-en-1-yl)ethenyl)-3,3-dimethyl-3H-indol-1-ium Chloride (11)

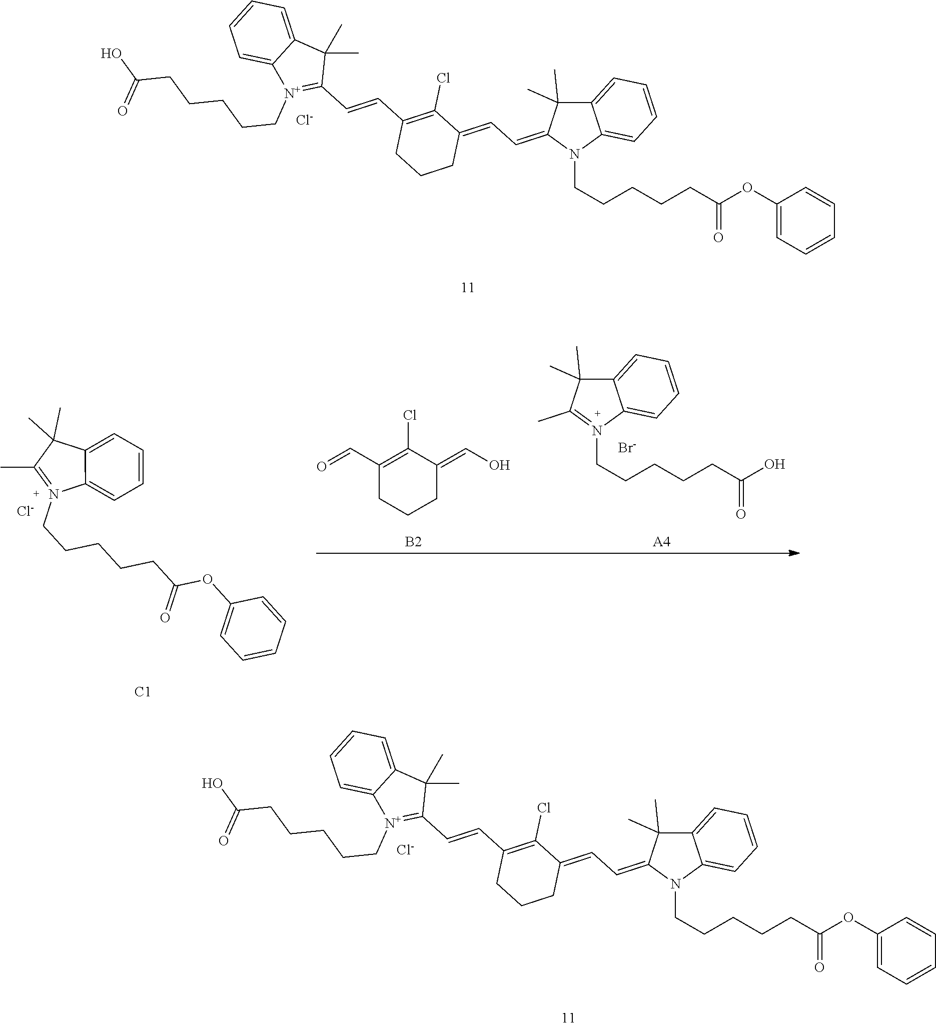

NaOAc (570 mg, 6.95 mmol, 3.00 eq) and Compound C0 (635 mg, 2.32 mmol, 1.00 eq) were added to a solution of Compound B2 (0.400 g, 2.32 mmol, 1.00 eq) and Compound C1 (812 mg, 2.32 mmol, 1.00 eq) in EtOH (2.00 mL). After stirring at 85° C. for 2 hours, the reaction solution was concentrated, and the residues were purified by preparative chromatography (column: YMC Triart C18 150 mm×25 mm×5 μm; mobile phase: [water (HCl)-ACN]; B %: 56%-86%, 8.5 min) to obtain Compound 11 (94.0 mg, 111 μmol, 4.81% yield, 90.2% purity) as a green solid.

¹H NMR (400 MHz, CDCl₃) δ 8.40 (d, J=14.0 Hz, 1H), 8.31 (d, J=14.0 Hz, 1H), 7.38-7.43 (m, 6H), 7.35-7.37 (m, 1H), 7.21-7.23 (m, 3H), 7.02-7.04 (m, 3H), 6.31 (d, J=14.0 Hz, 1H), 6.10 (d, J=14.0 Hz, 1H), 4.06-4.19 (m, 4H), 2.69-2.75 (m, 4H), 2.61-2.64 (m, 4H), 1.82-1.99 (m, 10H), 1.72 (d, J=5.6 Hz, 12H), 1.59-1.64 (m, 4H).

LCMS: m/z=759.4, C₄₈H₅₆ClN₂O₄⁺.

### Example 12: Preparation of 2-((E)-2-((E)-3-(2-((E)-3,3-dimethyl-1-(6-oxo-6-phenoxyhexyl)indolin-2-ylidene)ethenyl)-2-phenoxycyclohex-1-en-1-yl)ethenyl)-1-ethyl-3,3-dimethyl-3H-indol-1-ium Chloride (12)

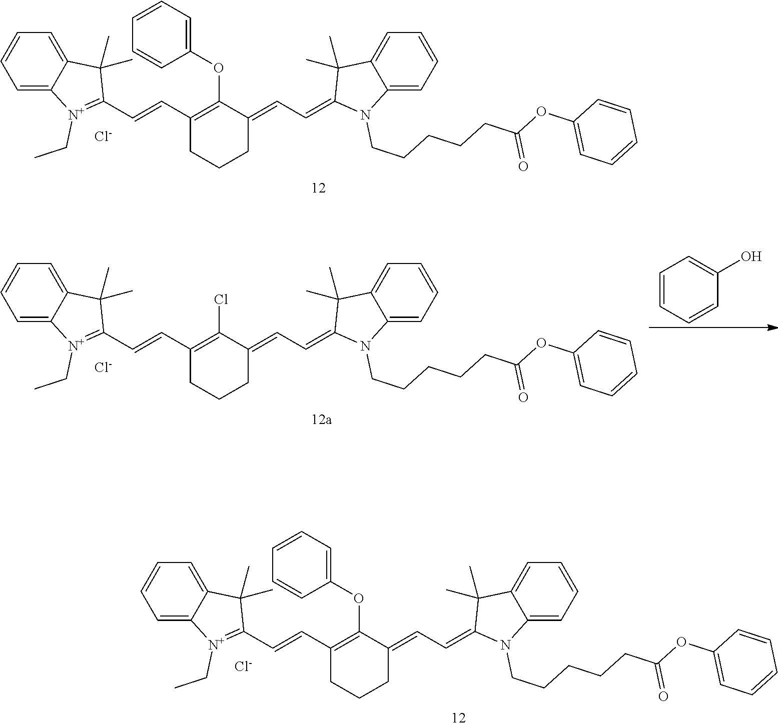

NaH (88.9 mg, 2.22 mmol, 60.0% purity, 2.50 eq) was added to a solution of phenol (209 mg, 2.22 mmol, 196 μL, 2.50 eq) in THF (50.0 mL) at 0° C., and then heated to 25° C. and stirred for 30 minutes. A solution of 2-((E)-2-((E)-2-chloro-3-(2-((E)-3,3-dimethyl-1-(6-oxo-6-phenoxyhexyl)indolin-2-ylidene)ethenyl)cyclohex-1-en-1-yl)ethenyl)-1-ethyl-3,3-dimethyl-3H-indol-1-ium chloride (12a) (see Comparative Example 1 for the preparation method) (600 mg, 890 μmol, 1.00 eq) in THF (10.0 mL) was added. After stirring for 4 hours, the resulting mixture was cooled to 0° C. and quenched by addition of saturated NH₄Cl solution (4.00 mL). The mixture was concentrated under reduced pressure, and the residues were purified by preparative chromatography (HCl condition: column: Phenomenex luna C18 150 mm×40 mm×15 μm; mobile phase: [water (HCl)-ACN]; B %: 60%-90%, 10 min) to obtain Compound 12 (84.7 mg, 110 μmol, 12.4% yield, purity 95.3%) as a green solid.

¹H NMR: (400 MHz, CDCl₃) δ 7.91 (t, J=13.8 Hz, 2H), 7.34-7.37 (m, 6H), 7.23-7.27 (m, 5H), 7.02-7.08 (m, 7H), 6.08 (t, J=14.4 Hz, 2H), 4.06-4.16 (m, 4H), 2.72 (s, 4H), 2.59-2.71 (m, 2H), 2.02-2.03 (m, 2H), 1.82-1.86 (m, 4H), 1.58-1.60 (m, 2H), 1.38-1.43 (m, 3H), 1.33 (d, J=2.0 Hz, 12H).

LCMS: m/z=731.6 (M+H)⁺.

### Example 13: Preparation of 2-((E)-2-((E)-2-((5-carboxypentyl)amino)-3-(2-((E)-1-ethyl-3,3-dimethylindolin-2-ylidene)ethenyl)cyclohex-1-en-1-yl)ethenyl)-1-ethyl-3,3-dimethyl-3H-indol-1-ium Formate (13)

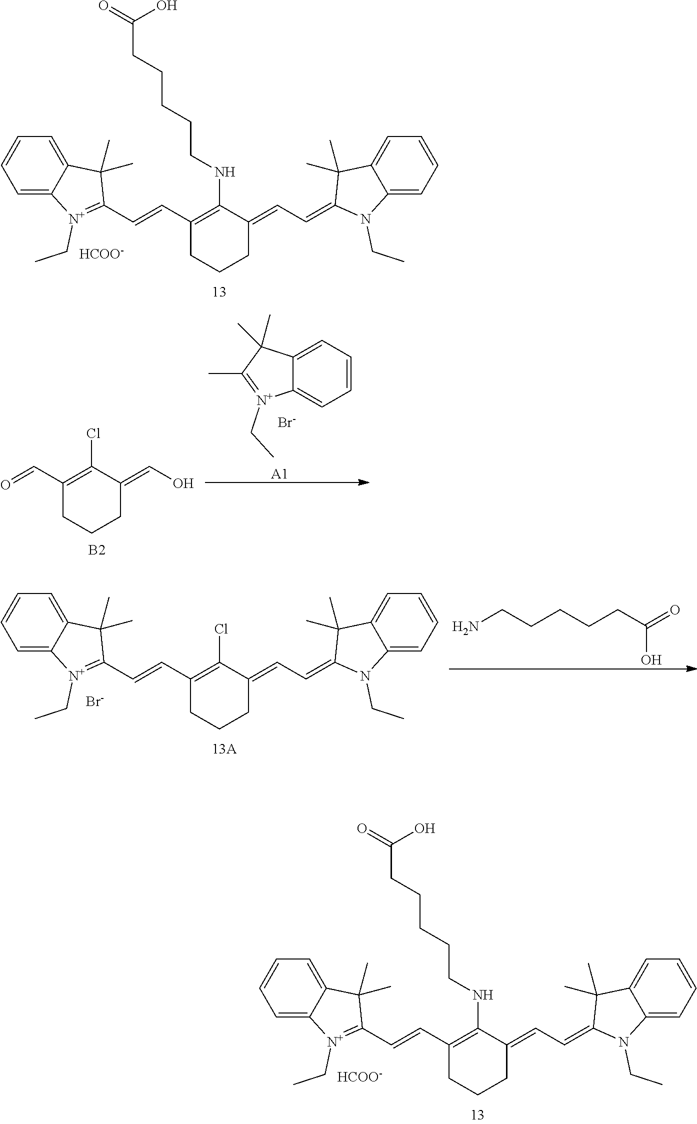

### Step 1: Preparation of 2-((E)-2-((E)-2-chloro-3-(2-((E)-1-ethyl-3,3-dimethyl-indolin-2-ylidene)ethenyl)cyclohex-1-en-1-yl]ethenyl]-1-ethyl-3,3-dimethyl-indol-1-ium Bromide (13A)

NaOAc (1.43 g, 17.4 mmol, 3.00 eq) and Compound B2 (1.00 g, 5.79 mmol, 1.00 eq) were added to a mixture of Compound A1 (3.88 g, 14.5 mmol, 2.50 eq) and Ac₂O (50.0 mL) at 70° C. The mixture was heated to 80° C. and stirred for 1 hour. The reaction solution was concentrated, and then purified by preparative chromatography (neutral condition) to obtain Compound 13A (1.30 g) as a green solid after lyophilization.

LCMS: m/z=511.2.

### Step 2: Preparation of Compound 13

Triethylamine (324 mg, 3.20 mmol, 446 μL, 16.4 eq) was added to a solution of Compound 13A (0.100 g, 195 μmol, 1.00 eq), 6-aminohexanoic acid (169 mg, 1.29 mmol, 166 μL, 6.60 eq) and MeOH (5.50 mL), and the reaction mixture was stirred at 70° C. for 4 hours. After concentrated under reduced pressure, the residues were purified by preparative chromatography (chromatographic column: Phenomenex luna C18 150 mm×25 mm×10 μm; mobile phase: [water (FA)-ACN]; B %: 38%-68%, 10 min) to obtain Compound 13 (56.0 mg, 83.9 μmol, 42.9% yield, purity 90.9%) as a green solid.

¹H NMR (400 MHz, CDCl₃) δ 8.70 (s, 1H), 7.64-7.67 (m, 2H), 7.28 (s, 1H), 7.23-7.26 (m, 3H), 7.02-7.06 (m, 2H), 6.81 (d, J=8.4 Hz, 2H), 5.52-5.55 (m, 2H), 3.79-3.83 (m, 6H), 2.49-2.53 (m, 6H), 1.95-1.98 (m, 2H), 1.81-1.84 (m, 2H), 1.75-1.79 (m, 2H), 1.66 (s, 12H), 1.45-1.49 (m, 2H), 1.31-1.37 (m, 6H).

LCMS: m/z=606.5.

### Example 14: Preparation of 2-((E)-2-((E)-4-chloro-5-(2-((E)-3,3-dimethyl-1-(6-oxo-6-phenoxyhexyl)indolin-2-ylidene)ethenyl)-1,1-dimethyl-1,2,5,6-tetrahydropyridin-1-ium-3-yl)ethenyl)-3,3-dimethyl-1-(6-oxo-6-phenoxyhexyl)-3H-indol-1-ium Dichloride (14)

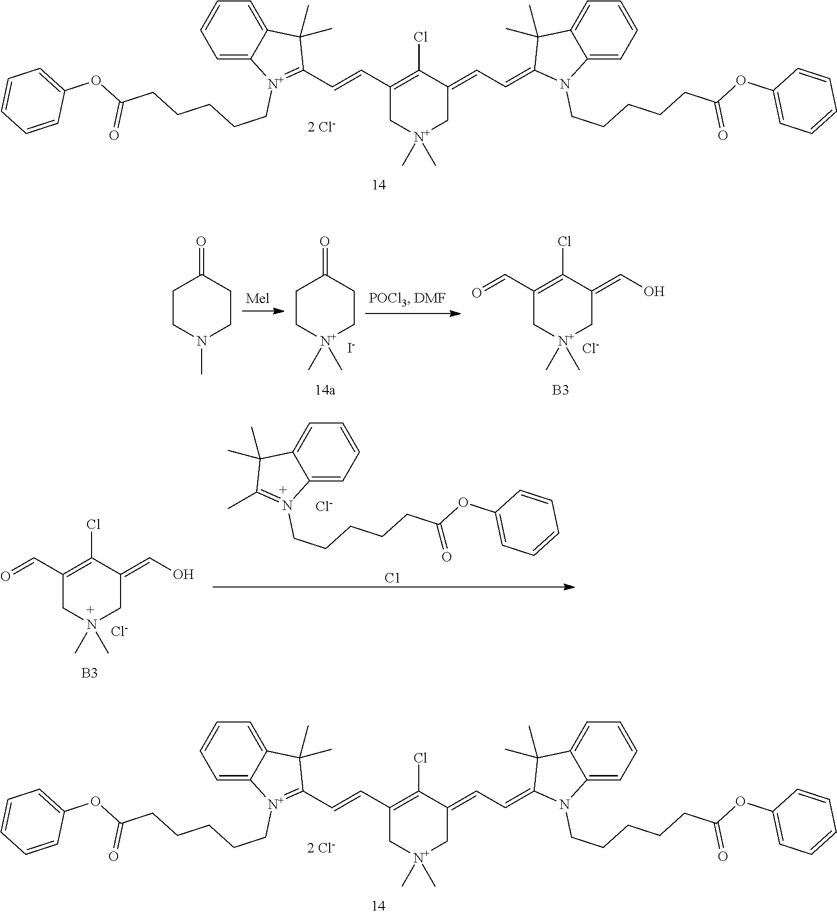

### Step 1: Preparation of 1,1-dimethyl-4-oxopiperidin-1-ium Iodide (14a)

MeI (41.02 g, 288.98 mmol, 17.99 mL, 2.18 eq) was added to a solution of 1-methyl-4-piperidone (15 g, 132.56 mmol, 15.42 mL, 1 eq) in acetone (210 mL) at 0° C., and the mixture was stirred at 25° C. for 4 hours. The reaction solution was filtered, and the filter cake was washed with iced acetone (20.0 mL). The filtrate was then concentrated under reduced pressure, and the residue was a product (33 g, 129.36 mmol, 97.59% yield) as a yellow solid.

¹H NMR (400 MHz, CDCl₃) δ 3.77-3.74 (t, J=6.8 Hz, 4H), 3.28 (s, 6H), 2.73-2.69 (m, 4H).

### Step 2: Preparation of (E)-4-chloro-5-formyl-3-(hydroxymethylene)-1,1-dimethyl-1,2,3,6-tetrahydropyridin-1-ium Chloride (B3)

A mixture of DMF (210 mL) and POCl₃ (209.17 g, 1.36 μmol, 126.77 mL, 3 eq) was stirred for 0.5 hour, and then the product (116 g, 454.73 mmol, 1 eq) obtained in the previous step was added to the mixture. The mixture was then stirred for at 80° C. 3 hours. TLC (ethyl acetate:methanol=5:1) showed the product obtained in the previous step was consumed completely. The reaction mixture was quenched with 20% HCl (700 mL) at 35 to 40° C., then the mixture was cooled to −20° C. quickly, and the suspension was filtered. The filtrate was left at 25° C. for 12 hours, then cooled to −20° C. and left for 4 hours. The suspended matter was filtered. The filter cake was concentrated under reduced pressure to obtain a residue. The residues were diluted with toluene (30.0 mL), and concentrated under reduced pressure to obtain a second residue, i.e., Compound B3 (24.2 g, 110.50 mmol, 26.26% yield) as a yellow solid.

¹H NMR (400 MHz, CDCl₃) δ 9.00 (s, 2H), 4.27 (s, 4H), 3.18 (s, 6H).

### Step 3: Preparation of Compound 14

After a mixture of Compound C1 (865 mg, 2.47 mmol, 2.00 eq) and Ac₂O (10.0 mL) was stirred at 70° C. for 30 minutes, NaOAc (304 mg, 3.70 mmol, 3.00 eq) and Compound B3 (250 mg, 1.23 mmol, 1.00 eq) were added. The mixture was kept at 80° C. and stirred for 1 hours. The reaction mixture was concentrated under reduced pressure, and the residues were purified by preparative chromatography (0.1% HCl condition) to obtain Compound 14 (182.57 mg, 190 μmol, 15.4% yield, 90.1% purity) as a green solid.

¹H NMR (400 MHz, CDCl₃) δ 8.28 (d, J=7.4 Hz, 2H), 7.34-7.44 (m, 10H), 7.19-7.25 (m, 4H), 7.04 (d, J=8.0 Hz, 4H), 6.22-6.26 (m, 2H), 4.39 (s, 4H), 4.24-4.33 (m, 4H), 3.08 (s, 6H), 2.60 (t, J=4.0 Hz, 4H), 2.02 (s, 2H), 1.88-1.95 (m, 4H), 1.82-1.86 (m, 4H), 1.73 (s, 12H), 1.66 (s, 2H).

LCMS: m/z=433.4, C₅₅H₆₄ClN₃O₄²⁺.

### Example 15: Preparation of 2-((E)-2-((E)-5-(2-((E)-1-(5-carboxypentyl)-3,3-dimethylindolin-2-ylidene)ethenyl)-4-chloro-1,1-dimethyl-1,2,5,6-tetrahydropyridin-1-ium-3-yl)ethenyl)-3,3-dimethyl-1-(6-oxo-6-phenoxyhexyl)-3H-indol-1-ium Ditrifluoroacetate (15)

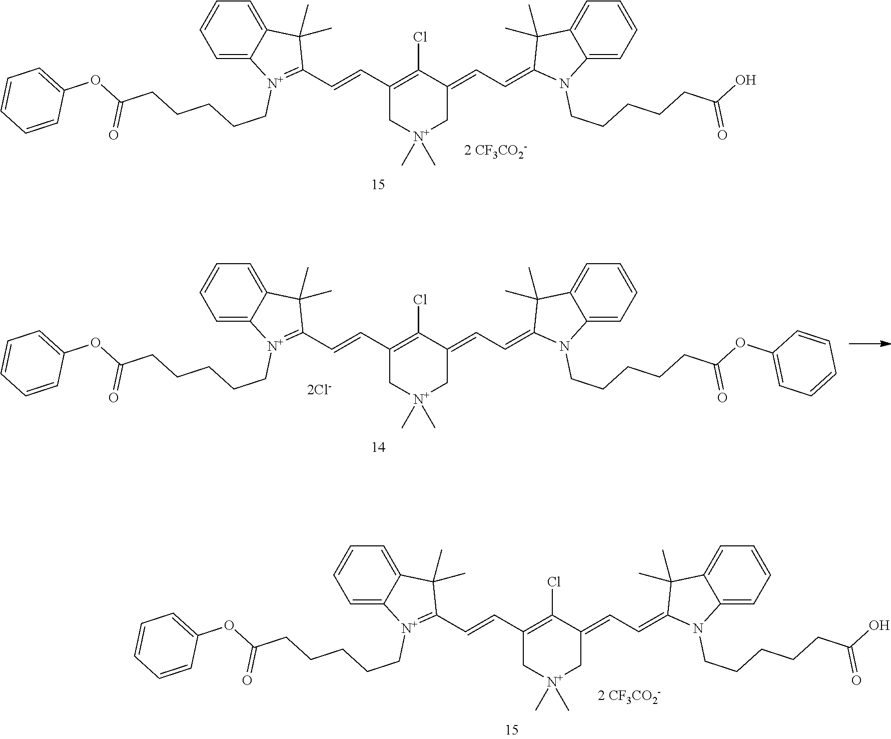 [SMILES: CC1(C)C(/C=C/C2=C(Cl)C(=C/C=C3/N(CCCCCC(=O)O)c4ccccc4C3(C)C)/C[N+](C)(C)C2)=[N+](CCCCCC(=O)Oc2ccccc2)c2ccccc21 ; CC1(C)C(/C=C/C2=C(Cl)C(=C/C=C3/N(CCCCCC(=O)Oc4ccccc4)c4ccccc4C3(C)C)/C[N+](C)(C)C2)=[N+](CCCCCC(=O)Oc2ccccc2)c2ccccc21 ; CC1(C)C(/C=C/C2=C(Cl)C(=C/C=C3/N(CCCCCC(=O)O)c4ccccc4C3(C)C)/C[N+](C)(C)C2)=[N+](CCCCCC(=O)Oc2ccccc2)c2ccccc21]

A solution of hydrogen chloride in dioxane (4.00 M, 86.6 μL, 3.00 eq) was added to a solution of Compound 14 (100 mg, 115 μmol, 1.00 eq) in MeCN (2.00 mL), and stirred at 25° C. for 12 hours, followed by the addition of a solution of hydrogen chloride in dioxane (4.00 M, 289 μL, 10.0 eq) again. After stirring at 25° C. for another 12 hours, the solvent was concentrated, and the residues were purified by preparative chromatography (TFA condition; column: Phenomenex Synergi Polar-RP 100 mm×25 mm×4 μm; mobile phase: [water (TFA)-ACN]; B %: 38%-68%, 9 min) to obtain Compound 15 (43.44 mg, 43.0 μmol, 37.3% yield, 89.5% purity, TFA) after lyophilization as a blue paste.

¹H NMR (400 MHz, CDCl₃) δ 8.30 (d, J=14.8 Hz, 2H), 7.34-7.42 (m, 8H), 7.18-7.23 (m, 3H), 7.00-7.23 (m, 2H), 6.39-6.45 (m, 2H), 4.76 (d, J=14.8 Hz, 4H), 4.24-4.25 (m, 4H), 3.34 (s, 6H), 2.53-2.63 (m, 2H), 2.32-2.42 (m, 2H), 1.82-1.87 (m, 7H), 1.72 (s, 12H), 1.54-1.59 (m, 5H).

LCMS: m/z=395.0 C₄₉H₆₀ClN₃O₄²⁺.

### Example 16: 2-((E)-2-((E)-4-chloro-5-(2-((E)-3,3-dimethyl-1-(6-oxo-6-phenoxyhexyl)indolin-2-ylidene)ethenyl)-1,1-dimethyl-1,2,5,6-tetrahydropyridin-1-ium-3-yl)ethenyl)-1-ethyl-3,3-dimethyl-3H-indol-1-ium Dichloride (16)

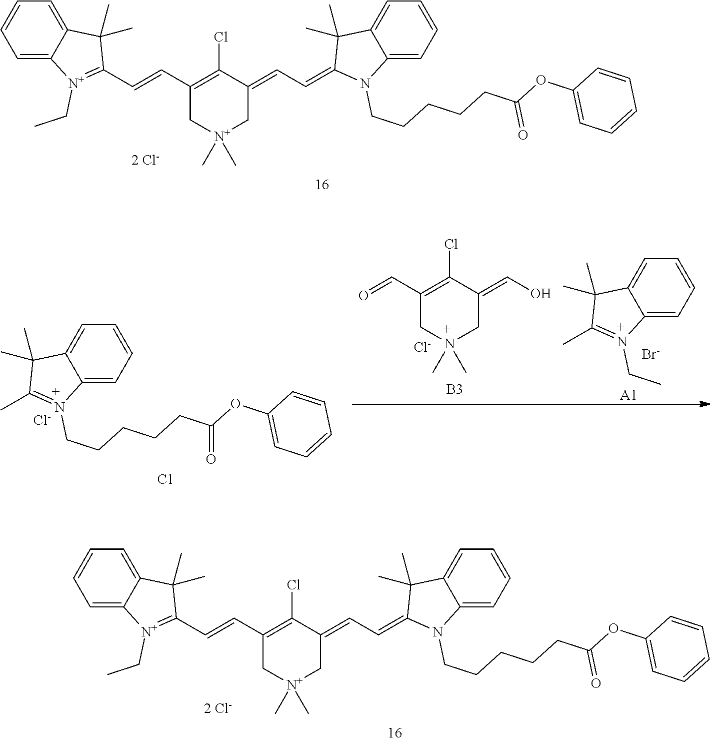

A mixture of Compound C1 (2.00 g, 5.71 mmol, 1.50 eq), Compound A1 (716 mg, 3.81 mmol, 1.00 eq, the amount used being only based on positive ions) and Ac₂O (30.0 mL) was heated to 80° C. and stirred for 1 hours. Sodium acetate (936 mg, 11.4 mmol, 3.00 eq) and Compound B3 (771 mg, 3.81 mmol, 1.00 eq) were added successively, and stirred at 80° C. for 1 hour. The reaction solution was concentrated under reduced pressure, and the residues were purified by preparative chromatography (column: Phenomenex luna C18 150 mm×40 mm×15 μm; mobile phase: [water (HCl)-ACN]; B %: 30%-60%, 10 min) to obtain Compound 16 (121 mg, 166 μmol, 4.38% yield, 97.1% purity) as a green solid.

¹H NMR: (400 MHz, CDCl₃) δ 8.28-8.33 (m, 2H), 7.34-7.45 (m, 9H), 7.24-7.26 (m, 2H), 7.04-7.06 (m, 2H), 6.20-6.28 (m, 2H), 4.43 (d, J=10.8 Hz, 4H), 4.27-4.32 (m, 4H), 3.20 (s, 6H), 2.61 (t, J=14.0 Hz, 2H), 1.83-1.93 (m, 4H), 1.74 (d, J=2.4 Hz, 12H), 1.59-1.64 (m, 2H), 1.47-1.50 (s, 3H).

LCMS: m/z=352.0, C₄₅H₅₄ClN₃O₂²⁺.

### Example 17: Preparation of 1-(2-carboxyethyl)-2-((E)-2-((E)-5-(2-((E)-1-(2-carboxyethyl)-3,3-dimethylindolin-2-ylidene)ethenyl)-4-chloro-1,1-dimethyl-1,2,5,6-tetrahydropyridin-1-ium-3-yl)ethenyl)-3,3-dimethyl-3H-indol-1-ium Ditrifluoroacetate (17)

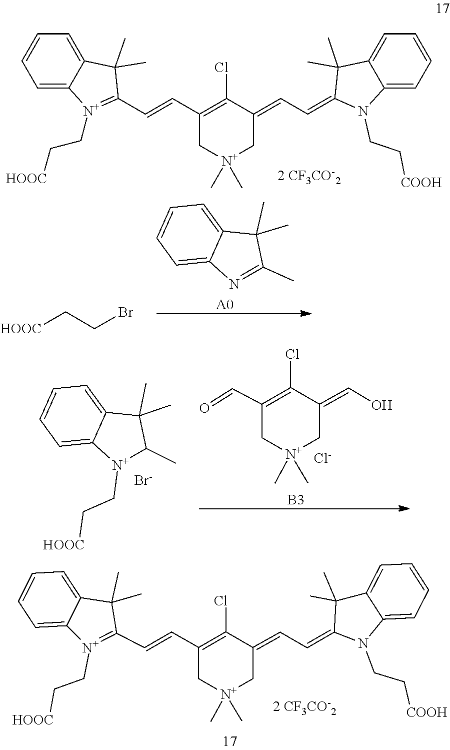

### Step 1: Preparation of 1-(2-carboxyethyl)-2,3,3-trimethyl-3H-indol-1-ium Bromide

2,3,3-Trimethylindole (A0) (3.00 g, 18.8 mmol, 1.00 eq) was added to a solution of 3-bromopropionic acid (5.76 g, 37.7 mmol, 3.89 mL, 2.00 eq) in ACN (30 mL), and stirred at 80° C. for 24 hours. The reaction mixture was concentrated under reduced pressure. The residues were pulped with DCM:MTBE=5:1 (10.0 mL) to obtain the title compound (1.50 g, 5.94 mmol, 31.5% yield, 92.0% purity) as a pink solid.

LCMS: m/z=232.2, C₁₄H₁₈NO₂⁺.

### Step 2: Preparation of Compound 17

NaOAc (353 mg, 4.30 mmol, 1.00 eq) was added to a solution of the compound (1.00 g, 4.30 mmol, 1.00 eq) obtained in Step 1 in EtOH (50.0 mL), and the reaction mixture was stirred at 25° C. for 0.5 hour. Compound B3 (436 mg, 2.15 mmol, 0.500 eq) was added, and subjected to the reaction at 80° C. for 5 hours. The resulting mixture was purified by preparative chromatography (column: Phenomenex Synergi Polar-RP 100 mm×25 mm×4 μm; mobile phase: [water (TFA)-ACN]; B %: 30%-60%, 9 min) to obtain Compound 17 (174 mg, 247 μmol, 5.73% yield, 89.4% purity) as a green solid.

¹H NMR (400 MHz, DMSO-d6) δ 8.30-8.26 (m, 2H), 7.44-7.37 (m, 4H), 7.32-7.28 (m, 4H), 6.56-6.52 (m, 2H), 4.78 (s, 4H), 4.54 (s, 4H), 3.43 (s, 6H), 2.91 (s, 4H), 1.71 (s, 12H).

LCMS: m/z=314.8, C₃₇H₄₄ClN₃O₄₂⁺.

### Example 18: Preparation of 2-((E)-2-((E)-4-chloro-5-(2-((E)-1-ethyl-3,3-dimethylindolin-2-ylidene)ethenyl)-1,1-dimethyl-1,2,5,6-tetrahydropyridin-1-ium-3-yl)ethenyl)-1-ethyl-3,3-dimethyl-3H-indol-1-ium Ditrifluoroacetate (18)

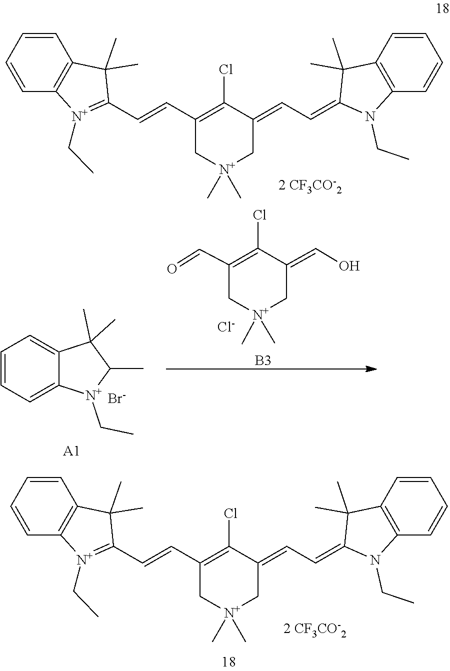

A solution of Compound A1 (1.86 g, 9.87 mmol, 2.00 eq) in Ac₂O (15.0 mL) was stirred for 0.5 h at 70° C. NaOAc (1.21 g, 14.8 mmol, 3.00 eq) and Compound B3 (1.00 g, 4.93 mmol, 1.00 eq) were added, and the reaction mixture was stirred at 80° C. for 1 hour. The reaction mixture was concentrated under reduced pressure. The residues were pulped with water for 15 minutes, and filtered. The resulting crude was purified by preparative chromatography (column: YMC Triart C18 150 mm×25 mm×5 μm; mobile phase: [water (TFA)-ACN]; B %: 41%-61%, 10 min) to obtain Compound 18 (150 mg, 272 μmol, 5.51% yield, 98.2% purity) as a green solid.

¹H NMR (400 MHz, CDCl₃) δ 8.32 (d, J=14.8 Hz, 2H), 7.41-7.46 (m, 4H), 7.36-7.34 (m, 2H), 7.22 (d, J=8.0 Hz, 2H), 6.68 (d, J=14.8 Hz, 2H), 4.98 (s, 4H), 4.38-4.43 (m, 4H), 3.53 (s, 6H), 1.76 (s, 12H), 1.46 (t, J=7.2 Hz, 6H).

LCMS: m/z=271.0, C₃₅H₄₄ClN₃²⁺.

### Example 19: Preparation of 2-((E)-2-((E)-4-chloro-5-(2-((E)-1-(2-hydroxyethyl)-3,3-dimethylindolin-2-ylidene)ethenyl)-1,1-dimethyl-1,2,5,6-tetrahydropyridin-1-ium-3-yl)ethenyl)-1-(2-hydroxyethyl)-3,3-dimethyl-3H-indol-1-ium Ditrifluoroacetate (19)

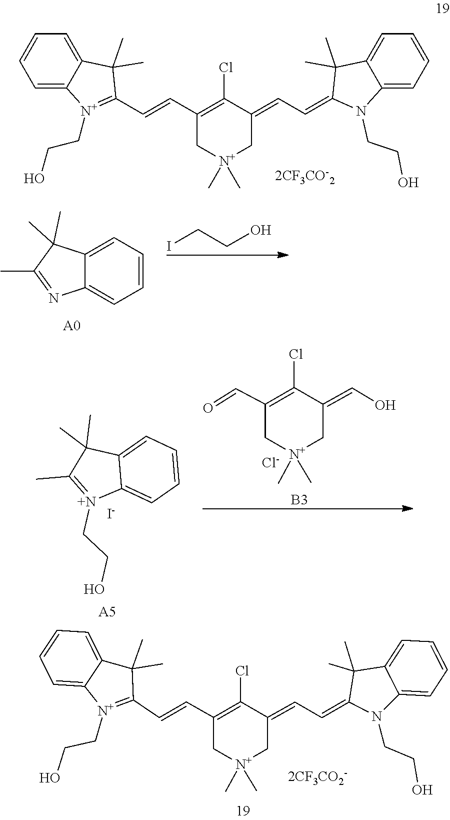

### Step 1: Preparation of 1-(2-hydroxyethyl)-2,3,3-trimethyl-3H-indol-1-ium Iodide (A5)

A mixture of Compound A0 (5.00 g, 31.4 mmol, 1.00 eq), 2-iodoethanol (5.40 g, 31.4 mmol, 2.45 mL, 1.00 eq) and EtOH (50.0 mL) was kept at 80° C. and stirred for 12 hours. Then, the reaction solution was concentrated under reduced pressure, and the residues were pulped with petroleum ether (100 mL) to obtain Compound A5 (2.80 g, 11.2 mmol, 35.8% yield, 82.0% purity) as a pink solid.

¹H NMR (400 MHz, CDCl₃) δ 7.81-7.79 (m, 1H), 7.60-7.57 (m, 3H), 4.90-4.88 (m, 2H), 4.21 (s, 2H), 3.12 (s, 3H), 1.66 (s, 6H).

LCMS: m/z=204.2 C₁₃H₁₈NO⁺.

### Step 2: Preparation of Compound 19

Compound A5 (1.00 g, 4.90 mmol, 1.00 eq) was dissolved in EtOH (50 mL), and NaOAc (402 mg, 4.90 mmol, 1.00 eq) was added and stirred at 25° C. for 0.5 hour. Then, Compound B3 (496 mg, 2.45 mmol, 0.500 eq) was added, and kept at 80° C. and stirred for 5 hours. The resulting mixture was concentrated under reduced pressure, and the residues were purified by preparative chromatography (column: Phenomenex Synergi Polar-RP 100 mm×25 mm×4 μm; mobile phase: [water (TFA)-ACN]; B %: 28%-58%, 9 min) to obtain Compound 19 (90.1 mg, 145.38 μmol, 3.34% yield, 92.65% purity) as a brown solid.

¹H NMR (400 MHz, DMSO-d6) δ 8.24-8.22 (m, 2H), 7.70-7.68 (m, 2H), 7.55-7.53 (m, 2H), 7.49-7.45 (m, 2H), 7.38-7.34 (m, 2H), 6.41-6.37 (m, 2H), 4.65 (s, 4H), 4.38-4.34 (m, 4H), 3.86-3.83 (m, 6H), 3.27 (s, 6H), 1.70 (s, 12H).

LCMS: m/z=286.9, C₃₅H₄₄ClN₃O₂²⁺.

### Example 20: Preparation of 2-((E)-2-((E)-3-(2-((E)-3,3-dimethyl-1-(6-oxo-6-phenoxyhexyl)indol-2-methylene)ethenyl)-2-(4-sulfophenoxy)cyclohex-1-en-1-yl)ethenyl)-1-ethyl-3,3-dimethyl-3H-indol-1-ium Formate (20)

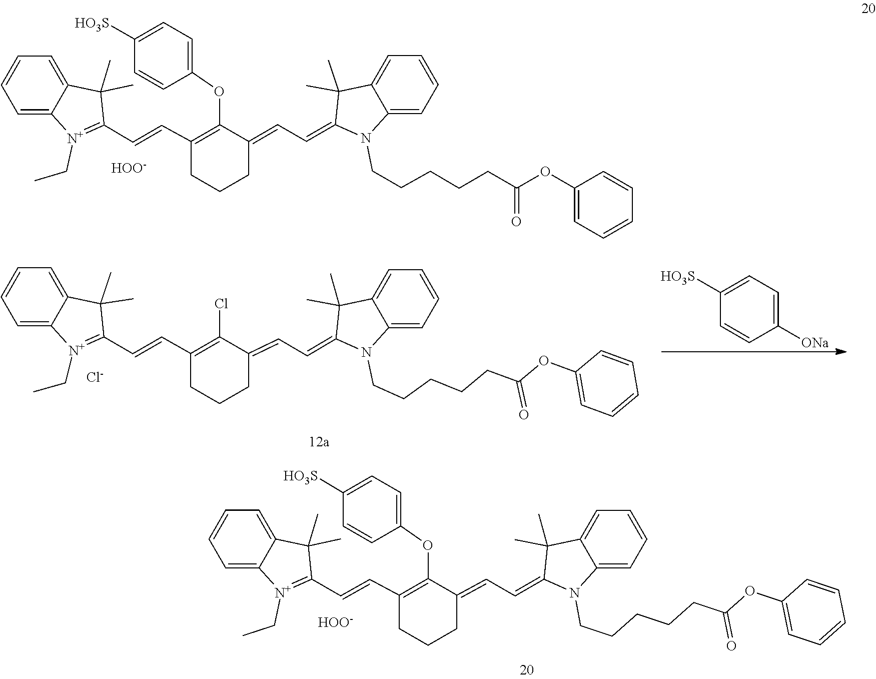

K₂CO₃ (30.7 mg, 222 μmol, 1.00 eq) was added to a mixture of (4-sulfophenoxy)sodium (52.3 mg, 266 μmol, 1.20 eq), Compound 12a (150 mg, 222 μmol, 1.00 eq) and DMSO (3.00 mL), and stirred at 40° C. for 12 hours. The reaction mixture was then filtered. The filtrate was purified by preparative chromatography (chromatographic column: UniSil 3-100 C18 Ultra (150 mm×25 mm×3 μm); mobile phase [water (FA)-ACN]; B %: 67%-97%, 10 min) to obtain Compound 20 (85.43 mg, 100 μmol, 45.2% yield, 95.5% purity) after lyophilization as a green solid.

¹H NMR (400 MHz, CDCl₃) δ 8.03 (d, J=8.8 Hz, 2H), 7.92 (dd, J=14.0, 9.6 Hz, 2H), 7.39-7.29 (m, 5H), 7.26-7.15 (m, 4H), 7.07-7.02 (m, 4H), 6.98-6.96 (m, 2H), 5.92 (dd, J=14.0, 11.6 Hz, 2H), 4.05-3.95 (m, 4H), 2.65-2.59 (m, 6H), 1.02-1.99 (m, 2H), 1.91-1.82 (m, 4H), 1.60-1.55 (m, 2H), 1.39 (t, J=5.4 Hz, 3H), 1.33 (s, 12H).

LCMS: m/z=811.37, C₅₀H₅₅N₂O₆S⁺.

### Example 21: Preparation of 2-((E)-2-((E)-2-chloro-3-(2-((E)-3,3-dimethyl-1-(2-(3-oxo-3-phenoxypropoxy)ethyl)indol-2-methylene)ethylidene)cyclopent-1-en-1-yl)ethenyl)-1-ethyl-3,3-dimethyl-3H-indol-1-ium Trifluoroacetate (21)

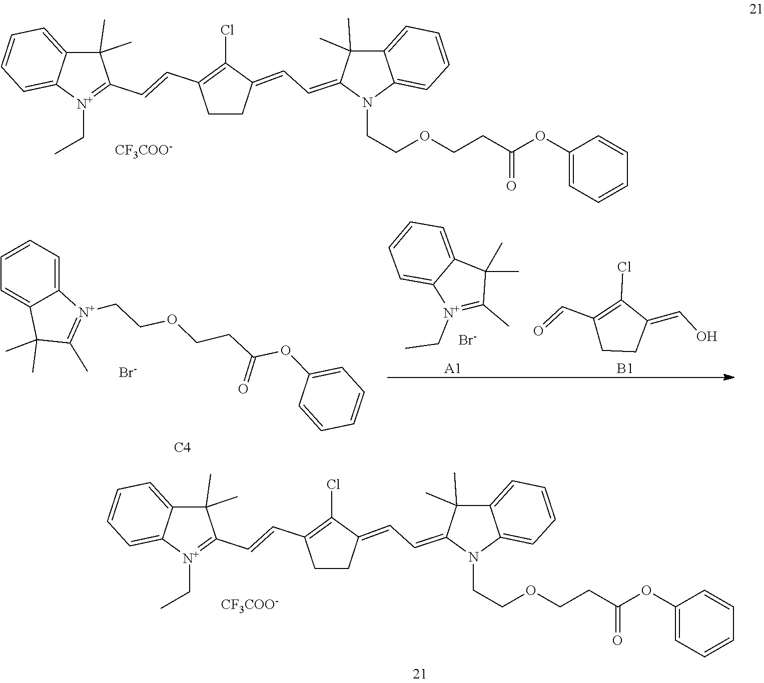

NaOAc (776 mg, 9.46 mmol, 3.00 eq) was added to a solution of Compound B1 (500 mg, 3.15 mmol, 1.00 eq), Compound A1 (994 mg, 2.29 mmol, 1.00 eq) and Compound C4 (1.36 g, 3.15 mmol, 1.00 eq) in acetic anhydride (20.0 mL). The mixture was stirred at 80° C. for 3 hours. The reaction mixture was concentrated under reduced pressure, and the residues were purified by reversed-phase HPLC (Phenomenex luna C18 150×40 mm×15 μm, mobile phase: [water (TFA)-ACN]; B %: 25%-55%, 10 min, 0.1% HCl condition) to obtain a crude product (400 mg). The crude product was further purified by reversed-phase HPLC (TFA condition, chromatographic column: Phenomenex luna C18 150 mm×40 mm×15 μm; mobile phase: [water (TFA)-ACN]; gradient: 60%-90% B 10 min) to obtain Compound 21 (66.41 mg, 80.4 μmol, 2.55% yield, 93.8% purity) after lyophilization as a green solid.

¹H NMR (400 MHz, CDCl₃) δ 7.88 (t, J=11.2 Hz, 2H), 7.40-7.47 (m, 4H), 7.31-7.36 (m, 5H), 7.17-7.24 (m, 2H), 6.90 (d, J=8.0 Hz, 2H), 5.96-6.21 (m, 2H), 4.38 (s, 2H), 4.13-4.21 (m, 2H), 4.02 (t, J=4.8 Hz, 2H), 3.88 (t, J=6.0 Hz, 2H), 2.97 (br, 2H), 2.78 (t, J=5.6 Hz, 2H), 1.80 (s, 2H), 1.75 (d, J=14 Hz, 12H), 1.49 (t, J=7.2 Hz, 2H).

LCMS: m/z=661.3, C₄₂H₄₆ClN₂O₃⁺.

### Example 22: Preparation of 2-((E)-2-((E)-2-chloro-3-(2-((E)-3,3-dimethyl-1-(6-oxo-6-phenoxyhexyl)indolin-2-ylidene)ethenyl)cyclopent-1-en-1-yl)ethenyl)-1-ethyl-3,3-dimethyl-5-sulfonyl-3H-indol-1-ium Chloride (22)

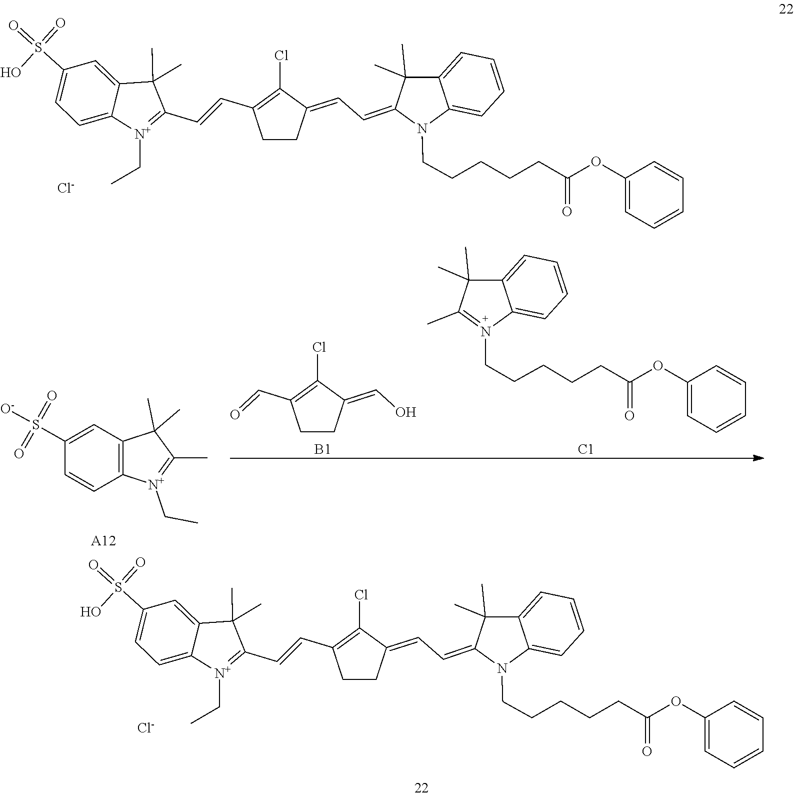

Compound 22 was prepared as a green solid according to the preparation method of Example 21 with Compounds A12, B1 and C1 as raw materials.

¹H NMR (400 MHz, DMSO-d6) δ 7.70-7.79 (m, 3H), 7.62-7.64 (m, 2H), 7.36-7.46 (m, 5H), 7.24-7.34 (m, 2H), 7.02 (d, J=7.6 Hz, 2H), 6.10-6.20 (m, 2H), 4.19-4.23 (m, 4H), 2.91-2.94 (m, 4H), 2.56-2.58 (m, 2H), 1.78-1.80 (m, 2H), 1.69-1.73 (m, 2H), 1.66 (d, J=1.6 Hz, 12H), 1.48-1.50 (m, 2H), 1.29 (d, J=7.2 Hz, 3H)

LCMS: m/z==739.4, C₄₃H₄₈ClN₂O₅S⁺.

### Example 23: Preparation of 2-((E)-2-((E)-2-chloro-3-(2-((E)-1-(6-(2,6-dimethylphenoxy)-6-oxohexyl)-3,3-dimethylindolin-2-ylidene)ethenyl)cyclopent-1-en-1-yl)ethenyl)-1-(6-(2,6-dimethylphenoxy)-6-oxohexyl)-3,3-dimethyl-3H-indol-1-ium Chloride (23)

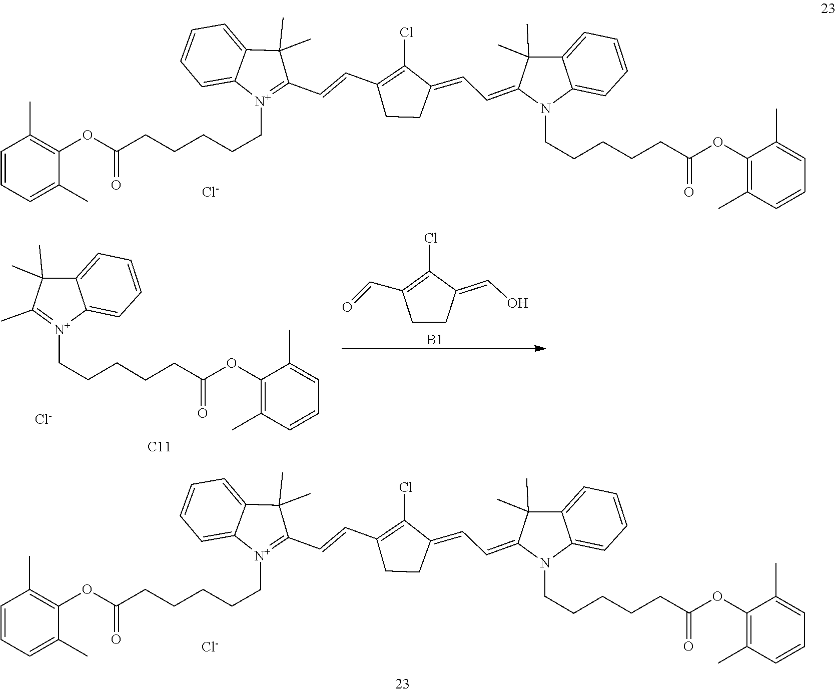

Compound 23 was prepared as a green solid according to the preparation method of Example 21 with Compounds B1 and C11 as raw materials.

LCMS: m/z=877.5, C₅₇H₆₆ClN₂O₄⁺.

### Example 24: Preparation of 2-((E)-2-((E)-2-chloro-3-(2-((E)-3,3-dimethyl-1-(6-oxo-6-phenoxyhexyl)indolin-2-ylidene)ethenyl)cyclopent-1-en-1-yl)ethenyl)-3,3-dimethyl-1-(6-oxo-6-phenoxyhexyl)-3H-indol-1-ium Chloride (24)

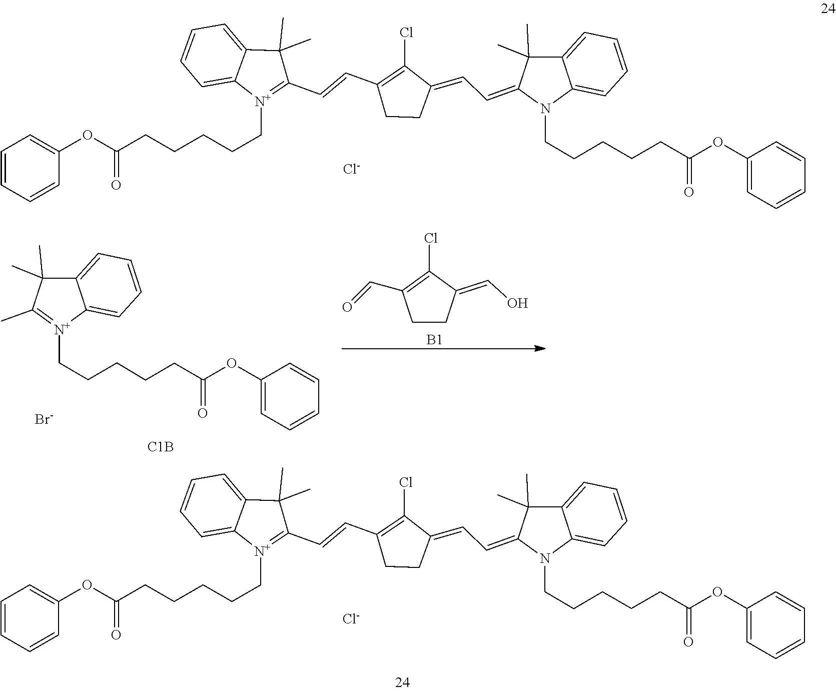

was used, and Compound 24 was prepared as a blue paste according to the preparation method of Example 21 with Compounds B1 and C1B (prepared according to the preparation method of Compound C1) as raw materials.

LCMS: m/z=821.3, C₅₃H₅₈ClN₂O₄⁺.

### Example 25: Preparation of Compound 25

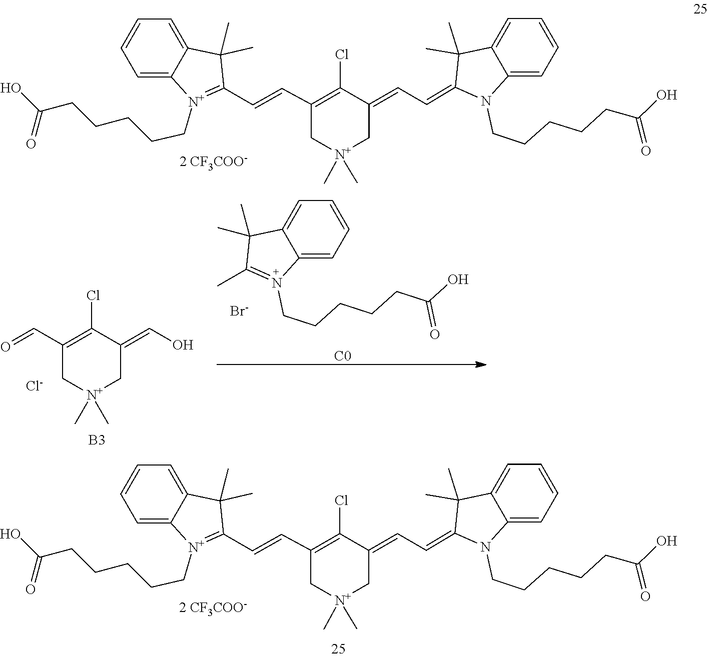

was used, and Compound 25 was prepared as a purple oil according to the preparation method of Example 21 with Compounds B3 and C0 as raw materials.

¹H NMR (400 MHz, CDCl₃) δ 8.30 (d, J=14.4 Hz, 2H), 7.40-7.45 (m, 4H), 7.30-7.34 (m, 2H), 7.18-7.20 (m, 2H), 6.38-6.42 (m, 2H), 4.83 (s, 4H), 4.24 (s, 4H), 4.48 (s, 6H), 2.33-2.37 (m, 4H), 1.78-1.87 (m, 5H), 1.73 (s, 12H), 1.44-1.54 (m, 7H).

LCMS: m/z=357.2, C₄₃H₅₆ClN₃O₄²⁺.

### Example 26: Preparation of 2-((E)-2-((E)-2-chloro-3-(2-((E)-3,3-dimethyl-1l-(6-oxo-6-(phenylamino)hexyl)indolin-2-ylidene)ethenyl)cyclohex-1-en-1-yl)ethenyl)-3,3-dimethyl-1-(4-sulfonylbutyl)-3H-indo-1-ium Formate (26)

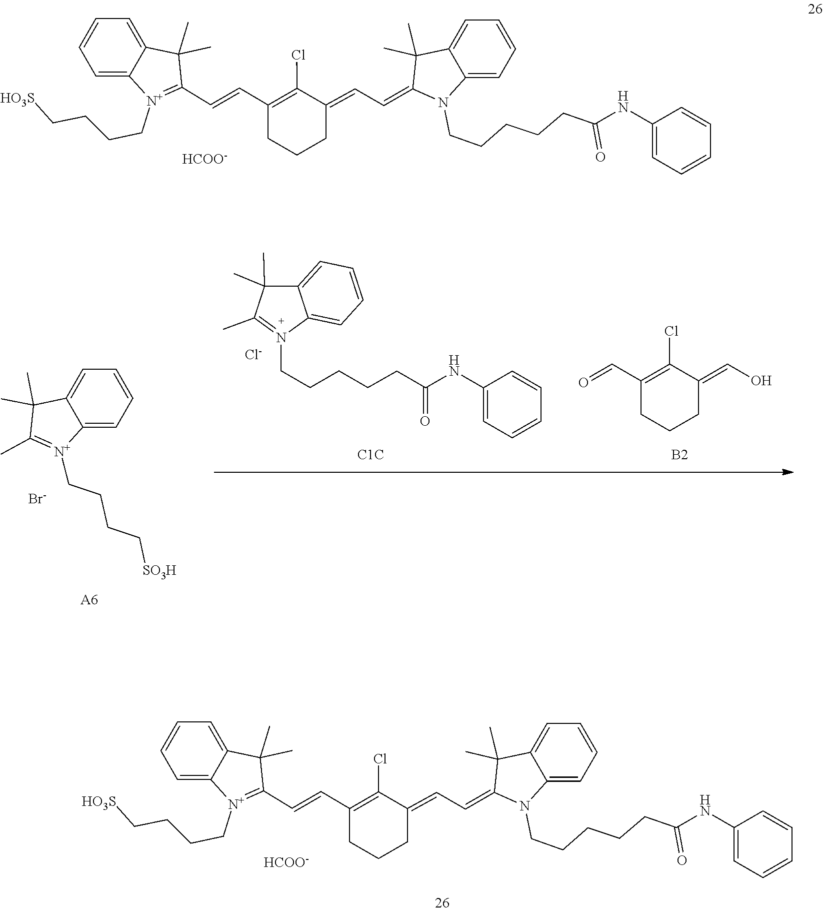

was used, and Compound 26 was prepared as a green solid according to the preparation method of Example 21 with Compounds A6, B2 and C1C as raw materials (Compounds A6 and C1C being purchased from Bide Pharm, batch number 221007).

¹H NMR: (400 MHz, CDCl₃) δ 9.62 (s, 1H), 8.32 (dd, J=14.0, 2.0 Hz, 2H), 7.77 (d, J=7.6 Hz, 2H), 7.40-7.33 (m, 4H), 7.25-7.20 (m, 4H), 7.15-7.11 (m, 2H), 7.02-6.99 (m, 1H), 6.20 (dd, J=13.6, 4.8 Hz, 2H), 4.13-4.04 (m, 4H), 3.04-3.01 (m, 2H), 2.67-2.60 (m, 6H), 2.11-2.01 (m, 4H), 1.95-1.91 (m, 4H), 1.85-1.83 (m, 1H), 1.69 (d, J=2.4 Hz, 12H), 1.64-1.60 (m, 3H).

LCMS: m/z=780.3, C₄₆H₅₅ClN₃O₄S⁺.

### Example 27A and 27B: Preparation of 1-(5-carboxypentyl)-2-((E)-2-((E)-2-chloro-3-(2-((E)-3,3-dimethyl-1-(6-oxo-6-(phenylamino)hexyl)indolin-2-ylidene)ethenyl)cyclohex-1-en-1-yl)ethenyl)-3,3-dimethyl-3H-indol-1-ium 2,2,2-trifluoroacetate (27A) and 2-((E)-2-((E)-2-chloro-3-(2-((E)-3,3-dimethyl-1-(6-oxo-6-(phenylamino)hexyl)indolin-2-ylidene)ethenyl)cyclohex-1-en-1-yl)ethenyl)-3,3-dimethyl-1-(6-oxo-6-(phenylamino) hexyl)-3H-indol-1-ium 2,2,2-trifluoroacetate (27B)

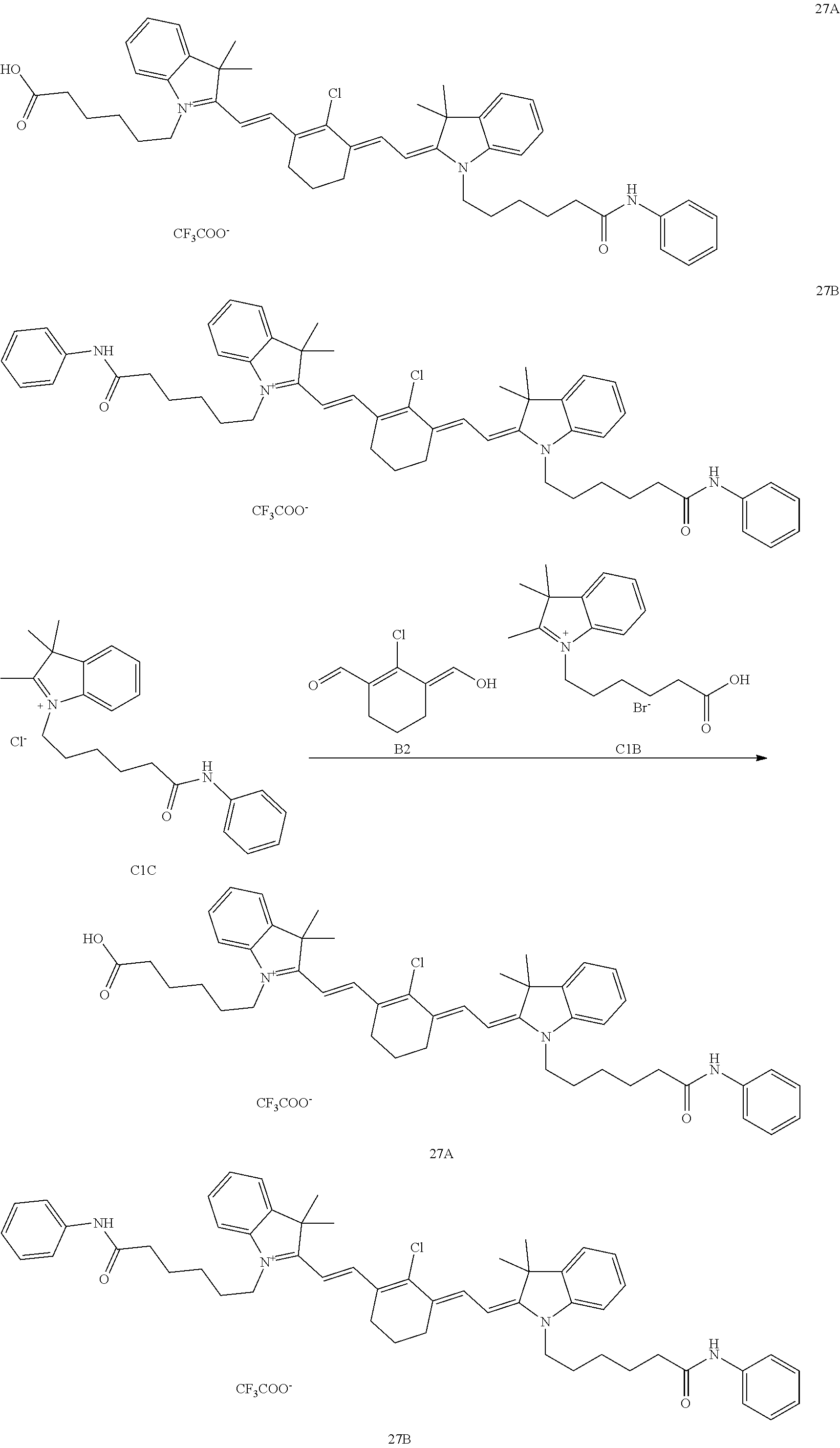

Compound 27A and Compound 27B were both prepared as a green solid according to the preparation method of Example 21 with Compounds C1B, B2 and C1C as raw materials.

### Compound 27A:

¹H NMR (400 MHz, CDCl₃) δ 9.11 (s, 1H), 8.34 (t, J=14.4 Hz, 2H), 7.67 (d, J=8.0 Hz, 2H), 7.36-7.40 (m, 4H), 7.28-7.29 (s, 1H), 7.21-7.25 (m, 3H), 7.15 (d, J=8.0 Hz, 1H), 7.07 (d, J=8.0 Hz, 1H), 6.99 (d, J=7.6 Hz, 1H), 6.03-6.02 (m, 2H), 3.98-4.09 (m, 4H), 2.63-2.64 (m, 4H), 2.51 (t, J=6.8 Hz, 2H), 2.42 (d, J=6.8 Hz, 2H), 1.76-1.92 (m, 9H), 1.70 (d, J=12.0 Hz, 12H), 1.53-1.55 (m, 5H).

LCMS: m/z=758.8, C₄₈H₅₇ClN₃O₃⁺.

### Compound 27B:

¹H NMR (400 MHz, CDCl₃) δ 9.19 (brs, 1H), 8.31-8.34 (m, 2H), 7.65 (d, J=6.8 Hz, 2H), 7.36-7.40 (m, 4H), 7.22-7.26 (m, 3H), 7.14 (d, J=8.4 Hz, 1H), 7.07 (d, J=7.6 Hz, 1H), 6.99 (d, J=6.4 Hz, 1H), 6.08-6.21 (m, 2H), 3.99-4.08 (m, 4H), 2.63 (s, 4H), 2.49 (t, J=6.8 Hz, 4H), 1.79-1.88 (m, 10H), 1.71 (d, J=14.4 Hz, 12H), 1.53-1.55 (m, 4H).

LCMS: m/z=758.4, C₄₈H₅₇ClN₃O₃⁺.

### Example 28: Preparation of Compound 28

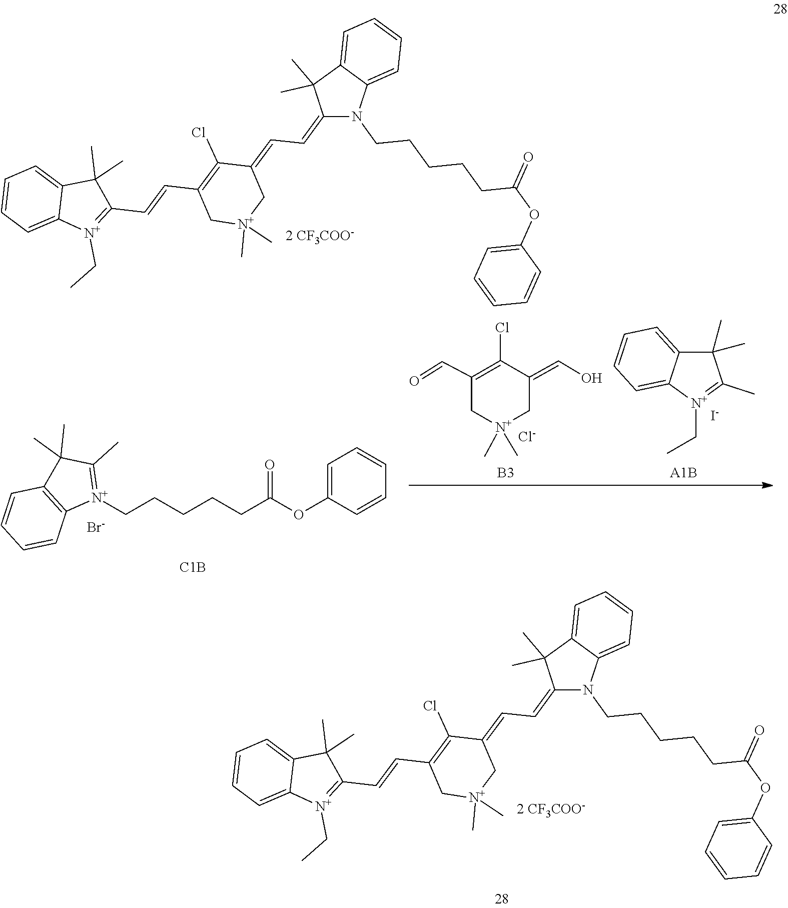

Compound 28 was prepared as a blue solid according to the preparation method of Example 21 with Compounds C1B, B3 and A1B (prepared according to the preparation method of Compound A1) as raw materials.

¹H NMR (400 MHz, CDCl₃) δ 8.30-8.28 (m, 2H), 7.47-7.30 (m, 8H), 7.29-7.28 (m, 2H), 7.25-7.18 (m, 3H), 7.04 (d, J=7.6 Hz, 2H), 5.28-5.20 (m, 2H), 4.69-4.59 (m, 2H), 3.62-3.54 (m, 4H), 2.62-2.59 (m, 1H), 1.98-1.81 (m, 5H), 1.72 (s, 20H), 1.53-1.48 (m, 3H).

LCMS: m/z=352.0, C₄₅H₅₄ClN₃O₂²⁺.

### Example 29: Preparation of 2-((E)-2-((E)-4-chloro-5-(2-((E)-3,3-dimethyl-1-(6-oxo-6-phenoxyhexyl)indolin-2-ylidene)ethenyl)-1,1-dimethyl-1,2,5,6-tetrahydropyridin-1-ium-3-yl)ethenyl)-3,3-dimethyl-1-(6-oxo-6-phenoxyhexyl)-3H-indol-1-ium Chloride (29)

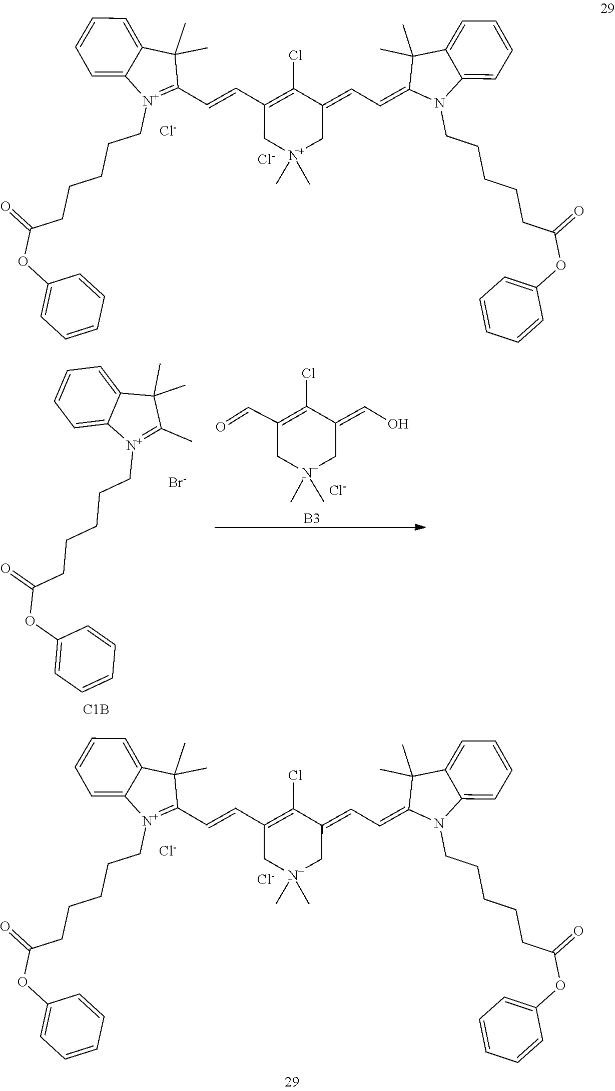

Compound 29 was prepared as a green paste according to the preparation method of Example 21 with Compounds C1B and B3 as raw materials.

LCMS: m/z=433.1, C₅₅H₆₄ClN₃O₄²⁺.

### Example 30: Preparation of 2-((E)-2-((E)-3-(2-((E)-1-(5-carboxypentyl)-3,3-dimethylindolin-2-ylidene)ethenyl)-2-chlorocyclohex-1-en-1-yl)ethenyl)-1-ethyl-3,3-dimethyl-3H-indol-1-ium Chloride (30)

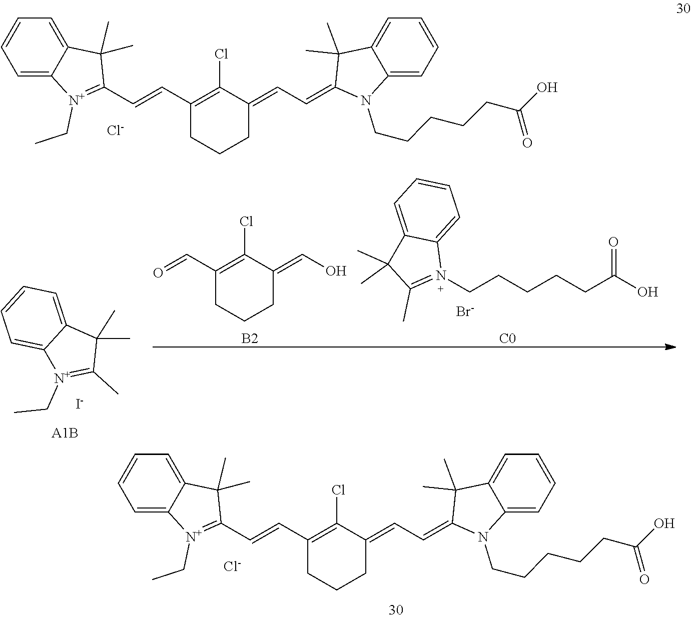

Compound 30 was prepared as a green solid according to the preparation method of Example 21 with Compounds A1B, B2 and C0 as raw materials.

LCMS: m/z=597.5, C₃₈H₄₆ClN₂O₂⁺.

### Example 33: Preparation of 2-((E)-2-((E)-2-chloro-3-(2-((E)-3,3-dimethyl-1-(5-oxo-5-phenoxypentyl)indolin-2-ylidene)ethenyl)cyclohex-1-en-1-yl)ethenyl)-3,3-dimethyl-1-(5-oxo-5-phenoxypentyl)-3H-indol-1-ium Chloride (33)

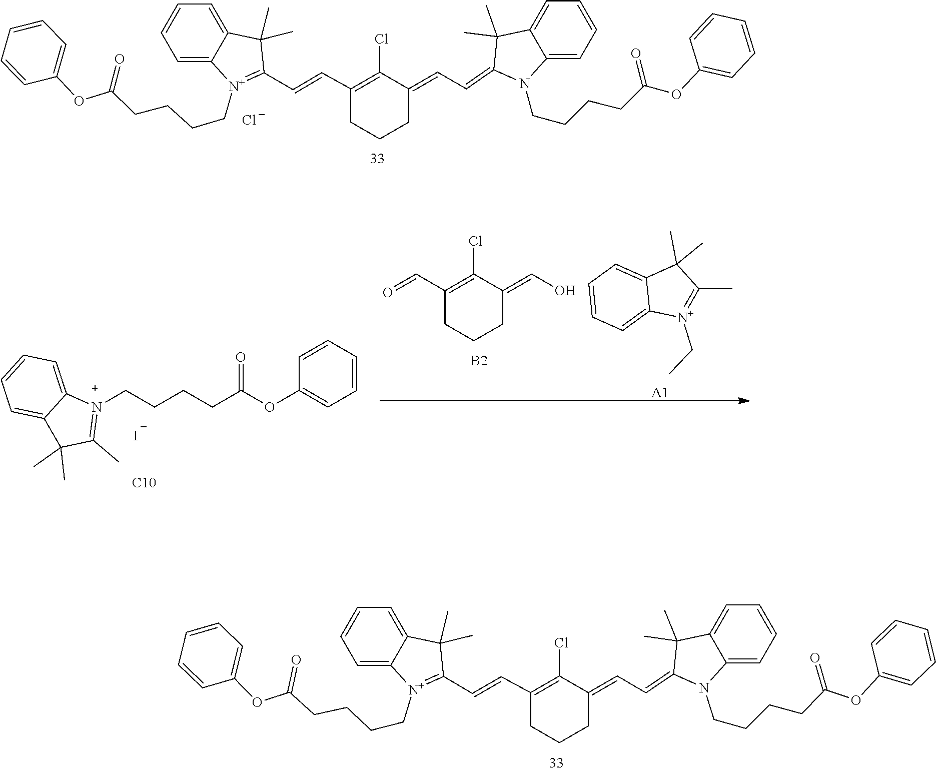

and Compound 33 was prepared as a green solid according to the preparation method of Example 21 with Compounds B2, A1 and C10 as raw materials.

¹H NMR (400 MHz, CDCl₃) δ 8.34 (d, J=14.0 Hz, 2H), 7.34-7.38 (m, 8H), 7.20-7.26 (m, 6H), 7.03 (d, J=7.6 Hz, 4H), 6.32 (d, J=14.0 Hz, 2H), 4.31-4.35 (m, 4H), 2.70-2.75 (m, 8H), 1.90-2.03 (m, 10H), 1.73 (s, 12H).

LCMS: m/z=807.4, C₅₂H₅₆ClN₂O₄⁺.

### Example 34: Preparation of 2-((E)-2-((E)-2-chloro-3-(2-((E)-3,3-dimethyl-1-(5-oxo-5-phenoxypentyl)indolin-2-ylidene)ethenyl)cyclohex-1-en-1-yl)ethenyl)-1-ethyl-3,3-dimethyl-3H-indol-1-ium Chloride (34)

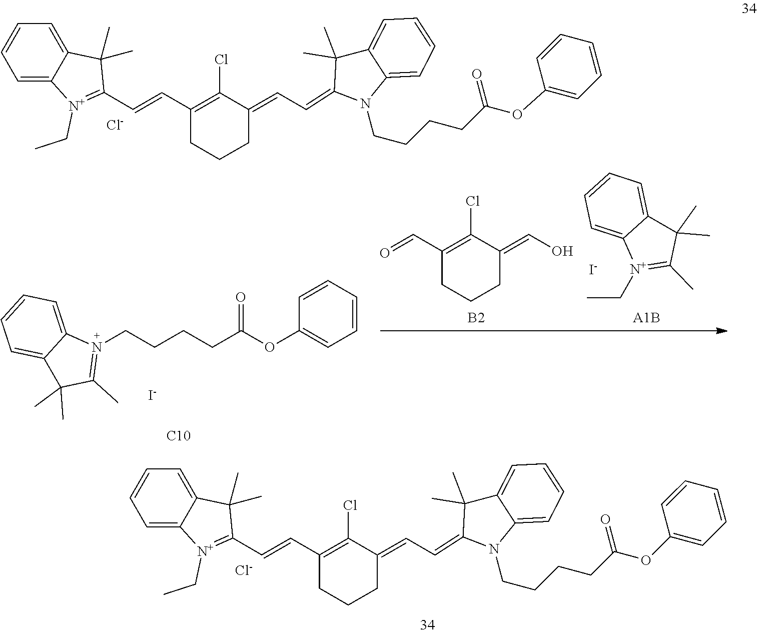

Compound 34 was prepared as a green solid according to the preparation method of Example 21 with Compounds B2, A1B and C10 as raw materials.

¹H NMR (400 MHz, CDCl₃) δ 8.32-8.37 (m, 2H), 7.34-7.42 (m, 6H), 7.20-7.25 (m, 5H), 7.02-7.04 (m, 2H), 6.25-6.35 (m, 2H), 4.26-4.35 (m, 4H), 2.70-2.78 (m, 6H), 1.94-2.02 (m, 6H), 1.73 (s, 12H), 1.47 (t, J=7.2 Hz, 3H).

LCMS: m/z=659.3, C₄₃H₄₈ClN₂O₂⁺.

### Example 35: Preparation of 2-((E)-2-((E)-2-chloro-3-(2-((E)-3,3-dimethyl-1-(2-((6-oxo-6-phenoxyhexyl)oxy)ethyl)indolin-2-ylidene)ethenyl)cyclohex-1-en-1-yl)ethenyl)-1-ethyl-3,3-dimethyl-3H-indol-1-ium 2,2,2-trifluoroacetate (35)

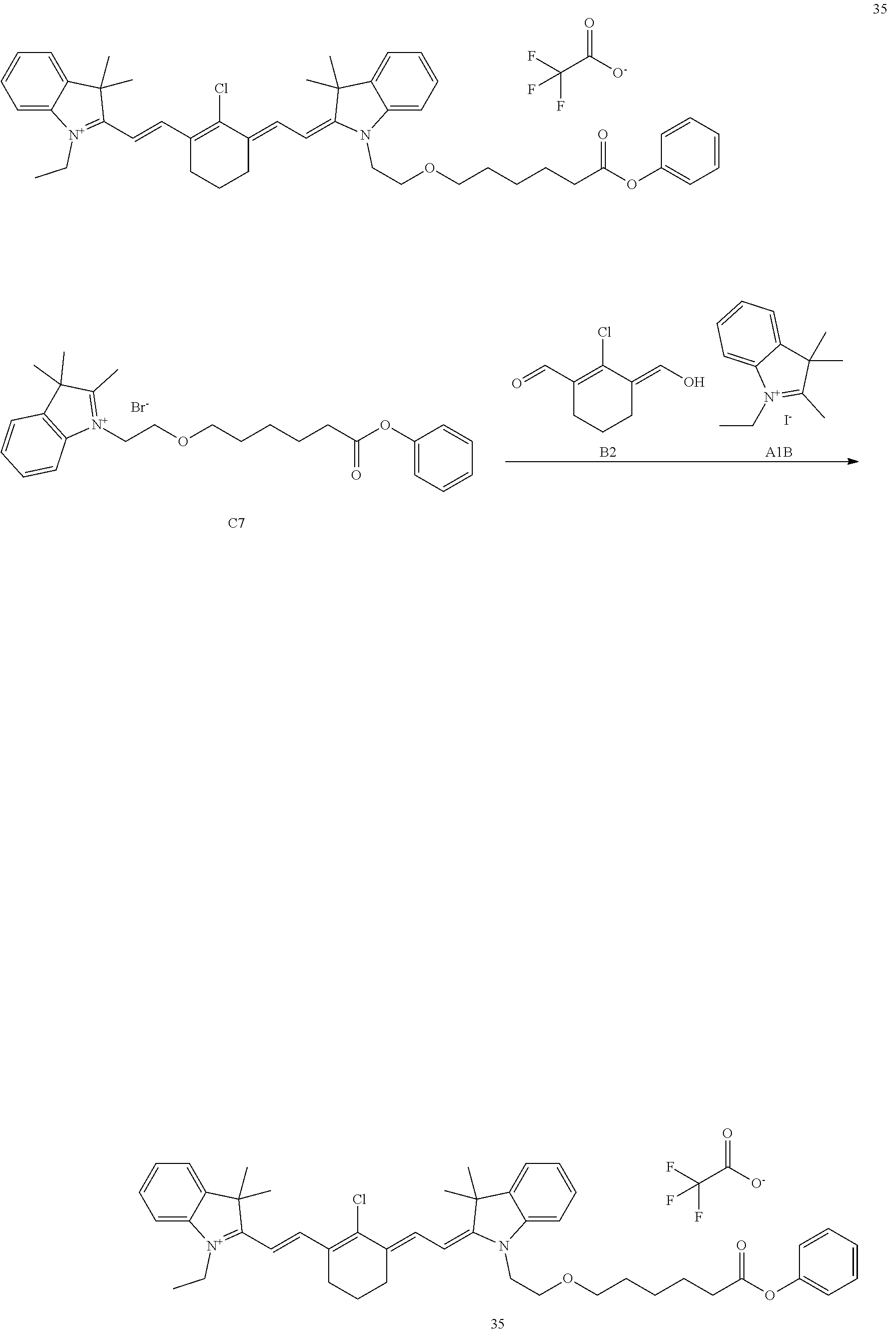

Compound 35 was prepared as a blue solid according to the preparation method of Example 21 with Compounds B2, A1B and C7 as raw materials.

¹H NMR: (400 MHz, CDCl₃) δ 8.37 (t, J=31.6 Hz, 2H), 7.37-7.40 (m, 6H), 7.32-7.34 (m, 4H), 7.24-7.25 (m, 1H), 7.02-7.04 (m, 2H), 6.33 (d, J=14.4 Hz, 1H), 6.10 (d, J=14 Hz, 1H), 4.31-4.34 (m, 2H), 4.09-4.14 (m, 2H), 3.83-3.86 (m, 2H), 3.42 (t, J=6.4 Hz, 2H), 2.67 (s, 4H), 2.44 (t, J=7.6 Hz, 2H), 1.95-1.97 (m, 2H), 1.71 (d, J=10 Hz, 12H), 1.64-1.66 (m, 2H), 1.62-1.64 (m, 2H), 1.42-1.44 (m, 3H), 1.31-1.40 (m, 2H).

LCMS: m/z=717.4, C₄₆H₅₄ClN₂O₃⁺.

### Example 36: Preparation of 2-((E)-2-((E)-2-chloro-3-(2-((E)-3,3-dimethyl-1-(2-(4-oxo-4-phenoxybutoxy)ethyl)indolin-2-ylidene)ethenyl)cyclohex-1-en-1-yl)ethenyl)-1-ethyl-3,3-dimethyl-3H-indol-1-ium 2,2,2-trifluoroacetate (36)

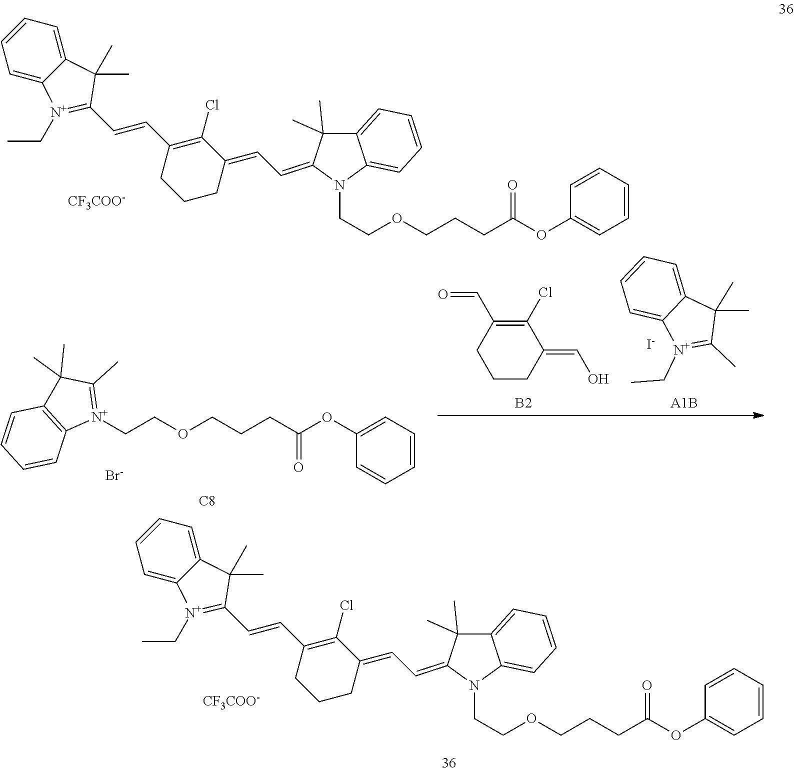

Compound 36 was prepared as a blue solid according to the preparation method of Example 21 with Compounds B2, A1B and C8 as raw materials.

¹H NMR (400 MHz, CDCl₃) δ 8.28-8.39 (m, 2H), 7.36-7.43 (m, 5H), 7.30-7.34 (m, 2H), 7.14-7.26 (m, 4H), 6.97-7.01 (m, 2H), 6.49-6.55 (m, 1H), 6.14-6.17 (m, 1H), 4.58 (s, 2H), 4.21 (s, 2H), 3.93-3.99 (m, 2H), 3.53 (t, J=6.0 Hz, 2H), 2.62-2.81 (m, 4H), 2.47 (t, J=7.6 Hz, 2H), 1.86-1.96 (m, 4H), 1.72 (d, J=8.4 Hz, 12H), 1.44 (t, J=7.2 Hz, 3H).

¹⁹F NMR (400 MHz, CDCl₃) δ 75.946.

LCMS: m/z=689.4, C₄₄H₅₀ClN₂O₃⁺.

### Example 37: 2-((E)-2-((E)-2-chloro-3-(2-((E)-3,3-dimethyl-1-(2-((5-oxo-5-phenoxypentyl)oxy)ethyl) indolin-2-ylidene)ethenyl)cyclohex-1-en-1-yl)ethenyl)-1-ethyl-3,3-dimethyl-3H-indol-1-ium 2,2,2-trifluoroacetate (37A) YH-0656 and 2-((E)-2-((E)-2-chloro-3-(2-((E)-3,3-dimethyl-1-(2-((5-oxo-5-phenoxypentyl)oxy)ethyl) indolin-2-ylidene)ethenyl)cyclohex-1-en-1-yl)ethenyl)-3,3-dimethyl-1-(2-((5-oxo-5-phenoxypentyl)oxy)ethyl)-3H-indol-1-ium (37B)

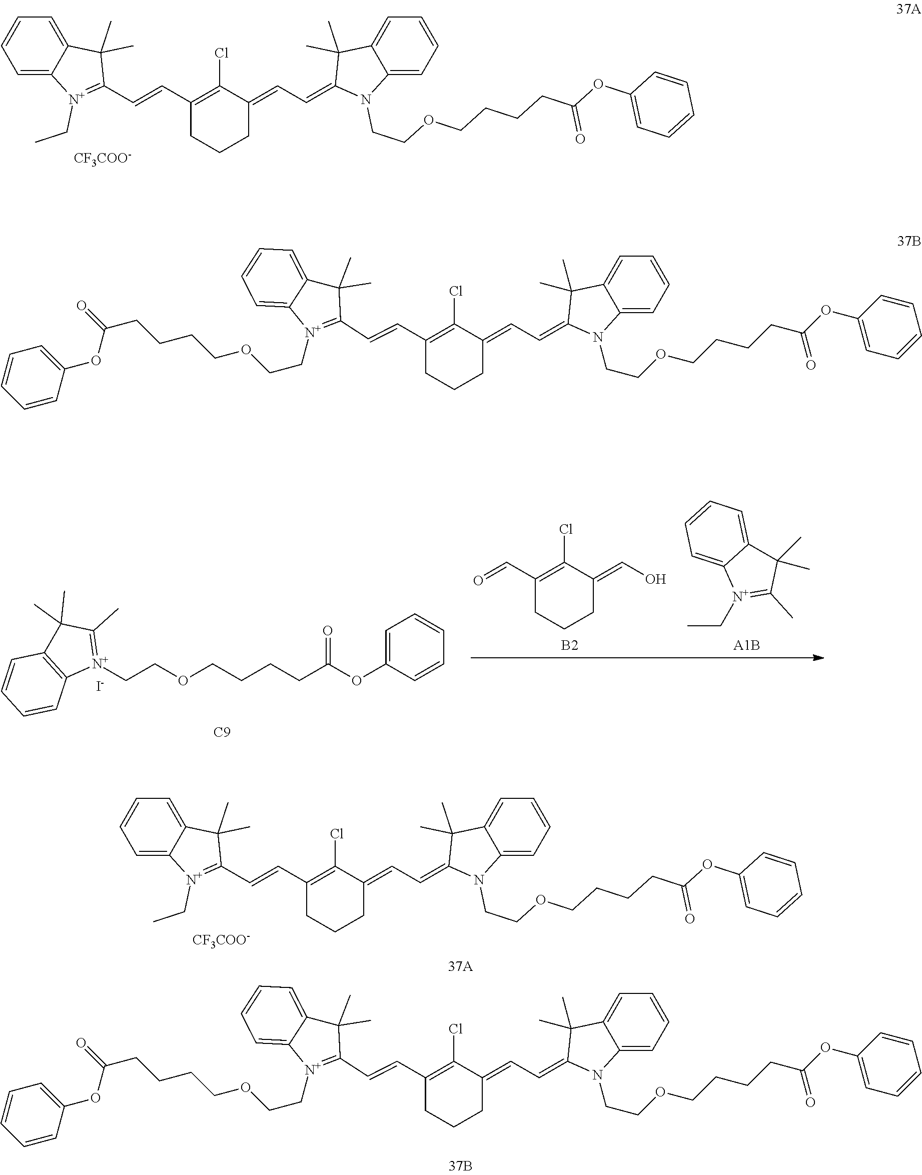

Compound 37A and Compound 37B were both prepared as a green solid according to the preparation method of Example 21 with Compounds B2, A1B and C9 as raw materials.

### Compound 37A:

LCMS: m/z=703.4, C₄₅H₅₂ClN₂O₃⁺.

### Compound 37B:

¹H NMR (400 MHz, CDCl₃) δ 8.27 (d, J=14.4 Hz, 2H), 7.28-7.31 (m, 7H), 7.16-7.20 (m, 7H), 6.95 (d, J=7.6 Hz, 4H), 6.24 (d, J=14.0 Hz, 2H), 4.27 (t, J=4.4 Hz, 4H), 3.79 (t, J=4.8 Hz, 4H), 3.40 (t, J=6.0 Hz, 4H), 2.59 (t, J=5.2 Hz, 4H), 2.39 (t, J=7.2 Hz, 4H), 1.85-1.86 (m, 2H), 1.65 (s, 12H), 1.58-1.62 (m, 4H), 1.53-1.54 (m, 4H).

LCMS: m/z=859.6, C₅₆H₆₄ClN₂O₆⁺.

### Example 38: Preparation of (E)-4-chloro-3-(2-((E)-3,3-dimethyl-1-(5-oxo-5-phenoxypentyl)indolin-2-ylidene)ethenyl)-1,1-dimethyl-5-(((Z)-1-methyl-2-oxoindolin-2-ylidene)methyl)-1,2,3,6-tetrahydropyridin-1-ium Chloride 38)

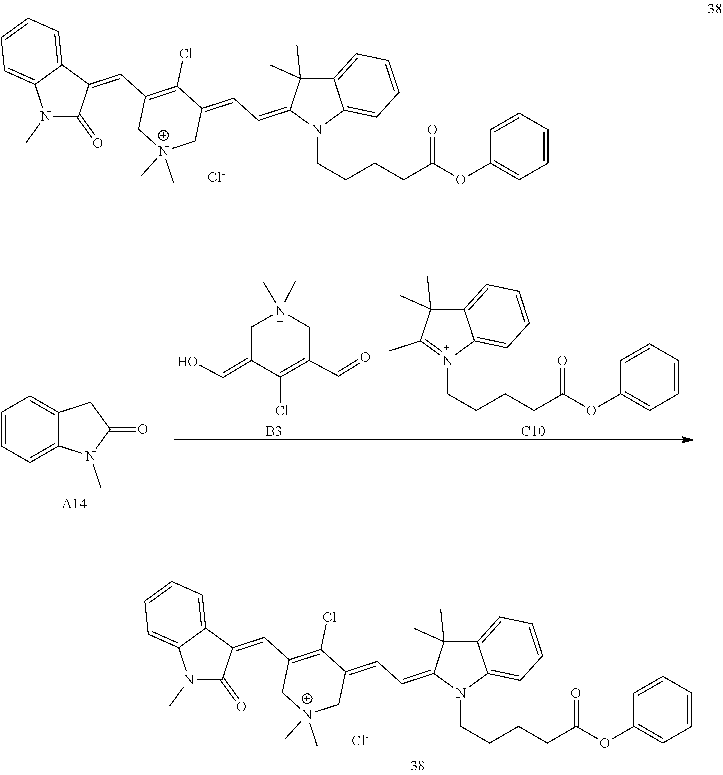

Compound 38 was prepared as a blue solid according to the preparation method of Example 21 with Compounds B3, A14 (purchased from Bide Pharm, batch number 220918) and C10 as raw materials.

¹H NMR (400 MHz, CDCl₃) δ 8.68 (s, 2H), 7.93 (d, J=13.6 Hz, 1H), 7.74 (s, 1H), 7.49 (d, J=7.6 Hz, 1H), 7.36 (t, J=7.6 Hz, 3H), 7.22-7.25 (m, 2H), 7.03-7.06 (m, 3H), 6.86-6.88 (m, 1H), 6.81-6.83 (m, 1H), 5.96-6.00 (m, 1H), 4.95 (s, 2H), 4.85 (s, 2H), 3.99-4.06 (m, 2H), 3.33 (s, 6H), 3.26 (s, 3H), 2.67-2.75 (m, 2H), 1.92 (s, 4H), 1.67 (s, 6H).

LCMS: m/z=648.4, C₄₀H₄₃ClN₃O₃⁺.

### Example 39: Preparation of 2-((E)-2-((E)-2-chloro-3-(2-((E)-1-(6-(2,6-dimethylphenoxy)-6-oxohexyl)-3,3-dimethylindolin-2-ylidene)ethenyl)cyclohex-1-en-1-yl)ethenyl)-1-ethyl-3,3-dimethyl-5-(methylsulfonyl)-3H-indol-1-ium Chloride (39)

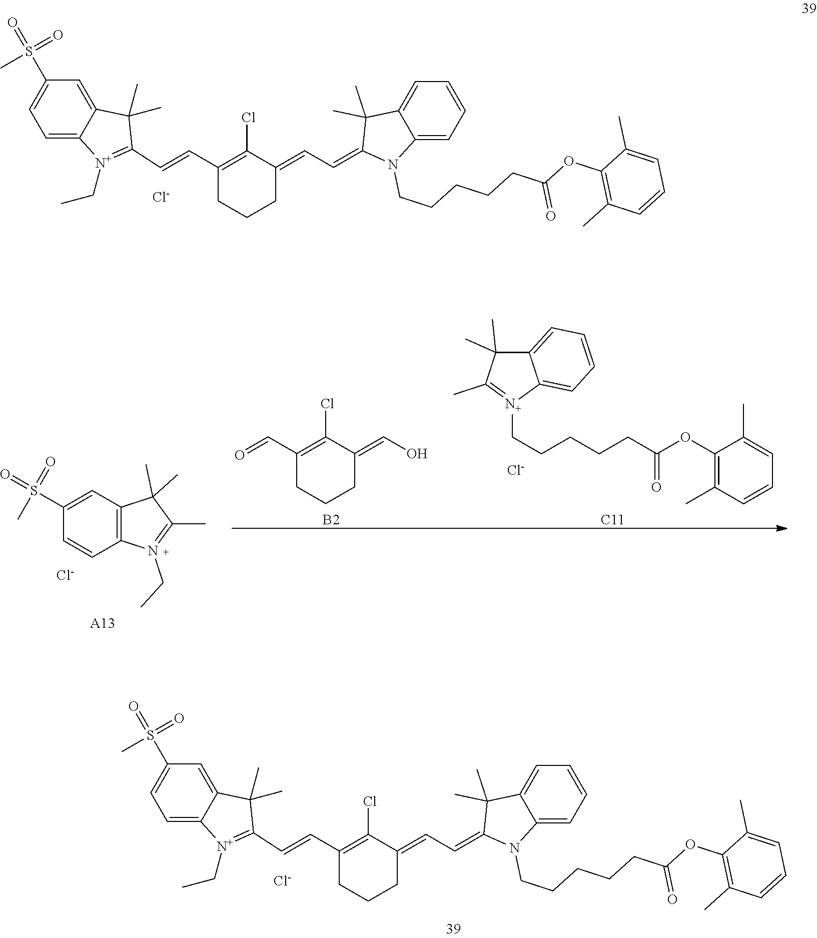

Compound 39 was prepared as a blue solid according to the preparation method of Example 21 with Compounds B2, A13 and C11 as raw materials.

¹H NMR (400 MHz, MeOD) δ 8.66 (d, J=14.8 Hz, 1H), 8.18 (d, J=13.2 Hz, 1H), 7.93-7.90 (m, 2H), 7.68-7.47 (m, 4H), 7.26 (d, J=8.4 Hz, 1H), 7.05-7.01 (m, 3H), 6.70 (d, J=14.8 Hz, 1H), 6.08 (d, J=13.2 Hz, 1H), 4.46 (t, J=6.8 Hz, 2H), 4.07 (dd, J₁=7.2 Hz, J₂=7.2 Hz, 2H), 3.14 (s, 3H), 2.73-2.67 (m, 6H), 2.05 (s, 6H), 2.03-1.99 (m, 2H), 1.91-1.81 (m, 4H), 1.78 (s, 6H), 1.74 (s, 6H), 1.65-1.59 (m, 2H), 1.35 (t, J=7.2 Hz, 3H).

LCMS: m/z=779.5, C₄₇H₅₆ClN₂O₄S⁺.

### Example 40: Preparation of 2-((E)-2-((E)-2-chloro-3-(2-((E)-3,3-dimethyl-1-(6-oxo-6-phenoxyhexyl)indolin-2-ylidene)ethenyl)cyclohex-1-en-1-yl)ethenyl)-1-ethyl-3,3-dimethyl-5-(methylsulfonyl)-3H-indol-1-ium Chloride (40)

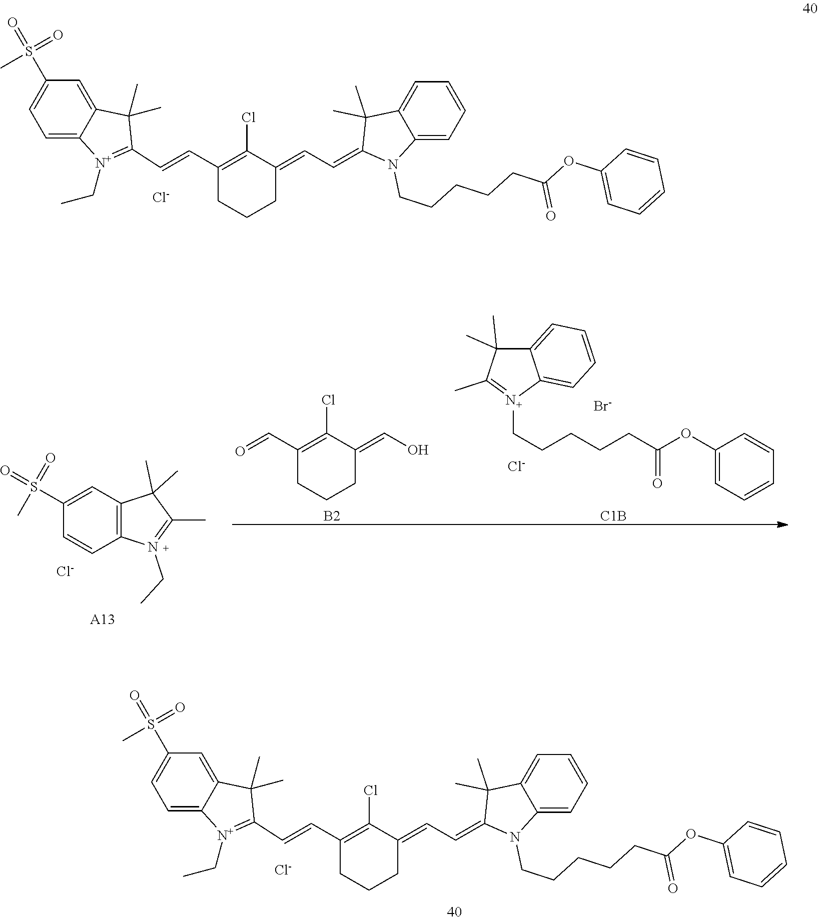

Compound 40 was prepared as a blue solid according to the preparation method of Example 21 with Compounds B2, A13 and C1B as raw materials.

¹H NMR (400 MHz, DMSO-d6) δ 8.45 (d, J=14.8 Hz, 1H), 8.05-8.01 (m, 2H), 7.87 (dd, J₁=1.6 Hz, J₂=6.4 Hz, 1H), 7.76 (dd, J₁=7.6 Hz, J₂=10.0 Hz, 2H), 7.55 (t, J=7.2 Hz, 1H), 7.48 (t, J=7.6 Hz, 1H), 7.41-7.37 (m, 3H), 7.27-7.25 (m, 1H), 7.03 (d, J=8.4 Hz, 2H), 6.73 (d, J=14.8 Hz, 1H), 6.10 (d, J=13.6 Hz, 1H), 4.48 (t J=2.8 Hz, 2H), 4.10-4.08 (m, 2H), 3.21 (s, 3H), 2.73-2.68 (m, 4H), 2.59 (t, J=7.2 Hz, 2H), 1.88-1.82 (m, 4H), 1.75-1.68 (m, 14H), 1.52-1.50 (m, 2H), 1.24 (t, J=7.2 Hz, 3H).

LCMS: m/z=751.4, C₄₅H₅₂ClN₂O₄S⁺.

### Example 41: Preparation of 5-(tert-butoxycarbonyl)-2-((E)-2-((E)-2-chloro-3-(2-((E)-3,3-dimethyl-1-(6-oxo-6-phenoxyhexyl)indolin-2-ylidene)ethenyl)cyclohex-1-en-1-yl)ethenyl)-1-ethyl-3,3-dimethyl-3H-indol-1-ium Bromide (41)

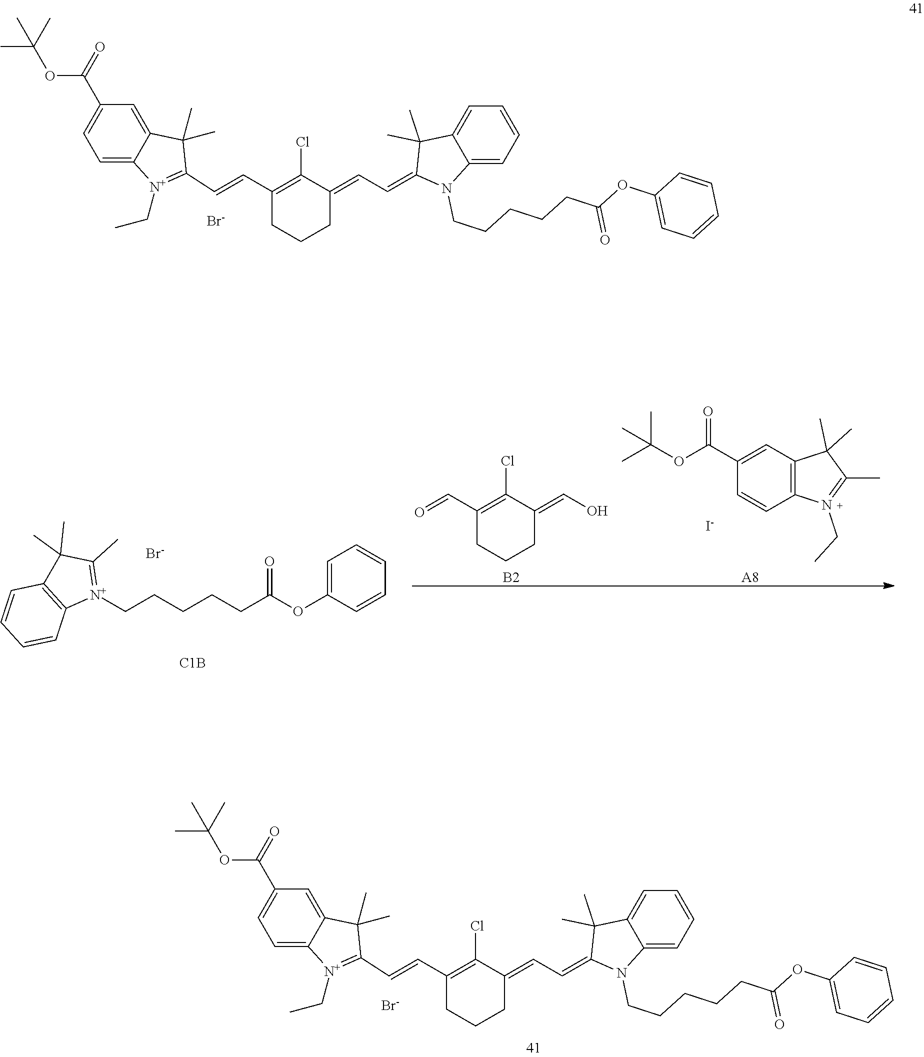

Compound 41 was prepared as a green oil according to the preparation method of Example 21 with Compounds B2, A8 and C1B as raw materials.

LCMS: m/z=773.5, C₄₉H₅₈ClN₂O₄⁺.

### Example 42: Preparation of 5-(tert-butoxycarbonyl)-2-((E)-2-((E)-2-chloro-3-(2-((E)-3,3-dimethyl-1-(6-oxo-6-phenoxyhexyl)indolin-2-ylidene)ethenyl)cyclopent-1-en-1-yl)ethenyl)-1-ethyl-3,3-dimethyl-3H-indol-1-ium Bromide (42)

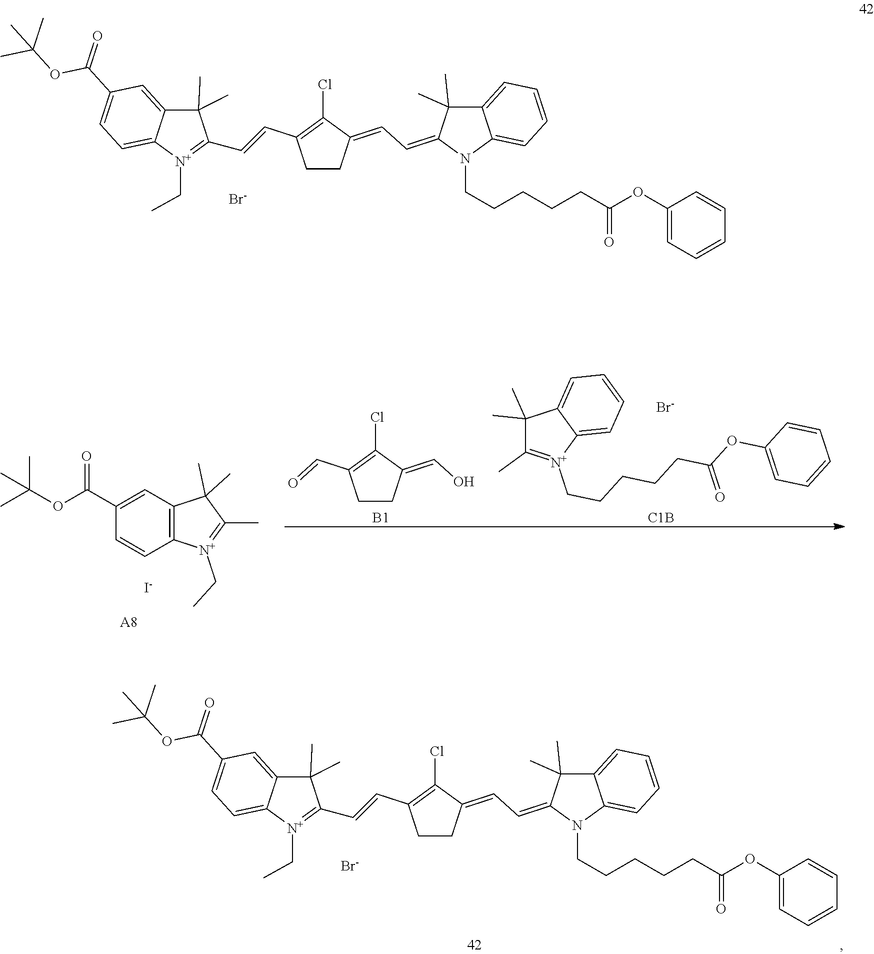

and Compound 42 was prepared as a green oil according to the preparation method of Example 21 with Compounds B1, A8 and C1B as raw materials.

LCMS: m/z=759.3, C₄₈H₅₆ClN₂O₄⁺.

### Example 43: Preparation of 5-(tert-butoxycarbonyl)-2-((E)-2-((E)-3-(2-((E)-1-(5-carboxypentyl)-3,3-dimethylindolin-2-ylidene)ethenyl)-2-chlorocyclohex-1-en-1-yl)ethenyl)-1,3,3-trimethyl-3H-indol-1-ium Bromide (43)

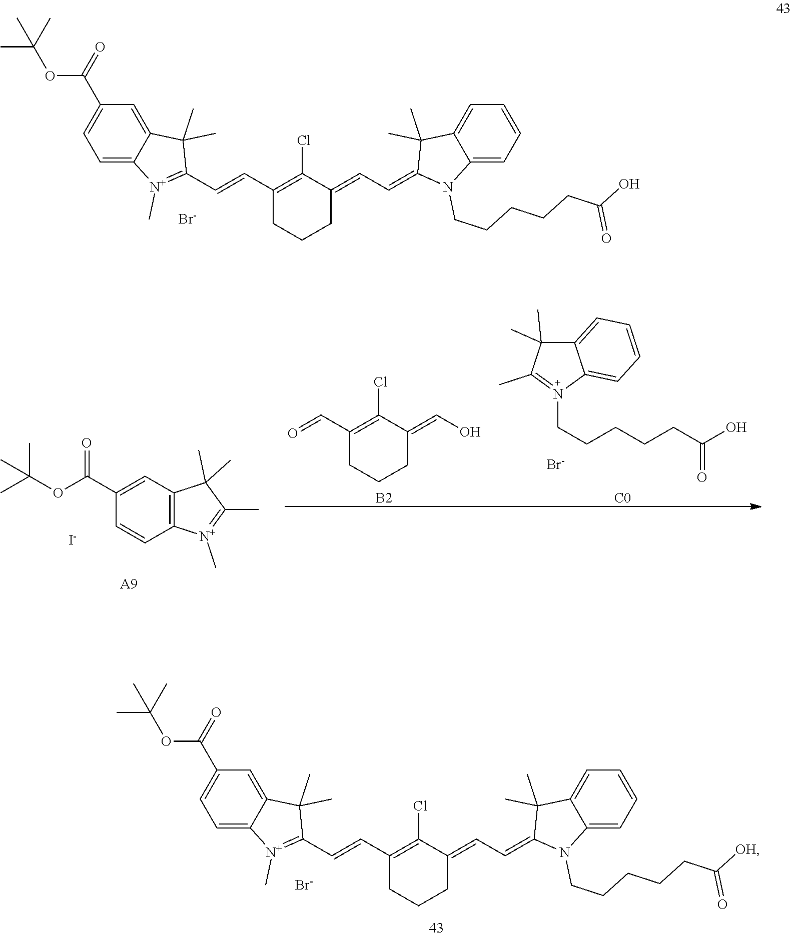

and Compound 43 was prepared as a green paste according to the preparation method of Example 21 with Compounds B1, A9 and C0 as raw materials.

LCMS: m/z=683.4, C₄₂H₅₂ClN₂O₄⁺.

### Example 44: Preparation of 5-(tert-butoxycarbonyl)-2-((E)-2-((E)-2-chloro-3-(2-((E)-3,3-dimethyl-1-(6-oxo-6-phenoxyhexyl)indolin-2-ylidene)ethenyl)cyclohex-1-en-1-yl)ethenyl)-1,3,3-trimethyl-3H-indol-1-ium Bromide (44)

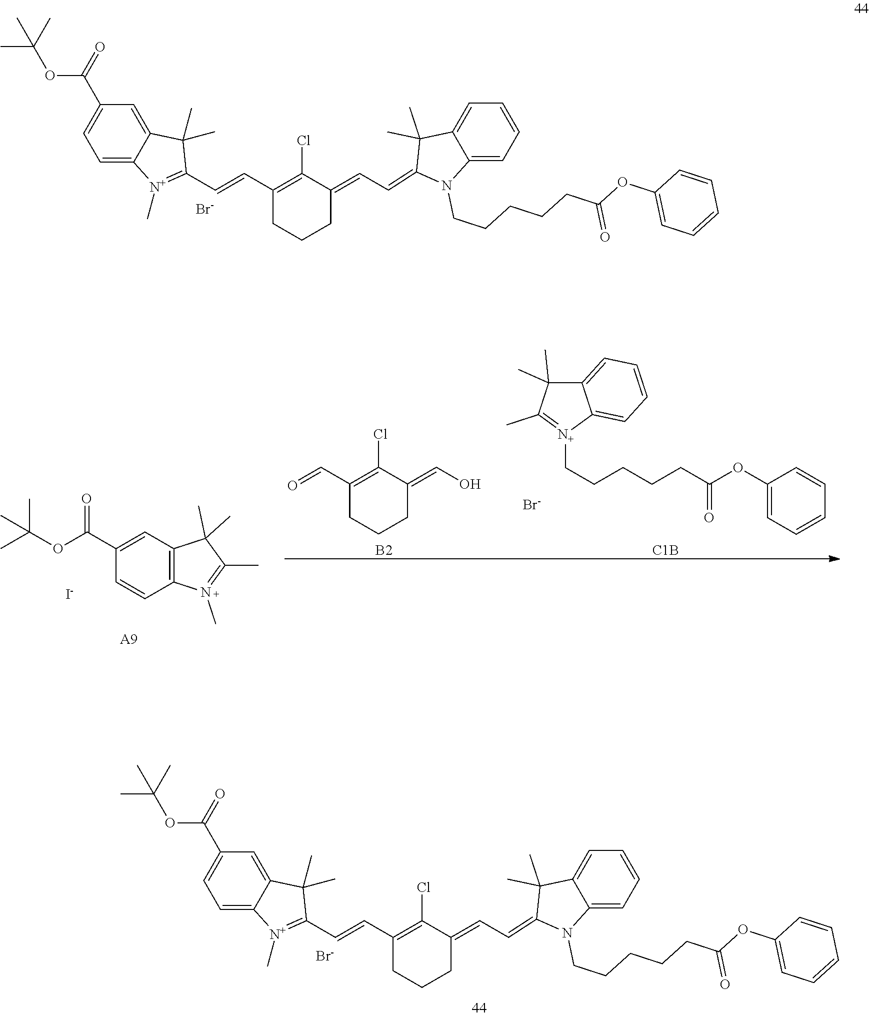

Compound 44 was prepared as a green paste according to the preparation method of Example 21 with Compounds B2, A9 and C1B as raw materials.

LCMS: m/z=759.6, C₄₈H₅₆ClN₂O₄⁺.

### Example 45: Preparation of 2-((E)-2-((E)-2-chloro-3-(2-((E)-1-(6-(2,6-dimethylphenoxy)-6-oxohexyl)-3,3-dimethylindolin-2-ylidene)ethenyl)cyclohex-1-en-1-yl)ethenyl)-1-ethyl-3,3-dimethyl-3H-indol-1-ium Chloride (45)

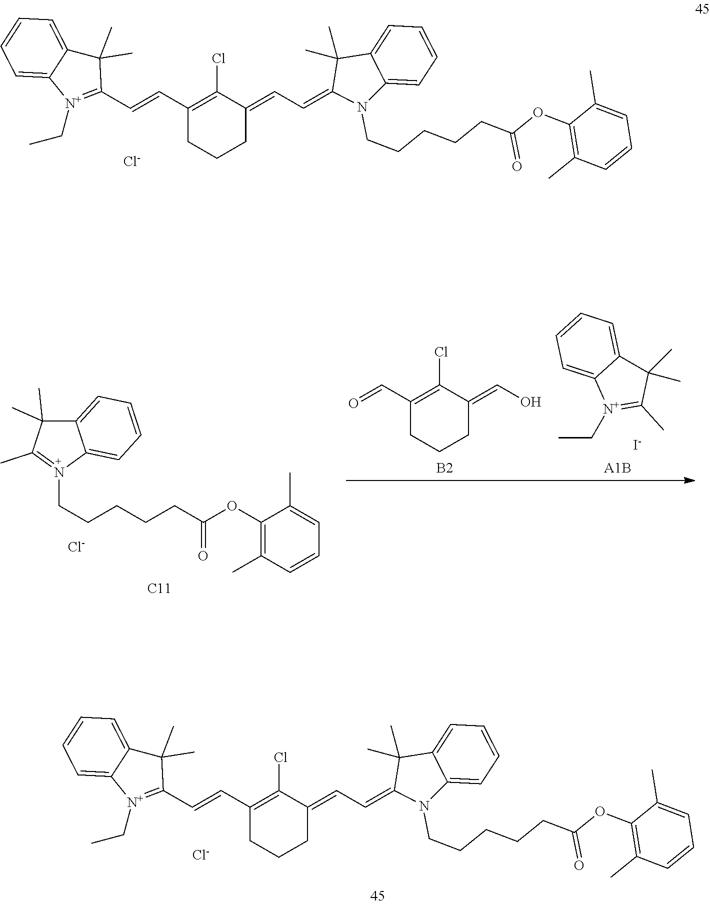

Compound 45 was prepared as a black-brown solid according to the preparation method of Example 21 with Compounds B2, A1B and C11 as raw materials.

LCMS: m/z=701.4, C₄₆H₅₄ClN₂O₂⁺.

### Example 46: Preparation of 5-(tert-butoxycarbonyl)-2-((E)-2-((E)-4-chloro-5-(2-((E)-3,3-dimethyl-1-(6-oxo-6-phenoxyhexyl)indolin-2-ylidene)ethenyl)-1,1-dimethyl-1,2,5,6-tetrahydropyridin-1-ium-3-yl) ethenyl)-1-ethyl-3,3-dimethyl-3H-indol-1-ium Chloride (46)

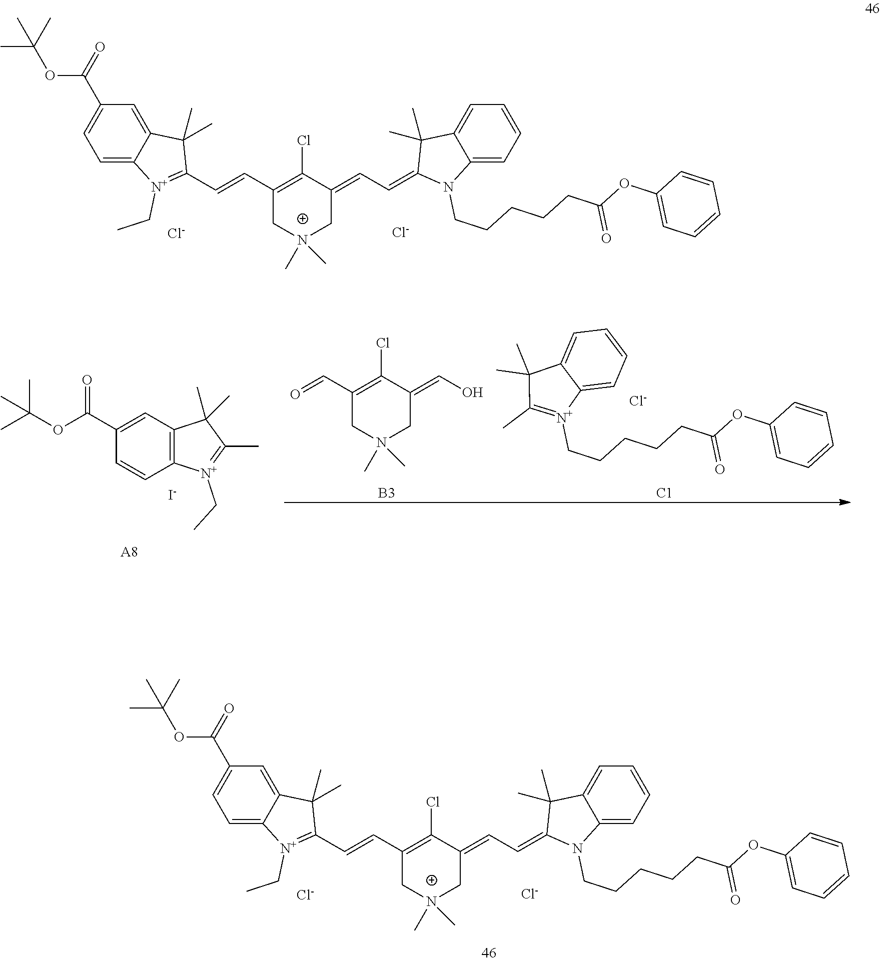

A crude of Compound 46 was prepared as a purple oil according to the preparation method of Example 21 with Compounds B3, A8 and C1 as raw materials.

LCMS: m/z=402.0, C₅₀H₆₂ClN₃O₄²⁺/2.

### Example 47: Preparation of 2-((E)-2-((E)-2-chloro-3-(2-((E)-5-methoxy-3,3-dimethyl-1-(6-oxo-6-phenoxyhexyl)indolin-2-ylidene)ethenyl)cyclohex-1-en-1-yl)ethenyl)-5-methoxy-1,3,3-trimethyl-3H-indol-1-ium (47)

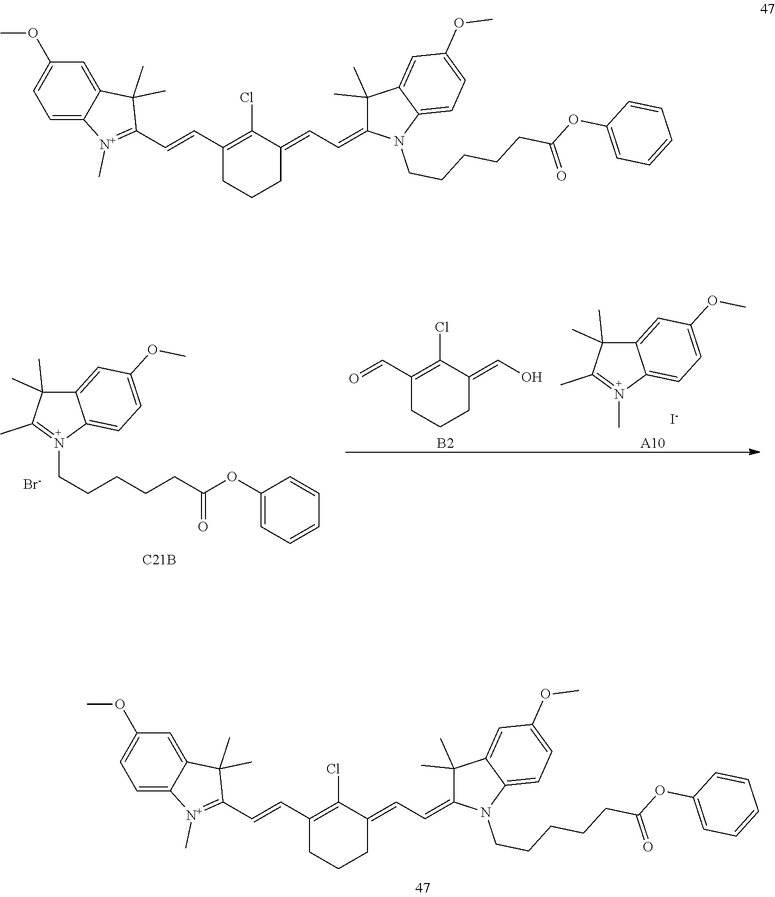

Compound 47 was prepared as a blue solid according to the preparation method of Example 21 with Compounds B2, A10 and C21B as raw materials.

¹H NMR (400 MHz, DMSO) δ 8.15-8.21 (m, 2H), 7.56-7.41 (m, 4H), 7.29-7.31 (m, 2H), 7.25 (t, J=7.2 Hz, 1H), 6.96-7.03 (m, 4H), 6.20-6.26 (m, 2H), 4.18-4.22 (m, 2H), 3.81 (d, J=2.8 Hz, 6H), 3.67 (s, 3H), 2.66-2.67 (m, 4H), 2.58 (t, J=7.2 Hz, 2H), 1.77-1.82 (m, 4H), 1.68-1.74 (m, 2H), 1.66 (d, J=3.6 Hz, 12H), 1.46-1.50 (m, 2H).

LCMS: m/z=719.3, C₄₅H₅₂ClN₂O₄⁺.

### Example 48: 2-((E)-2-((E)-2-chloro-3-(2-((E)-3,3-dimethyl-1-(6-oxo-6-phenoxyhexyl)indolin-2-ylidene)ethenyl)cyclohex-1-en-1-yl)ethenyl)-1-(2-hydroxyethyl)-3,3-dimethyl-3H-indol-1-ium Formate (48)

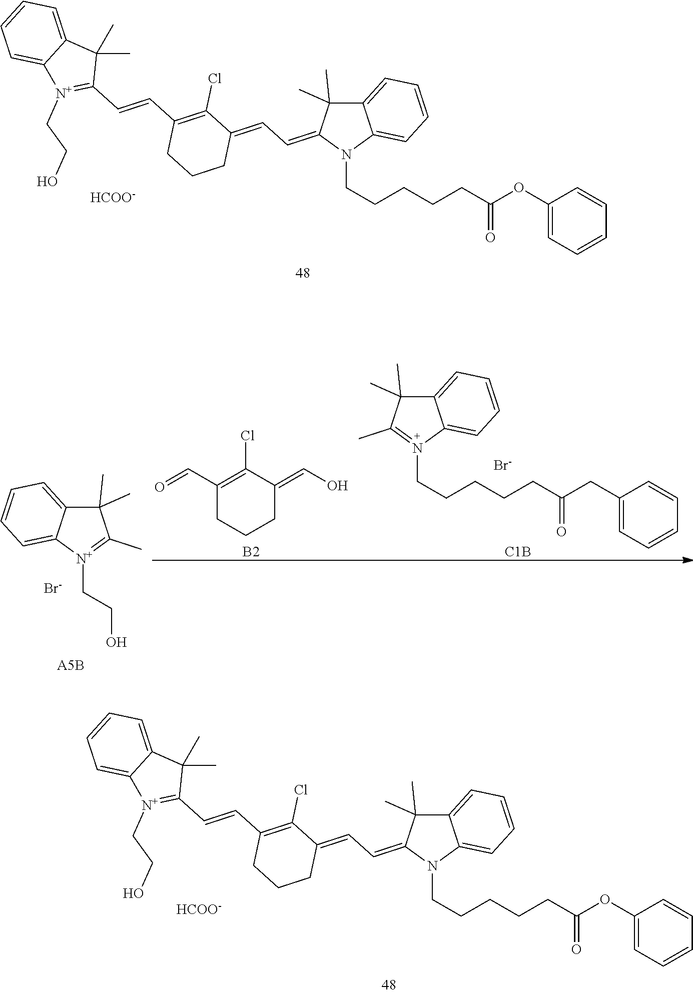

Compound 48 was prepared as a blue solid according to the preparation method of Example 21 with Compounds B2, A5B (prepared according to the preparation method of Compound A5) and C1B as raw materials.

¹H NMR (400 MHz, MeOD) δ 8.44 (dd, J₁=14.4 Hz, J₂=26.4 Hz, 2H), 7.53 (t, J=7.6 Hz, 2H), 7.41-7.45 (m, 2H), 7.26-7.38 (m, 6H), 7.19-7.23 (m, 1H), 6.98-7.00 (m, 2H), 6.45 (d, J=14.4 Hz, 1H), 6.27 (d, J=14.4 Hz, 1H), 4.32 (t, J=5.2 Hz, 2H), 4.20 (t, J=7.2 Hz, 2H), 3.97 (t, J=5.2 Hz, 2H), 2.67-2.72 (m, 4H), 2.61 (t, J=6.4 Hz, 2H), 1.88-1.93 (m, 4H), 1.81-1.85 (m, 2H), 1.77 (s, 6H), 1.72 (s, 6H), 1.55-1.61 (m, 2H).

LCMS: m/z=689.4, C₄₄H₅₀ClN₂O₃⁺.

### Example 49: Preparation of 2-((E)-2-((E)-3-(2-((E)-1-(5-carboxypentyl)-3,3-dimethylindolin-2-ylidene)ethenyl)-2-chlorocyclohex-1-en-1-yl)ethenyl)-1-(2-hydroxyethyl)-3,3-dimethyl-3H-indol-1-ium Chloride (49)

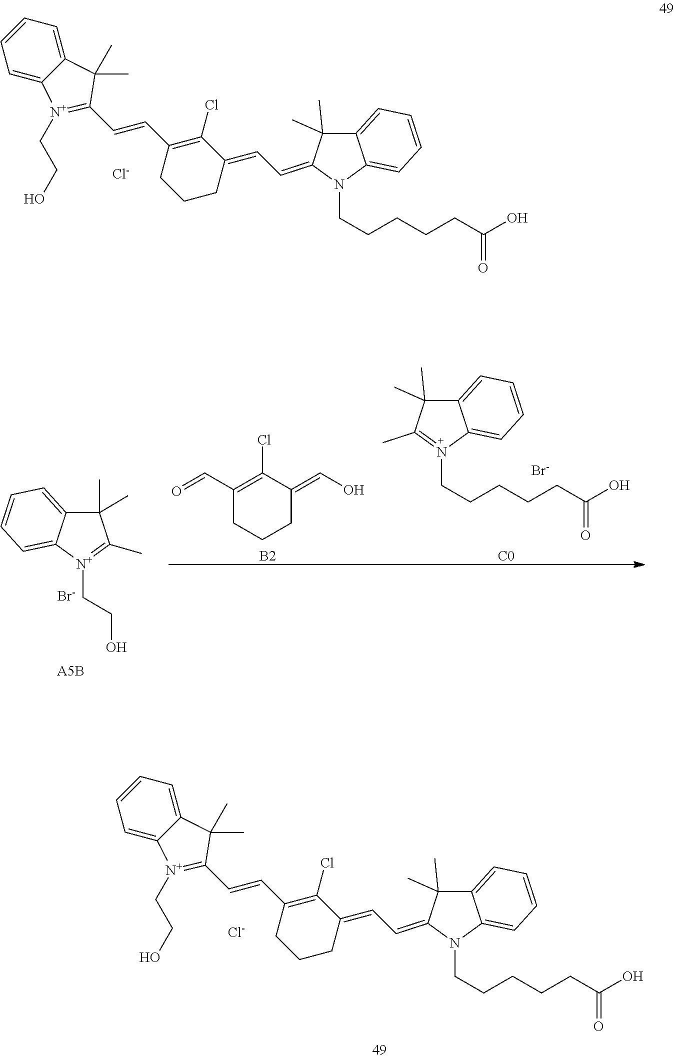

Compound 49 was prepared as a blue solid according to the preparation method of Example 21 with Compounds B2, A5B and C0 as raw materials.

¹H NMR (400 MHz, MeOD) δ 8.45 (br, 2H), 7.53 (d, J=8.0 Hz, 2H), 7.36-7.45 (m, 3H), 7.24-7.32 (m, 3H), 6.45 (br, 1H), 6.27 (br, 1H), 4.40 (s, 2H), 4.17 (s, 2H), 3.98 (t, J=5.2 Hz, 2H), 2.74 (br, 4H), 2.32 (t, J=7.6 Hz, 2H), 1.93-2.01 (m, 2H), 1.83-1.90 (m, 2H), 1.77 (s, 6H), 1.73 (s, 6H), 1.67-1.71 (m, 2H), 1.47-1.55 (m, 2H).

LCMS: m/z=613.3, C₃₈H₄₆ClN₂O₃.

### Example 50: Preparation of 2-((E)-2-((E)-2-chloro-3-(2-((E)-3,3-dimethyl-1-(6-oxo-6-phenoxyhexyl)indolin-2-ylidene)ethenyl)-5-(dimethylamino)cyclohex-1-en-1-yl)ethenyl)-1,3,3-trimethyl-3H-indol-1-ium Formate (50)

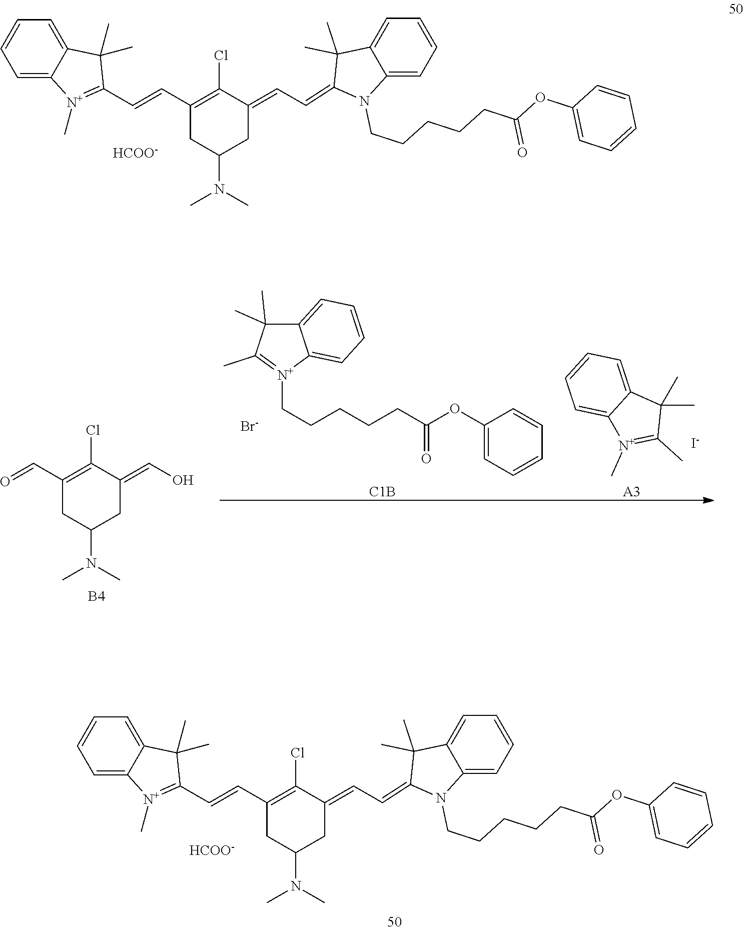

and Compound 50 was prepared as a green solid according to the preparation method of Example 21 with Compounds B4, A3 and C1B as raw materials.

¹H NMR (400 MHz, MeOD) δ 8.46 (t, J=14.0 Hz, 2H), 7.55-7.57 (m, 2H), 7.44-7.49 (m, 2H), 7.31-7.42 (m, 6H), 7.20-7.24 (m, 1H), 6.99-7.03 (m, 2H), 6.37-6.43 (m, 2H), 4.32-4.34 (m, 2H), 3.81 (s, 3H), 3.60-3.72 (m, 1H), 3.00 (s, 6H), 2.82-2.91 (m, 2H), 2.64 (t, J=7.2 Hz, 2H), 1.81-2.00 (m, 6H), 1.75 (dd, J₁=0.8 Hz, J₂=6.4 Hz, 12H), 1.61-1.66 (m, 2H).

LCMS: m/z=702.4, C₄₅H₅₃ClN₃O₂⁺.

### Example 51: Preparation of 2-((E)-2-((E)-2-chloro-3-(2-((E)-3,3-dimethyl-1-(2-(2-(3-oxo-3-phenoxypropoxy)ethoxy)ethyl)indolin-2-ylidene)ethenyl)cyclohex-1-en-1-yl)ethenyl)-1,3,3-trimethyl-3H-indol-1-ium Formate (51)

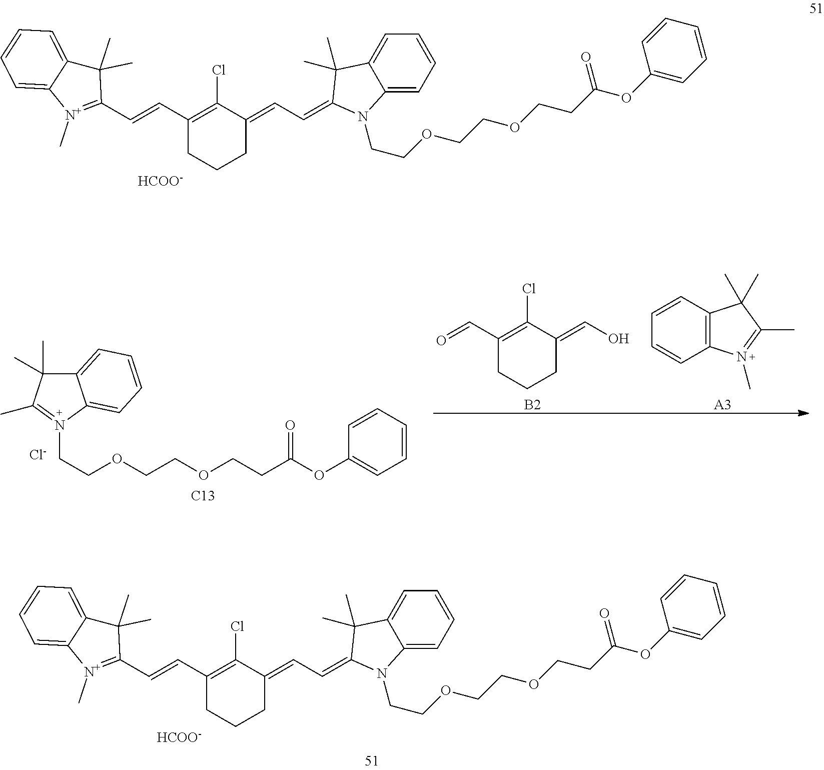

Compound 51 was prepared as a green solid according to the preparation method of Example 21 with Compounds B2, A3 and C13 as raw materials.

¹H NMR (400 MHz, CDCl₃) δ 8.26-8.31 (m, 2H), 7.29-7.31 (m, 5H), 7.15-7.22 (m, 6H), 7.00 (d, J=8.0 Hz, 2H), 6.27 (d, J=14.0 Hz, 1H), 6.10 (d, J=14.0 Hz, 1H), 4.31 (m, 2H), 3.87 (s, 2H), 3.67 (t, J=6.0 Hz, 2H), 3.62 (s, 3H), 3.52-3.56 (m, 4H), 2.62-2.68 (m, 6H), 1.89-1.90 (m, 2H), 1.65 (d, J=6.8 Hz, 12H).

LCMS: m/z=705.3, C₄₄H₅₀ClN₂O₄⁺.

### Example 52: Preparation of 2-((E)-2-((E)-2-chloro-3-(2-((E)-3,3-dimethyl-1-(2-(3-oxo-3-phenoxypropoxy)ethyl)indolin-2-ylidene)ethenyl)cyclohex-1-en-1-yl)ethenyl)-1,3,3-trimethyl-3H-indol-1-ium Chloride (52)

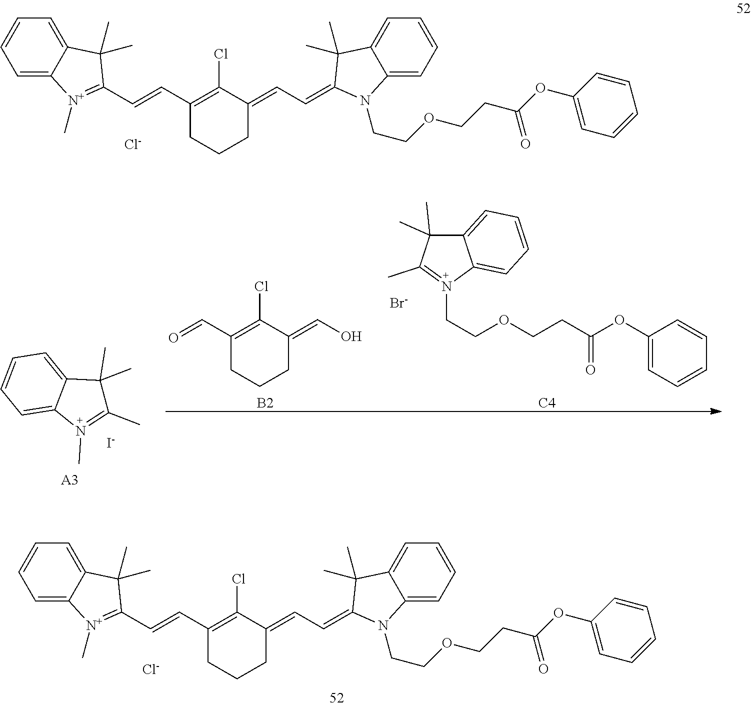

Compound 52 was prepared as a green solid according to the preparation method of Example 21 with Compounds B2, A3 and C4 as raw materials.

¹H NMR (400 MHz, MeOD); δ 7.52-7.42 (m, 8H), 7.29-7.26 (m, 4H), 7.18-7.14 (m, 2H), 6.85-6.83 (m, 3H), 3.98-3.90 (m, 2H), 3.80 (t, J=5.60 Hz, 4H), 2.74 (t, J=5.60 Hz, 4H), 1.73 (d, J=6.00 Hz, 10H), 1.69 (s, 9H).

LCMS: m/z=661.4, C₄₂H₄₆ClN₂O₃⁺.

### Example 53: Preparation of 2-((E)-2-((E)-2-chloro-3-(2-((E)-3,3-dimethyl-1-(6-oxo-6-phenoxyhexyl)indolin-2-ylidene)ethenyl)cyclohex-1-en-1-yl)ethenyl)-3,3,7-trimethyl-3H-pyrrolo[2,3-b]pyridin-7-ium 2,2,2-trifluoroacetate (53)

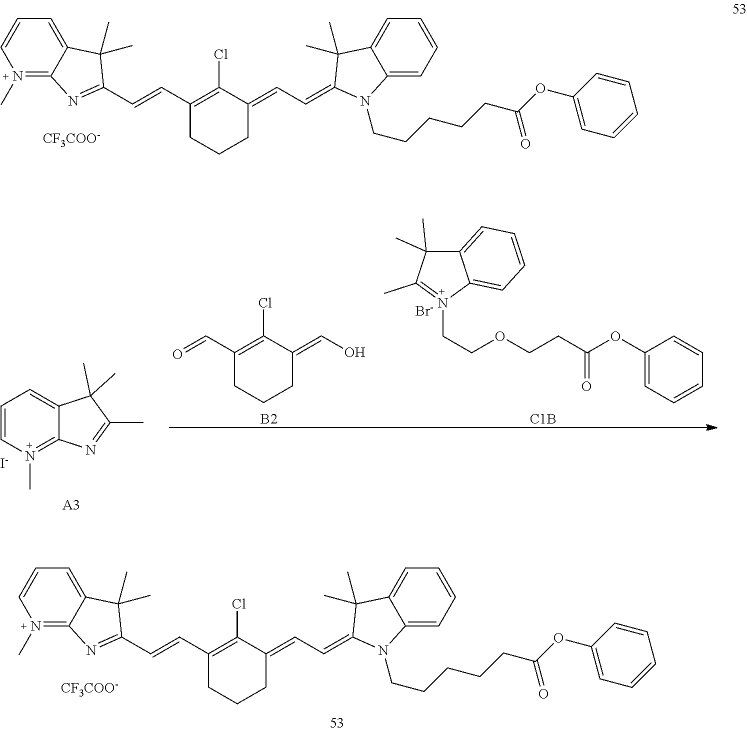

Compound 53 was prepared as a green solid according to the preparation method of Example 21 with Compounds B2, A3 and C1B as raw materials.

¹H NMR (400 MHz, DMSO-d₆) δ 8.42-8.47 (m, 2H), 8.30 (d, J=7.2 Hz, 1H), 7.94 (d, J=13.2 Hz, 1H), 7.49-7.51 (m, 2H), 7.32-7.41 (m, 3H), 7.25 (d, J=7.2 Hz, 2H), 7.14 (d, J=7.2 Hz, 1H), 7.03 (d, J=7.6 Hz, 2H), 6.57 (d, J=14.4 Hz, 1H), 6.05 (d, J=14.4 Hz, 1H), 4.23 (s, 3H), 4.07 (s, 2H), 2.66 (s, 4H), 2.58 (d, J=7.2 Hz, 2H), 1.67-1.78 (m, 6H), 1.62 (s, 6H), 1.49 (s, 8H).

LCMS: m/z=660.3, C₄₂H₄₇ClN₃O₂⁺.

### Example 54: Preparation of 2-((E)-2-((E)-2-bromo-3-(2-((E)-3,3-dimethyl-1-(6-oxo-6-phenoxyhexyl)indolin-2-ylidene)ethenyl)cyclohex-1-en-1-yl)ethenyl)-1-ethyl-3,3-dimethyl-3H-indol-1-ium Formate (54)

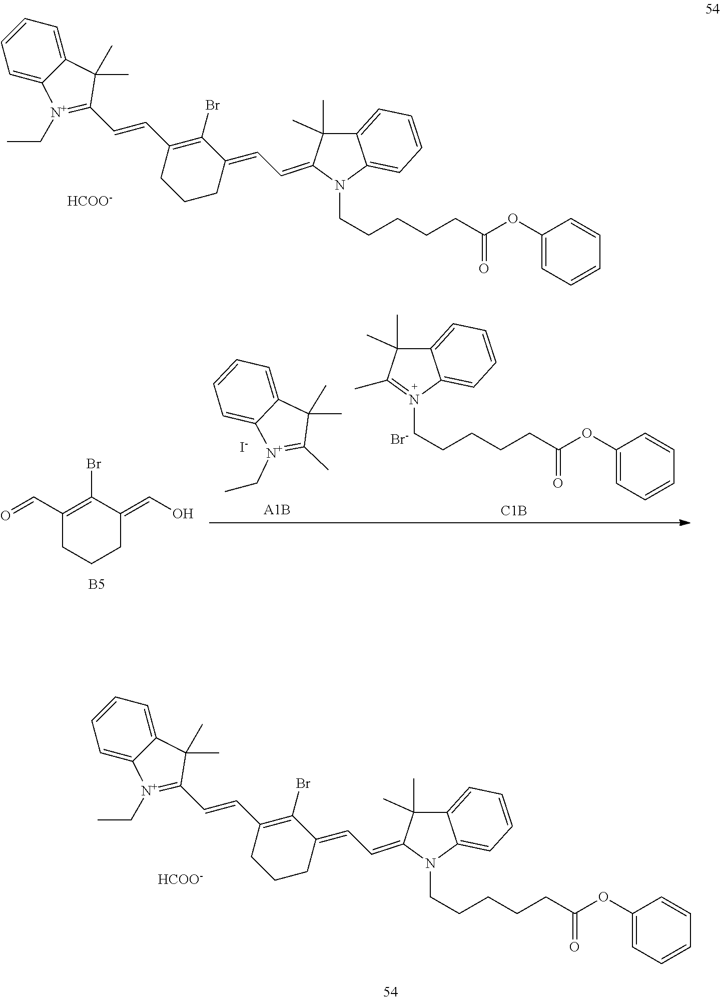

Compound 54 was prepared as a green solid according to the preparation method of Example 21 with Compounds B5, A1B and C1B as raw materials.

¹H NMR (400 MHz, MeOD) δ 8.46 (t, J=13.2 Hz, 2H), 7.53-7.56 (m, 2H), 7.42-7.47 (m, 2H), 7.30-7.37 (m, 6H), 7.19-7.23 (m, 1H), 6.98-7.01 (m, 2H), 6.31 (d, J=13.6 Hz, 2H), 4.15-4.30 (m, 4H), 2.72 (s, 4H), 2.62 (t, J=7.2 Hz, 2H), 1.90-1.97 (m, 4H), 1.81-1.84 (m, 2H), 1.75 (d, J=4.4 Hz, 12H), 1.55-1.61 (m, 2H), 1.42 (t, J=6.8 Hz, 3H).

LCMS: m/z=719.4, C₄₄H₅₀BrN₂O₂⁺.

### Example 55: Preparation of 2-((E)-2-((E)-3-(2-((E)-1-(2-(2-(2-(benzyloxy)ethoxy)ethoxy)ethyl)-3,3-dimethylindolin-2-ylidene)ethenyl)-2-chlorocyclopent-1-en-1-yl)ethenyl)-1,3,3-trimethyl-3H-indol-1-ium Formate (55)

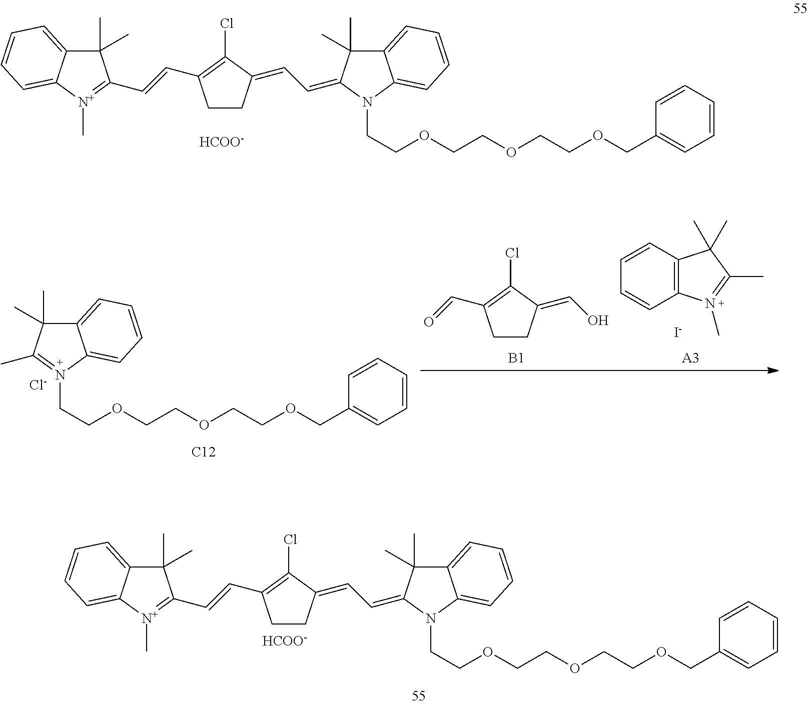

Compound 55 was prepared as a green solid according to the preparation method of Example 21 with Compounds B1, A3 and C12 as raw materials.

¹H NMR (400 MHz, MeOD) δ 7.95-7.92 (m, 2H), 7.51-7.49 (m, 2H), 7.43-7.39 (m, 2H), 7.35-7.34 (m, 2H), 7.32-7.29 (m, 6H), 7.28-7.25 (m, 1H), 6.28 (d, J=14.0 Hz, 1H), 6.04 (d, J=14.0 Hz, 1H), 4.58 (s, 1H), 4.47 (d, J=5.60 Hz, 2H), 4.36 (t, J=4.80 Hz, 2H), 3.92 (t, J=4.80 Hz, 2H), 3.64 (s, 3H), 3.61-3.55 (m, 2H), 3.53-3.51 (m, 5H), 2.95 (d, J=2.80 Hz, 4H), 1.71 (d, J=2.00 Hz, 12H).

LCMS: m/z=677.2, C₄₃H₅₀ClN₂O₃⁺.

### Example 56: Preparation of 2-((E)-2-((E)-3-(2-((E)-1-(2-(2-(2-(benzyloxy)ethoxy)ethoxy)ethyl)-3,3-dimethylindolin-2-ylidene)ethenyl)-2-chlorocyclohex-1-en-1-yl)ethenyl)-1,3,3-trimethyl-3H-indol-1-ium 2,2,2-trifluoroacetate (56)

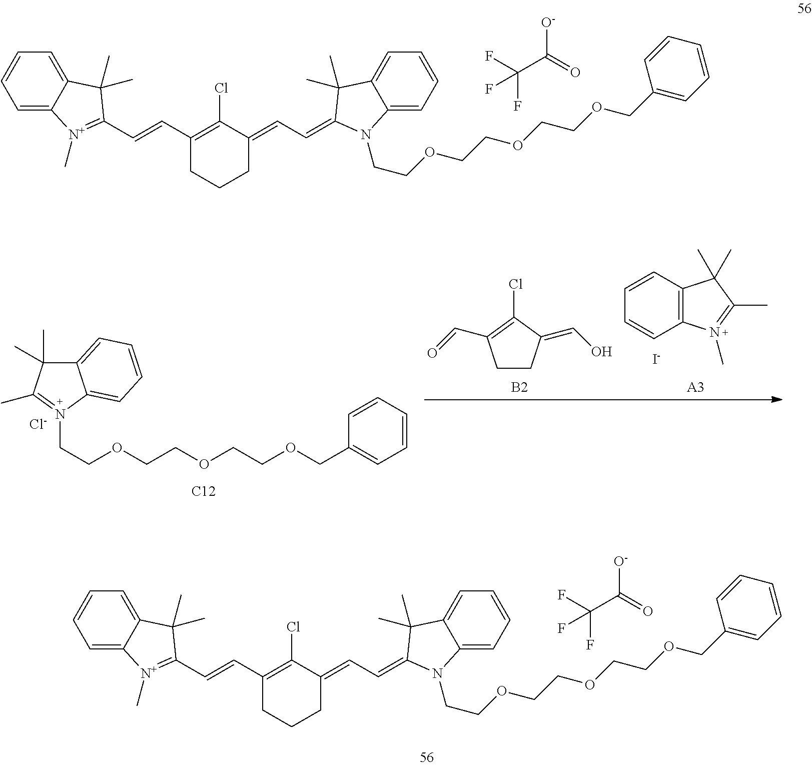

Compound 56 was prepared as a green solid according to the preparation method of Example 21 with Compounds B2, A3 and C12 as raw materials.

¹H NMVR (400 MHz, CDCl₃) δ 8.32 (d, J=13.2 Hz, 2H), 7.37-7.23 (m, 13H), 6.27-6.11 (m, 2H), 4.48 (s, 2H), 4.27 (s, 2H), 3.87 (t, J=4.80 Hz, 2H), 3.71-3.62 (m, 4H), 3.49-3.48 (m, 8H), 2.62 (d, J=1.60 Hz, 2H), 1.90-1.80 (m, 3H), 1.68 (d, J=2.80 Hz, 12H).

LCMS: m/z=691.6, C₄₄H₅₂ClN₂O₃⁺.

### Example 57: Preparation of 2-((E)-2-((E)-3-(2-((E)-1-(2-(2-(2-(benzyloxy)ethoxy)ethoxy)ethyl)-3,3-dimethylindolin-2-ylidene)ethenyl)-2-chloro-5-(dimethylamino)cyclohex-1-en-1-yl)ethenyl)-1,3,3-trimethyl-3H-indol-1-ium 2,2,2-trifluoroacetate (57)

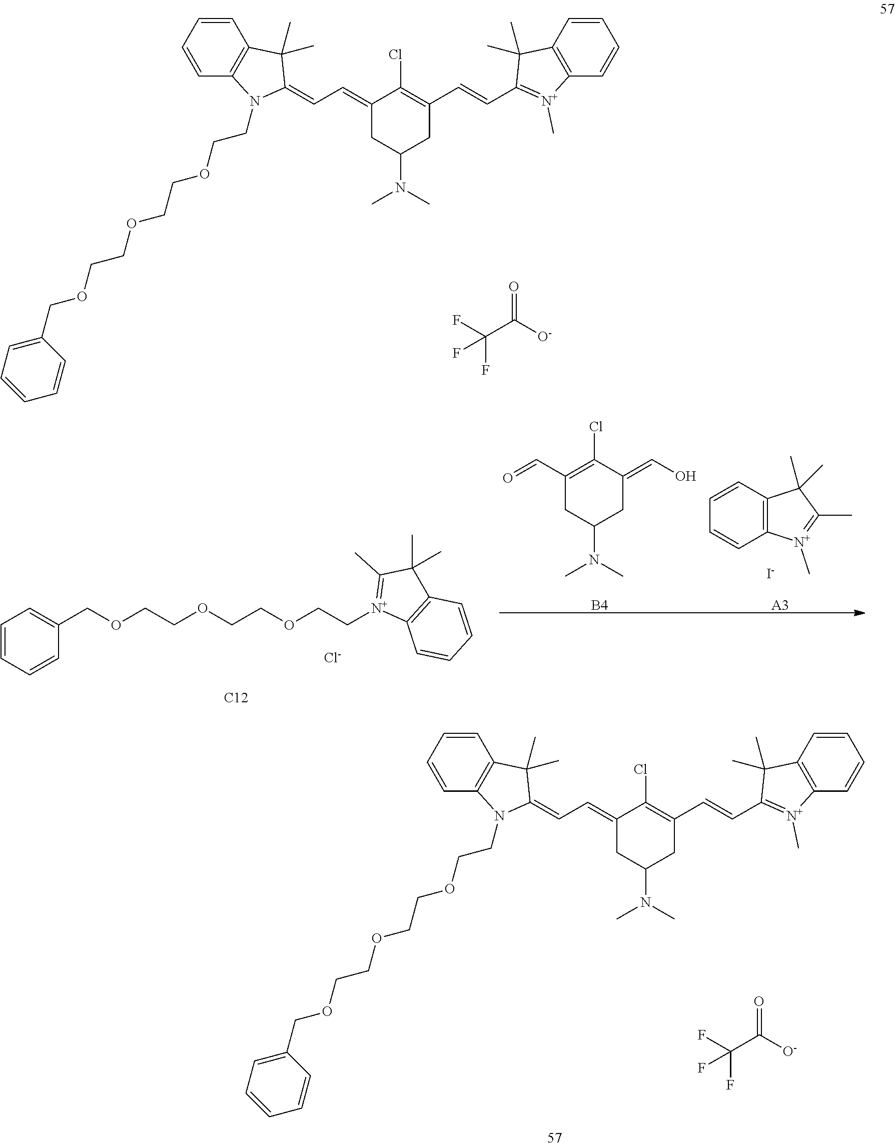

Compound 57 was prepared as a blue paste according to the preparation method of Example 21 with Compounds B4, A3 and C12 as raw materials.

¹H NMR: (400 MHz, MeOD) δ 8.70-8.42 (m, 2H), 7.50-7.52 (m, 2H), 7.30-7.32 (m, 4H), 7.30-7.28 (m, 7H), 6.50 (d, J=14.4 Hz, 1H), 6.34 (d, J=14.4 Hz, 1H), 4.48 (t, J=14.8 Hz, 2H), 4.44 (s, 2H), 3.9-3.92 (t, J=5.20 Hz, 2H), 3.75 (s, 3H), 2.70-3.67 (m, 1H), 3.60-3.58 (m, 8H), 3.3-3.33 (m, 2H), 3.05 (s, 6H), 2.88-2.79 (dd, J¹=12.0 Hz, J₂=26.4 Hz, 2H), 1.73 (d, J=2.00 Hz, 2H).

LCMS: m/z=734.4, C₄₆H₅₇ClN₃O₃⁺.

### Example 58: Preparation of 2-((E)-2-((E)-2-bromo-3-(2-((E)-3,3-dimethyl-1-(6-oxo-6-phenoxyhexyl)indolin-2-ylidene)ethenyl)cyclohex-1-en-1-yl)ethenyl)-1,3,3-trimethyl-3H-indol-1-ium 2,2,2-trifluoroacetate (58)

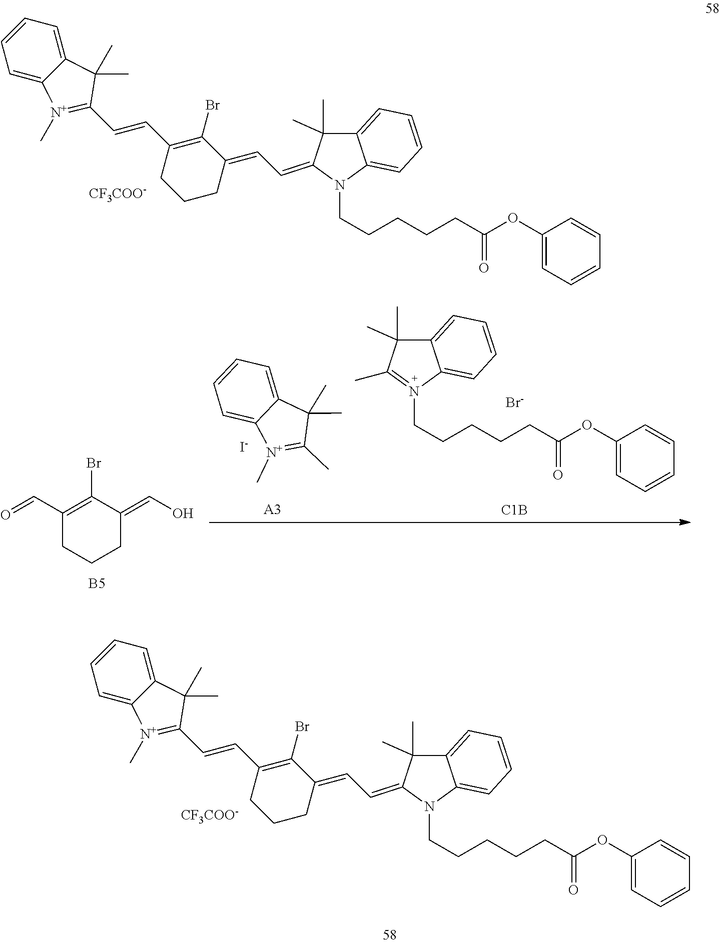

Compound 58 was prepared as a green solid according to the preparation method of Example 21 with Compounds B5, A3 and C1B as raw materials.

¹H NMR (400 MHz, CDCl₃) δ 8.34-8.41 (m, 2H), 7.37-7.42 (m, 4H), 7.27-7.30 (m, 2H), 7.14-7.19 (m, 2H), 6.18 (dd, J₁=14.4 Hz, J₂=33.2 Hz, 2H), 4.11 (t, J=7.2 Hz, 2H), 3.65 (s, 3H), 2.71 (t, J=5.6 Hz, 4H), 2.46 (t, J=6.8 Hz, 2H), 1.96-1.99 (m, 2H), 1.85-1.89 (m, 2H), 1.76-1.81 (m, 2H), 1.74 (s, 12H), 1.53-1.57 (m, 2H).

LCMS: m/z=629.3, C₃₇H₄₄BrN₂O₂⁺.

### Example 60: Preparation of 2-((E)-2-((E)-2-chloro-3-(2-((E)-3,3-dimethyl-1-(6-oxo-6-phenoxyhexyl)indolin-2-ylidene)ethenyl)cyclohex-1-en-1-yl)ethenyl)-3,3-dimethyl-1-(5-(trimethylammonium)pentyl)-3H-indol-1-ium 2,2,2-trifluoroacetate (60)

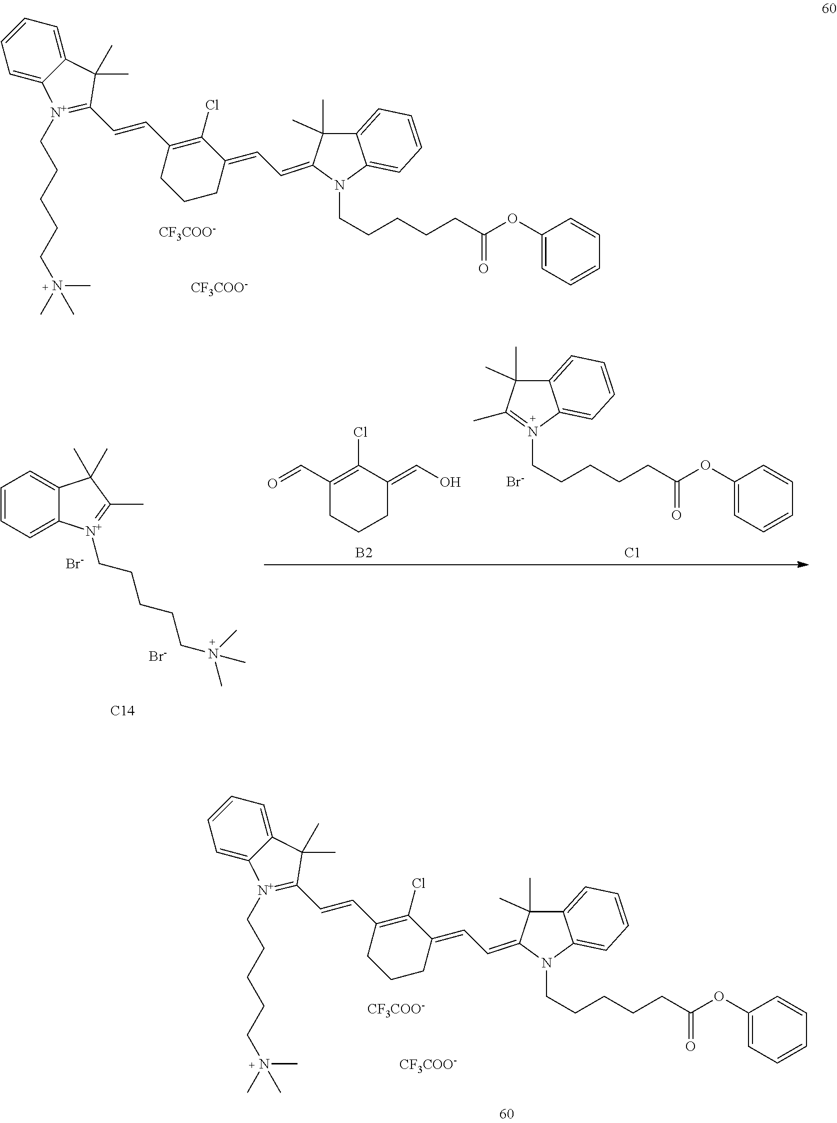

and Compound 60 was prepared as a green solid according to the preparation method of Example 21 with Compounds B2, C1 and C14 as raw materials.

¹H NMR (400 MHz, DMSO) δ 8.27 (t, J=14.8 Hz, 2H), 7.65 (dd, J₁=7.6 Hz, J₂=11.2 Hz, 2H), 7.37-7.52 (m, 6H), 7.23-7.34 (m, 3H), 7.02-7.04 (m, 2H), 6.35 (dd, J₁=14.0 Hz, J₂=33.2 Hz, 2H), 4.22-4.30 (m, 4H), 3.22-3.26 (m, 4H), 3.01 (s, 9H), 2.67-2.72 (m, 4H), 2.58 (t, J=7.2 Hz, 2H), 1.71-1.82 (m, 8H), 1.67 (s, 12H), 1.47-1.57 (m, 2H), 1.36-1.40 (m, 2H).

LCMS: m/z=387.6, C₅₀H₆₄ClN₃O₂²⁺/2.

### Example 61: Preparation of 2-((E)-2-((E)-2-chloro-3-(2-((E)-3,3-dimethyl-1-(6-oxo-6-phenoxyhexyl)indolin-2-ylidene)ethenyl)cyclohex-1-en-1-yl)ethenyl)-3,3-dimethyl-1-(4-(trimethylammonium)butyl)-3H-indol-1-ium 2,2,2-trifluoroacetate (61)

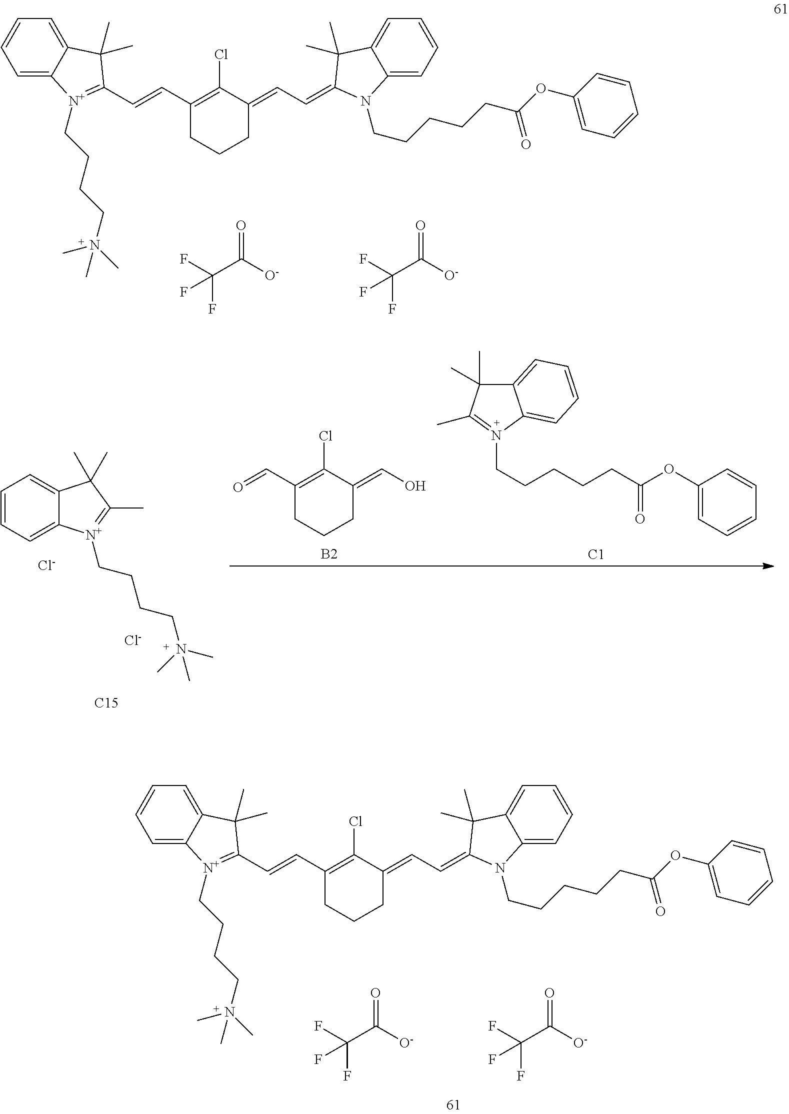

Compound 61 was prepared as a green solid according to the preparation method of Example 21 with Compounds B2, C1 and C15 as raw materials.

¹H NMR: (400 MHz, CDCl₃) δ 8.43 (d, J=14.4 Hz, 1H), 8.27 (t, J=13.2 Hz, 1H), 7.40-7.43 (m, 2H), 7.33-7.37 (m, 5H), 7.19-7.20 (m, 3H), 7.00-7.05 (m, 3H), 6.32 (t, J=13.6 Hz, 1H), 6.05 (t, J=13.6 Hz, 1H), 4.00-4.16 (m, 4H), 3.39-3.43 (m, 2H), 3.13 (s, 9H), 2.75 (s, 2H), 2.57-2.64 (m, 4H), 2.05-2.10 (m, 2H), 1.92-1.94 (m, 3H), 1.81-1.88 (m, 5H), 1.70 (s, 12H), 1.58-1.69 (m, 2H).

LCMS: m/z=380.2, C₄₉H₆₂ClN₃O₂₂⁺/2.

### Example 62: Preparation of 2-((E)-2-((E)-2-chloro-3-(2-((E)-1-(5-(diethoxyphosphonyl)pentyl)-3,3-dimethylindolin-2-ylidene)ethenyl)cyclohex-1-en-1-yl)ethenyl)-1,3,3-trimethyl-3H-indol-1-ium 2,2,2-trifluoroacetate (62)

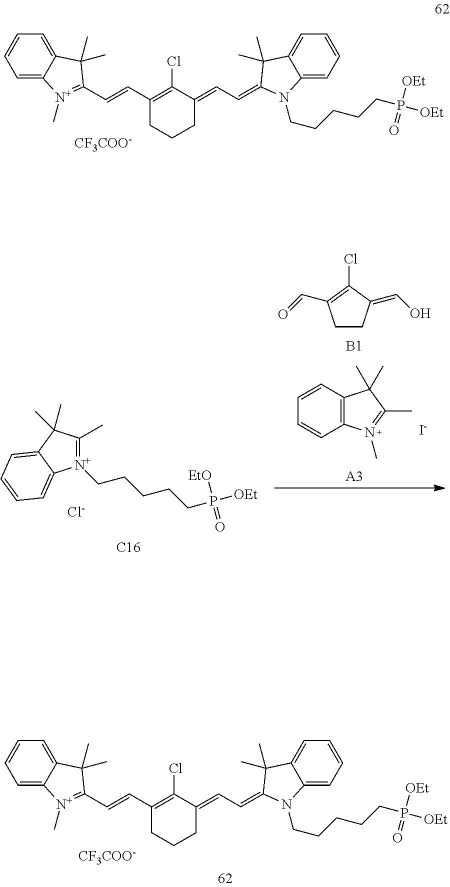

Compound 62 was prepared as a green solid according to the preparation method of Example 21 with Compounds B1, A3 and C16 as raw materials.

LCMS: m/z=675.4, C₄₀H₅₃ClN₂O₃P⁺.

### Example 63: Preparation of 2-((E)-2-((E)-3-(2-((E)-1-(5-carboxypentyl)-3,3-dimethylindolin-2-ylidene)ethenyl)-2-chlorocyclopent-1-en-1-yl)ethenyl)-3,3,7-trimethyl-3H-pyrrolo[2,3-b]pyridin-7-ium Chloride (63)

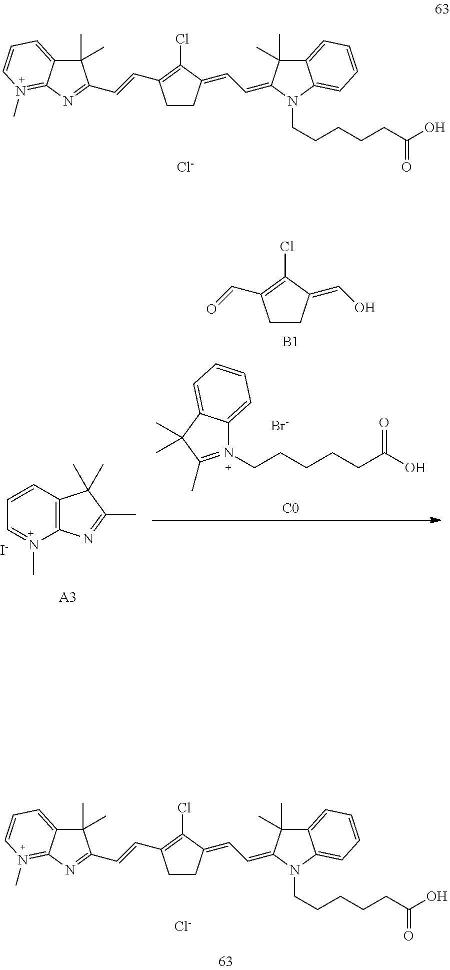

Compound 63 was prepared as a blue paste according to the preparation method of Example 21 with Compounds B1, A3 and C0 as raw materials.

¹H NMR (400 MHz, MeOD) δ 8.29 (d, J=15.2 Hz, 1H), 8.01 (s, 2H), 7.83-7.78 (m, 2H), 7.67-7.56 (m, 3H), 7.17 (s, 1H), 6.92 (d, J=15.6 Hz, 1H), 5.83 (d, J=11.6 Hz, 1H), 4.63 (s, 2H), 4.16 (s, 3H), 3.04 (d, J=20.0 Hz, 4H), 2.33 (t, J=11.2 Hz, 2H), 2.04-2.00 (m, 2H), 1.82 (s, 6H), 1.73-1.67 (m, 3H), 1.62-1.55 (m, 7H).

LCMS: m/z=570.4, C₃₅H₄₁ClN₃O₂⁺.

### Example 64: Preparation of 2-((E)-2-((E)-3-(2-((E)-1-(5-carboxypentyl)-3,3-dimethylindolin-2-ylidene)ethenyl)-2-chlorocyclohex-1-en-1-yl)ethenyl)-3,3,7-trimethyl-3H-pyrrolo[2,3-b]pyridin-7-ium Chloride (64)

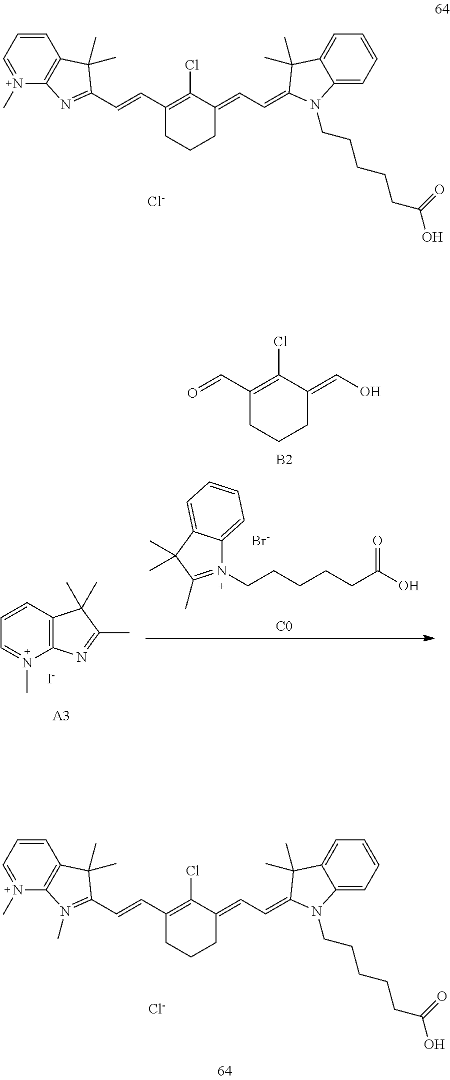

Compound 64 was prepared as a blue solid according to the preparation method of Example 21 with Compounds B2, A3 and C0 as raw materials.

¹H NMR (400 MHz, MeOD) 8.75 (d, J=15.6 Hz, 1H), 8.01 (d, J=7.20 Hz, 2H), 7.81-7.78 (m, 3H), 7.65-7.63 (m, 2H), 7.18 (t, J=6.80 Hz, 1H), 7.03 (d, J=15.6 Hz, 1H), 6.04 (t, J=5.60 Hz, 1H), 4.62 (t, J=7.20 Hz, 2H), 4.15 (s, 3H), 2.83-2.80 (m, 4H), 2.33 (t, J=7.20 Hz, 2H), 1.99-1.98 (m, 4H), 1.83 (s, 6H), 1.73-1.67 (m, 3H), 1.58-1.56 (m, 7H).

LCMS: m/z=584.4, C₃₆H₄₃ClN₃O₂⁺.

### Example 65: Preparation of 2-((E)-2-((E)-2-chloro-3-(2-((E)-3,3-dimethyl-1-(6-oxo-6-phenoxyhexyl)indolin-2-ylidene)ethenyl)cyclohex-1-en-1-yl)ethenyl)-3,3-dimethyl-1-(3-(trimethylammonium)propyl)-3H-indol-1-ium 2,2,2-trifluoroacetate (65)

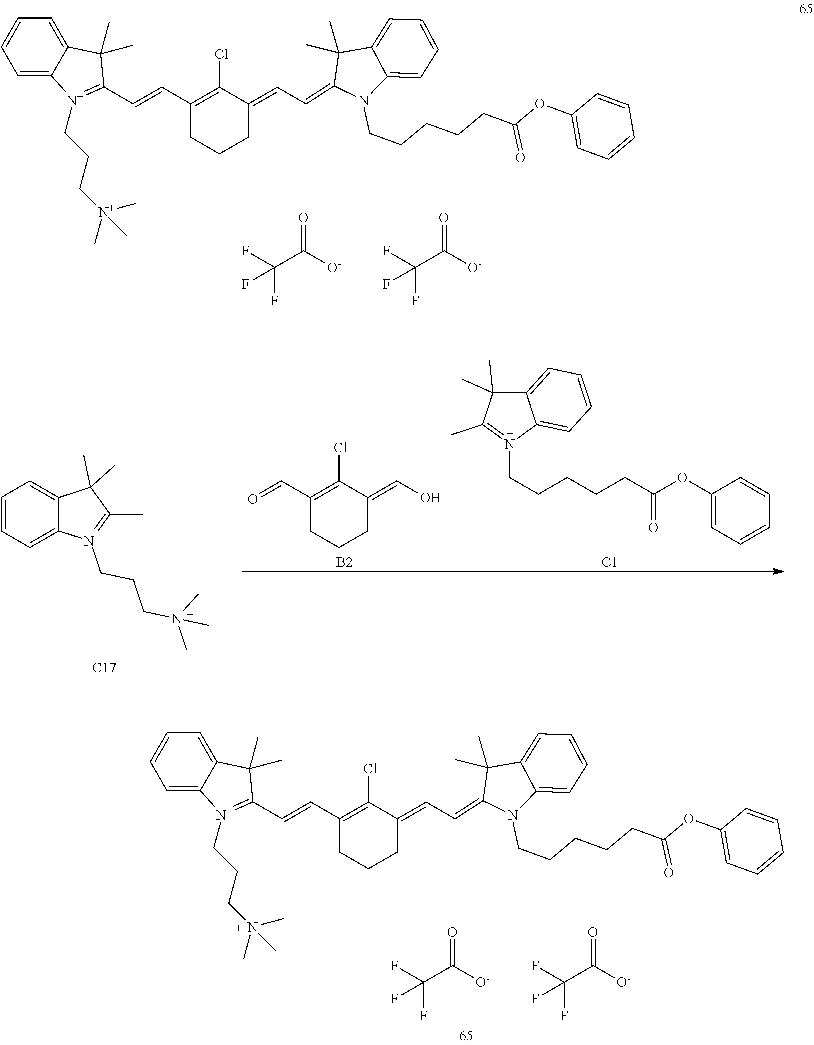

Compound 65 was prepared as a blue paste according to the preparation method of Example 21 with Compounds B2, C1 and C17 as raw materials.

¹H NMR: (400 MHz, CDCl₃) δ 8.46 (d, J=14.4 Hz, 1H), 8.29 (d, J=13.6 Hz, 1H), 7.61 (d, J=7.6 Hz, 1H), 7.46 (t, J=7.6 Hz, 1H), 7.34-7.38 (m, 5H), 7.29 (s, 1H), 7.19-7.21 (m, 2H), 7.00-7.03 (m, 3H), 6.54 (d, J=14.0 Hz, 1H), 6.01 (d, J=14.0 Hz, 1H), 4.36 (s, 2H), 4.00-4.02 (m, 4H), 3.24 (s, 9H), 2.77 (s, 2H), 2.59-2.62 (m, 4H), 2.38 (s, 2H), 1.82-1.91 (m, 6H), 1.72 (d, J=10.0 Hz, 12H), 1.56-1.58 (m, 2H).

LCMS: m/z=373.1, C₄₈H₆₀ClN₃O₂₂⁺/2.

### Example 66: Preparation of 2-((E)-2-((E)-2-chloro-3-(2-((E)-3,3-dimethyl-1-(6-oxo-6-phenoxyhexyl)indolin-2-ylidene)ethenyl)cyclohex-1-en-1-yl)ethenyl)-5-(dimethylamino)-1,3,3-trimethyl-3H-indol-1-ium Chloride (66)

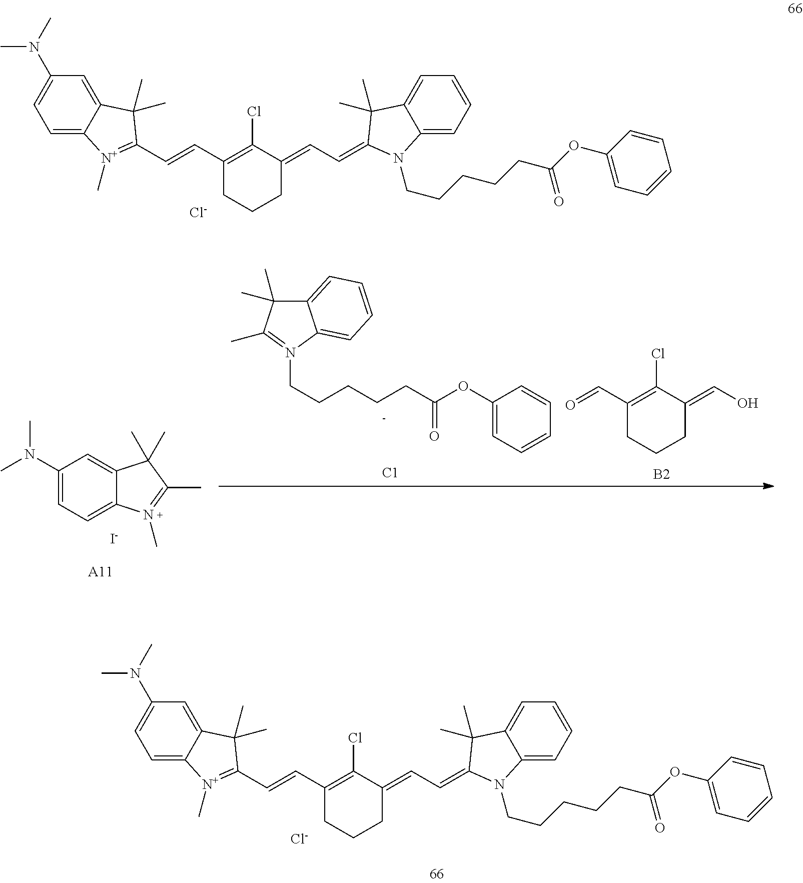

Compound 66 was prepared as a blue solid according to the preparation method of Example 21 with Compounds B2, A11 and C1 as raw materials.

¹H NMR (400 MHz, DMSO-_{d6}) δ 8.26 (d, J=14.8 Hz, 1H), 7.92 (d, J=13.6 Hz, 1H), 7.51-7.46 (m, 2H), 7.41-7.37 (m, 2H), 7.32-7.23 (m, 2H), 7.16 (d, J=8.0 Hz, 1H), 7.09-7.04 (m, 2H), 7.03 (d, J=7.6 Hz, 2H), 6.82 (d, J=8.0 Hz, 1H), 6.58 (d, J=15.2 Hz, 1H), 5.92 (d, J=13.2 Hz, 1H), 4.00 (s, 2H), 3.85 (s, 3H), 3.03 (s, 6H), 2.66 (s, 4H), 2.58 (t, J=7.2 Hz, 2H), 1.84-1.73 (m, 6H), 1.68 (s, 6H), 1.61 (s, 6H), 1.51-1.45 (m, 2H).

LCMS: m/z=702.4, C₄₅H₅₃ClN₃O₂⁺.

### Example 67: Preparation of 2-((E)-2-((E)-2-chloro-3-(2-((E)-1-(2-(2-methoxyethoxy)ethyl)-3,3-dimethylindolin-2-ylidene)ethenyl)cyclohex-1-en-1-yl)ethenyl)-3,3-dimethyl-1-(2-(2-(3-oxo-3-phenoxypropoxy)ethoxy)ethyl)-3H-indol-1-ium 2,2,2-trifluoroacetate (67)

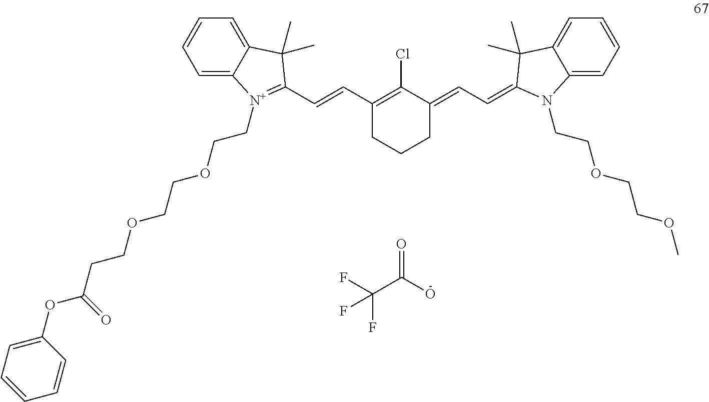 [SMILES: COCCOCCN1/C(=C/C=C2\CCCC(/C=C/C3=[N+](CCOCCOCCC(=O)Oc4ccccc4)c4ccccc4C3(C)C)=C2Cl)C(C)(C)c2ccccc21 ; O=C(O)C(F)(F)F]

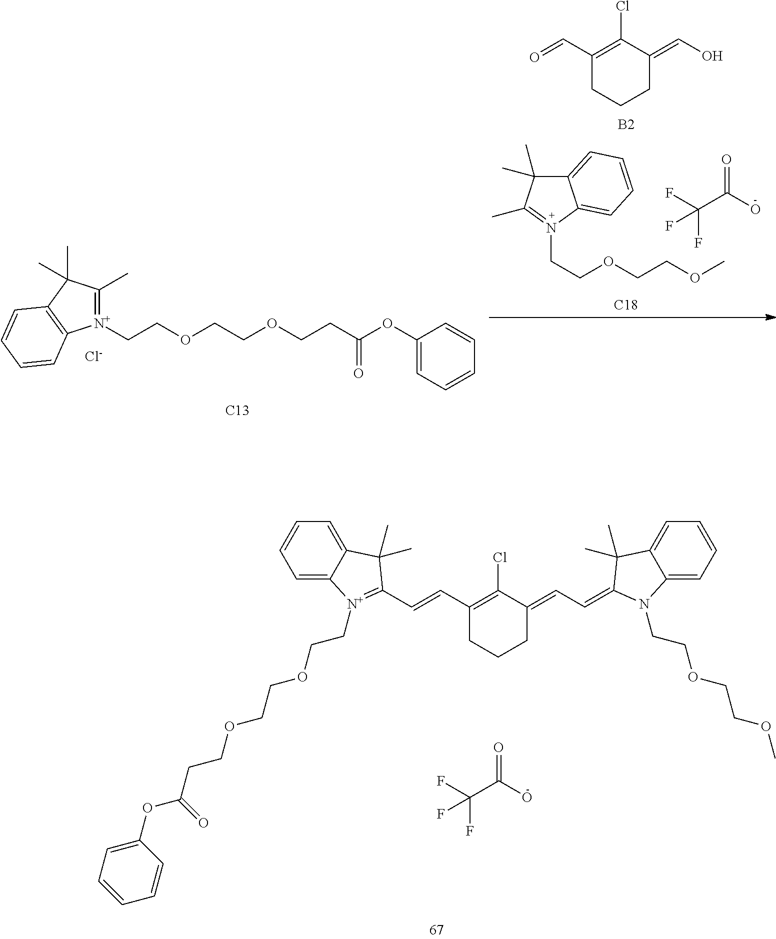

Compound 67 was prepared as a green paste according to the preparation method of Example 21 with Compounds B2, C13 and C18 as raw materials.

¹H NMR (400 MHz, CDCl₃) δ 8.35 (d, J=14.4 Hz, 2H), 7.36-7.39 (m, 6H), 7.23-7.25 (m, 5H), 7.06 (d, J=8.4 Hz, 2H), 6.30 (d, J=14.0 Hz, 2H), 4.33-4.35 (m, 4H), 3.89-3.93 (m, 4H), 3.76 (t, J=6.4 Hz, 2H), 3.58-3.63 (m, 6H), 3.43-3.46 (m, 2H), 3.28 (s, 3H), 2.67-2.75 (s, 6H), 1.95-1.96 (m, 2H), 1.73 (s, 12H).

LCMS: m/z=793.5, C₄₈H₅₈ClN₂O₆⁺.

### Example 68: Preparation of 1-(2-(2-(2-carboxyethoxy)ethoxy)ethyl)-2-((E)-2-((E)-2-chloro-3-(2-((E)-1-(2-(2-methoxyethoxy)ethyl)-3,3-dimethylindolin-2-ylidene)ethenyl)cyclohex-1-en-1-yl)ethenyl)-3,3-dimethyl-3H-indol-1-ium 2,2,2-trifluoroacetate (68)

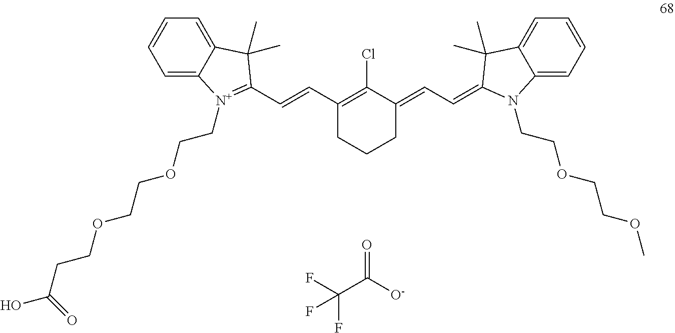 [SMILES: COCCOCCN1/C(=C/C=C2\CCCC(/C=C/C3=[N+](CCOCCOCCC(=O)O)c4ccccc4C3(C)C)=C2Cl)C(C)(C)c2ccccc21 ; O=C([O-])C(F)(F)F]

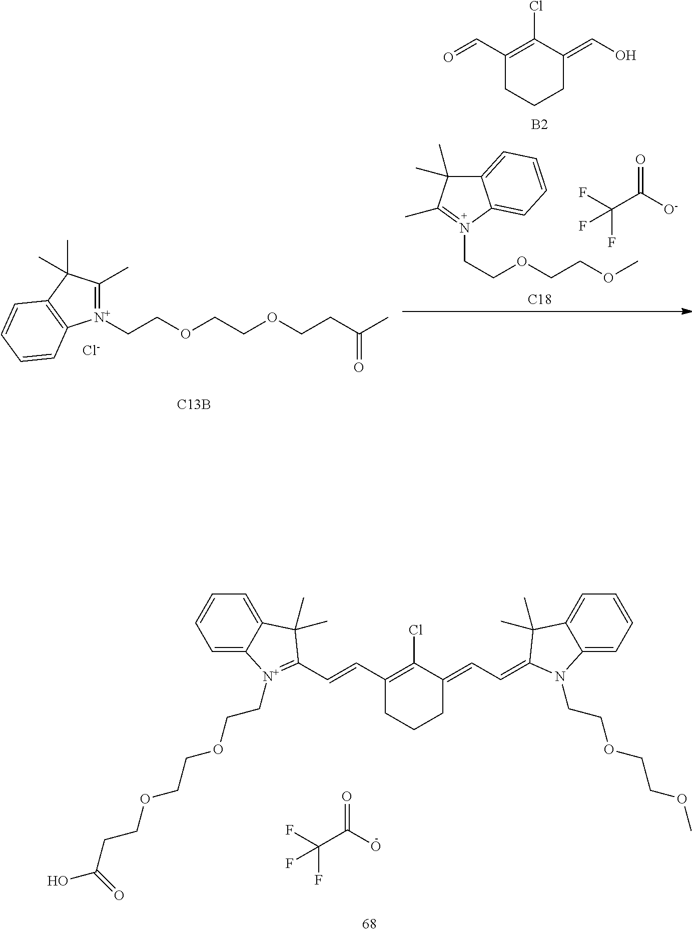

Compound 68 was prepared as a green paste according to the preparation method of Example 21 with Compounds B2, C13B (purchased from Bide Pharm, batch number 220503) and C18 as raw materials.

¹H NMR (400 MHz, CDCl₃) δ 8.43 (d, J=14.4 Hz, 1H), 8.25 (d, J=13.6 Hz, 1H), 7.30-7.43 (m, 6H), 7.19 (d, J=7.2 Hz, 1H), 7.11 (d, J=8.0 Hz, 1H), 6.57 (d, J=14.4 Hz, 1H), 6.14 (d, J=14.0 Hz, 1H), 4.46 (t, J=4.8 Hz, 2H), 4.22 (t, J=5.2 Hz, 2H), 4.00 (t, J=5.2 Hz, 2H), 3.88 (t, J=5.2 Hz, 2H), 3.74 (t, J=6.0 Hz, 2H), 3.57-3.63 (m, 4H), 3.48-3.49 (m, 4H), 3.31 (s, 3H), 2.73 (t, J=4.0 Hz, 2H), 2.60-2.66 (m, 4H), 1.95-1.97 (m, 2H), 1.73 (d, J=9.2 Hz, 12H).

LCMS: m/z=717.4 C₄₂H₅₄ClN₂O₆⁺.

### Example 69: Preparation of 2-((E)-2-((E)-2-chloro-3-(2-((E)-1-(2-(2-(2-methoxyethoxy)ethoxy)ethyl)-3,3-dimethylindolin-2-ylidene)ethenyl)cyclohex-1-en-1-yl)ethenyl)-3,3-dimethyl-1-(2-(2-(3-oxo-3-phenoxypropoxy)ethoxy)ethyl)-3H-indol-1-ium 2,2,2-trifluoroacetate (69)

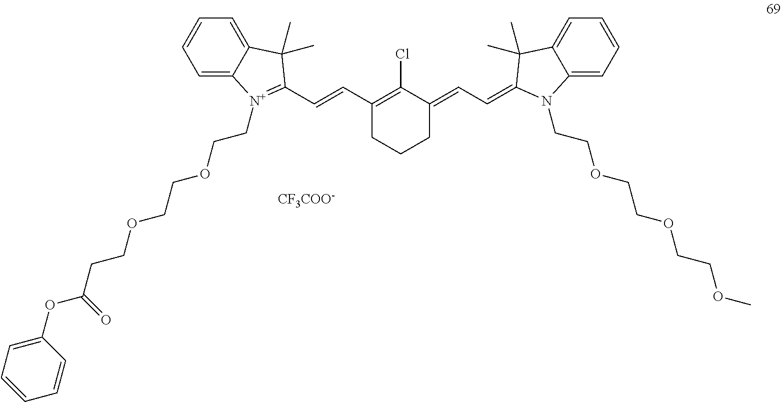

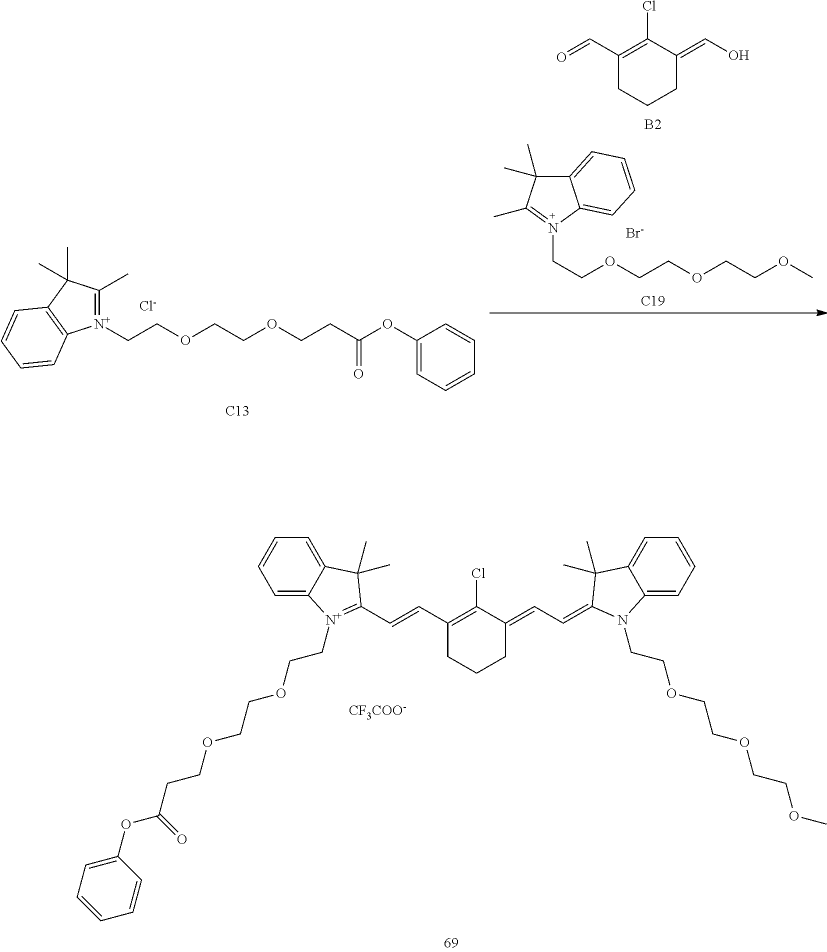

Compound 69 was prepared as a green paste according to the preparation method of Example 21 with Compounds B2, C13 and C19 as raw materials.

¹H NMR (400 MHz, CDCl₃) δ 8.35 (d, J=14.0 Hz, 2H), 7.34-7.40 (m, 6H), 7.20-7.26 (m, 5H), 7.05-7.07 (m, 2H), 6.29 (d, J=14.0 Hz, 2H), 4.29-4.35 (m, 4H), 3.87-3.95 (m, 4H), 3.75 (t, J=6.0 Hz, 2H), 3.60-3.64 (m, 4H), 3.55-3.59 (m, 4H), 3.51-3.53 (m, 2H), 3.44-3.46 (m, 2H), 3.33 (s, 3H), 2.73 (t, J=6.4 Hz, 2H), 2.62-2.69 (m, 4H), 1.88-1.99 (m, 2H), 1.73 (s, 12H).

LCMS: m/z=837.4, C₅₀H₆₂ClN₂O₇⁺.

### Example 70: Preparation of 1-(2-(2-(2-carboxyethoxy)ethoxy)ethyl)-2-((E)-2-((E)-2-chloro-3-(2-((E)-1-(2-(2-(2-methoxyethoxy)ethoxy)ethyl)-3,3-dimethylindolin-2-ylidene)ethenyl)cyclohex-1-en-1-yl)ethenyl)-3,3-dimethyl-3H-indol-1-ium 2,2,2-trifluoroacetate (70)

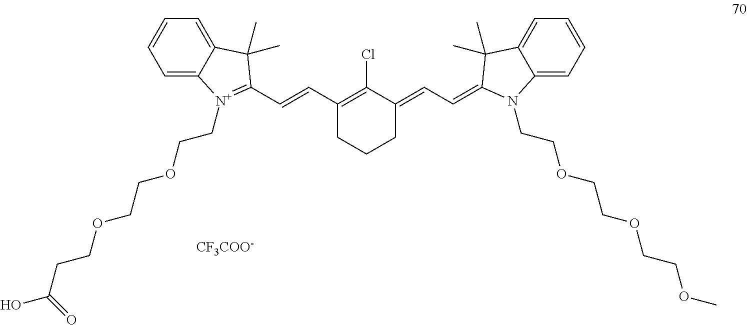

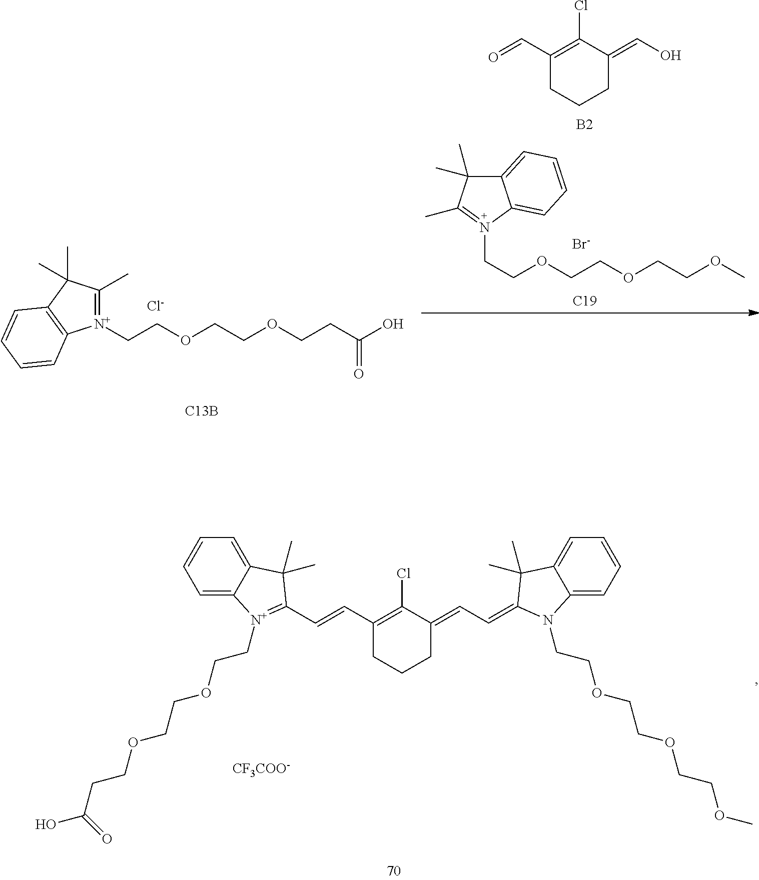

and Compound 70 was prepared as a blue paste according to the preparation method of Example 21 with Compounds B2, C13B and C19 as raw materials.

¹H NMR (400 MHz, CDCl3) δ 8.41 (d, J=14.0 Hz, 1H), 8.28 (d, J=14.0 Hz, 1H), 7.33-7.43 (m, 4H), 7.28-7.31 (m, 2H), 7.20 (d, J=7.2 Hz, 1H), 7.13-7.15 (m, 1H), 6.47 (d, J=14.4 Hz, 1H), 6.17 (d, J=14.0 Hz, 1H), 4.36-4.42 (m, 2H), 4.24 (t, J=5.2 Hz, 2H), 3.96 (t, J=4.8 Hz, 2H), 3.88 (t, J=4.4 Hz, 2H), 3.70 (t, J=6.0 Hz, 2H), 3.61-2.63 (m, 2H), 3.52-3.58 (m, 8H), 3.45-3.47 (m, 2H), 3.34 (s, 3H), 3.64-3.71 (m, 4H), 2.59 (t, J=6.0 Hz, 2H), 1.90-1.98 (m, 2H), 1.72 (d, J=6.8 Hz, 12H).

LCMS: m/z=761.5, C₄₄H₅₈ClN₂O₇⁺.

### Example 71: Preparation of 2-((E)-2-((E)-2-chloro-3-(2-((E)-1-(2-(2-methoxyethoxy)ethyl)-3,3-dimethylindolin-2-ylidene)ethenyl)cyclohex-1-en-1-yl)ethenyl)-1-(2-(2-(2-methoxyethoxy)ethoxy)ethyl)-3, 3-dimethyl-3H-indol-1-ium 2,2,2-trifluoroacetate (71)

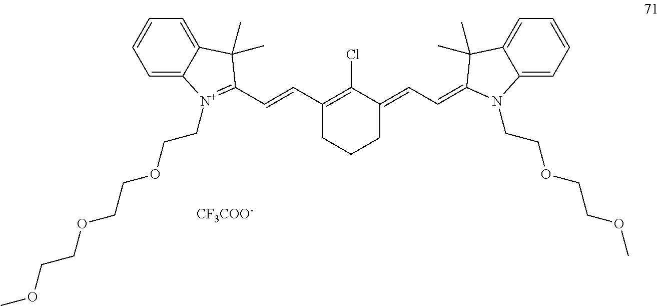

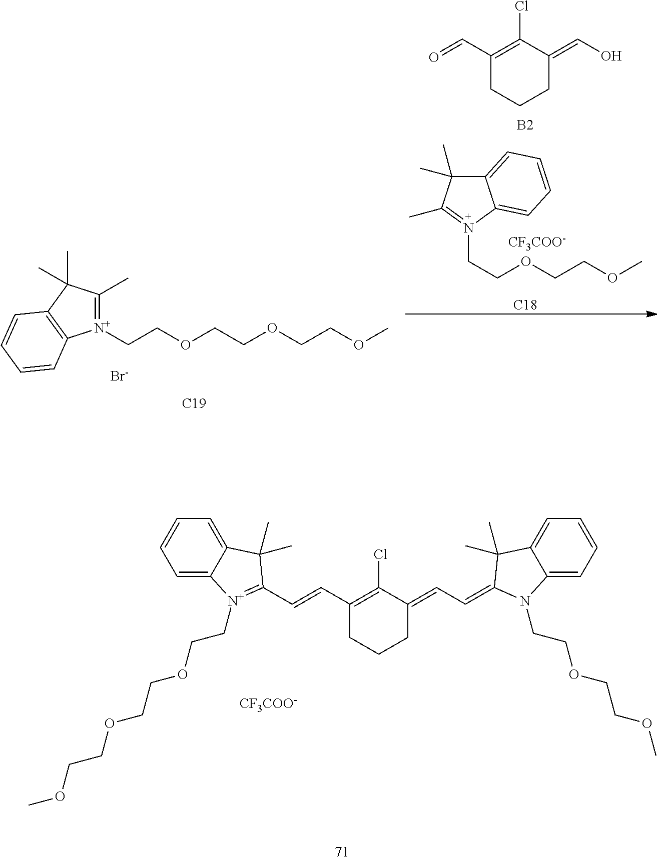

Compound 71 was prepared as a green paste according to the preparation method of Example 21 with Compounds B2, C18 and C19 as raw materials.

¹H NMR (400 MHz, CDCl₃) δ 8.33 (dd, J₁=5.6 Hz, J₂=14.0 Hz, 2H), 7.35-7.71 (m, 4H), 7.23-7.25 (m, 4H), 6.36 (t, J₁=13.6 Hz, 2H), 4.36-4.42 (m, 4H), 3.91-3.95 (m, 4H), 3.60-3.65 (m, 4H), 3.56-3.58 (m, 2H), 3.53-3.55 (m, 2H), 3.45-3.47 (m, 4H), 3.35 (s, 3H), 3.28 (s, 3H), 2.64-2.72 (m, 4H), 1.93-1.96 (m, 2H), 1.73 (s, 12H).

LCMS: m/z=703.3, C₄₂H₅₆ClN₂O₅⁺.

### Example 72: Preparation of 2-((E)-2-((E)-2-chloro-3-(2-((E)-3,3-dimethyl-1-(2,5,8,11-tetraoxatridecan-13-yl)indolin-2-ylidene)ethenyl)cyclohex-1-en-1-yl)ethenyl)-3,3-dimethyl-1-(2-(2-(3-oxo-3-phenoxypropoxy)ethoxy)ethyl)-3H-indol-1-ium 2,2,2-trifluoroacetate (72)

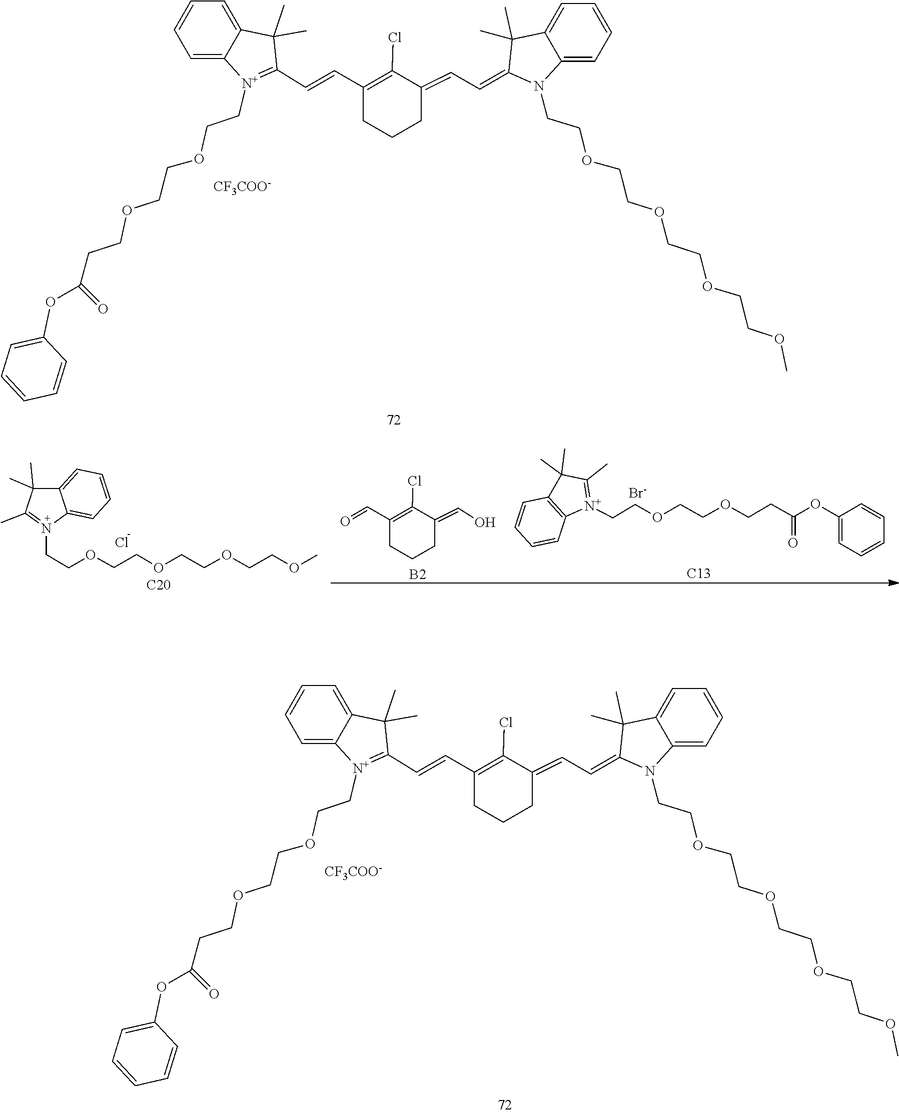

Compound 72 was prepared as a green paste according to the preparation method of Example 21 with Compounds B2, C13 and C20 as raw materials.

¹H NMR (400 MHz, CDCl₃) δ 8.35 (d, J=14.0 Hz, 2H), 7.33-7.38 (m, 6H), 7.20-7.26 (m, 5H), 7.05-7.07 (m, 2H), 6.27 (d, J=14.0 Hz, 2H), 4.25-4.34 (m, 4H), 3.88-3.96 (m, 4H), 3.74 (t, J=6.4 Hz, 2H), 2.58-2.63 (m, 8H), 2.52-2.57 (m, 8H), 3.35 (s, 3H), 2.72 (t, J=6.0 Hz, 2H), 2.63-2.69 (m, 4H), 1.91-1.99 (m, 2H), 1.72 (s, 12H).

LCMS: m/z=881.6, C₅₂H₆₆ClN₂O₈⁺.

### Example 73: Preparation of 1-(2-(2-(2-carboxyethoxy)ethoxy)ethyl)-2-((E)-2-((E)-2-chloro-3-(2-((E)-3,3-dimethyl-1-(2,5,8,11-tetraoxatridecan-13-yl)indolin-2-ylidene)ethenyl)cyclohex-1-en-1-yl)ethenyl)-3,3-dimethyl-3H-indol-1-ium Chloride (73)

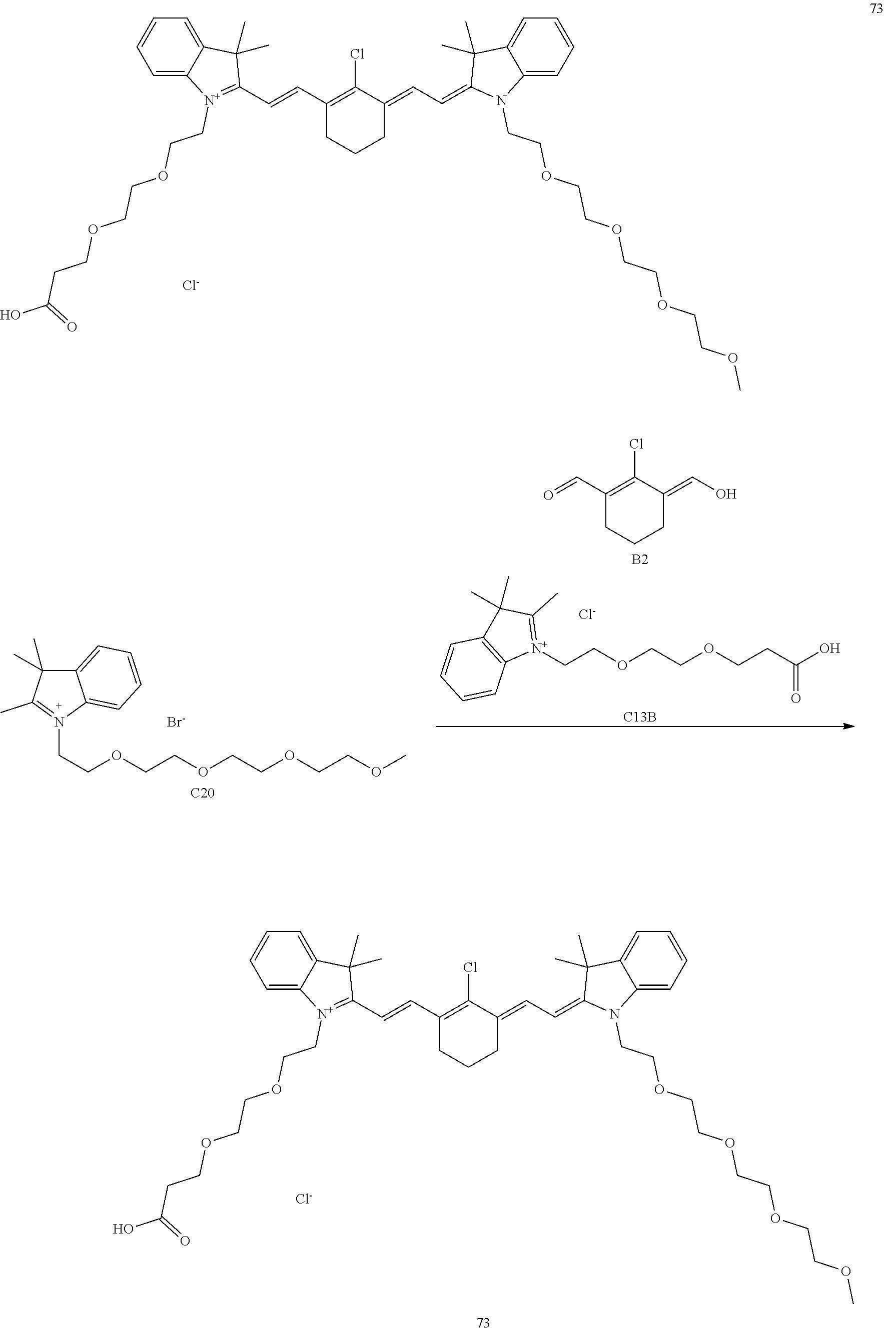

Compound 73 was prepared as a green paste according to the preparation method of Example 21 with Compounds B2, C13B and C20 as raw materials.

¹H NMR (400 MHz, CDCl₃) δ 8.42 (d, J=14.0 Hz, 1H), 8.23 (d, J=13.6 Hz, 1H), 7.29-7.45 (m, 6H), 7.13-7.19 (m, 2H), 6.61 (d, J=14.4 Hz, 1H), 6.17 (d, J=13.6 Hz, 1H), 4.53-4.60 (m, 2H), 4.23-4.30 (m, 2H), 4.00-4.07 (m, 2H), 3.86-3.96 (m, 2H), 3.73-3.76 (m, 2H), 3.57-3.63 (m, 14H), 3.51-3.54 (m, 2H), 3.36 (s, 3H), 2.76-2.83 (m, 2H), 2.64-2.72 (m, 4H), 1.93-2.02 (m, 2H), 1.72 (d, J=10.0 Hz, 12H).

LCMS: m/z=805.5, C₄₆H₆₂ClN₂O₈⁺.

### Example 74: Preparation of 5-(tert-butoxycarbonyl)-2-((E)-2-((E)-2-chloro-3-(2-((E)-3,3-dimethyl-1-(2-(3-oxo-3-phenoxypropoxy)ethyl)indolin-2-ylidene)ethenyl)cyclohex-1-en-1-yl)ethenyl)-1-ethyl-3,3-dimethyl-3H-indol-1-ium Bromide (74)

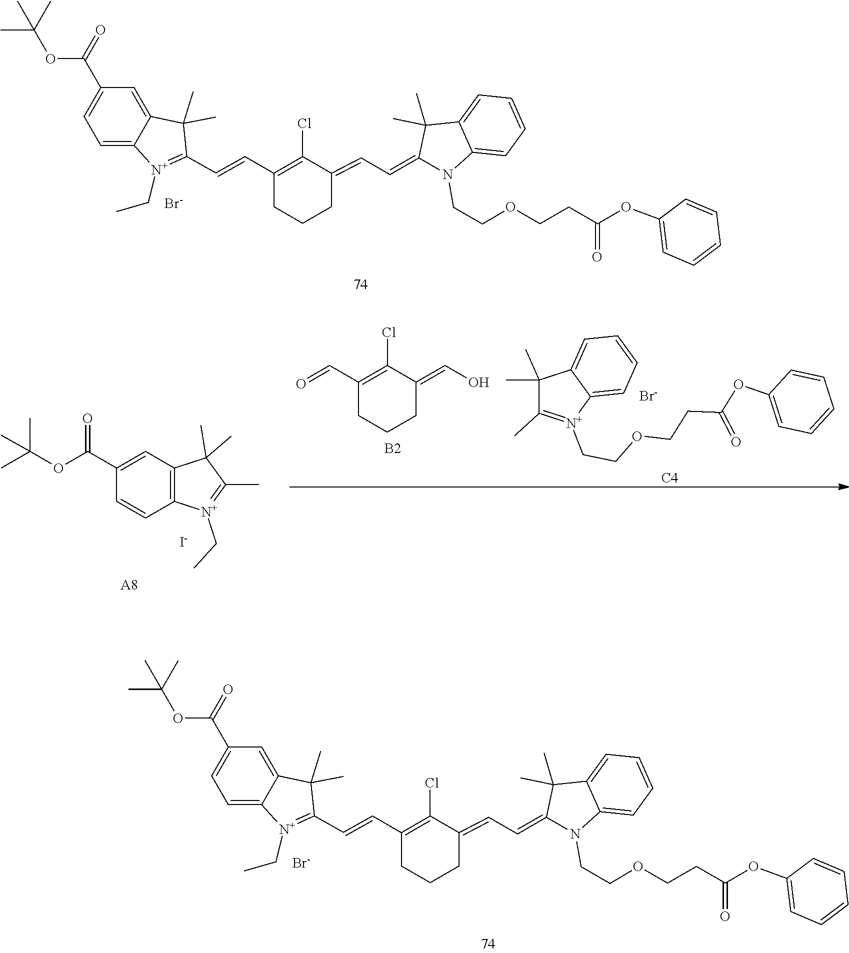

Compound 74 was prepared as a green oil according to the preparation method of Example 21 with Compounds B2, A8 and C4 as raw materials.

LCMS: m/z=775.3, C₄₈H₅₆ClN₂O₅⁺.

### Example 75A and 75B: Preparation of 2-((E)-2-((E)-3-(2-((E)-1-(2,5,8,11,14-pentaoxahexadecan-16-yl)-3,3-dimethylindolin-2-ylidene)ethenyl)-2-chlorocyclohex-1-en-1-yl)ethenyl)-3,3-dimethyl-1-(2-(2-(3-oxo-3-phenoxypropoxy)ethoxy)ethyl)-3H-indol-1-ium 2,2,2-trifluoroacetate (75A) and 2-((E)-2-((E)-2-chloro-3-(2-((E)-3,3-dimethyl-1-(2-(2-(3-oxo-3-phenoxypropoxy)ethoxy)ethyl)indolin-2-ylidene)ethenyl)cyclohex-1-en-1-yl)ethenyl)-3,3-dimethyl-1-(2-(2-(3-oxo-3-phenoxypropoxy)ethoxy)ethyl)-3H-indol-1-ium 2,2,2-trifluoroacetate (75B)

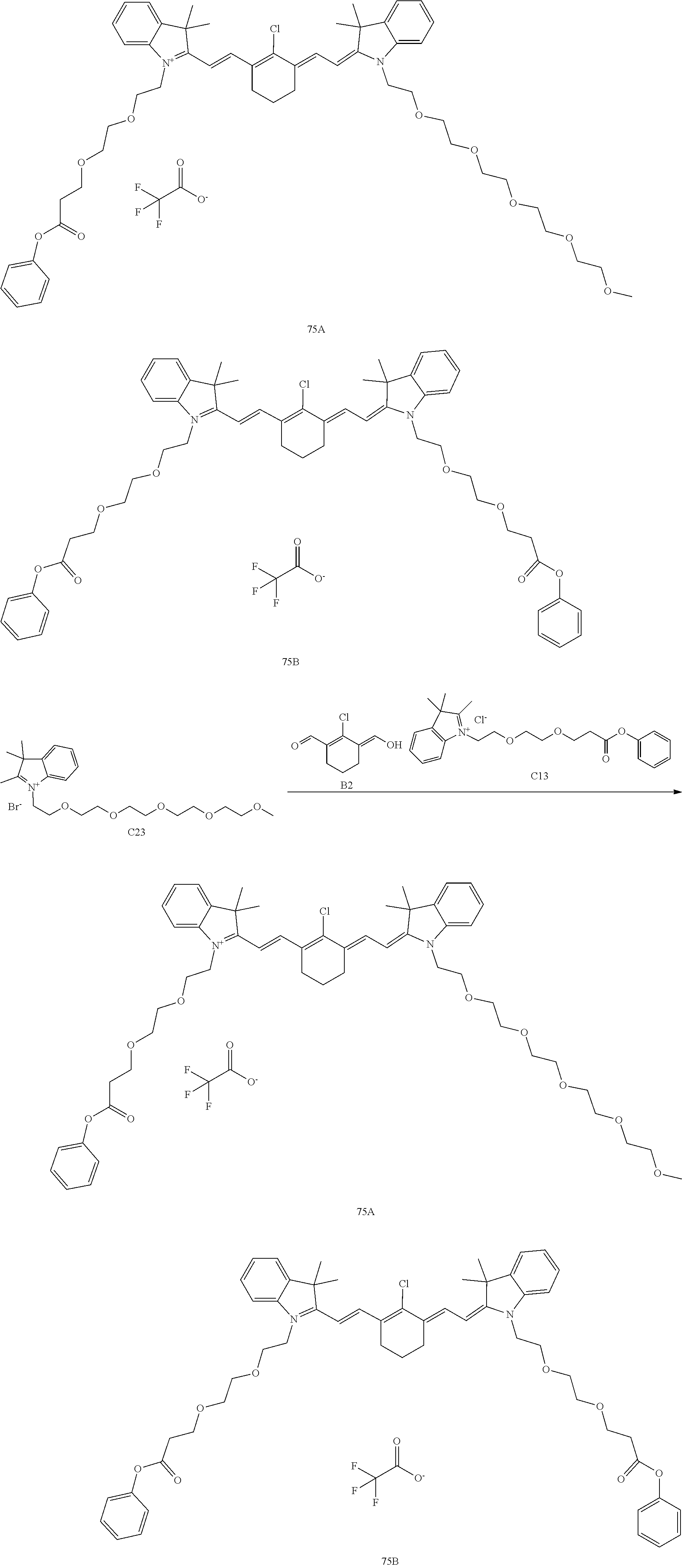

Compound 75A and Compound 75B were both prepared as a blue paste according to the preparation method of Example 21 with Compounds B2, C13 and C23 as raw materials.

### Compound 75A:

¹H NMR (400 MHz, DMSO-d₆); δ 8.25 (d, J=14.0 Hz, 2H), 7.62 (d, J=7.2 Hz, 2H), 7.36-7.44 (m, 6H), 7.23-7.28 (m, 3H), 7.05 (d, J=7.6 Hz, 2H), 6.42 (d, J=14.4 Hz, 2H), 4.42 (d, J=4.8 Hz, 4H), 3.81-3.82 (m, 4H), 3.62 (d, J=6.0 Hz, 2H), 3.50-3.55 (m, 4H), 3.37-3.47 (m, 16H), 3.21 (s, 3H), 2.68 (t, J=2.8 Hz, 6H), 1.83-1.86 (m, 2H) 1.66 (t, J=2.4 Hz, 12H).

LCMS: m/z=925.6, C₅₄H₇₀ClN₂O₉⁺.

### Compound 75B:

¹H NMR (400 MHz, CDCl₃) δ 8.34 (d, J=13.6 Hz, 2H), 7.34-7.38 (m, 8H), 7.22-7.26 (m, 6H), 7.06 (d, J=7.6 Hz, 4H), 6.29 (d, J=14.0 Hz, 2H), 4.32 (s, 4H), 3.92 (s, 4H), 3.75 (t, J=6.4 Hz, 4H), 3.58-3.63 (m, 8H), 2.73 (t, J=6.0 Hz, 4H), 2.66 (s, 4H), 1.94 (s, 2H), 1.72 (s, 12H).

LCMS: m/z=928.0, C₅₆H₆₄ClN₂O₈⁺.

### Example 76: Preparation of 2-((E)-2-((E)-3-(2-((E)-1-(2,5,8,11,14-pentaoxahexadecan-16-yl)-3,3-dimethylindolin-2-ylidene)ethenyl)-2-chlorocyclohex-1-en-1-yl)ethenyl)-1-(2-(2-(2-carboxyethoxy)ethoxy)ethyl)-3,3-dimethyl-3H-indol-1-ium 2,2,2-trifluoroacetate (76)

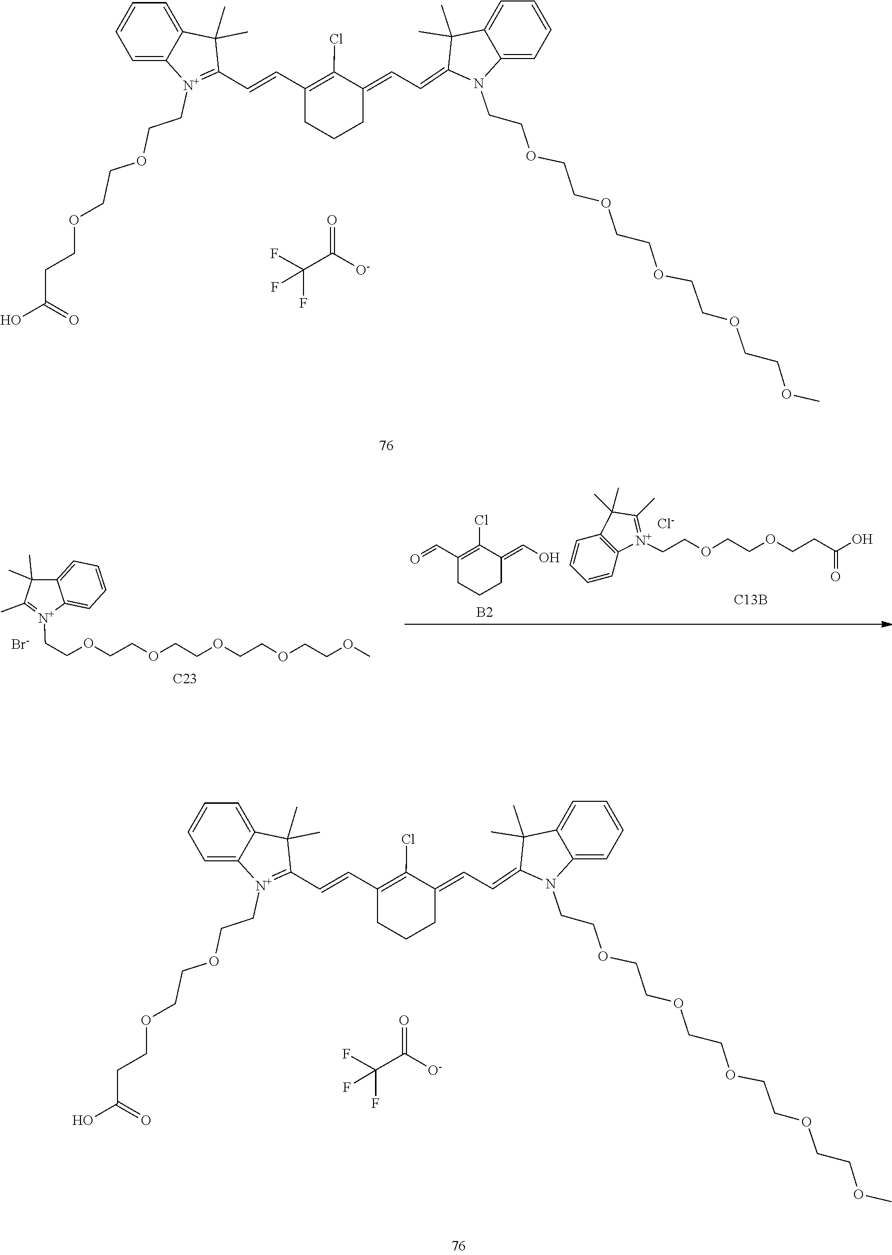

Compound 76 was prepared as a green paste according to the preparation method of Example 21 with Compounds B2, C13B and C23 as raw materials.

¹H NMR (400 MHz, CDCl₃); δ 8.35 (dd, J₁=14.0 Hz, J₂=29.2 Hz, 2H), 7.37-7.42 (m, 4H), 7.33-7.35 (m, 2H), 7.15-7.21 (m, 2H), 6.42 (d, J=14.4 Hz, 1H), 6.18 (d, J=14.0 Hz, 1H), 4.25-4.37 (m, 4H), 3.87-3.96 (m, 4H), 3.69 (t, J=6.0 Hz, 2H), 3.60-3.64 (m, 9H), 3.52-3.56 (m, 11H), 3.36 (s, 3H), 2.65-2.69 (m, 4H), 2.56 (t, J=6.4 Hz, 2H), 1.94 (t, J=5.6 Hz, 2H), 1.72 (d, J=5.6 Hz, 12H).

LCMS: m/z=849.5, C₄₈H₆₆ClN₂O₉⁺.

### Example 77: Preparation of 2,2′-((1E,1′E)-((3E,3′E)-(((2E,2′E)-((pentane-1,5-diylbis(azanediyl))bis(6-oxohexane-6, 1-diyl))bis(3,3-dimethylindolin-1-yl-2-ylidene))bis(ethane-2,1-diylidene))bis(2-chlorocyclohex-1-en-1-yl-3-ylidene))bis(ethene-2,1-diyl))bis(1-ethyl-3,3-dimethyl-3H-indol-1-ium) Chloride (77)

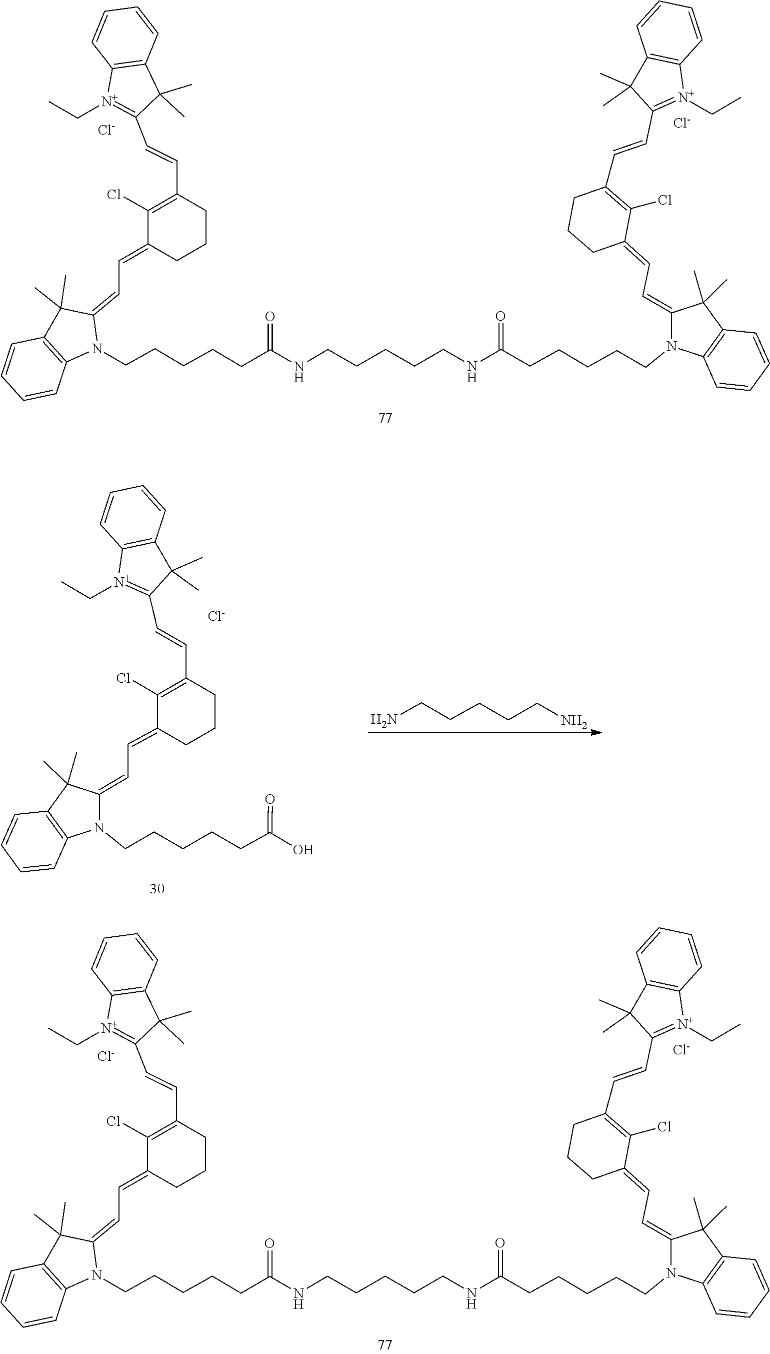

A mixture of Compound 30 (206 mg, 324 μmol, 2.00 eq), pentane-1,5-diamine (16.6 mg, 162 μmol, 19.0 μL, 1.00 eq), DIEA (62.9 mg, 486 μmol, 84.7 μL, 3.00 eq), 2-chloro-1-methyl-pyridin-1-ium iodide (62.1 mg, 243 μmol, 1.50 eq) and DMF (3.00 mL) was stirred at 25° C. under nitrogen atmosphere for 12 hours. 2-Chloro-N-methyl-pyridin-1-ium iodide (20.7 mg, 81.0 μmol, 0.500 eq) was added again, and stirring was continued for 6 hours. The resulting mixture was filtered to obtain a crude product (400 mg). The crude product was purified by reversed-phase HPLC (HCl condition; column: Phenomenex luna C18 150 mm×40 mm×15 μm; mobile phase: [water (HCl)-ACN]; gradient: 60%-90% B, 10 min) to obtain Compound 77 (25.27 mg, 18.4 μmol, 6.13% yield, 97.0% purity) as a blue solid.

¹H NMR: (400 MHz, MeOD) δ 8.44 (t, J=16.0 Hz, 4H), 7.51-7.55 (m, 4H), 7.42-7.45 (m, 4H), 7.26-7.37 (m, 8H), 6.24-6.33 (m, 4H), 4.21-4.26 (m, 4H), 4.15-4.19 (m, 4H), 3.11 (t, J=6.8 Hz, 4H), 2.73-2.77 (m, 8H), 2.19 (t, J=7.2 Hz, 4H), 1.93-1.99 (m, 4H), 1.82-1.89 (m, 4H), 1.73 (d, J=2.4 Hz, 24H), 1.65-1.70 (m, 4H), 1.40-1.49 (m, 14H), 1.27-1.33 (m, 2H).

LCMS: m/z=631.3, C₈₁H₁₀₂Cl₂N₆O₂²⁺/2.

According to the preparation method of Example 77 above, the following compounds were prepared using corresponding acids and diamines:

| Example | | |
|---|---|---|
| No. | Compound structure and reaction formula | NMR or MS, and appearance |
| 78 | 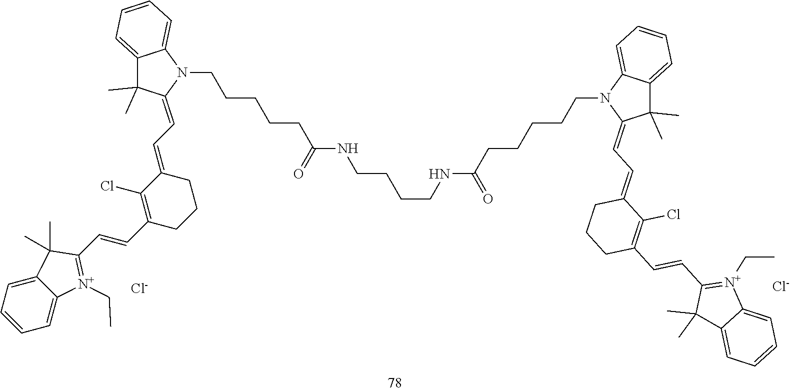 [SMILES: CC[N+]1=C(/C=C/C2=C(Cl)C(=C/C=C3/N(CCCCCC(=O)NCCCCNC(=O)CCCCCN4/C(=C/C=C5\CCCC(/C=C/C6=[N+](CC)c7ccccc7C6(C)C)=C5Cl)C(C)(C)c5ccccc54)c4ccccc4C3(C)C)/CCC2)C(C)(C)c2ccccc21 ; [Cl-] ; [Cl-]] | ¹H NMR (400 MHz, CDCl₃) δ 8.56- 8.55 (m, 1H), 8.38 (t, J = 12.8 Hz, 4H), 7.44-7.39 (m, 9H), 7.28 (s, 1H), 7.24 (t, J = 8.8 Hz, 3H), 7.18 (d, J = 7.6 Hz, 2H), 6.22-6.13 (m, 4H), 4.21-4.19 (m, 4H), 4.15-4.13 (m, 4H), 3.93- 3.52 (m, 4H), 3.32 (s, 4H), 2.73 (s, 8H), 2.53 (t, J = 6.8 Hz 4H), 1.99 (s, 4H), 1.89-1.82 (m, 10H), 1.73 (s, 24H), 1.57-1.56 (m, 4H), 1.48-1.47 (m, 6H). LCMS: m/z = 624.9 C₈₀H₁₀₀C₁₂N₆O₂²⁺/2. Green solid. |
| | 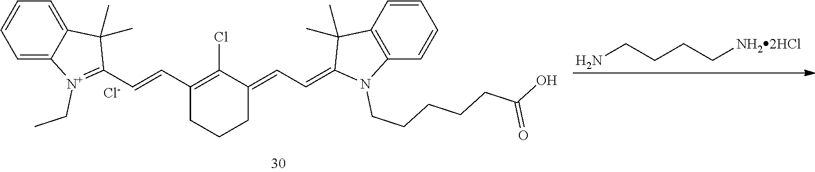 | |
| | 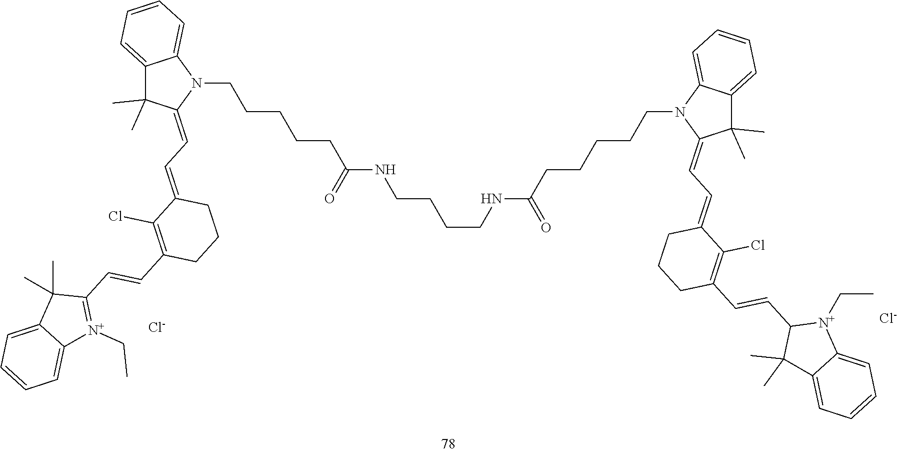 [SMILES: CC[N+]1=C(/C=C/C2=C(Cl)C(=C/C=C3/N(CCCCCC(=O)NCCCCNC(=O)CCCCCN4/C(=C/C=C5\CCCC(/C=C/C6[NH+](CC)c7ccccc7C6(C)C)=C5Cl)C(C)(C)c5ccccc54)c4ccccc4C3(C)C)/CCC2)C(C)(C)c2ccccc21 ; [Cl-] ; [Cl-]] | |
| 79 | 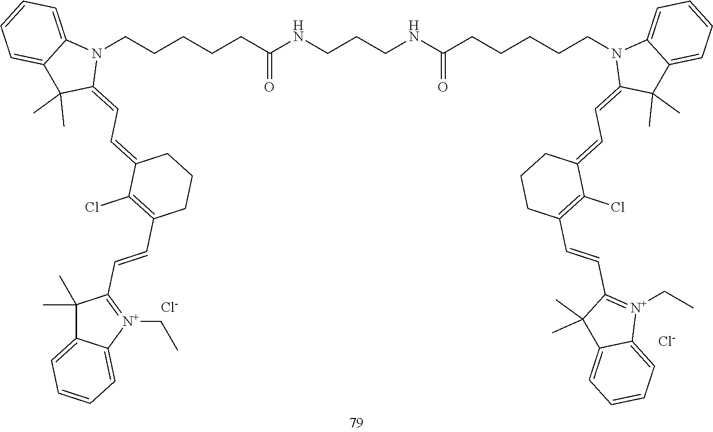 [SMILES: CC[N+]1=C(/C=C/C2=C(Cl)C(=C/C=C3/N(CCCCCC(=O)NCCCNC(=O)CCCCCN4/C(=C/C=C5\CCCC(/C=C/C6=[N+](CC)c7ccccc7C6(C)C)=C5Cl)C(C)(C)c5ccccc54)c4ccccc4C3(C)C)/CCC2)C(C)(C)c2ccccc21 ; [Cl-] ; [Cl-]] | ¹H NMR (400 MHz, CDCl₃) δ 8.39- 8.34 (m, 4H), 8.11 (s, 1H), 7.44-7.38 (m, 9H), 7.25-7.8 (m, 6H), 6.18 (t, J = 16.0 Hz 4H), 4.26-4.21 (m, 4H), 4.17- 4.10 (m, 4H), 3.30-3.29 (m, 4H), 2.73 (s, 8H), 2.40 (t, J = 6.80 Hz, 4H), 2.01-1.99 (m, 4H), 1.88-1.78 (m, 10H), 1.73 (s, 24H), 1.56-1.53 (m, 4H), 1.48-1.47 (m, 6H). LCMS: m/z 617.5 C₇₉H₉₈C₁₂N₆O₂²⁺/2. Green solid. |
| | 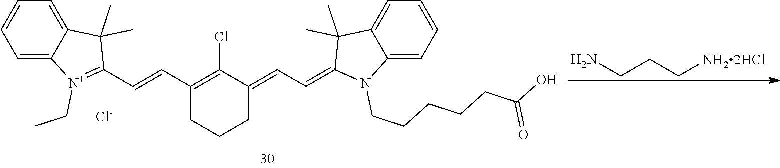 | |
| | 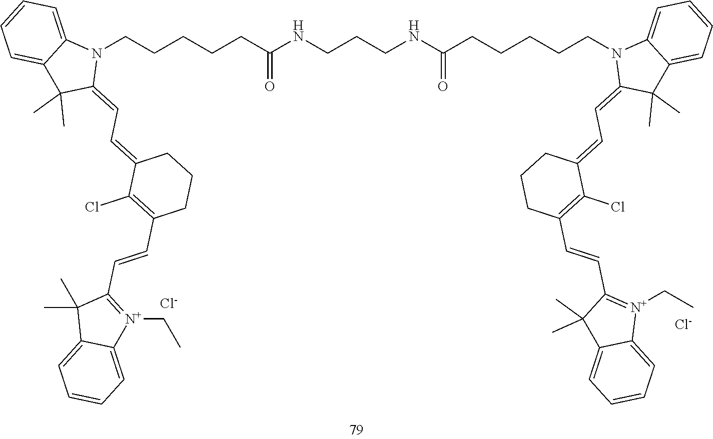 [SMILES: CC[N+]1=C(/C=C/C2=C(Cl)C(=C/C=C3/N(CCCCCC(=O)NCCCNC(=O)CCCCCN4/C(=C/C=C5\CCCC(/C=C/C6=[N+](CC)c7ccccc7C6(C)C)=C5Cl)C(C)(C)c5ccccc54)c4ccccc4C3(C)C)/CCC2)C(C)(C)c2ccccc21 ; [Cl-] ; [Cl-]] | |
| 80 | 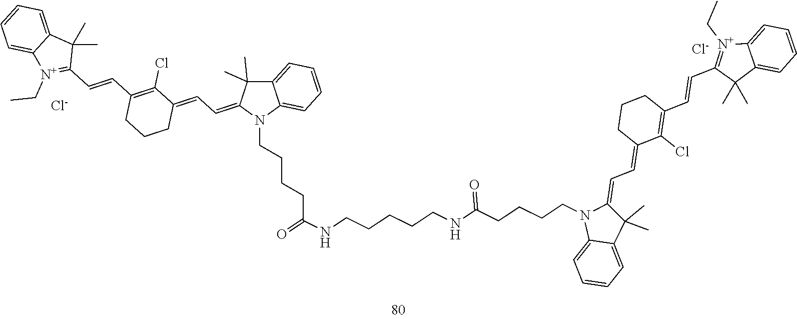 [SMILES: CC[N+]1=C(/C=C/C2=C(Cl)C(=C/C=C3/N(CCCCC(=O)NCCCCCNC(=O)CCCCN4/C(=C/C=C5\CCCC(/C=C/C6=[N+](CC)c7ccccc7C6(C)C)=C5Cl)C(C)(C)c5ccccc54)c4ccccc4C3(C)C)/CCC2)C(C)(C)c2ccccc21 ; [Cl-] ; [Cl-]] | ¹H NMR: (400 MHz, CDCl₃) δ 8.30- 8.41 (m, 4H), 8.09-8.11 (m, 2H), 7.37- 7.41 (m, 10H), 7.28-7.29 (m, 1H), 7.23-7.26 (m, 3H), 7.14-7.16 (m, 2H), 6.28-6.31 (m, 2H), 6.10 (t, J = 14.0 Hz, 2H), 4.10-4.19 (m, 8H), 3.33 (s, 4H), 2.79 (s, 4H), 2.70 (t, J = 5.2 Hz, 8H), 1.94-2.02 (m, 12H), 1.72 (s, 24H), 1.62-1.65 (m, 6H), 1.45 (t, J = 7.2 Hz, 6H). LCMS: m/z = 617.1 C₇₉H₉₈C₁₂N₆O₂²⁺/2. Green solid. |
| | 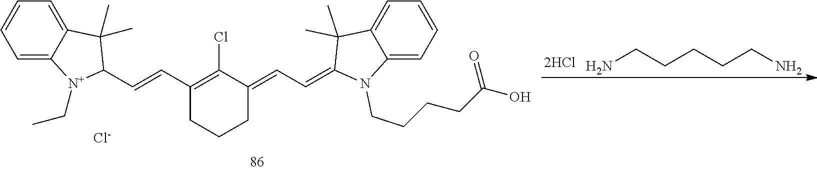 [SMILES: CC[NH+]1c2ccccc2C(C)(C)C1/C=C/C1=C(Cl)C(=C/C=C2/N(CCCCC(=O)O)c3ccccc3C2(C)C)/CCC1 ; NCCCCCN ; [Cl-]] | |
| | 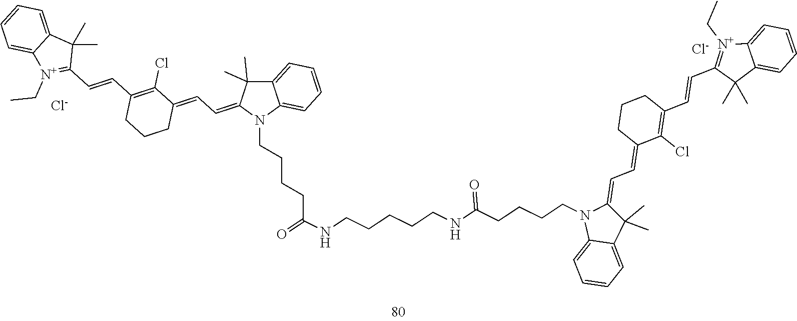 [SMILES: CC[N+]1=C(/C=C/C2=C(Cl)C(=C/C=C3/N(CCCCC(=O)NCCCCCNC(=O)CCCCN4/C(=C/C=C5\CCCC(/C=C/C6=[N+](CC)c7ccccc7C6(C)C)=C5Cl)C(C)(C)c5ccccc54)c4ccccc4C3(C)C)/CCC2)C(C)(C)c2ccccc21 ; [Cl-] ; [Cl-]] | |
| 81 | 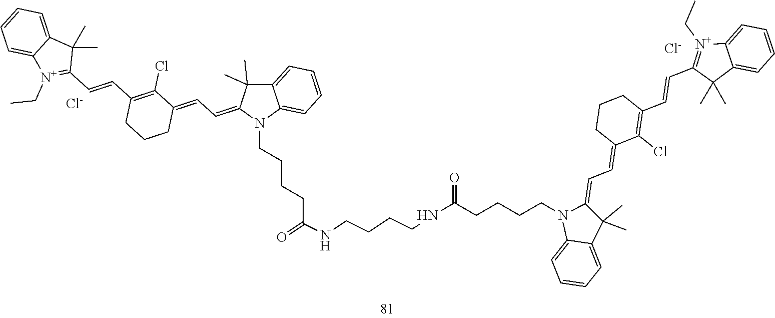 | ¹H NMR (400 MHz, CDCl₃) δ 8.31- 8.40 (m, 4H), 7.71-7.72 (m, 2H), 7.38- 7.43 (m, 9H), 7.37 (d, J = 3.6 Hz, 2H), 7.23-7.25 (m, 3H), 7.17-7.19 (m, 2H), 6.25 (t, J = 14.4 Hz, 2H), 6.11 (d, J = 14.0 Hz, 2H), 4.15-4.21 (m, 8H), 3.34-3.39 (m, 4H), 2.70-2.77 (m, 8H), 2.56 (s, 4H), 2.13-2.27 (m, 4H), 1.90-2.00 (m, 12H), 1.71 (s, 24H), 1.45 (d, J = 7.2 Hz, 6H). LCMS: m/z = 609.6 C₇₈H₉₆C₁₂N₆O₂²⁺/2. Green solid |
| | 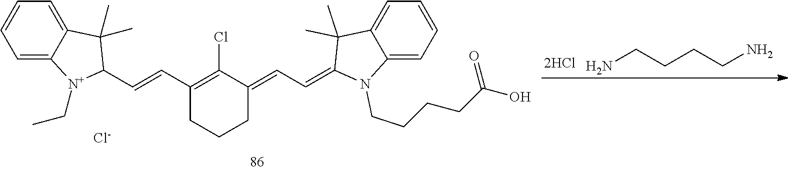 [SMILES: CC[NH+]1c2ccccc2C(C)(C)C1/C=C/C1=C(Cl)C(=C/C=C2/N(CCCCC(=O)O)c3ccccc3C2(C)C)/CCC1 ; NCCCCN ; [Cl-]] | |
| | 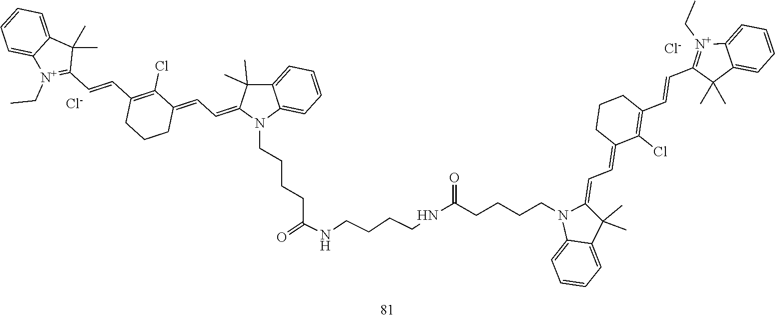 [SMILES: CC[N+]1=C(/C=C/C2=C(Cl)C(=C/C=C3/N(CCCCC(=O)NCCCCNC(=O)CCCCN4/C(=C/C=C5\CCCC(/C=C/C6=[N+](CC)c7ccccc7C6(C)C)=C5Cl)C(C)(C)c5ccccc54)c4ccccc4C3(C)C)/CCC2)C(C)(C)c2ccccc21 ; [Cl-] ; [Cl-]] | |
| 82 | 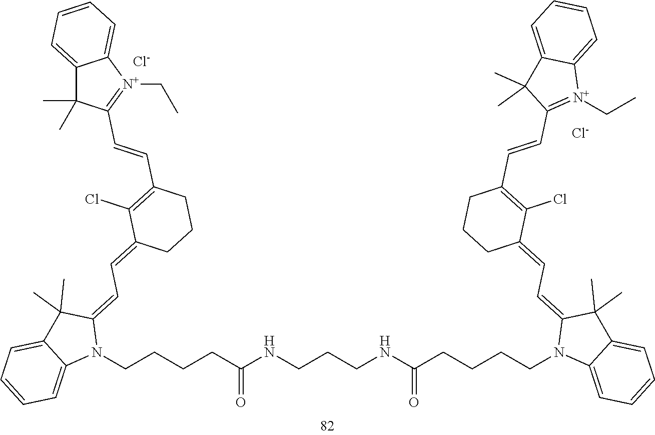 [SMILES: CC[N+]1=C(/C=C/C2=C(Cl)C(=C/C=C3/N(CCCCC(=O)NCCCNC(=O)CCCCN4/C(=C/C=C5\CCCC(/C=C/C6=[N+](CC)c7ccccc7C6(C)C)=C5Cl)C(C)(C)c5ccccc54)c4ccccc4C3(C)C)/CCC2)C(C)(C)c2ccccc21 ; [Cl-] ; [Cl-]] | ¹H NMR (400 MHz, CDCl₃) δ 8.33-8.41 (m, 4H), 8.03 (s, 2H), 7.37-7.42 (m, 8H), 7.28-7.30 (m, 2H), 7.26-7.27 (m, 2H), 7.24-7.26 (m, 2H), 7.16-7.24 (m, 2H), 6.26 (d, J = 14.4 Hz, 2H), 6.13 (d, J = 14 Hz, 2H), 4.19-4.21 (m, 8H), 3.31-3.33 (m, 4H), 2.77-2.97 (m, 4H), 2.70-2.73 (m, 4H), 2.53 (s, 4H), 2.00-2.03 (m, 4H), 1.93 (s, 8H), 1.79-1.92 (m, 2H), 1.72 (s, 24H), 1.46 (t, J = 7.2 Hz, 6H). LCMS: m/z = 603.3, C₇₇H₉₄Cl₂N₆O₂²⁺/2. Blue solid. |
| | 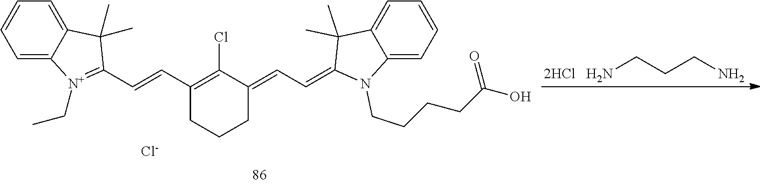 [SMILES: CC[N+]1=C(/C=C/C2=C(Cl)C(=C/C=C3/N(CCCCC(=O)O)c4ccccc4C3(C)C)/CCC2)C(C)(C)c2ccccc21 ; NCCCN ; [Cl-]] | |
| | 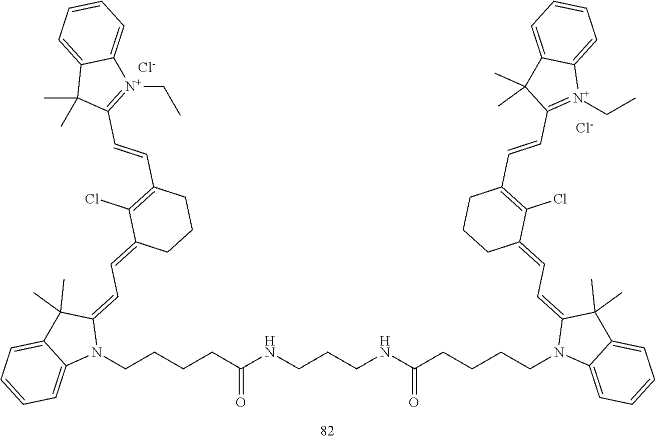 [SMILES: CC[N+]1=C(/C=C/C2=C(Cl)C(=C/C=C3/N(CCCCC(=O)NCCCNC(=O)CCCCN4/C(=C/C=C5\CCCC(/C=C/C6=[N+](CC)c7ccccc7C6(C)C)=C5Cl)C(C)(C)c5ccccc54)c4ccccc4C3(C)C)/CCC2)C(C)(C)c2ccccc21 ; [Cl-] ; [Cl-]] | |

### Example 83: Preparation of 2-((E)-2-((E)-2-chloro-3-(2-((E)-3,3-dimethyl-1-(6-oxo-6-(anilino)hexyl)indol-2-methylene)ethylidene)cyclopent-1-en-1yl)ethenyl)-1,3,3-trimethyl-3H-indol-1-ium Chloride (83)

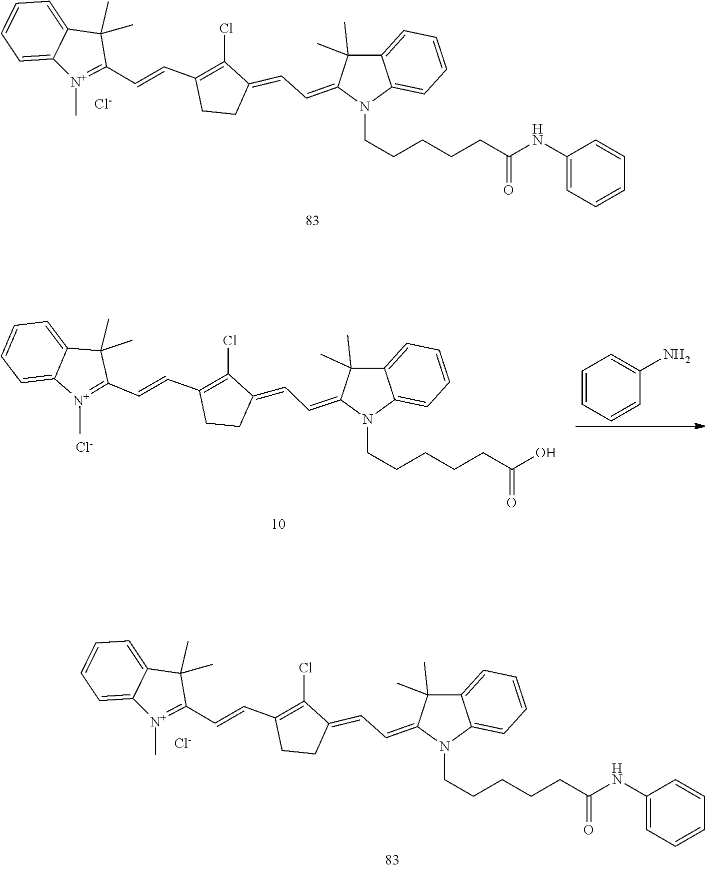

Aniline (16.3 mg, 175 μmol, 16.0 μL, 1.00 eq) and DIEA (113 mg, 877 umol, 153 uL, 5.00 eq) were added to a mixture of Compound 10 (100 mg, 175 μmol, 1.00 eq) and T₃P (3.36 g, 5.28 mmol, 3.14 mL, 50% purity, 30.1 eq). The mixture was stirred at 50° C. for 12 hours. The resulting mixture was concentrated under reduced pressure, and the residues were purified by preparative HPLC (HCl condition; column: YMC Triart C18 150 mm×25 mm×5 μm; mobile phase: [water (HCl)-ACN]; B %: 56%-86%, 8.5 min) to obtain Compound 83 (21.57 mg, 30.3 μmol, 9.76% yield, 90.5% purity) after lyophilization as a green solid.

¹H NMR (400 MHz, CDCl₃) δ 10.8 (s, 1H), 7.96-7.98 (m, 2H), 7.86 (d, J=14 Hz, 1H), 7.67 (d, J=13.6 Hz, 1H), 7.37-7.44 (m, 3H), 7.30-7.34 (m, 2H), 7.17-7.24 (m, 4H), 6.95-7.03 (m, 2H), 6.39 (d, J=14.4 Hz, 1H), 5.68 (d, J=14.4 Hz, 1H), 4.31-4.34 (m, 2H), 3.52 (s, 3H), 3.04-3.12 (m, 2H), 2.69-2.77 (m, 4H), 1.61-1.71 (m, 18H).

LCMS: m/z=644.3, C₄₂H₄₇ClN₃O⁺.

### Example 84: Preparation of 1-(5-carboxypentyl)-2-((E)-2-((E)-3-(2-((E)-1-(5-carboxypentyl)-3,3-dimethylindolin-2-ylidene)ethenyl)-2-chlorocyclopent-1-en-1-yl)ethenyl)-3,3-dimethyl-3H-indol-1-ium Chloride (84)

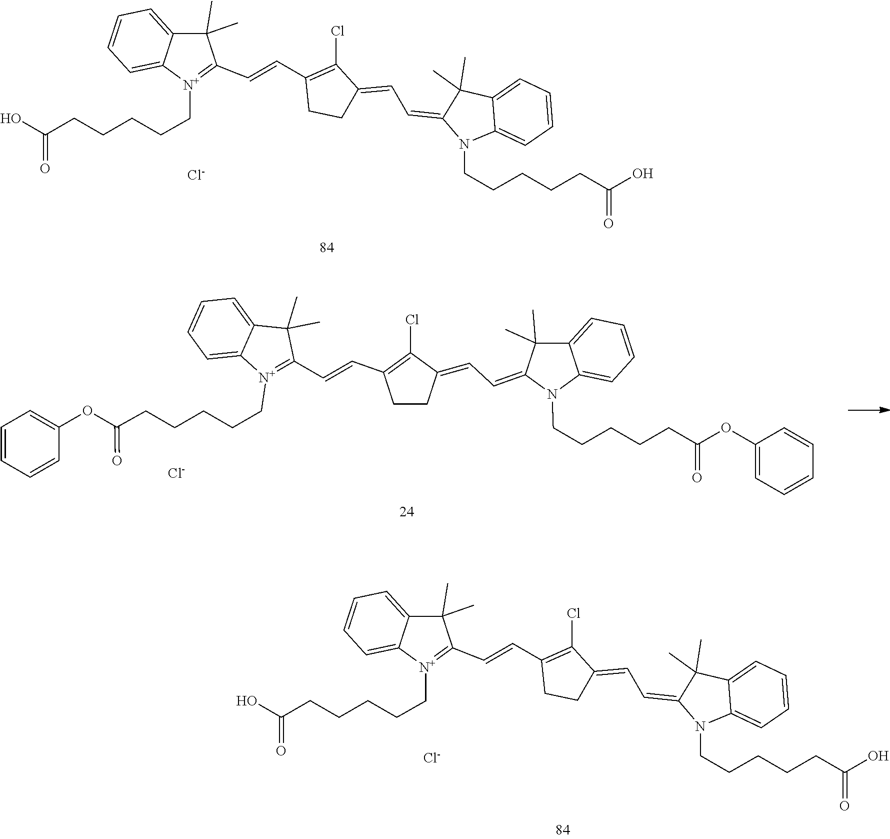 [SMILES: CC1(C)C(/C=C/C2=C(Cl)C(=C/C=C3/N(CCCCCC(=O)O)c4ccccc4C3(C)C)/CC2)=[N+](CCCCCC(=O)O)c2ccccc21 ; [Cl-] ; CC1(C)C(/C=C/C2=C(Cl)C(=C/C=C3/N(CCCCCC(=O)Oc4ccccc4)c4ccccc4C3(C)C)/CC2)=[N+](CCCCCC(=O)Oc2ccccc2)c2ccccc21 ; [Cl-] ; CC1(C)C(/C=C/C2=C(Cl)C(=C/C=C3/N(CCCCCC(=O)O)c4ccccc4C3(C)C)/CC2)=[N+](CCCCCC(=O)O)c2ccccc21 ; [Cl-]]

HCl/dioxane (4.00 M, 5.10 mL, 50.0 eq) solution was added to a solution of Compound 24 (350 mg, 407 μmol, 1.00 eq) in ACN (1.00 mL) and H₂O (73.5 mg, 4.08 mmol, 73.5 μL, 10.0 eq), and stirred at 15° C. for 2 hours. The resulting mixture was concentrated under reduced pressure, and then purified by preparative chromatography (column: Phenomenex luna C18 150 mm×40 mm×15 μm; mobile phase: [water (HCl)-ACN]; gradient: 45%-75% B, 10 min) to obtain Compound 84 (55.0 mg, 76.5 μmol, 18.7% yield, 98.2% purity) as a green solid.

¹H NMR (400 MHz, DMSO-d6) δ 12.04 (brs, 2H), 7.76 (d, J=13.6 Hz, 2H), 7.61 (d, J=7.2 Hz, 2H), 7.42-7.44 (m, 4H), 7.25-7.29 (m, 2H), 6.15 (d, J=14.0 Hz, 2H), 4.19 (t, J=6.8 Hz, 4H), 2.94 (s, 4H), 2.21 (t, J=7.2 Hz, 4H), 1.72-1.76 (m, 4H), 1.66 (s, 12H), 1.55-1.58 (m, 4H), 1.40-1.42 (m, 4H).

LCMS: m/z=699.4, C₄₁H₅₀ClN₂O₄⁺.

According to the preparation method of Example 84 above, the following compounds were prepared using corresponding raw materials:

| Example | | |
|---|---|---|
| No. | Compound structure and reaction formula | NMR or MA, and appearance |
| 85A and 85B | 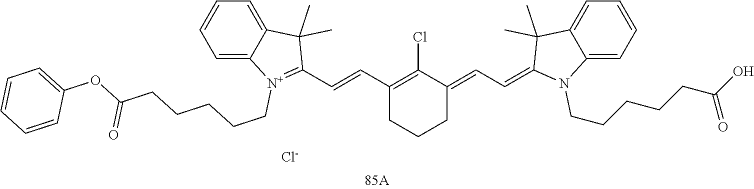 [SMILES: CC1(C)C(/C=C/C2=C(Cl)C(=C/C=C3/N(CCCCCC(=O)O)c4ccccc4C3(C)C)/CCC2)=[N+](CCCCCC(=O)Oc2ccccc2)c2ccccc21 ; [Cl-]] | 85A: ¹H NMR (400 MHz, CDCl₃) δ 8.42-8.3 (m, 2H), 7.44-7.34 (m, 6H), 7.30-7.28 (m, 1H), 7.25-7.19 (m, 3H), 7.11-7.09 (m, 1H), 7.03-7.01 (m, 2H), 6.27-6.24 (m, 1H), 6.11-6.08 (m, 1H), 4.15-4.06 (m, 4H), 2.72-2.60 (m, 6H), 2.54-2.51 (m, 2H), 1.99-1.52 (m, 27H); LCMS: m/z = 759.5 (M)⁺. 85B: LCMS: m/z = |
| | | 683.4 (M)⁺. |
| | 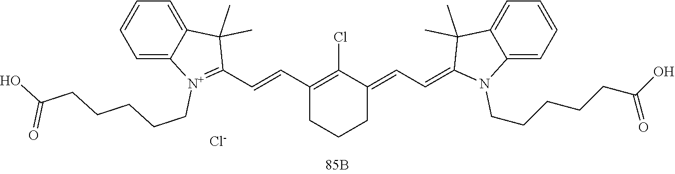 [SMILES: CC1(C)C(/C=C/C2=C(Cl)C(=C/C=C3/N(CCCCCC(=O)O)c4ccccc4C3(C)C)/CCC2)=[N+](CCCCCC(=O)O)c2ccccc21 ; [Cl-]] | Both were green solid. |
| | 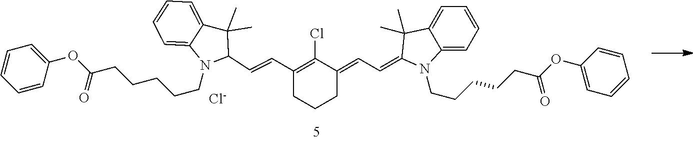 | |
| | 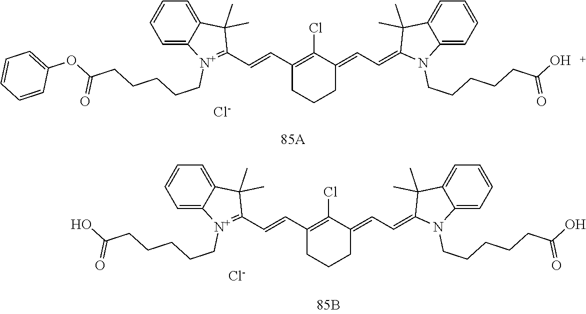 [SMILES: CC1(C)C(/C=C/C2=C(Cl)C(=C/C=C3/N(CCCCCC(=O)O)c4ccccc4C3(C)C)/CCC2)=[N+](CCCCCC(=O)Oc2ccccc2)c2ccccc21 ; [Cl-] ; CC1(C)C(/C=C/C2=C(Cl)C(=C/C=C3/N(CCCCCC(=O)O)c4ccccc4C3(C)C)/CCC2)=[N+](CCCCCC(=O)O)c2ccccc21 ; [Cl-]] | |
| 86 | 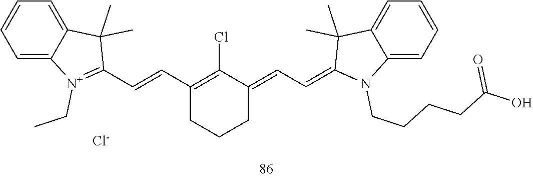 [SMILES: CC[N+]1=C(/C=C/C2=C(Cl)C(=C/C=C3/N(CCCCC(=O)O)c4ccccc4C3(C)C)/CCC2)C(C)(C)c2ccccc21 ; [Cl-]] | LCMS: m/z = 583.5 C₃₇H₄₄ClN₂O₂⁺. Green solid. |
| | 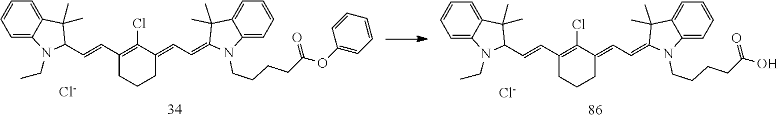 | |
| 87 | 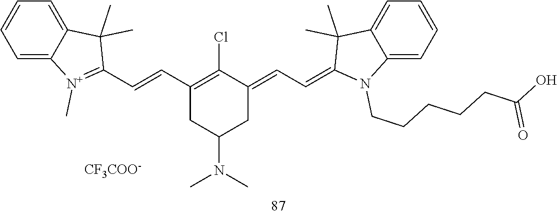 | ¹H NMR (400 MHz, MeOD) δ 8.49 (dd, J₁ = 8.0 Hz, J₂ = 14.0 Hz, 2H), 7.54-7.57 (m, 2H), 7.31-7.50 (m, 6H), 6.32-6.41 (m, 2H), 4.27 (t, J = 7.2 Hz, 2H), 3.79-3.85 (m, 1H), 3.77 (s, 3H), 3.33-3.39 (m, 2H), 3.10 (s, 6H), 2.88-2.94 (m, 2H), 2.33 (t, J = 7.2 Hz, 2H), 1.86-1.94 (m, 2H), 1.75 (t, J = 2.0 Hz, 12H), 1.67-1.71 (m, 2H), 1.50-1.56 (m, 2H); LCMS: m/z = 626.5 |
| | | C₃₉H₄₉ClN₃O₂⁺. Green solid. |
| | 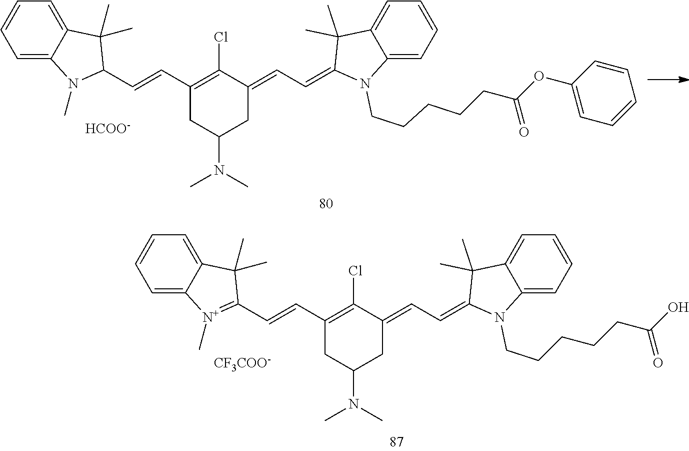 | |

### Example 88: Preparation of 2-((E)-2-((E)-3-(2-((E)-1-(5-carboxypentyl)-3,3-dimethylindolin-2-ylidene)ethenyl)-2-chlorocyclopent-1-en-1-yl)ethenyl)-1-(6-(2,6-dimethylphenoxy)-6-oxohexyl)-3,3-dimethyl-3H-indol-1-ium Acetate (88)

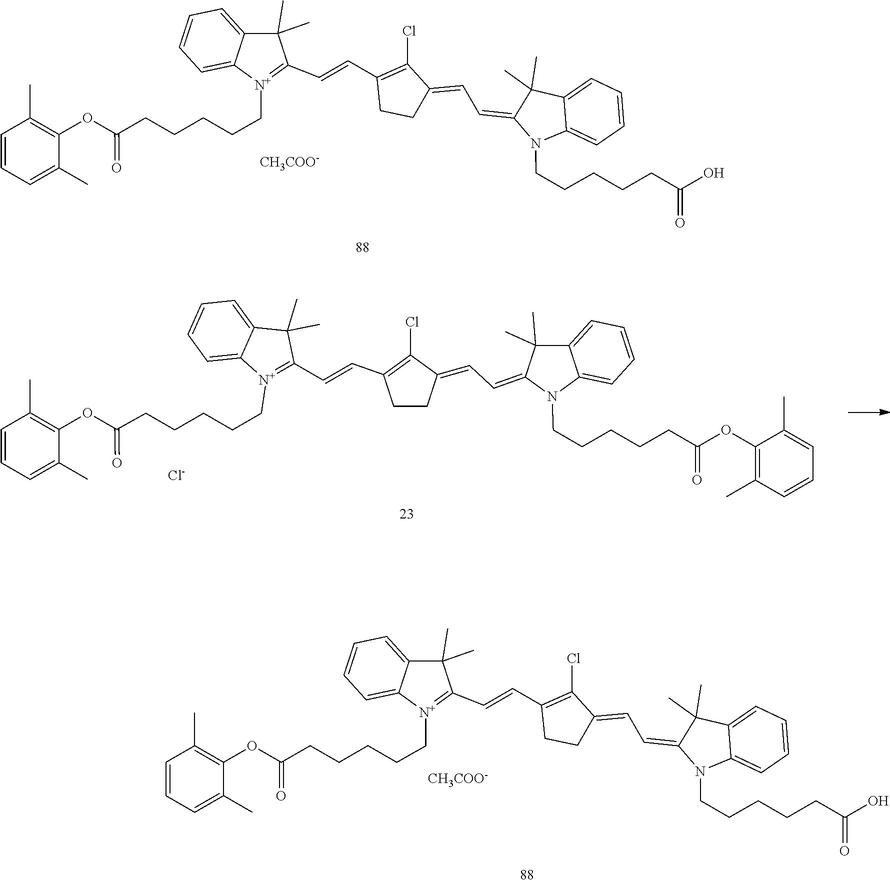

HCl/dioxane solution (4.00 M, 2.05 mL, 50.0 eq) and H₂O (29.6 mg, 1.64 mmol, 29.5 μL, 10.0 eq) were added to a solution of Compound 23 (150 mg, 164 μmol, 1.00 eq) in MeCN (3 mL), and stirred at 40° C. for 12 hours. The resulting mixture was filtered, and the filtrate was concentrated and purified by preparative chromatography HPLC (column: Phenomenex luna C18 150 mm×25 mm×10 μm; mobile phase: [water (TFA)-ACN]; gradient: 55%-85% B, 10 min) to obtain Compound 88 (24.0 mg, 24.5 μmol, 14.9% yield, 90.6% purity) as a green solid.

¹H NMR (400 MHz, CDCl₃) δ 7.81-7.87 (m, 2H), 7.36-7.40 (m, 4H), 7.23-7.25 (m, 2H), 7.07-7.11 (m, 2H), 7.03 (s, 3H), 6.00-6.05 (m, 2H), 4.05-4.08 (m, 4H), 2.95 (s, 2H), 2.65-24.67 (m, 2H), 2.44 (s, 2H), 2.10 (s, 6H), 1.85-1.90 (m, 8H), 1.71-1.76 (m, 12H), 1.5-1.61 (m, 6H).

LCMS: m/z=773.4, C₄₉H₅₈ClN₂O₄⁺.

According to the preparation method of Example 88 above, the following compounds were prepared using corresponding raw materials:

| Example | | |
|---|---|---|
| No. | Compound structure and reaction formula | NMR or MS, and appearance |
| 89 | 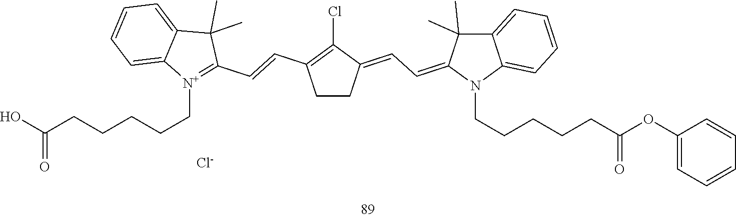 [SMILES: CC1(C)C(/C=C/C2=C(Cl)C(=C/C=C3/N(CCCCCC(=O)Oc4ccccc4)c4ccccc4C3(C)C)/CC2)=[N+](CCCCCC(=O)O)c2ccccc21 ; [Cl-]] | ¹H NMR (400 MHz, CDCl₃) δ 7.83- 7.87 (m, 2H), 7.33-7.38 (m, 6H), 7.19-7.22 (m, 3H), 7.13 (s, 2H), 7.02 (d, J = 8.0 Hz, 2H), 5.95-6.08 (m, 2H), 3.93-4.20 (m, 4H), 2.62 (s, 2H), 2.47 (s, 2H), 1.77-1.86 (m, 9H), 1.70 (s, 12H), 1.56-1.66 (m, 7H). LCMS: m/z 745.4 C₄₇H₅₄ClN₂O₄⁺. Green solid. |
| | 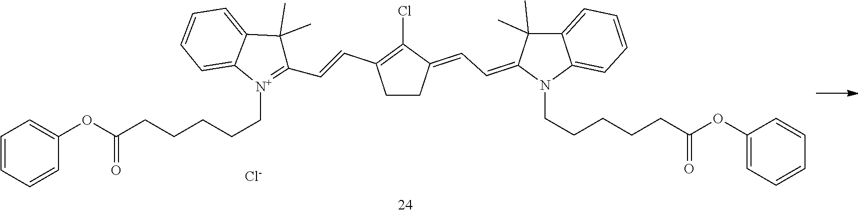 [SMILES: CC1(C)C(/C=C/C2=C(Cl)C(=C/C=C3/N(CCCCCC(=O)Oc4ccccc4)c4ccccc4C3(C)C)/CC2)=[N+](CCCCCC(=O)Oc2ccccc2)c2ccccc21 ; [Cl-]] | |
| | 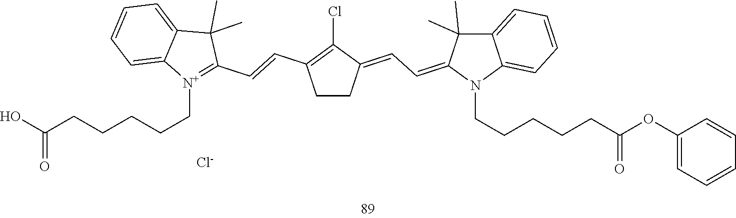 [SMILES: CC1(C)C(/C=C/C2=C(Cl)C(=C/C=C3/N(CCCCCC(=O)Oc4ccccc4)c4ccccc4C3(C)C)/CC2)=[N+](CCCCCC(=O)O)c2ccccc21 ; [Cl-]] | |
| 91 | 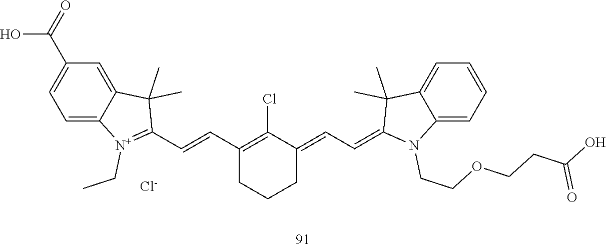 [SMILES: CC[N+]1=C(/C=C/C2=C(Cl)C(=C/C=C3/N(CCOCCC(=O)O)c4ccccc4C3(C)C)/CCC2)C(C)(C)c2cc(C(=O)O)ccc21 ; [Cl-]] | ¹H NMR (400 MHz, MeOD) δ 8.58 (d, J = 14.8 Hz, 1H), 8.27 (d, J = 13.6 Hz, 1H), 8.02-8.05 (m, 2H), 7.60 (d, J = 7.2 Hz, 1H), 7.48-7.50 (m, 2H), 7.38-7.42 (m, 1H), 7.18 (d, J = 8.4 Hz, 1H), 6.68 (d, J = 14.8 Hz, 1H), 6.14 (d, J = 13.6 Hz, 1H), 4.52 (t, J = 4.8 Hz, 2H), 4.11 (q, J = 7.2 Hz, 2H), 3.91 (t, J = 4.8 Hz, 2H), 3.67 (t, J = 6.0 Hz, 2H), 2.74 (t, J = 6.0 Hz, 4H), 2.39 (t, J = 6.0 Hz, 2H). 1.94-1.98 (m, 2H), 1.78 (s, 6H), 1.72 (s, 6H), 1.37 (t, J = 6.8 Hz, 3H). LCMS: m/z 643.2 C₃₈H₄₄ClN₂O₅⁺. Green solid. |
| | 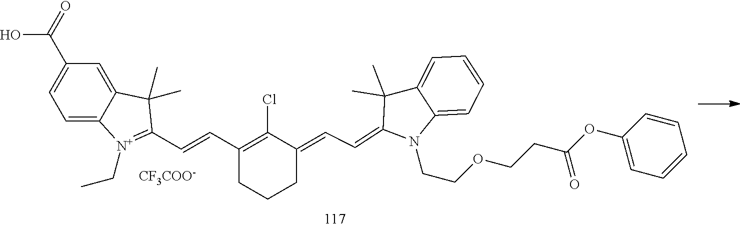 | |
| | 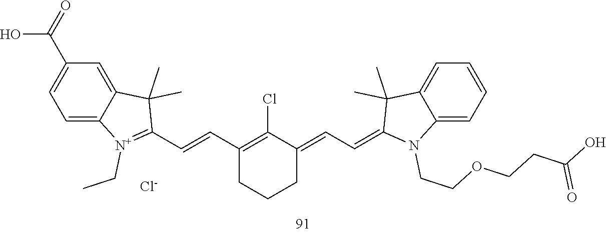 [SMILES: CC[N+]1=C(/C=C/C2=C(Cl)C(=C/C=C3/N(CCOCCC(=O)O)c4ccccc4C3(C)C)/CCC2)C(C)(C)c2cc(C(=O)O)ccc21 ; [Cl-]] | |
| 92 | 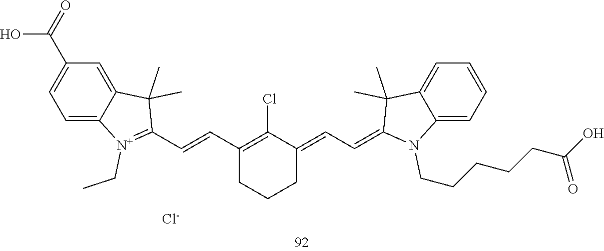 [SMILES: CC[N+]1=C(/C=C/C2=C(Cl)C(=C/C=C3/N(CCCCCC(=O)O)c4ccccc4C3(C)C)/CCC2)C(C)(C)c2cc(C(=O)O)ccc21 ; [Cl-]] | ¹H NMR (400 MHz, MeOD) δ 8.63 (d, J = 14.8 Hz, 1H), 8.28 (d, J = 13.6 Hz, 1H), 8.04-8.08 (m, 2H), 7.64 (d, J = 7.6 Hz, 1H), 7.53-7.54 (m, 2H), 7.43-7.46 (m, 1H), 7.22 (d, J = 8.0 Hz, 1H), 6.58 (d, J = 14.8 Hz, 1H), 6.14 (d, J = 13.6 Hz, 1H), 4.36 (t, J = 7.6 Hz, 2H), 4.08-4.14 (m, 2H), 2.76-2.77 (m, 4H), 2.33 (t, J = 7.2 Hz, 2H), 1.97-2.01 (m, 2H), 1.91-1.94 (m, 2H), 1.76 (d, J = 18.8 Hz, 12H), 1.67-1.71 (m, 2H), 1.50-1.56 (m, 2H), 1.38 (t, J = 7.2 Hz, 3H). LCMS: m/z = 641.4 |
| | | C₃₉H₄₆ClN₂O₄⁺. |
| | 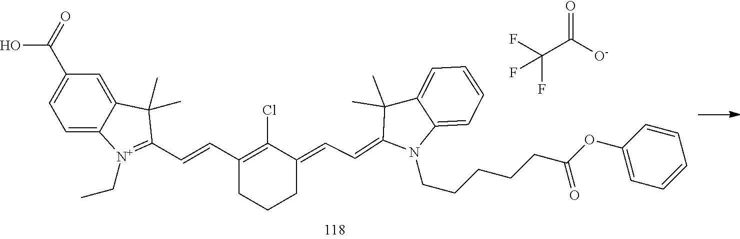 [SMILES: CC[N+]1=C(/C=C/C2=C(Cl)C(=C/C=C3/N(CCCCCC(=O)Oc4ccccc4)c4ccccc4C3(C)C)/CCC2)C(C)(C)c2cc(C(=O)O)ccc21 ; O=C([O-])C(F)(F)F] | Green solid. |
| | 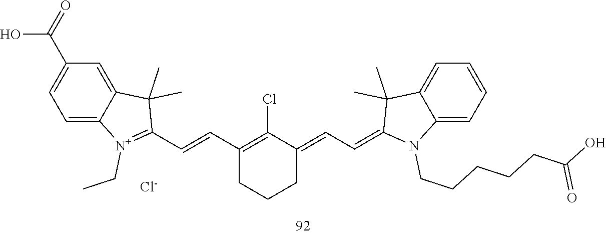 [SMILES: CC[N+]1=C(/C=C/C2=C(Cl)C(=C/C=C3/N(CCCCCC(=O)O)c4ccccc4C3(C)C)/CCC2)C(C)(C)c2cc(C(=O)O)ccc21 ; [Cl-]] | |
| 93 | | ¹H NMR (400 MHz, MeOD) δ 8.45 |
| | | (d, J = 15.2 Hz, 1H), 8.39 (d, J = |
| | | 14.8 Hz, 1H), 8.13-8.15 (m, 2H), |
| | | 7.63-7.70 (m, 2H), 7.49-7.59 (m, |
| | | 2H), 7.37-7.42 (m, 3H), 7.24 (t, J = |
| | | 7.2 Hz, 1H), 7.02 (d, J = 7.6 Hz, |
| | | 2H), 6.51 (d, J = 14.8 Hz, 1H), 6.19 |
| | | (d, J = 14.4 Hz, 1H), 4.60-4.66 (m, |
| | | 4H), 4.45-4.80 (m, 2H), 4.23-4.28 |
| | | (m, 2H), 3.25 (s, 6H), 2.63-2.67 (m, |
| | | 2H), 1.97-2.04 (m, 2H), 1.83-1.90 |
| | | (m, 2H), 1.78 (d, J = 8.8 Hz, 12H), |
| | | 1.61-1.67 (m, 2H), 1.43 (t, J = 7.2 |
| | | Hz, 3H). |
| | | LCMS: m/z = 374.0 |
| | | C₄₆H₅₄ClN₃O₄²⁺/2. |
| | | Blue solid. |
| 94 | | ¹H NMR (400 MHz, DMSO) δ |
| | | 8.15-8.21 (m, 2H), 7.29-7.39 (m, |
| | | 4H), 6.96-7.01 (m, 2H), 6.22 (t, J = |
| | | 14.0 Hz, 2H), 4.16-4.17 (m, 2H), |
| | | 3.81-3.82 (m, 6H), 3.67 (s, 3H), |
| | | 2.67-2.68 (m, 4H), 2.20 (t, J = 7.2 |
| | | Hz, 2H), 1.85-1.86 (m, 2H), |
| | | 1.70-1.75 (m, 2H), 1.66 (d, J = 2.8 |
| | | Hz, 12H), 1.52-1.59 (m, 2H), |
| | | 1.36-1.41 (m, 2H). |
| | | LCMS: m/z = 643.4 |
| | | C₃₉H₄₈ClN₂O₄⁺. |
| | | Green solid. |
| 95 | 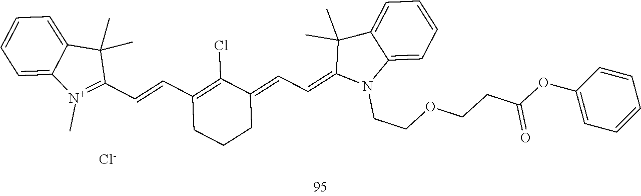 | ¹H NMR (400 MHz, CDCl₃); δ 8.27-8.35 (m, 1H), 7.31-7.37 (m, 7H), 7.14-7.21 (m, 5H), 4.37 (s, 2H), 3.90 (s, 3H), 3.55-3.72 (m, 4H), 2.63 (t, J = 5.6 Hz, 3H), 1.94 (s, 2H), 1.68 (d, J = 7.6 Hz, 15H); LCMS: m/z = 585.3 C₄₂H₄₆ClN₂O₃⁺. Green solid. |
| | 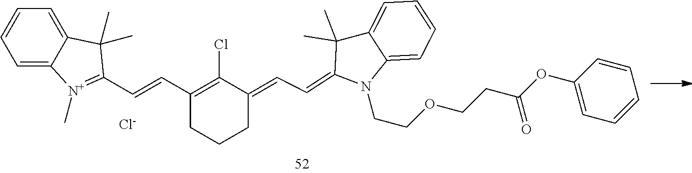 [SMILES: C[N+]1=C(/C=C/C2=C(Cl)C(=C/C=C3/N(CCOCCC(=O)Oc4ccccc4)c4ccccc4C3(C)C)/CCC2)C(C)(C)c2ccccc21 ; [Cl-]] | |
| | 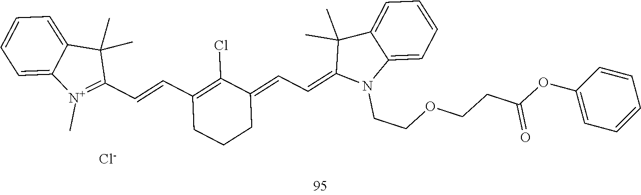 [SMILES: C[N+]1=C(/C=C/C2=C(Cl)C(=C/C=C3/N(CCOCCC(=O)Oc4ccccc4)c4ccccc4C3(C)C)/CCC2)C(C)(C)c2ccccc21 ; [Cl-]] | |
| 96 | 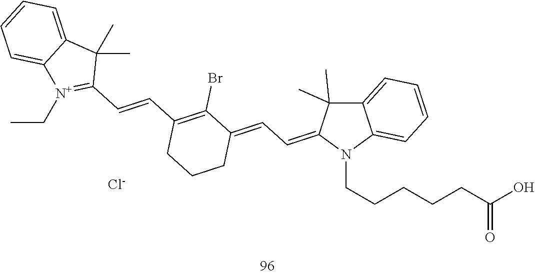 [SMILES: CC[N+]1=C(/C=C/C2=C(Br)C(=C/C=C3/N(CCCCCC(=O)O)c4ccccc4C3(C)C)/CCC2)C(C)(C)c2ccccc21 ; [Cl-]] | ¹H NMR (400 MHz, CDCl₃) δ 8.34-8.42 (m, 2H), 7.38-7.42 (m, 4H), 7.28-7.30 (m, 2H), 7.15 (dd, J1 = 8.0 Hz, J2 = 20.8 Hz, 2H), 6.23 (d, J = 14.0 Hz, 1H), 6.12 (d, J = 14.0 Hz, 1H), 4.08-4.13 (m, 4H), 2.70 (q, J = 6.0 Hz, 4H), 2.47 (t, J = 7.2 Hz, 2H), 1.97-2.00 (m, 2H), 1.85-1.89 (m, 2H), 1.75-1.79 (m, 2H), 1.74 (d, J = 0.8 Hz, 12H), 1.53-1.57 (m, 2H), 1.45 (t, J = 7.2 Hz, 3H). LCMS: m/z = 643.3 C₃₈H₄₆BrN₂O₂⁺. Green solid. |
| | 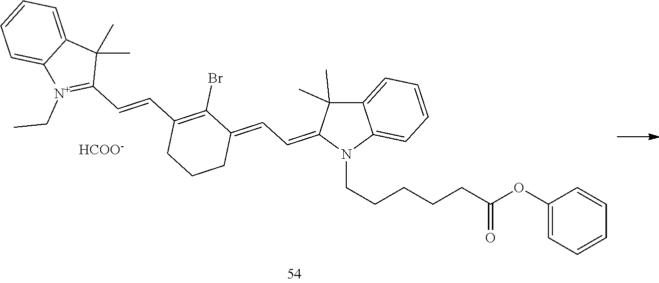 | |
| | 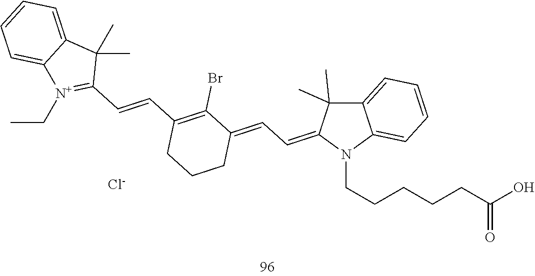 [SMILES: CC[N+]1=C(/C=C/C2=C(Br)C(=C/C=C3/N(CCCCCC(=O)O)c4ccccc4C3(C)C)/CCC2)C(C)(C)c2ccccc21 ; [Cl-]] | |
| 97 | 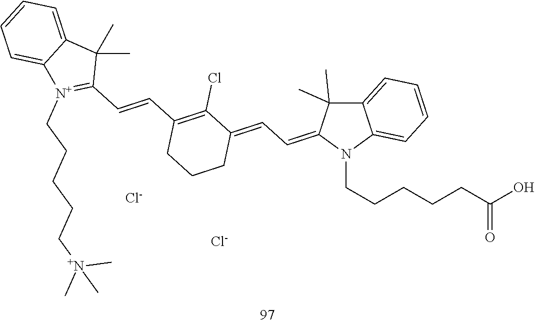 [SMILES: CC1(C)C(/C=C/C2=C(Cl)C(=C/C=C3/N(CCCCCC(=O)O)c4ccccc4C3(C)C)/CCC2)=[N+](CCCCC[N+](C)(C)C)c2ccccc21 ; [Cl-] ; [Cl-]] | ¹H NMR (400 MHz, DMSO) δ 8.23-8.31 (m, 2H), 7.64 (t, J = 8.0 Hz, 2H), 7.43-7.50 (m, 4H), 7.28-7.34 (m, 2H), 6.33 (dd, J1 = 14.4 Hz, J2 = 27.2 Hz, 2H), 4.19-4.27 (m, 4H), 3.22-3.27 (m, 4H), 3.01 (s, 9H), 2.67-2.74 (m, 4H), 2.21 (t, J = 7.2 Hz, 2H), 1.84-1.88 (m, 2H), 1.74-1.80 (m, 6H), 1.68 (s, 12H), 1.54-1.58 (m, 2H), 1.36-1.42 (m, 2H). LCMS: m/z = 349.2 C₄₄H₆₀ClN₃O₂²⁺/2. Green solid. |
| | 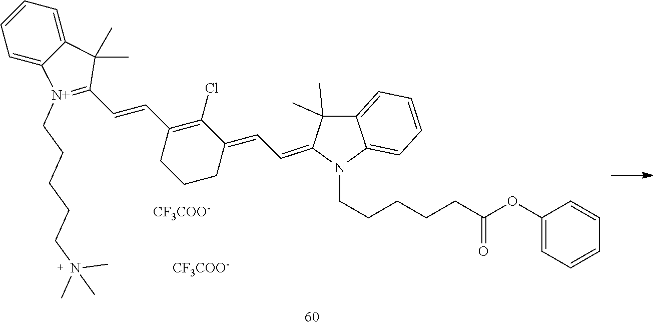 | |
| | 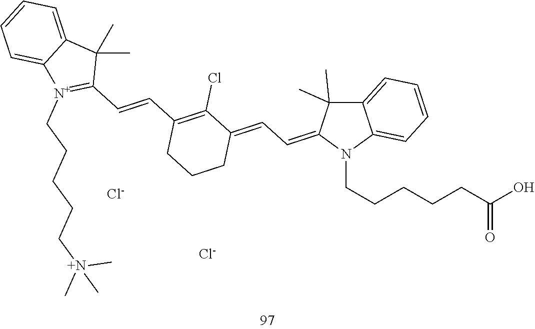 [SMILES: CC1(C)C(/C=C/C2=C(Cl)C(=C/C=C3/N(CCCCCC(=O)O)c4ccccc4C3(C)C)/CCC2)=[N+](CCCCC[N+](C)(C)C)c2ccccc21 ; [Cl-] ; [Cl-]] | |
| 98 | 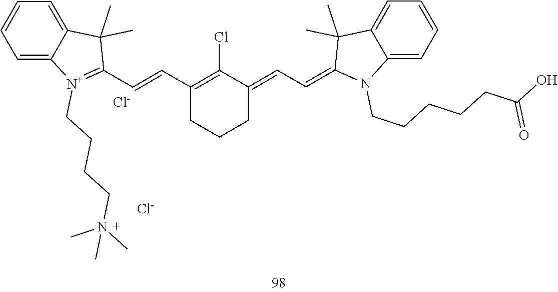 | ¹H NMR (400 MHz, DMSO-d₆) δ 12.01 (s, 1H), 8.22-8.33 (m, 2H), 7.61-7.68 (m, 2H), 7.46-7.51 (m, 4H), 7.26-7.35 (m, 2H), 6.23-6.42 (m, 2H), 4.20-4.28 (m, 4H), 3.36-3.38 (m, 2H), 3.05 (s, 9H), 2.71 (d, J = 4.8 Hz, 4H), 2.20 (t, J = 7.2 Hz, 2H), 1.86-1.87 (m, 4H), 1.68-1.77 (m, 16H), 1.55-1.58 (m, 2H), 1.37-1.40 (m, 2H). LCMS: m/z = 342.2 C₄₃H₅₈ClN₃O₂²⁺/2. Green solid. |
| | 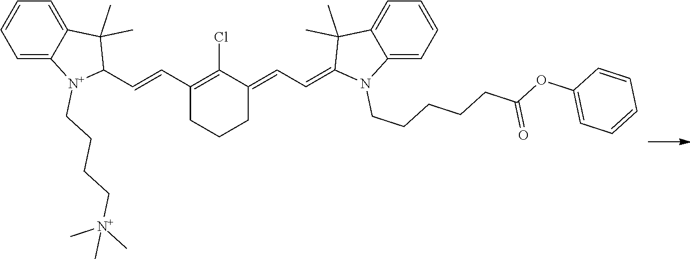 [SMILES: CC1(C)/C(=C\C=C2/CCCC(/C=C/C3[NH+](CCCC[N+](C)(C)C)c4ccccc4C3(C)C)=C2Cl)N(CCCCCC(=O)Oc2ccccc2)c2ccccc21] | |
| | 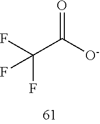 | |
| | 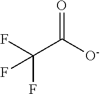 | |
| | 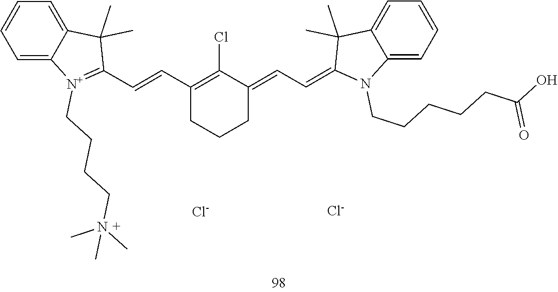 | |
| 99 | 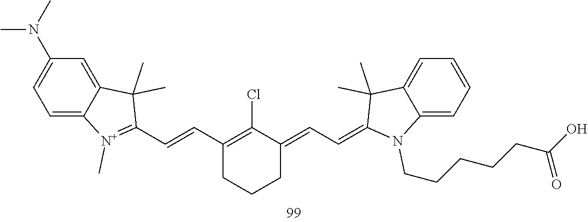 [SMILES: CN(C)c1ccc2c(c1)C(C)(C)C(/C=C/C1=C(Cl)C(=C/C=C3/N(CCCCCC(=O)O)c4ccccc4C3(C)C)/CCC1)=[N+]2C] | ¹H NMR (400 MHz, MeOD) δ 8.61 (t, J = 10.0 Hz, 1H), 8.22 (t, J = 8.80 Hz, 1H), 7.91 (d, J = 2.00 Hz, 1H), 7.72-7.64 (m, 2H), 7.58- 7.51 (m, 2H), 7.44 (t, J = 7.60 Hz, 1H), 7.30 (d, J = 14.8 Hz, 1H), 6.61 (d, J = 14.8 Hz, 1H), 6.08 (d, J = 13.6 Hz, 1H), 4.39 (t, J = 7.20 Hz, 2H), 3.54 (s, 3H), 3.33 (s, 6H), 2.76 (dd, J1 = 6.00 Hz, J2 = 12.4 Hz, 4H), 2.33 (t, J = 7.20 Hz, 2H), 1.98-1.93 (m, 4H), 1.77 (d, J = 8.00 Hz, 12H), 1.73-1.69 (m, 2H), 1.54- 1.51(m, 2H). |
| | 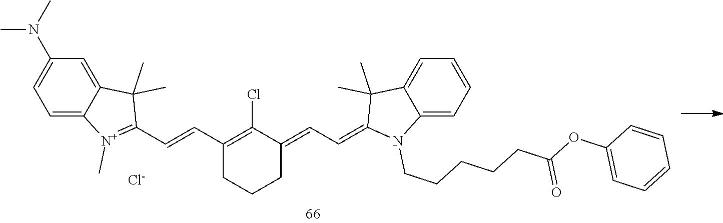 | LCMS: m/z = 626.4 C₃₉H₄₉ClN₃O₂⁺. Green paste. |
| | 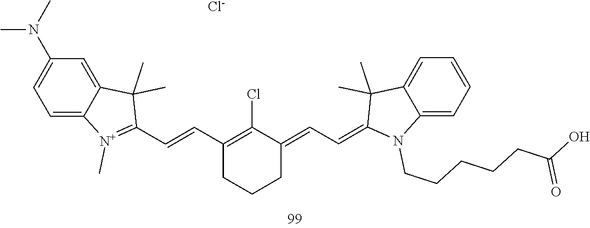 [SMILES: CN(C)c1ccc2c(c1)C(C)(C)C(/C=C/C1=C(Cl)C(=C/C=C3/N(CCCCCC(=O)O)c4ccccc4C3(C)C)/CCC1)=[N+]2C ; [Cl-]] | |
| 100 | 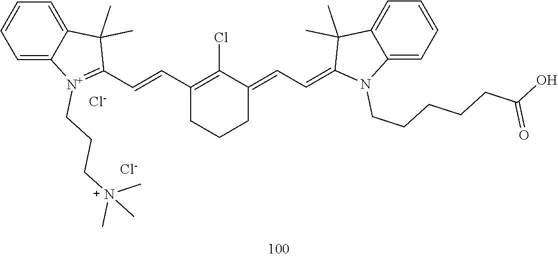 | ¹H NMR (400 MHz, CDCl₃) δ 8.30- 8.42 (m, 2H), 7.30-7.44 (m, 5H), 7.09-7.13 (m, 3H), 6.41-6.44 (m, 1H), 6.04-6.12 (m, 1H), 3.85-4.37 (m, 6H), 3.25 (d, J = 34.0 Hz, 9H), 2.64-2.74 (m, 4H), 2.27-2.38 (m, 12H), 1.81-1.86 (m, 4H), 1.72 (t, J = 4.8 Hz, 4H), 1.41-1.63 (m, 4H). LCMS: m/z = 335.0 C₄₂H₅₆ClN₃O₂₂⁺/2. Green solid. |
| | 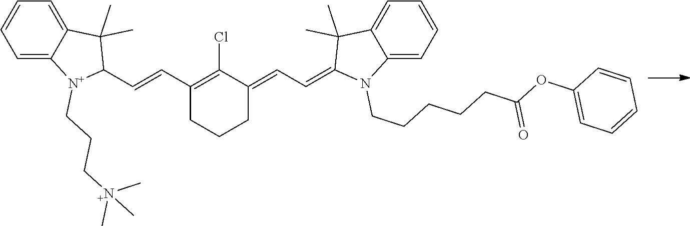 [SMILES: CC1(C)/C(=C\C=C2/CCCC(/C=C/C3[NH+](CCC[N+](C)(C)C)c4ccccc4C3(C)C)=C2Cl)N(CCCCCC(=O)Oc2ccccc2)c2ccccc21] | |
| | 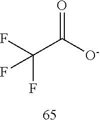 | |
| | 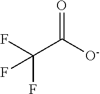 | |
| | 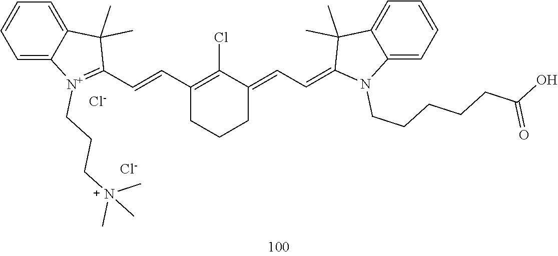 [SMILES: CC1(C)C(/C=C/C2=C(Cl)C(=C/C=C3/N(CCCCCC(=O)O)c4ccccc4C3(C)C)/CCC2)=[N+](CCC[N+](C)(C)C)c2ccccc21 ; [Cl-] ; [Cl-]] | |

### Example 101: Preparation of 6-[(2E)-2-[(2E)-2-[3-[(E)-2-(1-ethyl-3,3-dimethyl-indol-1-ium-2-yl)ethenyl]-2-thiophenol-cyclopent-2-en-1-methylene]ethylidene]-3,3-dimethyl-indol-1-yl]hexanoate; 2,2,2-trifluoroacetate (101)

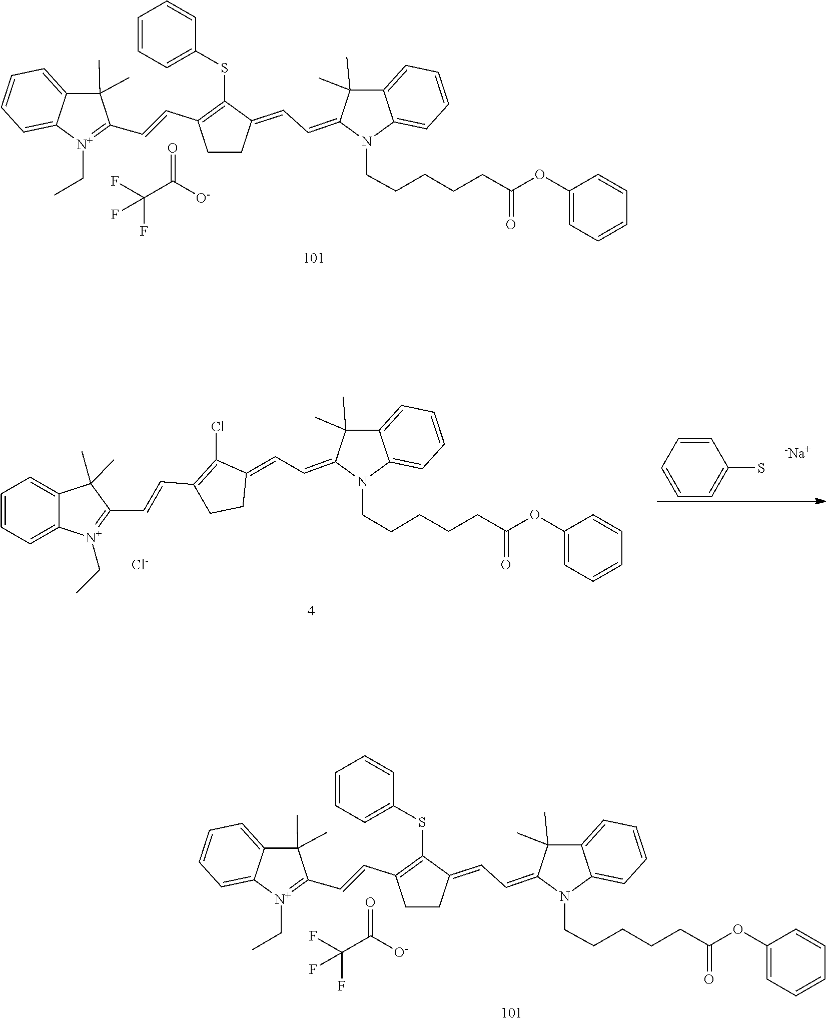

Triethylamine (78.5 mg, 775 μmol, 107 μL, 3.00 eq) and sodium thiophenolate (41.0 mg, 310 μmol, 1.20 eq) were added to a solution of Compound 4 (200 mg, 258 μmol, 1.00 eq) in THF (1 mL). The mixture was stirred at 25° C. for 3 hours. The resulting mixture was concentrated under reduced pressure, and the residues were purified by preparative HPLC (chromatographic column: Phenomenex luna C18 150 mm×40 mm×15 μm; mobile phase: [water (TFA)-ACN]; gradient: 60%-90% B, 10 min) to obtain Compound 101 (86.0 mg, 96.0 μmol, 37.1% yield, 94.6% purity) as a blue solid.

¹H NMR (400 MHz, MeOD) δ 8.06-8.12 (m, 2H), 7.36-7.45 (m, 10H), 7.29-7.32 (m, 6H), 6.99-7.01 (m, 2H), 6.07-6.13 (m, 2H), 4.41-4.19 (m, 4H), 2.96-3.02 (m, 4H), 2.61 (t, J=14.4 Hz, 2H), 1.81-1.92 (m, 4H), 1.56-1.57 (s, 2H), 1.48 (d, J=2.8 Hz, 12H), 1.39 (t, J=14.4 Hz, 3H).

LCMS: m/z=733.3, C₄₉H₅₃N₂O₂S⁺.

According to the preparation method of Example 101 above, the following compounds were prepared using corresponding raw materials:

| Example | | |
|---|---|---|
| No. | Compound structure and reaction formula | NMR or MS, and appearance |
| 102 | 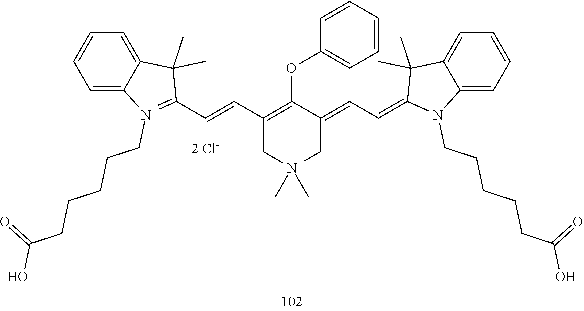 | ¹H NMR (400 MHz, MeOD) δ 8.04 (d, J = 15.2 Hz, 2H), 7.42- 7.50 (m, 6H), 7.38-7.40 (m, 2H), 7.29-7.33 (m, 2H), 7.25- 7.27 (m, 2H), 7.13-7.17 (m, 1H), 6.21 (d, J = 14.8 Hz, 2H), 4.79 (s, 4H), 4.24 (t, J = 14.8 Hz, 4H), 3.50 (s, 6H), 2.33 (t, J = 14.4 Hz, 4H), 1.83-1.87 (m, 4H), 1.68-1.72 (m, 4H), 1.48-1.52 (m, 4H), 1.37 (s, 12H). LCMS: m/z = 385.9, C₄₉H₆₁N₃O₅²⁺. Green solid. |
| | 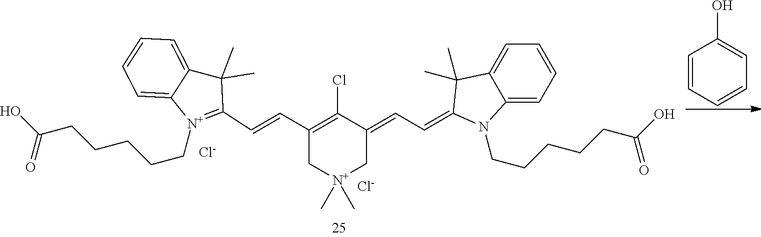 [SMILES: CC1(C)C(/C=C/C2=C(Cl)C(=C/C=C3/N(CCCCCC(=O)O)c4ccccc4C3(C)C)/C[N+](C)(C)C2)=[N+](CCCCCC(=O)O)c2ccccc21 ; [Cl-] ; [Cl-] ; Oc1ccccc1] | |
| | 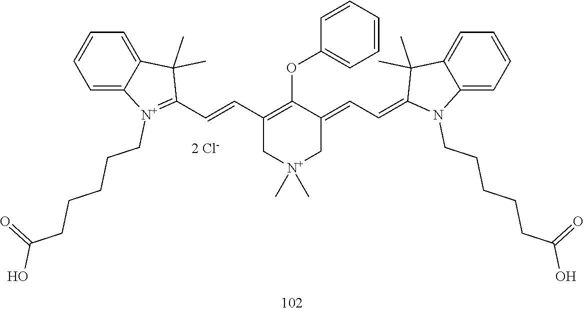 [SMILES: CC1(C)C(/C=C/C2=C(Oc3ccccc3)C(=C/C=C3/N(CCCCCC(=O)O)c4ccccc4C3(C)C)/C[N+](C)(C)C2)=[N+](CCCCCC(=O)O)c2ccccc21] | |
| 103 | 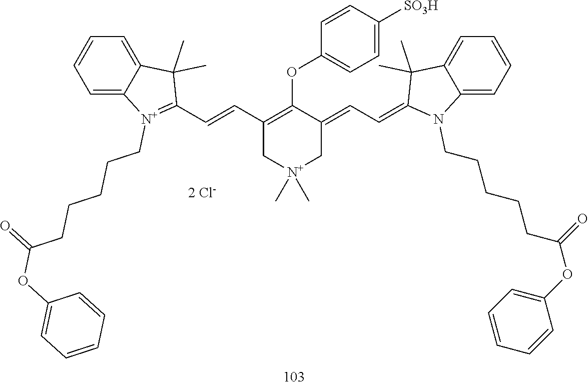 | ¹H NMR (400 MHz, MeOD) δ 7.89-7.96 (m, 4H), 7.46-7.47 (m, 2H), 7.37-7.42 (m, 8H), 7.25-7.31 (m, 6H), 7.02-7.04 (m, 4H), 6.14 (d, J = 14.8 Hz, 2H), 4.62 (s, 4H), 4.21-4.25 (m, 4H), 3.19 (s, 6H), 2.64 (t, J = 7.4 Hz, 4H), 1.81-1.89 (m, 8H), 1.55-1.60 (m, 4H), 1.34 (s, 12H). LCMS: m/z = 502.0, C₆₁H₆₉N₃O₈S²⁺. Green solid. |
| | 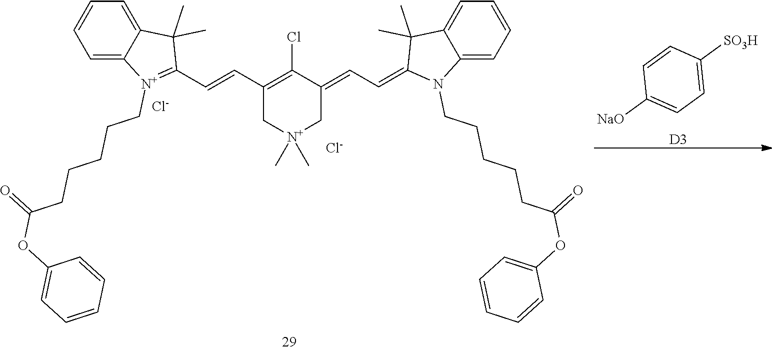 | |
| | 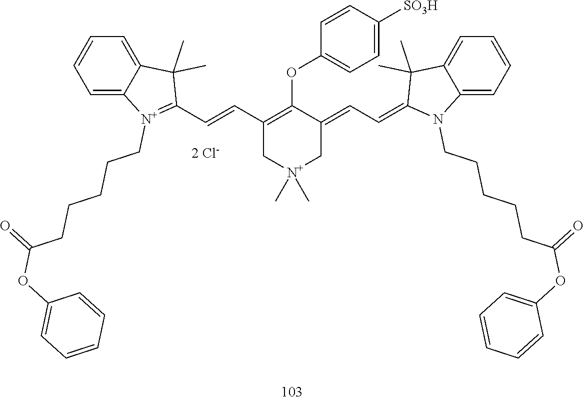 [SMILES: CC1(C)C(/C=C/C2=C(Oc3ccc(S(=O)(=O)O)cc3)C(=C/C=C3/N(CCCCCC(=O)Oc4ccccc4)c4ccccc4C3(C)C)/C[N+](C)(C)C2)=[N+](CCCCCC(=O)Oc2ccccc2)c2ccccc21] | |
| 104 | 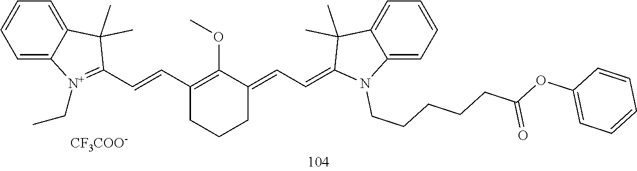 | ¹H NMR (400 MHz, CDCl₃) δ 7.94-7.86 (m, 2H), 7.39-7.35 (m, 4H), 7.24-7.06 (m, 9H), 6.10 (dd, J = 22.0, 14.0 Hz, 2H), 4.22-4.09 (m, 4H), 3.66 (s, 3H), 2.79-2.76 (m, 4H), 2.35 (t, J = 7.2 Hz,2H), 2.09- 2.06 (m, 2H), 1.84-1.78 (m, 2H), 1.73-1.67 (m,2H), 1.55- 1.48 (m, 2H), 1.42 (t, J = 7.2 Hz, 3H), 1.33-1.32 (m,12H). |
| | | LCMS: m/z=669.41, |
| | 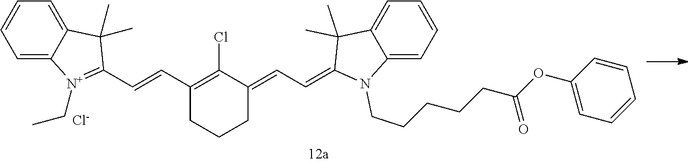 [SMILES: CC[N+]1=C(/C=C/C2=C(Cl)C(=C/C=C3/N(CCCCCC(=O)Oc4ccccc4)c4ccccc4C3(C)C)/CCC2)C(C)(C)c2ccccc21 ; [Cl-]] | C₄₅H₅₃N₂O₃⁺. Green solid. |
| | 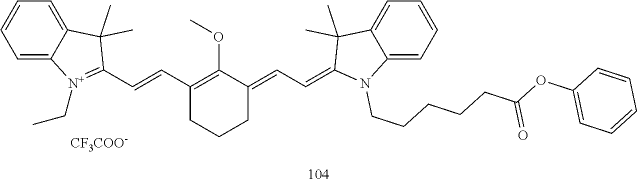 | |
| 106 | 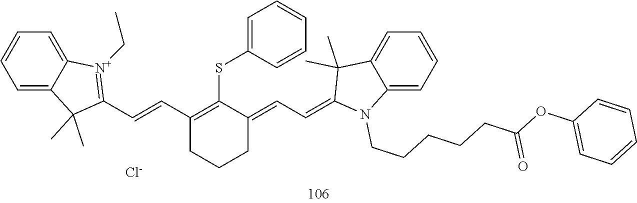 [SMILES: CC[N+]1=C(/C=C/C2=C(Sc3ccccc3)C(=C/C=C3/N(CCCCCC(=O)Oc4ccccc4)c4ccccc4C3(C)C)/CCC2)C(C)(C)c2ccccc21 ; [Cl-]] | ¹H NMR (400 MHz, CDCl₃) δ 8.66-8.72 (m, 2H), 7.35-7.38 (m, 4H), 7.28-7.32 (m, 2H), 7.22-7.25 (m, 7H), 7.07-7.16 (m, 3H), 7.03 (d, J = 7.6 Hz, 2H), 6.27-6.32 (m, 2H), 4.19-4.26 (m, 4H), 2.81 (s, 4H), 2.62 (t, J = 7.2 Hz, 2H), 2.05 (m, 2H), 1.83-1.90 (m, 4H), 1.62-1.66 (m, 2H), 1.43- 1.47 (m, 15H). LCMS: m/z = 747.6, |
| | | C₅₀H₅₅N₂O₂S⁺. |
| | 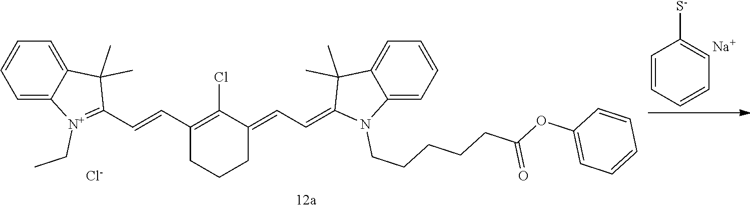 | Blue solid. |
| | 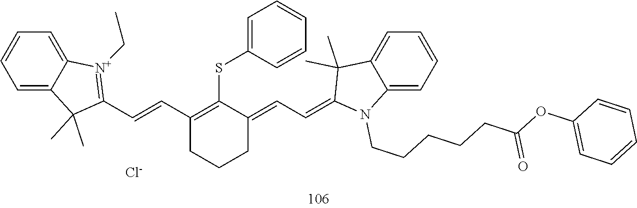 [SMILES: CC[N+]1=C(/C=C/C2=C(Sc3ccccc3)C(=C/C=C3/N(CCCCCC(=O)Oc4ccccc4)c4ccccc4C3(C)C)/CCC2)C(C)(C)c2ccccc21 ; [Cl-]] | |
| 107 | 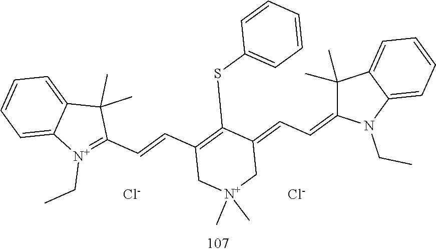 [SMILES: CCN1/C(=C/C=C2\C[N+](C)(C)CC(/C=C/C3=[N+](CC)c4ccccc4C3(C)C)=C2Sc2ccccc2)C(C)(C)c2ccccc21 ; [Cl-] ; [Cl-]] | ¹H NMR (400 MHz, MeOD) δ 8.79 (d, J = 14.8 Hz, 2H), 7.53- 7.36 (m, 12H), 7.34-7.21 (m, 1H), 6.37 (d, J = 14.8 Hz, 2H), 4.81 (s, 4H), 4.34 (dd, J1 = 7.2 Hz, J2 = 14.4 Hz, 4H), 3.43 (s, 6H), 1.52 (s, 12H), 1.44 (t, J = 7.2 Hz, 6H). LCMS: m/z = 614.4, C₄₁H₄₉N₃S²⁺ Blue solid. |
| | 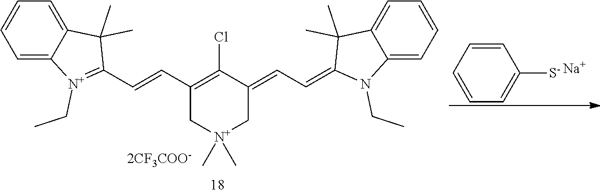 [SMILES: CCN1/C(=C/C=C2\C[N+](C)(C)CC(/C=C/C3=[N+](CC)c4ccccc4C3(C)C)=C2Cl)C(C)(C)c2ccccc21 ; [S-]c1ccccc1 ; [Na+]] | |
| | 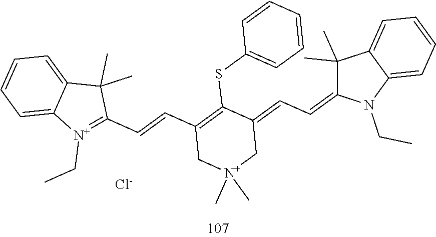 [SMILES: CCN1/C(=C/C=C2\C[N+](C)(C)CC(/C=C/C3=[N+](CC)c4ccccc4C3(C)C)=C2Sc2ccccc2)C(C)(C)c2ccccc21 ; [Cl-]] | |
| 108 | 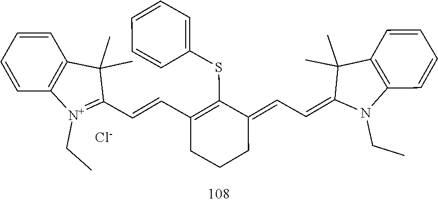 [SMILES: CCN1/C(=C/C=C2\CCCC(/C=C/C3=[N+](CC)c4ccccc4C3(C)C)=C2Sc2ccccc2)C(C)(C)c2ccccc21 ; [Cl-]] | ¹H NMR: (400 MHz, MeOD) δ 8.80-8.83 (m, 2H), 7.40-7.45 (m, 4H), 7.26-7.30 (m, 9H), 7.11-7.24 (m, 1H), 6.28-6.32 (m, 1H), 4.15-4.21 (m, 4H), 2.78-2.81 (m, 4H), 2.03-2.06 (m, 2H), 1.49 (s, 12H), 1.38 (t, J = 7.2 Hz, 6H). LCMS: m/z = 585.4, C₄₀H₄₅N₂S⁺. Blue solid. |
| | 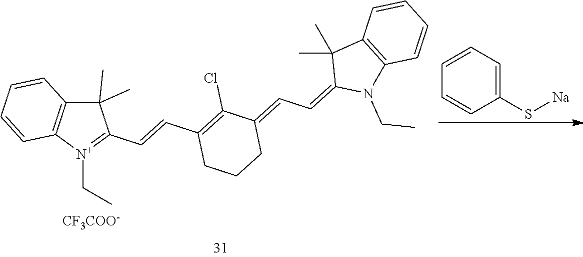 | |
| | 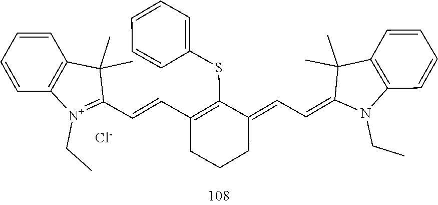 [SMILES: CCN1/C(=C/C=C2\CCCC(/C=C/C3=[N+](CC)c4ccccc4C3(C)C)=C2Sc2ccccc2)C(C)(C)c2ccccc21 ; [Cl-]] | |
| 112 | 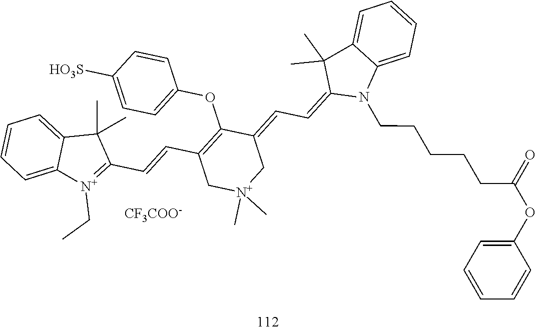 | ¹H NMR (400 MHz, CDCl₃) δ 7.91 (d, J = 8.0 Hz, 2H), 7.80 (d, J = 14.0 Hz, 2H), 7.39-7.35 (m, 2H), 7.29-7.28 (m, 1H), 7.22-7.15 (m, 5H), 7.09-6.96 (m, 7H), 6.38-6.21 (m, 2H), 4.94-4.76 (m, 2H), 4.27-4.10 (m, 2H), 3.31 (s, 4H), 2.73- 2.61 (m, 6H), 2.57-2.54 (m, 2H), 1.85-1.75 (m, 2H), 1.58- 1.53 (m, 2H), 1.24-1.22 (m, 12H), 0.88-0.86 (m, 5H). LCMS: m/z = 841.0, C₅₁H₅₉N₃O₆S²⁺. Blue solid. |
| | 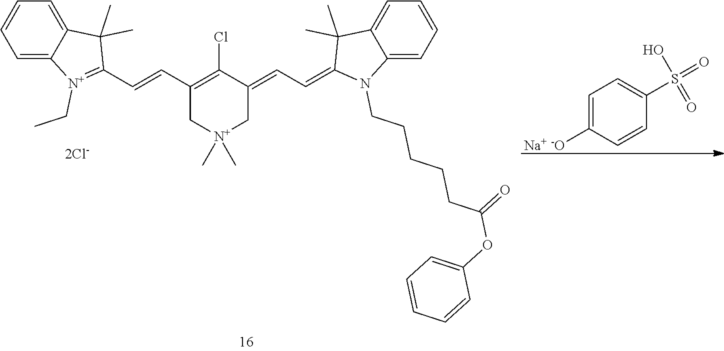 | |
| | 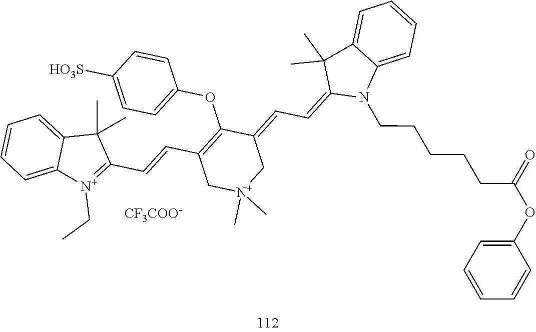 | |
| 113 | 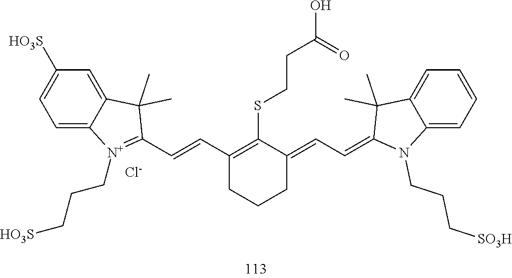 [SMILES: CC1(C)C(/C=C/C2=C(SCCC(=O)O)C(=C/C=C3/N(CCCS(=O)(=O)O)c4ccccc4C3(C)C)/CCC2)=[N+](CCCS(=O)(=O)O)c2ccc(S(=O)(=O)O)cc21 ; [Cl-]] | ¹H NMR (400 MHz, CDCl₃) δ 904 (d, J = 14.4 Hz, 1H), 8.73 (d, J = 13.6 Hz, 1H), 7.86-7.84 (m, 2H), 7.57-7.52 (m, 2H), 7.36-7.30 (m, 2H), 6.62 (d, J = 14.4 Hz, 1H), 6.37 (d, J = 13.6 Hz, 1H), 4.49-4.45 (m, 2H), 4.31-4.27 (m, 2H), 3.08 (t, J = 6.4 Hz, 2H), 3.00-2.96 (m, 4H), 2.75-2.72 (m, 4H), 2.60 (t, J = 6.8 Hz, 2H), 2.32- 2.20 (m, 4H), 1.95-1.92 (m, 2H), 1.79 (s, 6H), 1.77 (s, 6H). LCMS: m/z = 849.2, C₃₉H₄₉N₂O₁₁S₄. Green solid. |
| | 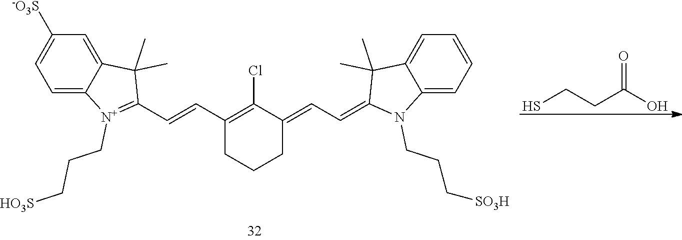 | |
| | 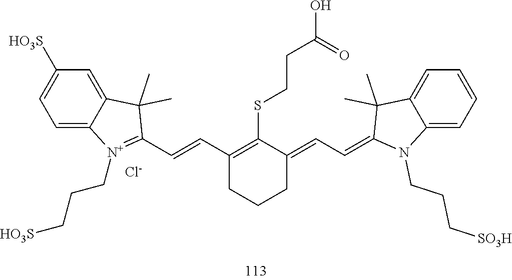 [SMILES: CC1(C)C(/C=C/C2=C(SCCC(=O)O)C(=C/C=C3/N(CCCS(=O)(=O)O)c4ccccc4C3(C)C)/CCC2)=[N+](CCCS(=O)(=O)O)c2ccc(S(=O)(=O)O)cc21 ; [Cl-]] | |
| 114 | 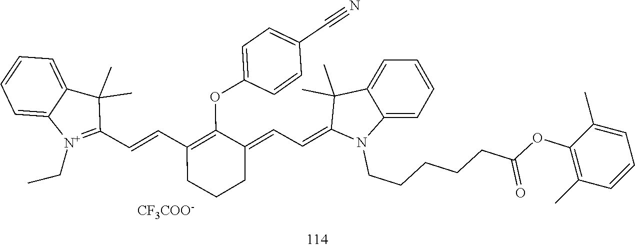 | ¹H NMR (400 MHz, CDCl₃) δ 7.70-7.75 (m, 4H), 7.35-7.37 (m, 2H), 7.28-7.30 (m, 1H), 7.20-7.26 (m, 5H), 7.11-7.15 (m, 2H), 7.05 (s, 3H), 6.02- 6.08 (m, 2H), 4.05-4.13 (m, 4H),2.66 (t, J = 7.2 Hz, 4H), 2.11 (s, 6H), 2.02-2.04 (m, 2H), 1.85-1.89 (s, 5H), 1.56- 1.59 (m, 1H), 1.52-1.53 (m, 2H), 1.40-1.43 (m, 3H), 1.34 (d, J = 2.0 Hz, 12H). LCMS: m/z = 784.6, C₅₃H₅₈N₃O₃⁺. Blue solid. |
| | 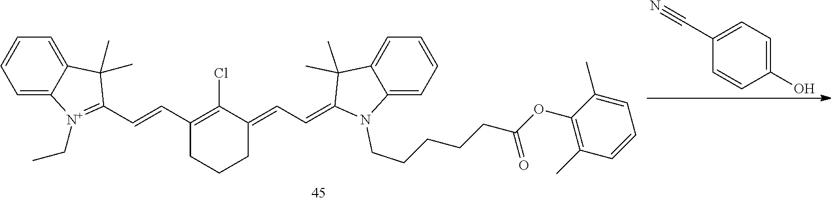 [SMILES: CC[N+]1=C(/C=C/C2=C(Cl)C(=C/C=C3/N(CCCCCC(=O)Oc4c(C)cccc4C)c4ccccc4C3(C)C)/CCC2)C(C)(C)c2ccccc21 ; N#Cc1ccc(O)cc1] | |
| | 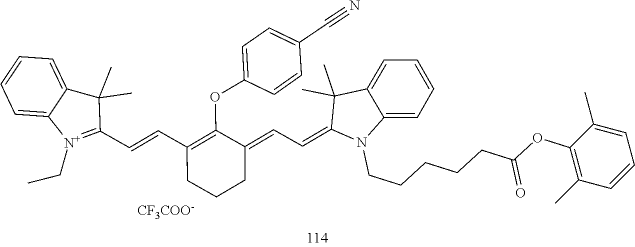 | |
| 115 | 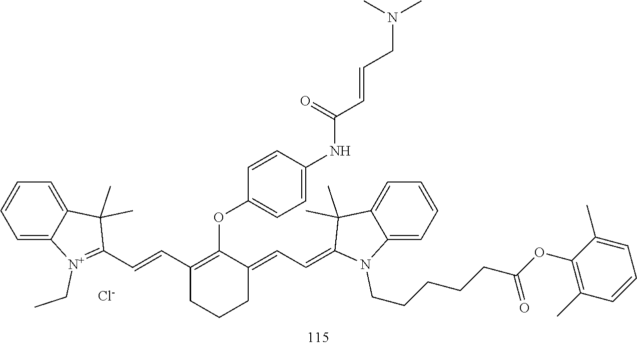 [SMILES: CC[N+]1=C(/C=C/C2=C(Oc3ccc(NC(=O)/C=C/CN(C)C)cc3)C(=C/C=C3/N(CCCCCC(=O)Oc4c(C)cccc4C)c4ccccc4C3(C)C)/CCC2)C(C)(C)c2ccccc21 ; [Cl-]] | ¹H NMR (400 MHz, MeOD) δ 7.98-8.09 (m, 2H), 7.70 (d, J = 9.2 Hz, 2H), 7.39 (s, 5H), 7.14-7.24 (m, 2H), 7.03-7.12 (m, 6H), 6.80-6.84 (m, 1H), 6.54 (d, J = 15.2 Hz, 1H), 4.84 (s, 8H), 4.04-4.16 (m, 2H), 3.98 (d, J = 7.6 Hz, 2H), 2.91 (s, 6H), 2.67 (t, J = 7.2 Hz, 2H ), 2.06 (s, 6H), 1.81- 1.90 (m, 6H), 1.57-1.58 (m, 2H), 1.34-1.37 (m, 15H). LCMS: m/z = 886.7, C₅₈H₆₉N₄O₄⁺. Green solid. |
| | 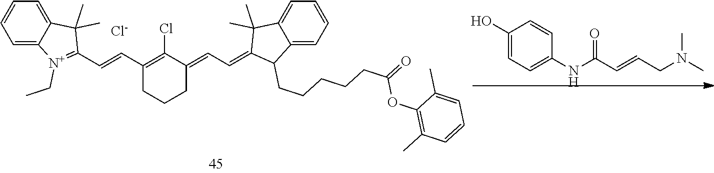 [SMILES: CC[N+]1=C(/C=C/C2=C(Cl)C(=C/C=C3/C(CCCCCC(=O)Oc4c(C)cccc4C)c4ccccc4C3(C)C)/CCC2)C(C)(C)c2ccccc21 ; [Cl-] ; CN(C)C/C=C/C(=O)Nc1ccc(O)cc1] | |
| | 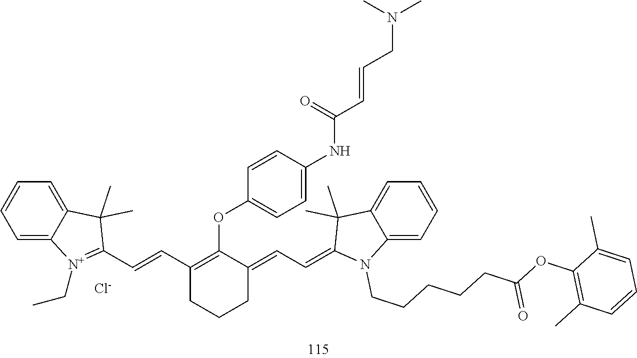 [SMILES: CC[N+]1=C(/C=C/C2=C(Oc3ccc(NC(=O)/C=C/CN(C)C)cc3)C(=C/C=C3/N(CCCCCC(=O)Oc4c(C)cccc4C)c4ccccc4C3(C)C)/CCC2)C(C)(C)c2ccccc21 ; [Cl-]] | |
| 116 | 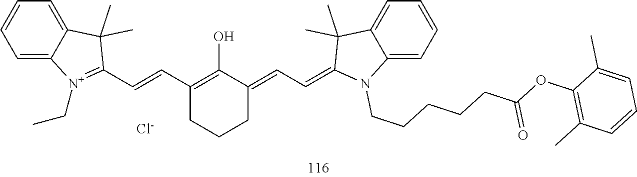 [SMILES: CC[N+]1=C(/C=C/C2=C(O)C(=C/C=C3/N(CCCCCC(=O)Oc4c(C)cccc4C)c4ccccc4C3(C)C)/CCC2)C(C)(C)c2ccccc21 ; [Cl-]] | LCMS: m/z = 683.3, C₄₆H₅₅N₂O₃⁺. Black oil. |
| | 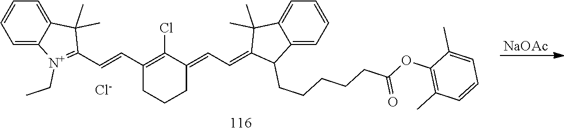 [SMILES: CC[N+]1=C(/C=C/C2=C(Cl)C(=C/C=C3/C(CCCCCC(=O)Oc4c(C)cccc4C)c4ccccc4C3(C)C)/CCC2)C(C)(C)c2ccccc21 ; [Cl-] ; "NaOAc"] | |
| | 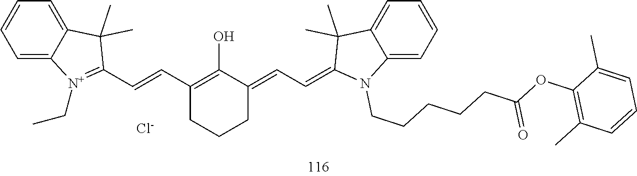 [SMILES: CC[N+]1=C(/C=C/C2=C(O)C(=C/C=C3/N(CCCCCC(=O)Oc4c(C)cccc4C)c4ccccc4C3(C)C)/CCC2)C(C)(C)c2ccccc21 ; [Cl-]] | |

### Example 117: Preparation of 5-carboxy-2-((E)-2-((E)-2-chloro-3-(2-((E)-3,3-dimethyl-1-(2-(3-oxo-3-phenoxypropoxy)ethyl)indolin-2-ylidene)ethenyl)cyclohex-1-en-1-yl)ethenyl)-1-ethyl-3,3-dimethyl-3H-indol-1-ium 2,2,2-trifluoroacetate (117)

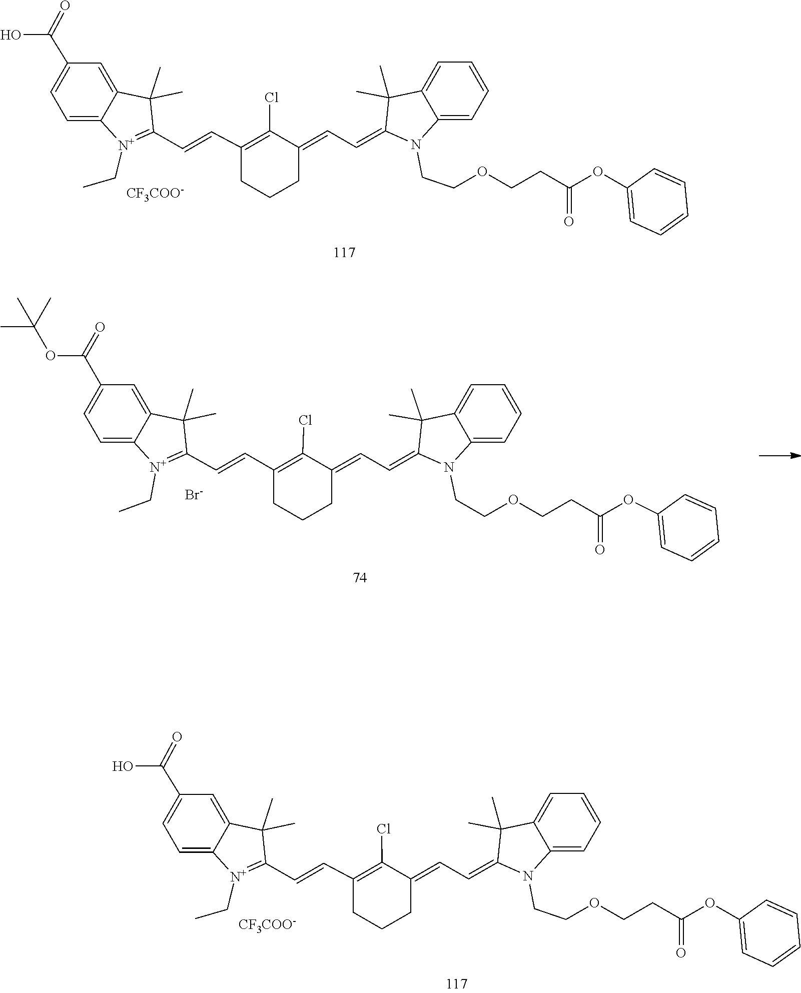

TFA (8.81 g, 77.3 mmol, 5.74 mL, 20.0 eq) was added to a solution of Compound 74 (3.00 g, 3.86 mmol, 1.00 eq) in DCM (10.0 mL), and stirred at 20° C. for 4 hours. The resulting mixture was concentrated under reduced pressure, and the residues were purified by reversed-phase HPLC (Phenomenex Synergi Polar-RP 100 mm×25 mm×4 μm; mobile phase: [water (TFA)-ACN]; B %: 25%-85%; 9 min) to obtain Compound 117 as a blue solid after lyophilization, with a yield of 9.63%.

¹H NMR (400 MHz, MeOD) δ 8.50-8.65 (m, 1H), 8.16-8.27 (m, 1H), 8.03-8.07 (m, 2H), 7.62 (d, J=7.2 Hz, 1H), 7.51-7.57 (m, 2H), 7.22-7.25 (m, 1H), 7.42-7.46 (m, 2H), 7.26-7.30 (m, 2H), 7.14-7.19 (m, 2H), 6.83-6.85 (m, 1H), 6.09 (br, 1H), 4.60 (s, 2H), 4.08 (s, 2H), 3.99 (t, J=4.4 Hz, 2H), 3.79 (t, J=5.6 Hz, 2H), 2.64-2.77 (m, 6H), 1.85-1.88 (m, 2H), 1.72 (d, J=6.4 Hz, 12H), 1.36 (t, J=7.2 Hz, 3H).

LCMS: m/z=719.4, C₄₄H₄₈ClN₂O₅.

### Example 118: Preparation of 5-carboxy-2-((E)-2-((E)-2-chloro-3-(2-((E)-3,3-dimethyl-1-(6-oxo-6-phenoxyhexyl)indolin-2-ylidene)ethenyl)cyclohex-1-en-1-yl)ethenyl)-1-ethyl-3,3-dimethyl-3H-indol-1-ium 2,2,2-trifluoroacetate (118)

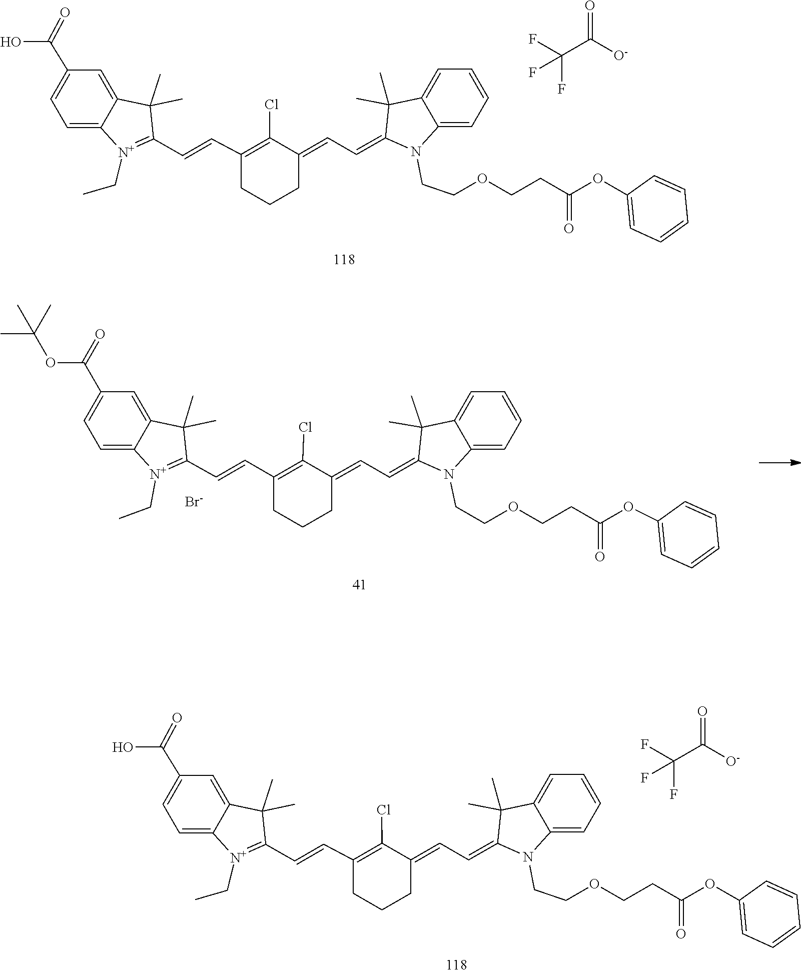 [SMILES: CC[N+]1=C(/C=C/C2=C(Cl)C(=C/C=C3/N(CCOCCC(=O)Oc4ccccc4)c4ccccc4C3(C)C)/CCC2)C(C)(C)c2cc(C(=O)O)ccc21 ; O=C([O-])C(F)(F)F ; CC[N+]1=C(/C=C/C2=C(Cl)C(=C/C=C3/N(CCOCCC(=O)Oc4ccccc4)c4ccccc4C3(C)C)/CCC2)C(C)(C)c2cc(C(=O)OC(C)(C)C)ccc21 ; [Br-] ; CC[N+]1=C(/C=C/C2=C(Cl)C(=C/C=C3/N(CCOCCC(=O)Oc4ccccc4)c4ccccc4C3(C)C)/CCC2)C(C)(C)c2cc(C(=O)O)ccc21 ; O=C([O-])C(F)(F)F]

Compound 118 was prepared as a blue solid according to the preparation method similar to Example 117, except that Compound 41 was used instead of Compound 74.

¹H NMR (400 MHz, MeOD) δ 8.61 (d, J=14.4 Hz, 1H), 8.28 (d, J=13.6 Hz, 1H), 8.05-8.10 (m, 2H), 7.63-7.65 (m, 1H), 7.50-7.57 (m, 2H), 7.42-7.46 (m, 1H), 7.36 (t, J=8.0 Hz, 2H), 7.20-7.24 (m, 2H), 6.99 (d, J=7.6 Hz, 2H), 6.59 (d, J=14.8 Hz, 1H), 6.14 (d, J=13.6 Hz, 1H), 4.36-4.41 (m, 2H), 4.08-4.17 (m, 2H), 2.70-2.75 (m, 4H), 2.62 (t, J=7.2 Hz, 2H), 1.90-2.00 (m, 4H), 1.80-1.87 (m, 2H), 1.75 (d, J=13.2 Hz, 12H), 1.58-1.64 (m, 2H), 1.38 (t, J=7.2 Hz, 3H).

LCMS: m/z=717.4, C₄₅H₅₀ClN₂O₄⁺.

### Example 119: Preparation of 5-carboxy-2-((E)-2-((E)-3-(2-((E)-1-(5-carboxypentyl)-3,3-dimethylindolin-2-ylidene)ethenyl)-2-chlorocyclohex-1-en-1-yl)ethenyl)-1,3,3-trimethyl-3H-indol-1-ium 2,2,2-trifluoroacetate (119)

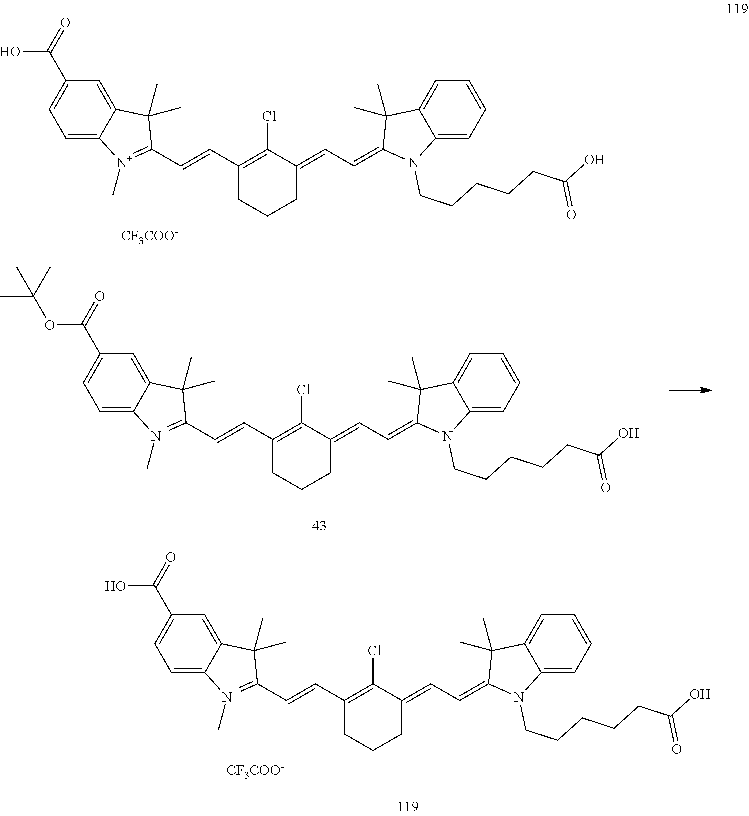

Compound 119 was prepared according to the preparation method similar to Example 117, except that Compound 43 was used instead of Compound 74.

¹H NMR (400 MHz, MeOD) δ 8.63 (d, J=14.8 Hz, 1H), 8.27 (d, J=13.6 Hz, 1H), 8.03-8.08 (m, 2H), 7.64 (d, J=7.6 Hz, 1H), 7.53-7.54 (m, 2H), 7.44-7.46 (m, 1H), 7.21 (d, J=8.4 Hz, 1H), 6.59 (d, J=14.8 Hz, 1H), 6.11 (d, J=13.6 Hz, 1H), 4.36 (t, J=7.2 Hz, 2H), 3.55 (s, 3H), 2.72-2.80 (m, 4H), 2.33 (t, J=7.2 Hz, 2H), 1.91-1.99 (m, 4H), 1.78 (s, 6H), 1.74 (s, 6H), 1.67-1.71 (m, 2H), 1.50-1.54 (m, 2H).

LCMS: m/z=627.3, C₃₈H₄₄ClN₂O₄⁺.

### Example 120: Preparation of 5-carboxy-2-((E)-2-((E)-2-chloro-3-(2-((E)-3,3-dimethyl-1-(6-oxo-6-phenoxyhexyl)indolin-2-ylidene)ethenyl)cyclohex-1-en-1-yl)ethenyl)-1,3,3-trimethyl-3H-indol-1-ium 2,2,2-trifluoroacetate (120)

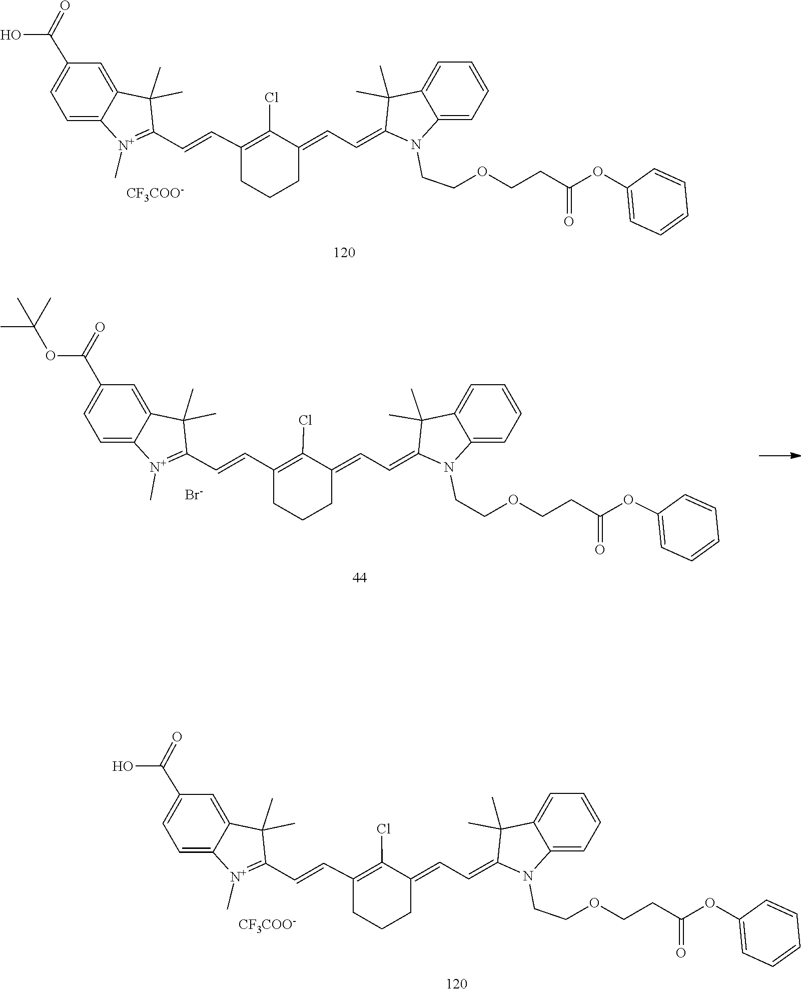

Compound 120 was prepared according to the preparation method similar to Example 117, except that Compound 44 was used instead of Compound 74.

¹H NMR (400 MHz, MeOD) δ 8.61 (d, J=14.8 Hz, 1H), 8.27 (d, J=14.0 Hz, 1H), 8.03-8.08 (m, 2H), 7.64 (d, J=7.2 Hz, 1H), 7.50-7.57 (m, 2H), 7.43-7.46 (m, 1H), 7.34-7.37 (m, 2H), 7.20-7.23 (m, 2H), 6.98-7.00 (m, 2H), 6.59 (d, J=14.8 Hz, 1H), 6.10 (d, J=13.6 Hz, 1H), 4.39 (t, J=6.8 Hz, 2H), 3.55 (s, 3H), 2.67-2.76 (m, 4H), 2.62 (t, J=7.2 Hz, 2H), 1.96-2.00 (m, 2H), 1.87-1.94 (m, 2H), 1.80-1.85 (m, 2H), 1.76 (s, 6H), 1.73 (s, 6H), 1.58-1.64 (m, 2H).

LCMS: m/z=703.3, C₄₄H₄₈ClN₂O₄⁺.

### Example 121: Preparation of 2-((E)-2-((E)-2-(((E)-4-(dimethylamino)but-2-enoyl)oxy)-3-(2-((E)-1-(6-(2,6-dimethylphenoxy)-6-oxohexyl)-3,3-dimethylindolin-2-ylidene)ethenyl)cyclohex-1-en-1-yl)ethenyl)-1-ethyl-3,3-dimethyl-3H-indol-1-ium 2,2,2-trifluoroacetate (121)

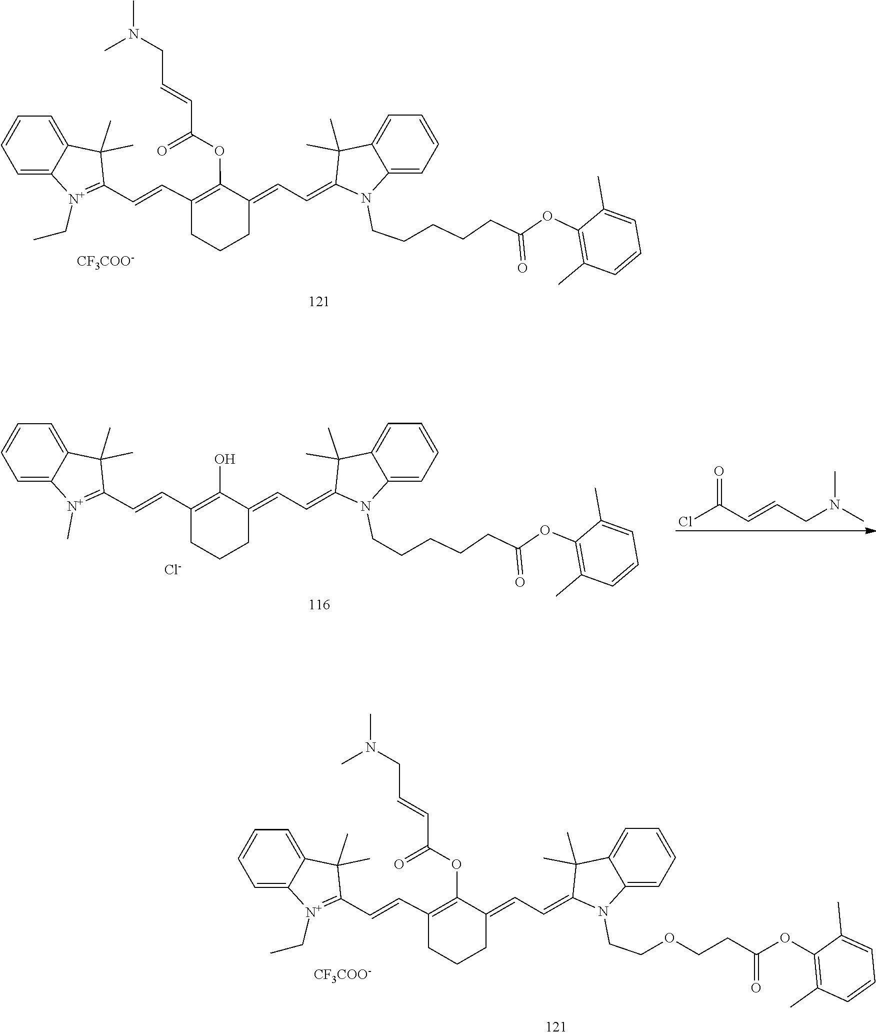

Pyridine (27.5 mg, 347 μmol, 28.1 μL, 0.500 eq) was added to a solution of Compound 116 (500 mg, 695 μmol, 1.00 eq), (E)-4-(dimethylamino)but-2-enyl hydrochloride (820 mg, 5.56 mmol, 8.00 eq) in DCM (10 mL). The reaction mixture was stirred at 45° C. for 12 hours. The mixture was concentrated under reduced pressure, and the residues were purified by reversed-phase HPLC (column: Phenomenex luna C18 150 mm×40 mm×15 μm; mobile phase: [water (HCl)-ACN]; gradient: 35%-65% B, 10 min) to obtain Compound 121 (40.0 mg, 30.6 μmol, 4.40% yield, 69.5% purity) as a green solid.

¹H NMR (400 MHz, CDCl₃) δ 7.70-7.77 (m, 2H), 7.55-7.61 (m, 1H), 7.42-7.45 (m, 4H), 7.36-7.38 (m, 1H), 7.23-7.25 (m, 1H), 7.03-7.07 (m, 5H), 6.83-6.87 (m, 1H), 5.94-6.00 (m, 2H), 4.33 (d, J=3.6 Hz, 2H), 3.97-4.06 (m, 4H), 3.00 (s, 6H), 2.61-2.68 (m, 6H), 2.12 (s, 6H), 1.86-2.07 (m, 8H), 1.59 (s, 12H), 1.43 (t, J=6.8 Hz, 3H).

LCMS: m/z=794.5, C₅₂H₆₄N₃O₄⁺.

### Example 122: Preparation of 2-((E)-2-((E)-2-chloro-3-(2-((E)-3,3-dimethyl-1-(5-phosphonylpentyl)indolin-2-ylidene)ethenyl)cyclohex-1-en-1-yl)ethenyl)-1,3,3-trimethyl-3H-indol-1-ium Chloride (122)

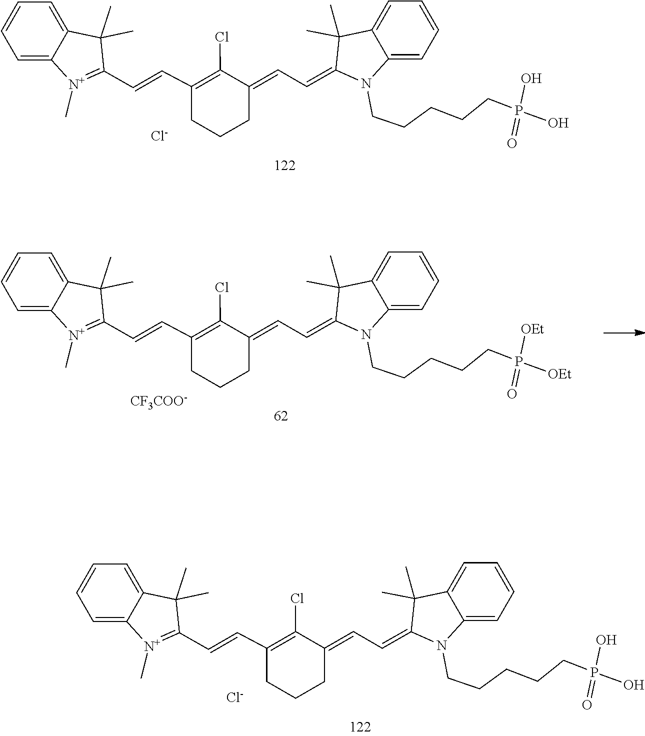

HCl/dioxane (6.00 M, 1.27 mL, 10.0 eq) was added to a solution of Compound 62 (0.600 g, 760 μmol, 1.00 eq) in MeCN (3.00 mL) at 0° C. The mixture was heated to 100° C. and stirred for 24 hours. The resulting mixture was filtered, and the filtrate was concentrated. The residues were purified by reversed-phase HPLC (column: Phenomenex luna C18 150 mm×40 mm×15 μm; mobile phase: [water (HCl)-ACN]; gradient: 45%-75% B, 10 min) to obtain Compound 122 (27.0 mg, 39.5 μmol, 5.20% yield, 95.9% purity) as a green solid.

¹H NMR (400 MHz, DMSO-d6) δ 8.24-8.28 (m, 2H), 7.63 (t, J=7.6 Hz, 2H), 7.42-7.46 (m, 4H), 7.27-7.30 (m, 2H), 6.31 (t, J=14.4 Hz, 2H), 4.17-4.20 (m, 2H), 3.70 (s, 3H), 2.70-2.72 (m, 4H), 1.86-1.87 (m, 2H), 1.71-1.73 (m, 2H), 1.67 (s, 12H), 1.47-1.55 (m, 8H).

LCMS: m/z=619.3, C₃₆H₄₅ClN₂O₃P⁺.

### Example 123: Preparation of 5-carboxy-2-((E)-2-((E)-2-chloro-3-(2-((E)-3,3-dimethyl-1-(2-(3-oxo-3-phenoxypropoxy)ethyl)indolin-2-ylidene)ethenyl)cyclohex-1-en-1-yl)ethenyl)-1-ethyl-3,3-dimethyl-3H-indol-1-ium 2,2,2-trifluoroacetate (123)

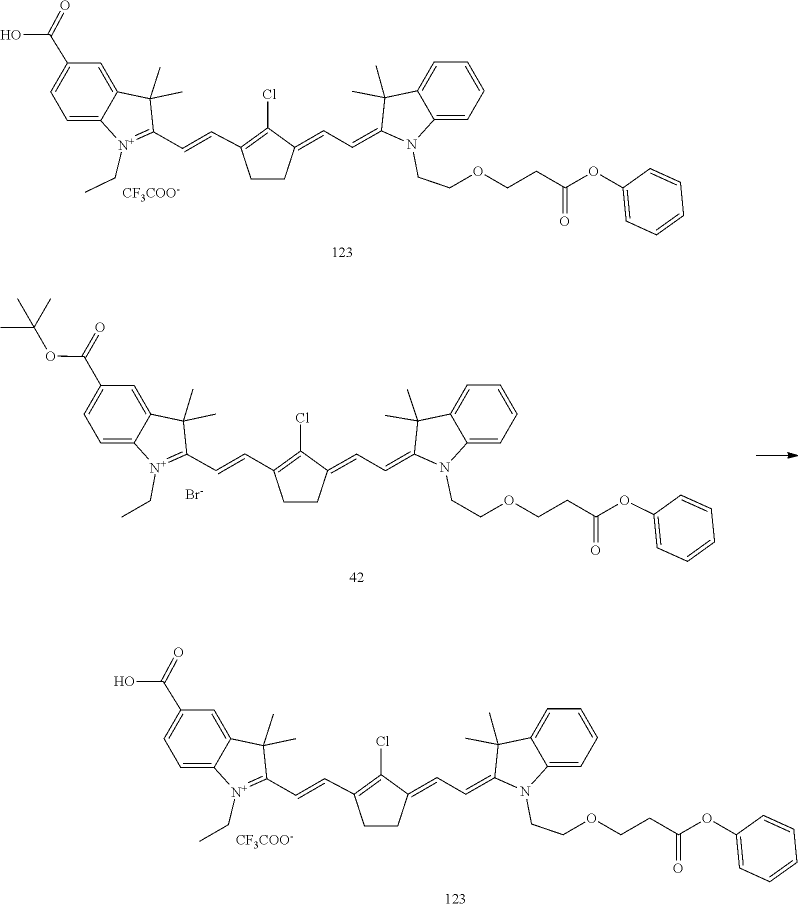

Compound 123 was prepared as a blue solid according to the preparation method similar to Example 118, except that Compound 42 was used instead of Compound 41.

¹H NMR (400 MHz, CDCl₃) δ 8.04 (d, J=8.4 Hz, 1H), 7.98-8.01 (m, 2H), 7.62 (d, J=13.2 Hz, 1H), 7.41-7.53 (m, 2H), 7.30-7.37 (m, 4H), 7.21 (t, J=16.0 Hz, 1H), 7.01 (d, J=7.6 Hz, 2H), 6.95 (d, J=8.0 Hz, 1H), 6.71 (d, J=14.0 Hz, 1H), 5.87 (d, J=13.6 Hz, 1H), 4.66 (s, 2H), 4.07 (br, 2H), 3.18 (br, 2H), 2.96 (br, 2H), 2.60-2.67 (m, 2H), 1.98 (br, 2H), 1.76-1.87 (m, 4H), 1.71 (d, J=19.2 Hz, 12H), 1.35-1.45 (m, 3H).

LCMS: m/z=703.3, C₄₄H₄₈ClN₂O₄⁺.

In the present invention, the following compounds are also synthesized:

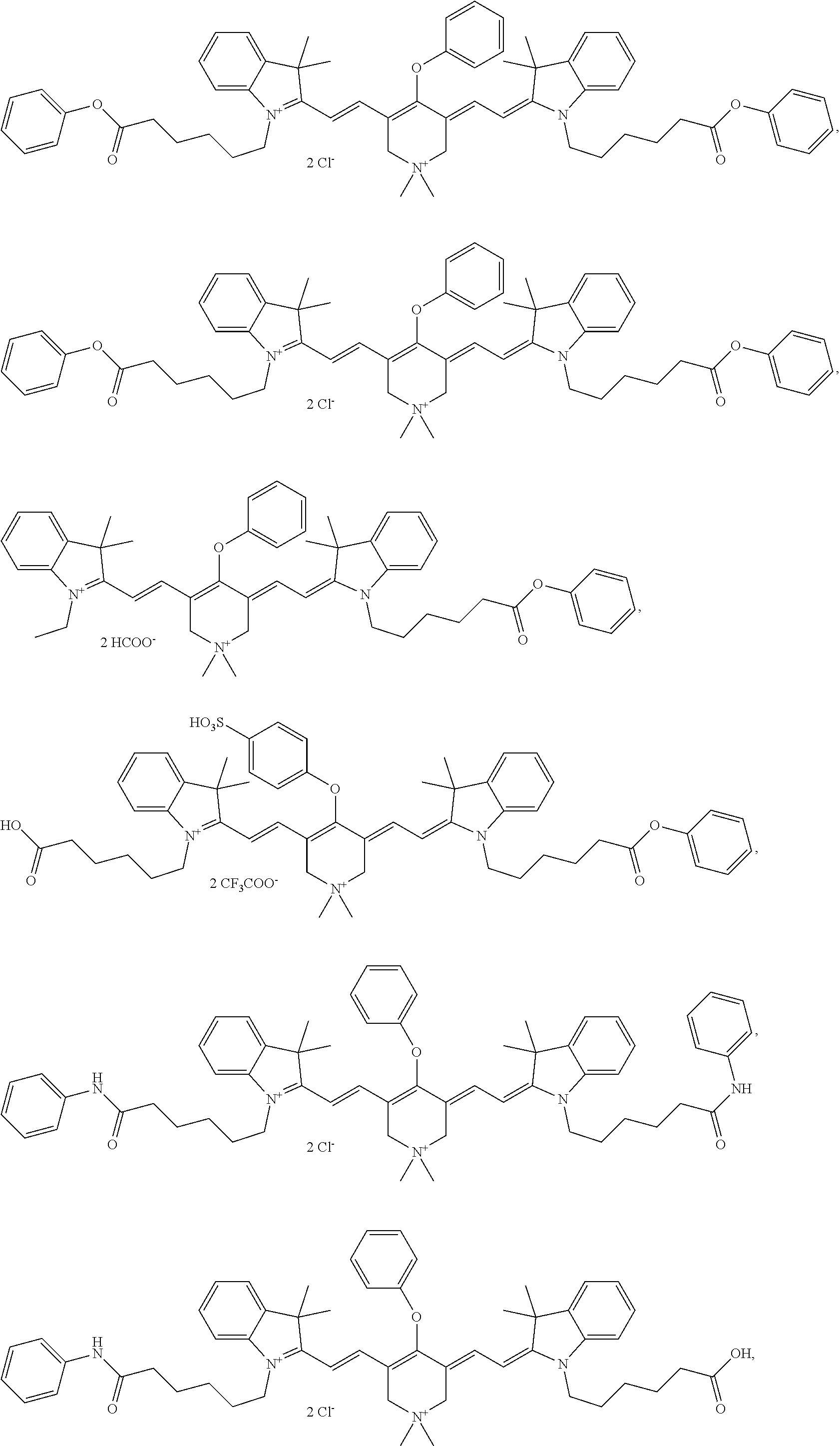

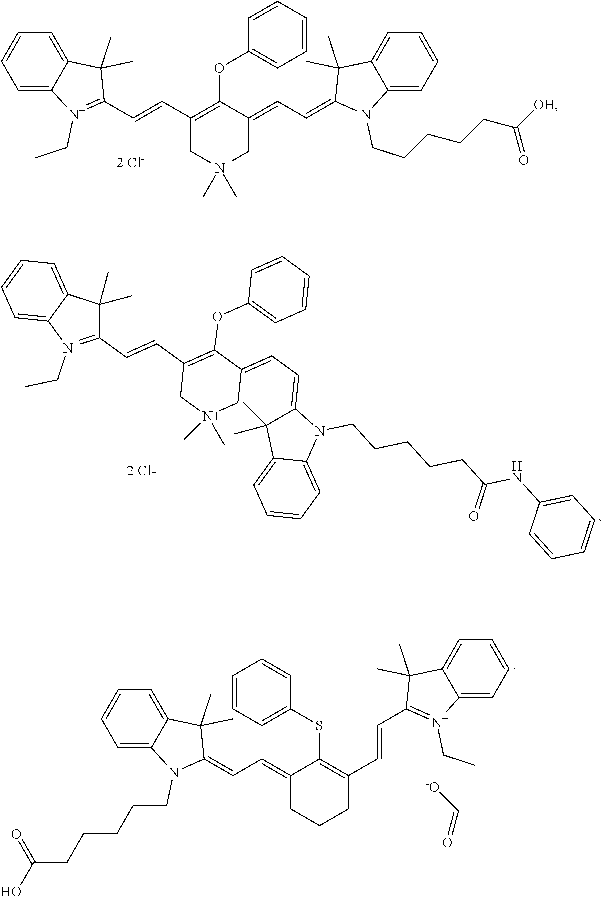

### Comparative Example 1: Preparation of 2-((E)-2-((E)-2-chloro-3-(2-((E)-3,3-dimethyl-1-(6-oxo-6-phenoxyhexyl)indolin-2-ylidene)ethenyl)cyclohex-1-en-1-yl)ethenyl)-1-ethyl-3,3-dimethyl-3H-indol-1-ium Chloride (12a)

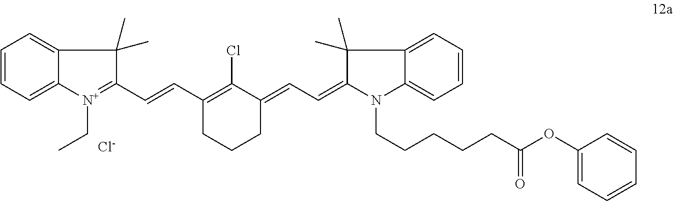 [SMILES: CC[N+]1=C(/C=C/C2=C(Cl)C(=C/C=C3/N(CCCCCC(=O)Oc4ccccc4)c4ccccc4C3(C)C)/CCC2)C(C)(C)c2ccccc21 ; [Cl-]]

Compound 12a was prepared according to the same preparation method as Example 34, except that Compound C1 was used instead of Compound C10.

¹H NMR (400 MHz, CDCl₃) δ 8.35 (t, J=13.2 Hz, 2H), 7.43-7.35 (m, 6H), 7.25-7.13 (m, 5H), 7.03 (d, J=8.4 Hz, 2H), 6.25-6.19 (m, 2H), 4.22-4.16 (m, 4H), 2.71-2.66 (m, 4H), 2.62 (t, J=6.8 Hz, 2H), 2.01-1.88 (m, 4H), 1.87-1.83 (m, 2H), 1.73-1.72 (m, 12H), 1.67-1.62 (m, 2H), 1.46 (t, J=6.8 Hz, 3H).

LCMS: m/z=673.4, C₄₄H₅₀N₂O₂Cl₂.

### TEST EXAMPLES

### Test Example 1. Solubility, hERG, Optical Property, and MRC5 Tests

### (1) Method for Solubility Test

The compounds were weighed in an appropriate amount and dissolved in DMSO to prepare a 10 mM stock solution. 30 μL of the stock solution was taken and 970 μL of phosphate buffered saline solution (PBS) was added thereto. The solution was shaken at 25° C. on a thermostatic mixer for 2 hours, and centrifuged at 12000 rpm for 10 minutes. The supernatant was taken, and the kinetic solubility of each compound in phosphate buffered saline solution (PBS) was determined by Waters Acquity Arc HPLC. The test results are shown in Table 1 below.

### (2) Method for HERG Test

### 1) Materials and Instruments

| Material | Manufacturer (batch number) |
|---|---|
| 6 cm cell-culture dish | Nunc (150288) |
| 3.5 cm cell-culture dish | Nunc (153066) |
| Dialyzed fetal bovine serum | BIOSUN (BS-0005-500) |
| DMSO | Merk (102952) |
| DMEM medium | Gibco (10569) |
| HEPES | Gibco (15630080) |
| TrypLE ™ trypsin | Gibco (12604) |
| 1 × PBS, free of Ca²⁺/Mg²⁺ | Gibco (14190) |
| Penicillin - streptomycin | Gibco (15140-122) |
| MEM + NEAA | Gibco (11140) |
| Geneticin, G418 | Gibco (11811031) |
| Blasticidin | Gibco (R21001) |
| Polylysine | Sigma (P4832) |
| Dofetilide | TRC (D525700) |
| Doxycycline | Sigma (D9891) |
| Instrument | Manufacturer |
| Steri-Cycle CO₂ incubator | Thermo(371) |
| Micropipette puller | Sutter (P-97 Model) |
| Micromanipulator | Siskiyou Controller (MC1000e) |
| | Sutter (ROE-200; MP285) |
| Multilamp700B amplifier | HEKA (EPC10) |
| EPC10 amplifier | AXON (Multiclamp 700B) |
| Microscope | Olympus (IX51/71/73) |

### 2) Cell Line and Cell Culture

The HEK 293 cell line (K1236) stably expressing hERG was purchased from Invitrogen. Cells were cultured in 85% DMEM, 10% dialyzed FBS, 0.1 mM NEAA, 25 mM HEPES, 100 U/mL penicillin-streptomycin, 5 μg/mL blasticidin and 400 μg/mL geneticin. Cells were split using TrypLE™ about three times a week, and maintained at a confluence of about 40% to about 80%. Before measurement, cells were cultured at a density of 5×10⁵ in a 6 cm culture dish and induced with 1 μg/mL doxycycline for 48 hours.

### 3) Solution Preparation

Extracellular solution (mM): 132 NaCl, 4 KCl, 13 CaCl₂, 0.5 MgCl₂, 11.1 glucose, 10 HEPES (pH 7.35 adjusted with NaOH);

Intercellular solution (mM): 140 KCl, 2 MgCl₂, 10 EGTA, 10 HEPES, 5 MgATP (pH 7.35 adjusted with KOH).

### 4) Preparation of Working Solution of Test Compounds

The test compounds were formulated in DMSO to prepare a stock solution with a final concentration of 10 mM.

Then, the stock solution of each compound was diluted with DMSO in a 1:3 serial dilution to prepare five intermediate solutions with concentrations of 3, 1, 0.33, 0.12, 0.04 mM or 3.33, 1.11, 0.37, 0.12 mM, respectively.

Before testing, the final working solutions were prepared by diluting the intermediate solutions 1000 times with the extracellular solution. The concentration of DMSO in the working solutions was within a range around 0.1%.

The working solutions at five different concentrations of 3, 1, 0.33, 0.12 and 0.04 μM were used to determine the potential inhibition effect of the compounds on hERG channels and to fit concentration-response curves and calculate IC₅₀.

### 5) Experimental Procedures

The coverslip was removed from the cell culture dish and placed on a microscope stage.

A 10× objective was used to locate cells to be observed. An electrode tip was located under the microscope using the 10× objective by focusing above the plane of the cells. Once the tip was in focus, the electrode was advanced downward towards the cells under rough control of the manipulator, while the objective was moved so as to keep the tip in focus.

When the electrode was directly over a cell, the objective was switched to a 40× objective and fine control of the manipulator was used to enable to gradually approach the cell surface.

With gentle suction through a side port of an electrode holder, a giga-ohm seal was formed.

C_{fast} was used to eliminate the capacitive current consistent with the voltage step. Brief and strong suction was applied repeatedly until the membrane patch ruptured and the whole-cell configuration was obtained.

At this point, membrane potential was set to −60 mV in order to ensure that hERG channels were not open. Then, C_{slow} on the amplifier was used to remove the spikes of capacitive current.

Holding potential was set to −90 mV for 500 ms. Current was recorded at 20 kHz and filtering was carried out at 10 kHz. Leak current was tested at −80 mV for 500 ms.

The hERG current was elicited by depolarizing at +30 mV for 4.8 seconds, and then the voltage was restored to −50 mV for 5.2 seconds to remove the inactivation and observe the deactivating tail current. The maximum of tail current size was used to determine hERG current amplitude.

Current was recorded for 120 seconds to evaluate the current stability. Only stable cells with recording parameters above thresholds were used for drug administrations.

Firstly, vehicle control was applied to the cells to establish the baseline. Once the hERC current was found to be stabilized for 5 minutes, the compound was applied. The hERG current in the presence of the test compound was recorded for about 5 minutes to reach a steady state, and then 5 sweeps were captured. For dose-response testing, multiple compounds were applied to the cells cumulatively from low to high concentrations (multiple compounds were detected simultaneously). The positive control, Dofetilide at a concentration of 150 nM, was also applied to each cell post hERG current measurement at the highest concentration of the test compound, which was used as an internal low control for normalization of percentage inhibition. In order to ensure the good performance of cultured cells and operations, 5 doses of Dofetilide as a positive control was also used to test the same batch of cells for the compound.

### 6) Data Analysis

### Data Acceptance Criteria:

The following criteria were used to determine data acceptability:
Initial seal resistance >1 GΩ;
Leak current <50% of the control peak tail current at any time;
Peak tail amplitude >250 pA;
Membrane resistance Rm >200 MΩ;
Access resistance Ra >15 MΩ;
Apparent drop of peak current <2.5% per minute.

After perfusing the blank solvent or compound gradient solution, the average of 5 consecutive current values obtained stably was taken and used as “Tail current size_{blank}” or “Tail current size_{compound}” respectively.

The data that met the above criteria for hERG current quality were further analyzed according to the following procedure:

The percentage of current inhibition was calculated using the following formula (wherein PatchMaster or Clampfit software was used to extract the peak currents from raw data), where the blank control was 0.1% DMSO as the blank solvent perfused;

$$Tail current inhibition = ( 1 - \frac{\begin{matrix}Tail current {size}_{compound} - \\ Tail current {size}_{positive drug}\end{matrix}}{\begin{matrix}Tail current {size}_{compound} - \\ Tail current {size}_{positive drug}\end{matrix}} ) \times 100$$

Dose-effect curves of the test compounds were obtained by fitting using Graphpad Prism 8.0, and the concentrations corresponding to 50% inhibition, i.e., the concentrations in Table 1 were derived from the curves.

The hERG test results are shown in Table 1.

### 7) References

[1] 1: Roche et al. A Virtual Screening Method for Prediction of the hERG Potassium Channel Liability of Compound Libraries. (2002) Chem Bio Chem. 3, 455-459.
[2] Glenn E. Kirsch et al. Variability in the measurement of hERG potassium channel inhibition: effects of temperature and stimulus patter. (2004) Journal of Pharmacological and Toxicological Methods 50, 93-101.
[3] Roger Marrannes et al. Computer programs to facilitate the estimation of time-dependent drug effects on ion channels. (2004) Computer Methods and Programs in Biomedicine 74, 167-181.

TABLE 1
Solubility and hERG values of the example compounds
of the present invention and the compound of comparative example

| | Kinetic solubility | hERG IC₅₀ |
|---|---|---|
| Example number | (mg/mL) | (μM) |
| Comparative | 0.005 | <0.12 |
| Example 1 | | |
| 10 | 0.026 | 1.33 |
| 87 | 0.038 | 1.417 |
| 95 | 0.099 | 0.98 |
| 68 | >0.25 | 0.89 |
| 70 | >0.25 | 0.568 |
| 73 | >0.25 | 1.662 |

### (3) Method for Optical Property Test

The UV-VIS absorption spectra of the compounds (10 μM, DMSO solution) were detected using Shimadzu UV2600 spectrophotometer, and the maximum absorption peak and absorbance of each compound were obtained. The results are shown in Table 2.

TABLE 2

| Example number | Absorption peak nm | Absorbance × 10⁴ |
|---|---|---|
| Comparative | 794.5 | 2.16 |
| Example 1 | | |
| 1A | 860 | 2.82 |
| 1B | 859.5 | 2.03 |
| 2 | 833.5 | 2.09 |
| 3 | 835 | 1.88 |
| 4 | 821 | 2.36 |
| 5 | 796.5 | 1.17 |
| 6 | 795 | 1.8 |
| 7 | 794.5 | 1.92 |
| 8 | 794.5 | 1.96 |
| 10 | 821 | 2.08 |
| 11 | 796.5 | 2.27 |
| 12 | 781.5 | 2.07 |
| 13 | 632 | 0.68 |
| 14 | 767.5 | 1.71 |
| 16 | 766 | 1.71 |
| 17 | 766 | 0.88 |
| 18 | 764.5 | 2.12 |
| 20 | 782 | 2.60 |
| 21 | 821 | 2.466 |
| 22 | 827 | 1.97 |
| 25 | 767.5 | 2.265 |
| 26 | 797.5 | 2.44 |
| 27A | 796.5 | 2.29 |
| 27B | 797 | 2.117 |
| 28 | 766 | 1.856 |
| 35 | 794.5 | 2.21 |
| 36 | 795.5 | 3.368 |
| 37A | 794.5 | 2.118 |
| 37B | 801 | 1.563 |
| 38 | 584 | 0.364 |
| 40 | | 0.93 |
| 47 | | 2 |
| 48 | | 2.02 |
| 49 | | 2.3 |
| 50 | | 1.97 |
| 51 | | 1.9 |
| 52 | | 2.14 |
| 53 | | 0.6 |
| 54 | | 2.57 |
| 55 | | 3.48 |
| 56 | | 1.85 |
| 58 | | 2.21 |
| 63 | | 0.31 |
| 64 | | 0.5 |
| 68 | 796 | 2.74 |
| 70 | 796.5 | 2.09 |
| 73 | 796.5 | 2.15 |
| 77 | 794.5 | 4.344 |
| 78 | 794.5 | 4.082 |
| 79 | 787.5 | 5.223 |
| 80 | 789 | 4.457 |
| 81 | 794 | 3.867 |
| 82 | 793.5 | 3.686 |
| 83 | 820 | 3.079 |
| 84 | | 2.41 |
| 85A | 796.5 | 2.239 |
| 85B | 796.5 | 1.397 |
| 87 | 785 | 1.91 |
| 88 | | 1.75 |
| 89 | | 1.86 |
| 91 | | 0.46 |
| 92 | | 1.49 |
| 93 | | 1.3 |
| 94 | | 1.86 |
| 95 | 793.5 | 2.41 |
| 96 | | 2.45 |
| 101 | 836.5 | 1.971 |
| 103 | 757.5 | 2.483 |
| 104 | 781.5 | 2.054 |
| 106 | 807.5 | 1.997 |
| 107 | 778.5 | 2.028 |
| 108 | 805.5 | 1.752 |
| 112 | 756 | 0.911 |
| 114 | | 2.46 |
| 115 | | 2.24 |
| 117 | | 1.36 |
| 118 | | 1.73 |
| 119 | | 1.52 |
| 120 | | 1.73 |
| 123 | | 1.3 |

### (4) MRC5 Test

### 1. Cell Line

MRC5 (ATCC; Cat. No.: CCL-171)

### 2. Reagents

Culture medium EMEM (ATCC, 30-2003)
Fetal bovine serum (Gibco, 10091148)
Penicillin-streptomycin double antibody (Gibco, S110JV)
DPBS (Gibco, 14190-144)
CellTiter-Glo® assay kit (Promega, G7573)
Trypsin (0.25%) (Gibco, 25200-056)
DMSO (Sigma, D2650)

### 3. Instruments

Biosafety cabinet (AIRTECH, BSC-1300A II)
Automated cell counter (Life Technologies, countess II)
Multifunctional microplate reader (Biotek, H1FM)
Shaker (Hangzhou Allsheng Instruments Co., Ltd., OS-100)
XLFIT 5.3 (Shanghai Inforstack Co., Ltd.)

### 4. Test Method

For this test method, the following experiment was performed in a biosafety cabinet with the specific steps as follows.
(1) Cells were seeded in a 96-well plate (in which the culture medium was EMEM). One day later, the previous culture medium EMEM was poured off, and 5 mL of DPBS was added to wash the cells. Then DPBS was pipetted and discarded. An appropriate amount of trypsin was then added. The plate was placed in a 5% CO₂ cell incubator at 37° C. to digestion for about 2 to 5 minutes, and then taken out. New culture medium EMEM was added. The cells were pipetted up and down to resuspend evenly. The cells were then counted by an automated cell counter.
(2) Cells were seeded in columns 1 to 11 of a 96-well plate (Corning, Cat. No. 3610) at a density of 3000 cells per well in 100 μL of culture medium EMEM, and 100 μL of culture medium EMEM free of cells was added into column 12 of the wells. Then, the plate was placed in the 5% CO₂ incubator at 37° C. for 24 hours.
(3) Next, the compound of the present invention was 2-fold serially diluted into 8 dose points starting at 10 μM. 10 μL of the serially diluted compound was pipetted and added to 100 μL of cells in columns 1 to 10, and 10 μL of the culture medium EMEM containing 2% DMSO was added to both columns 11 and 12. The plate was placed in the 5% CO₂ incubator at 37° C. and incubated for 3 days (Note: column 11 was used as a MAX well with cells but without the compound; column 12 was used as a MIN well without both cell and compound).
(4) The cell viability detection reagent in the above CellTiter-Glo® assay kit was then added to the 96-well plate at 50 μL per well according to the instruction, and shaken on a shaker in dark for 5 to 10 minutes. The cell viability was then measured using a multifunctional microplate reader. Finally, the inhibition of the compound of the present invention on cell proliferation was plotted by XLFIT software, and the half inhibitory concentration, IC₅₀ value of the compound of the present invention was calculated therefrom.

The test results of the inhibition of the compounds of the present invention on MRC5 are shown in Table 3 below.

TABLE 3
Inhibitory effect of the compounds of the present invention on
proliferation of normal cells (IC₅₀, nM)

| | MRC5 |
|---|---|
| Example number | (IC₅₀, nM) |
| Comparative | 2698.2 |
| Example 1 | |
| 87 | >10000 |
| 95 | >10000 |
| 68 | >10000 |
| 70 | >10000 |
| 73 | >10000 |

The results indicated that the compounds of the present invention significantly improved solubility compared to the compound of Comparative Example 1. This offers great convenience for application of drug injection. In addition, the compounds of the present invention significantly reduced hERG values, i.e. cardiac toxicity, making them safer for blood medication or other systemic medications. Furthermore, the present inventors evaluated the killing effect of some compounds on normal cells and found that they were all greater than 10 μM, indicating that they would not have adverse effects on normal tissues or cells.

### Test Example 2. In Vitro Cellular Uptake Test of Contrast Agent Molecules

2.1. Fluorescence Localization Imaging of Comparative Example 1 and Examples 2, 6, 4, 7, 8 and 10 in Human Bladder Cancer Cell Line RT112 (Nanjing Cobioer; Cat. No.: CBP60316).

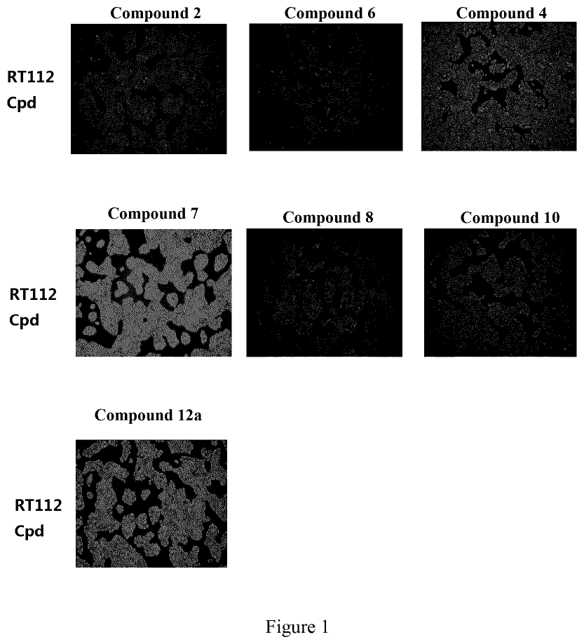

RT112 cells (1.5×10⁴/well) were cultured in DMEM medium (containing 10% fetal bovine serum (FBS), 1% antibiotics (100 U/mL penicillin and 100 U/mL streptomycin)) and seeded in a 96-well plate in advance. Then, the plate was cultured in a 5% CO₂ cell culture incubator at 37° C. overnight. The following day, the culture medium was sucked away, and the culture medium containing an appropriate concentration (1-20 μM) of the compound and surfactin (the molar ratio of the test compound to the surfactin (OKA: Cat. No.: A55976-1g) being 1:5) was added to the test wells, and incubated for 30 minutes. The cells were rinsed three times with PBS solution, and the near-infrared fluorescence intensity in the bladder cancer cells was observed using an integrated fluorescence microscopic imaging system from Keyence. The conditions for near-infrared photography were Ex=775/50 nm and Em=845/55 nm. The results are shown in FIG. 1.

2.2. Fluorescence Localization Imaging of Compounds 12a, 2, 6, 4, 7, 8 and 10 in Human Bladder Cancer Cell Line UM-UC-3 (ATCC; Cat. No.: CRL-1749).

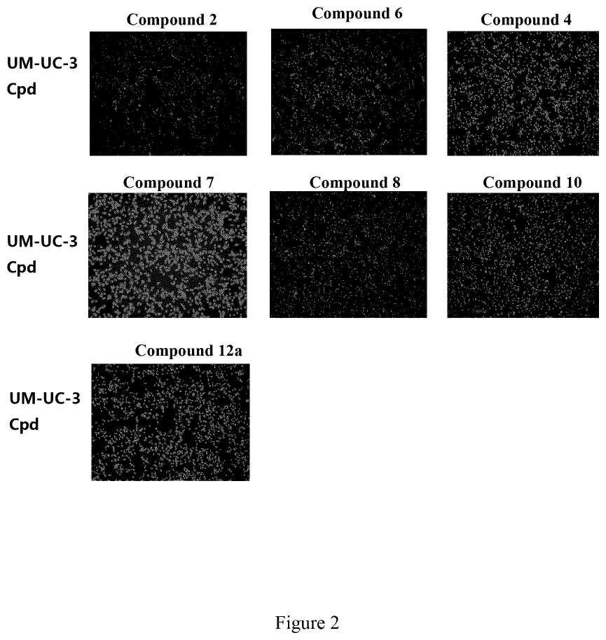

UM-UC-3 cells (1.5×10⁴/well) were cultured in EMEM medium (containing 10% fetal bovine serum (FBS) and 1% antibiotics (100 U/mL penicillin and 100 U/mL streptomycin)) and seeded in a 96-well plate in advance. The plate was cultured in a 5% CO₂ cell culture incubator at 37° C. overnight. The following day, the culture medium was sucked away, and the culture medium containing an appropriate concentration (1-20 μM) of the compound as well as surfactin (the molar ratio of the test compound to the surfactin being 1:5) was added to the test wells, and incubated for 30 minutes. The cells were rinsed three times with PBS solution, and the near-infrared fluorescence intensity in the bladder cancer cells was observed using an integrated fluorescence microscopic imaging system from Keyence. The conditions for near-infrared photography were Ex=775/50 nm and Em=845/55 nm. The results are shown in FIG. 2.

### Test Example 3. In Vitro Imaging Effect Test with Addition of BSP

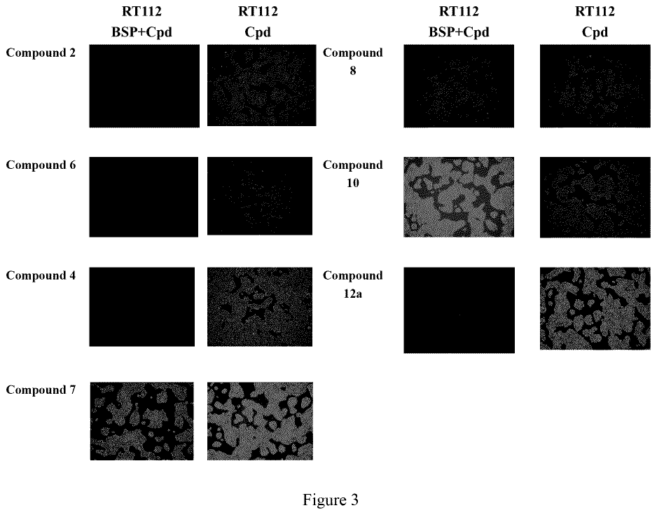

RT112 cells (1.5×10⁴/well) were cultured in DMEM medium (containing 10% fetal bovine serum (FBS), 1% antibiotics (100 U/mL penicillin and 100 U/mL streptomycin)) and seeded in a 96-well plate in advance. The plate was cultured in a 5% CO₂ cell culture incubator at 37° C. overnight. The following day, the culture medium was sucked away, and 250 μM of BSP (bromosulfophthalein, purchased from MACKLIN; Cat. No.: S914901) was added to the test wells and preconditioned for 10 minutes, followed by addition of the culture medium containing an appropriate concentration (1-20 μM) of the compound and surfactin (the molar ratio of the test compound to the surfactin being 1:5) with or without 250 μM of BSP, and then incubated for 30 minutes. The cells were rinsed three times with PBS solution, and the near-infrared fluorescence intensity in the bladder cancer cells was observed using an integrated fluorescence microscopic imaging system from Keyence. The conditions for near-infrared photography were Ex=775/50 nm and Em=845/55 nm. The results are shown in FIG. 3.

It can be seen from FIG. 3 that the uptake of Compounds 12a, 2, 6, 4, 7, 8 and 10 by human bladder cancer cell line RT112 may be weakened or enhanced by BSP.

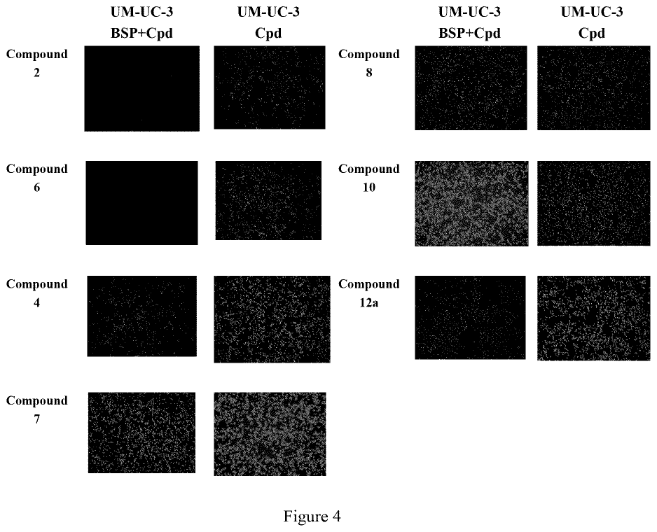

UM-UC-3 (1.5×10⁴/well) were cultured in EMEM medium (containing 10% fetal bovine serum (FBS) and 1% antibiotics (100 U/mL penicillin and 100 U/mL streptomycin)) and seeded in a 96-well plate in advance. The plate was cultured in a 5% CO₂ cell culture incubator at 37° C. overnight. The following day, the culture medium was sucked away, and 250 μM of BSP was added to the test wells and preconditioned for 10 minutes, followed by addition of the culture medium containing an appropriate concentration (1-20 μM) of the compound and surfactin (the molar ratio of the test compound to the surfactin being 1:5) with or without 250 μM of BSP, and then incubated for 30 minutes. The cells were rinsed three times with PBS solution, and the near-infrared fluorescence intensity in the bladder cancer cells was observed using an integrated fluorescence microscopic imaging system from Keyence. The conditions for near-infrared photography were Ex=775/50 nm and Em=845/55 nm. The results are shown in FIG. 4.

It can be seen from FIG. 4 that the uptake of Compounds 12a, 2, 6, 4, 7, 8 and 10 by human bladder cancer cell line UM-UC-3 may be weakened or enhanced by BSP.

### Test Example 4. Imaging of the Compounds of the Invention in a Bladder Cancer Tumor Model

The imaging of Compounds 7, 8 and 10 in a bladder cancer tumor model was detected.

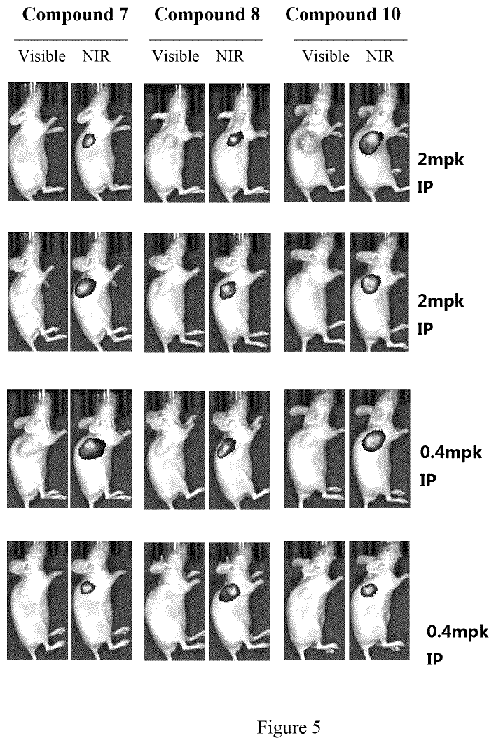

All animal experiments were conducted exactly in accordance with the guidelines for the care and use of animals issued by the regional animal committee. A mouse model of xenograft RT112 tumor was established by subcutaneous injection of RT112 cell suspension (2×10⁶ cells/mouse, 0.1 ml PBS) into the upper right limb of 8- to 10-week-old BALB/C female nude mice (20 to 25 g). BABL/c nude mice (15 to 20 g) were purchased from Beijing Vital River Laboratory Animal Technology Co., Ltd. For in vivo near-infrared fluorescence (NIRF) imaging, the RT112 tumor-bearing mice were intraperitoneally injected with different doses of free compounds (2 mg/kg or 0.4 mg/kg), respectively. At 48 hours after injection, near-infrared imaging was performed using an IVIS Lumina spectroscopic imaging system (excitation/emission, 780/845 nm). The results are shown in FIG. 5.

As shown in FIG. 5, Compounds 7, 8 and 10 were all specifically enriched at the tumor sites under the administration dose condition of 2 mg/kg or 0.4 mg/kg (NIR standing for near-infrared fluorescence signal). The ratio of fluorescence intensity at the tumor site to background fluorescence intensity was greater than 1.5 times.

### Test Example 5. Irradiance of the Compounds of the Invention

The compounds of the present invention, such as Compound 10, were superior to ICG (a dye used for angiography).

### Test Example 6. Tumor/Normal Cell Signal Ratio of the Compounds of the Invention

The compounds of the present invention exhibited good contrast at different concentrations (1-20 μM), i.e., the fluorescence intensity of tumor cells was significantly higher than that of normal cells.

### Test Example 7. Tumor/Background Ratio of the Compounds of the Invention

The subcutaneous tumor model mice inoculated with RT112 tumor were selected. Each compound was administrated at a dose of 2 mpk, and intraperitoneally injected. At 48 hours after injection, the ratio of fluorescence intensity at the tumor site to background fluorescence intensity was observed using a small animal imaging device. The results are shown in Table 4.

TABLE 4

| Example | |
|---|---|
| number | Tumor/background ratio |
| Comparative | 2.4 |
| Example 1 | |
| 10 | 3.3 |
| 87 | 3.3 |
| 95 | 2.8 |
| 68 | 2.5 |
| 70 | 2.7 |
| 73 | 2.7 |

In addition, the experimental results in an in situ tumor model showed that the compounds of the present invention exhibited a good tumor/background ratio, and the fluorescence intensity in the bladder with tumor was significantly higher than that in the normal bladder.

1. A compound with a structure of general formula (I) or a pharmaceutically acceptable salt thereof, 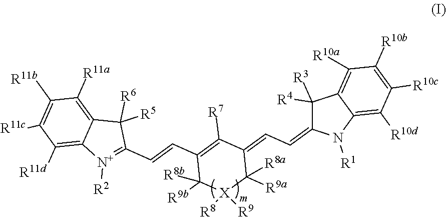
wherein:
X is C, O, S or N⁺;
R¹ and R² are each independently L₁-L₂-L₃-Q;
L₁ is selected from the group consisting of bond, —(CH₂)ₚ—, —(CH₂O)_{q}—, —(CH₂CH₂O)ᵥ—, —(CH₂O)_{q}—(CH₂)ₚ—, —(CH₂)ₚ—(CH₂O)_{q}—, —(CH₂)ₚ—(CH₂O)_{q}—(CH₂)ₚ—, —(CH₂CH₂O)ᵥ—(CH₂)ₚ— and —(CH₂CH₂O)ᵥ—(CH₂O)_{q}—;
L₂ is selected from the group consisting of bond, aryl, heteroaryl, cycloalkyl and heterocyclyl;
L₃ is selected from the group consisting of bond, —(CH₂)ₚ—, —(CH₂O)_{q}—, —(CH₂CH₂O)ᵥ—, —(CH₂O)_{q}—(CH₂)ₚ—, —(CH₂)ₚ—(CH₂O)_{q}—, —(CH₂)ₚ—(CH₂O)_{q}—(CH₂)ₚ—, —(CH₂CH₂O)ᵥ—(CH₂)ₚ— and —(CH₂CH₂O)ᵥ—(CH₂O)_{q}—;
Q is selected from the group consisting of R¹², —OR¹³, —C(O)R¹², —C(O)OR¹³, —C(O)NHR¹³, —SO₃H and 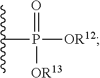
R¹² is selected from the group consisting of hydrogen, halogen, amino, hydroxy, thiol, alkyl, alkylamino, alkenyl, alkynyl, cycloalkyl, heterocyclyl, aryl and heteroaryl; wherein the alkyl, alkenyl, alkynyl, cycloalkyl, heterocyclyl, aryl or heteroaryl is optionally further substituted by one or more groups selected from the group consisting of halogen, amino, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R¹³ is selected from the group consisting of hydrogen, alkyl, alkenyl, alkynyl, cycloalkyl, heterocyclyl, aryl and heteroaryl; wherein the alkyl, alkenyl, alkynyl, cycloalkyl, heterocyclyl, aryl or heteroaryl is optionally further substituted by one or more groups selected from the group consisting of halogen, amino, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R³ and R⁴ are each independently selected from the group consisting of hydrogen, halogen, amino, nitro, cyano, hydroxy, thiol, oxo, alkyl, alkoxy, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R⁵ and R⁶ are each independently selected from the group consisting of hydrogen, halogen, amino, nitro, cyano, hydroxy, thiol, oxo, alkyl, alkoxy, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R⁷ is selected from the group consisting of hydrogen, halogen, nitro, cyano, hydroxy, thiol, oxo, alkyl, OR^{a}, SR^{a}, NR^{b}R^{c}, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R⁸ and R⁹ are each independently selected from the group consisting of hydrogen, halogen, nitro, cyano, hydroxy, thiol, oxo, alkyl, OR^{a}, NR^{b}R^{c}, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
R^{8a} and R^{9a}, are each independently selected from the group consisting of hydrogen, halogen, amino, nitro, cyano, hydroxy, thiol, oxo, alkyl, alkoxy, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R^{8b} and R^{9b} are each independently selected from the group consisting of hydrogen, halogen, amino, nitro, cyano, hydroxy, thiol, oxo, alkyl, alkoxy, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R^{10a}, R^{10b}, R^{10c} and R^{10d} are each independently selected from the group consisting of hydrogen, halogen, nitro, cyano, hydroxy, thiol, oxo, alkyl, OR^{a}, NR^{b}R^{c}, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
or any two adjacent ones of R^{10a}, R^{10b}, R^{10c} and R^{10d}, together with the atoms to which they are attached, form an aryl or heteroaryl, and the aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
R^{11a}, R^{11b}, R^{11c} and R^{11d} are each independently selected from the group consisting of hydrogen, halogen, nitro, cyano, hydroxy, thiol, oxo, alkyl, OR^{a}, NR^{b}R^{c}, —SO₃H, —S(O)₂R^{a}, —C(O)OR^{a}, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
or any two adjacent ones of R^{11a}, R^{11b}, R^{11c} and R^{11d}, together with the atoms to which they are attached, form an aryl or heteroaryl, and the aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
R^{a} is selected from the group consisting of hydrogen, alkyl, alkenyl, alkynyl, cycloalkyl, heterocyclyl, aryl and heteroaryl, wherein the alkyl, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, sulfonic group, —NHC(O)-L₄-NR^{b}R^{c}, —OC(O)-L₄-NR^{b}R^{c}, alkyl, haloalkyl, alkoxy, haloalkoxy, hydroxyalkyl, alkenyl, alkynyl, cycloalkyl, heterocyclyl, aryl and heteroaryl; wherein -L₄- is alkenylene;
R^{b} and R^{c} are each independently selected from the group consisting of hydrogen, alkyl, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
m is 0 or 1;
p is an integer from 1 to 10;
q is an integer from 1 to 10;
v is an integer from 1 to 10.

2. A compound represented by general formula (IA) or a pharmaceutically acceptable salt thereof, 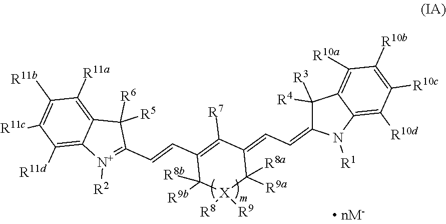
wherein:
X is C, O, S or N⁺;
M⁻ is an acid ion;
n is 1 or 2, when X is one N⁺, n is 2, and when X is C, O or S, n is 1;
R¹ and R² are each independently L₁-L₂-L₃-Q;
L₁ is selected from the group consisting of bond, —(CH₂)ₚ—, —(CH₂O)_{q}—, —(CH₂CH₂O)ᵥ—, —(CH₂O)_{q}—(CH₂)ₚ—, —(CH₂)ₚ—(CH₂O)_{q}—, —(CH₂)ₚ—(CH₂O)_{q}—(CH₂)ₚ—, —(CH₂CH₂O)ᵥ—(CH₂)ₚ— and —(CH₂CH₂O)ᵥ—(CH₂O)_{q}—;
L₂ is selected from the group consisting of bond, aryl, heteroaryl, cycloalkyl and heterocyclyl;
L₃ is selected from the group consisting of bond, —(CH₂)ₚ—, —(CH₂O)_{q}—, —(CH₂CH₂O)ᵥ—, —(CH₂O)_{q}—(CH₂)ₚ—, —(CH₂)ₚ—(CH₂O)_{q}—, —(CH₂)ₚ—(CH₂O)_{q}—(CH₂)ₚ—, —(CH₂CH₂O)ᵥ—(CH₂)ₚ— and —(CH₂CH₂O)ᵥ—(CH₂O)_{q}—;
Q is selected from the group consisting of R¹², —OR¹³, —C(O)R¹², —C(O)OR¹³, —C(O)NHR¹³, —SO₃H and 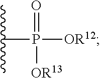
R¹² is selected from the group consisting of hydrogen, halogen, amino, hydroxy, thiol, alkyl, alkylamino, alkenyl, alkynyl, cycloalkyl, heterocyclyl, aryl and heteroaryl; wherein the alkyl, alkenyl, alkynyl, cycloalkyl, heterocyclyl, aryl or heteroaryl is optionally further substituted by one or more groups selected from the group consisting of halogen, amino, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R¹³ is selected from the group consisting of hydrogen, alkyl, alkenyl, alkynyl, cycloalkyl, heterocyclyl, aryl and heteroaryl; wherein the alkyl, alkenyl, alkynyl, cycloalkyl, heterocyclyl, aryl or heteroaryl is optionally further substituted by one or more groups selected from the group consisting of halogen, amino, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R³ and R⁴ are each independently selected from the group consisting of hydrogen, halogen, amino, nitro, cyano, hydroxy, thiol, oxo, alkyl, alkoxy, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R⁵ and R⁶ are each independently selected from the group consisting of hydrogen, halogen, amino, nitro, cyano, hydroxy, thiol, oxo, alkyl, alkoxy, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R⁷ is selected from the group consisting of hydrogen, halogen, nitro, cyano, hydroxy, thiol, oxo, alkyl, OR^{a}, SR^{a}, NR^{b}R^{c}, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R⁸ and R⁹ are each independently selected from the group consisting of hydrogen, halogen, nitro, cyano, hydroxy, thiol, oxo, alkyl, OR^{a}, NR^{b}R^{c}, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
R^{8a} and R^{9a} are each independently selected from the group consisting of hydrogen, halogen, amino, nitro, cyano, hydroxy, thiol, oxo, alkyl, alkoxy, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R^{8b} and R^{9b} are each independently selected from the group consisting of hydrogen, halogen, amino, nitro, cyano, hydroxy, thiol, oxo, alkyl, alkoxy, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, alkyl, haloalkyl, alkoxy, haloalkoxy, hydroxyalkyl, alkenyl, alkynyl, cycloalkyl, heterocyclyl, aryl and heteroaryl;
R^{10a}, R^{10b}, R^{10c} and R^{10d} are each independently selected from the group consisting of hydrogen, halogen, nitro, cyano, hydroxy, thiol, oxo, alkyl, OR^{a}, NR^{b}R^{c}, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
or any two adjacent ones of R^{10a}, R^{10b}, R^{10c} and R^{10d}, together with the atoms to which they are attached, form an aryl or heteroaryl, and the aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
R^{11a}, R^{11b}, R^{11c} and R^{11d} are each independently selected from the group consisting of hydrogen, halogen, nitro, cyano, hydroxy, thiol, oxo, alkyl, OR^{a}, NR^{b}R^{c}, —SO₃H, —S(O)₂R^{a}, —C(O)OR^{a}, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
or any two adjacent ones of R^{11a}, R^{11b}, R^{11c} and R^{11d}, together with the atoms to which they are attached, form an aryl or heteroaryl, and the aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
R^{a} is selected from the group consisting of hydrogen, alkyl, alkenyl, alkynyl, cycloalkyl, heterocyclyl, aryl and heteroaryl, wherein the alkyl, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, sulfonic group, —NHC(O)-L₄-NR^{b}R^{c}, —OC(O)-L₄-NR^{b}R^{c}, alkyl, haloalkyl, alkoxy, haloalkoxy, hydroxyalkyl, alkenyl, alkynyl, cycloalkyl, heterocyclyl, aryl and heteroaryl; wherein -L₄- is alkenylene;
R^{b} and R^{c} are each independently selected from the group consisting of hydrogen, alkyl, alkenyl, alkynyl, cycloalkyl, heterocyclyl, aryl and heteroaryl, wherein the alkyl, alkoxy, alkenyl, alkynyl, cycloalkyl, heterocyclyl, aryl or heteroaryl is optionally further substituted by one or more substituents selected from the group consisting of halogen, amino, oxo, thioxo, nitro, cyano, hydroxy, thiol, carboxy, ester, alkyl, haloalkyl, alkoxy, haloalkoxy, hydroxyalkyl, alkenyl, alkynyl, cycloalkyl, heterocyclyl, aryl and heteroaryl;
m is 0 or 1;
p is an integer from 1 to 10;
q is an integer from 1 to 10;
v is an integer from 1 to 10.

3.-4. (canceled)

5. The compound represented by general formula (IA) or the pharmaceutically acceptable salt thereof according to claim 2, being a compound represented by general formula (IIA) or (IIIA) or a pharmaceutically acceptable salt thereof, 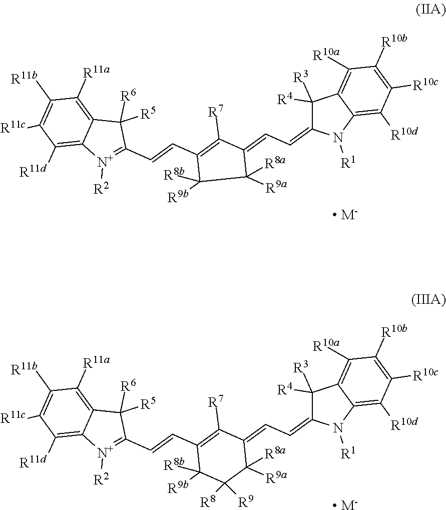
wherein, M⁻, R¹ to R⁹, R^{8a}, R^{8b}, R^{9a}, R^{9b}, R^{10a}, R^{10b}, R^{10c}, R^{10d}, R^{11a}, R^{11b}, R^{11c} and R^{11d} are as defined in claim 2.

6. (canceled)

7. The compound represented by general formula (IA) or the pharmaceutically acceptable salt thereof according to claim 2, being a compound represented by general formula (IVA) or a pharmaceutically acceptable salt thereof, 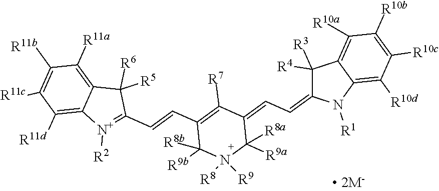
wherein, M⁻, R¹ to R⁹, R^{8a}, R^{8b}, R^{9a}, R^{9b}, R^{10a}, R^{10b}, R^{10c}, R^{10d}, R^{11a}, R^{11b}, R^{11c} and R^{11d} are as defined in claim 2.

8. The compound or a pharmaceutically acceptable salt thereof according to claim 1, wherein, R¹ is L₁-L₂-Q;
L₁ is selected from the group consisting of —(CH₂)ₚ— and —(CH₂O)_{q}—(CH₂)ₚ—;
L₂ is selected from the group consisting of bond, C₆₋₁₀ aryl and 5 to 10 membered heteroaryl;
Q is selected from the group consisting of R¹², —C(O)OR¹³, —C(O)NHR¹³, —SO₃H and 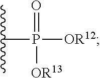
R¹² is selected from the group consisting of hydrogen, hydroxy and C₁₋₆ alkyl;
R¹³ is selected from the group consisting of hydrogen, C₆₋₁₀ aryl and 5 to 10 membered heteroaryl;
p is an integer from 1 to 10;
q is an integer from 1 to 10.

9. The compound or the pharmaceutically acceptable salt thereof according to claim 1, wherein, R² is L₁-Q;
L₁ is —(CH₂)ₚ—;
Q is selected from the group consisting of R¹², —C(O)OR¹³, —C(O)NHR¹³, —SO₃H and 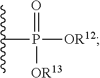
R¹² is selected from the group consisting of hydrogen, hydroxy and C₁₋₆ alkyl;
R¹³ is selected from the group consisting of hydrogen, C₆₋₁₀ aryl and 5 to 10 membered heteroaryl;
p is an integer from 1 to 10.

10. The compound or the pharmaceutically acceptable salt thereof according to claim 1, wherein R^{10a}, R^{10b}, R^{10c} and R^{10d} are each independently selected from the group consisting of hydrogen and OR^{a}, and R^{a} is C₁₋₆ alkyl.

11. The compound or a pharmaceutically acceptable salt thereof according to claim 1, wherein R^{11a}, R^{11b}, R^{11c} and R^{11d} are each independently selected from the group consisting of hydrogen, carboxy, OR^{a}, NR^{b}R^{c}, —SO₃H, —S(O)₂R^{a} and —C(O)OR^{a},
R^{a} is C₁₋₆ alkyl;
R^{b} and R^{c} are each independently C₁₋₆ alkyl.

12.-14. (canceled)

15. The compound or the pharmaceutically acceptable salt thereof according to claim 1, wherein,
R¹ is L₁-Q;
L₁ is —(CH₂)ₚ—;
Q is selected from the group consisting of —C(O)OR¹³, —C(O)NHR¹³, —SO₃H and 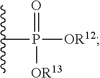
R¹³ is selected from the group consisting of hydrogen, C₆₋₁₀ aryl and 5 to 10 membered heteroaryl;
p is an integer from 1 to 10;
R² is C₁₋₆ alkyl.

16. The compound or the pharmaceutically acceptable salt thereof according to claim 1, wherein,
R¹ is L₁-Q;
L₁ is —(CH₂O)_{q}—(CH₂)ₚ—;
Q is selected from the group consisting of —C(O)OR¹³ and —C(O)NHR³;
R¹³ is selected from the group consisting of hydrogen, C₆₋₁₀ aryl and 5 to 10 membered heteroaryl;
p is an integer from 1 to 6;
q is an integer from 1 to 6;
R² is C₁₋₆ alkyl.

17. The compound or the pharmaceutically acceptable salt thereof according to claim 1, wherein,
R¹ is L₁-L₂-Q;
L₁ is —(CH₂)ₚ—;
L₂ is selected from the group consisting of C₆₋₁₀ aryl and 5 to 10 membered heteroaryl,
Q is —C(O)OR¹³;
R¹³ is selected from the group consisting of hydrogen, C₆₋₁₀ aryl and 5 to 10 membered heteroaryl;
p is an integer from 1 to 4;
R² is C₁₋₆ alkyl.

18. The compound or the pharmaceutically acceptable salt thereof according to claim 1, wherein,
R¹ is C₁₋₆ alkyl;
R² is C₁₋₆ alkyl.

19. The compound or a pharmaceutically acceptable salt thereof according to claim 1, wherein,
R¹ and R² are each independently selected from L₁-Q;
L₁ is —(CH₂)ₚ—;
Q is selected from the group consisting of R¹², —C(O)OR¹³, —C(O)NHR¹³, —SO₃H and 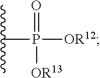
R¹² is selected from the group consisting of hydrogen, hydroxy and C₁₋₆ alkyl;
R¹³ is selected from the group consisting of hydrogen, C₆₋₁₀ aryl and 5 to 10 membered heteroaryl;
p is an integer from 1 to 10.

20. The compound or the pharmaceutically acceptable salt thereof according to claim 1, wherein, R⁷ is selected from the group consisting of hydrogen, halogen, hydroxy, OR^{a}, SR^{a} and NR^{b}R^{c};
R^{a} is selected from the group consisting of C₆₋₁₀ aryl and 5 to 10 membered heteroaryl; wherein, the aryl or heteroaryl is optionally further substituted by one or more substituents selected from the group consisting of halogen, amino, oxo, thioxo, nitro, cyano, hydroxy, thiol, carboxy, ester, sulfonic group, —NHC(O)-L₄-NR^{b}R^{c}, —OC(O)-L₄-NR^{b}R^{c}, alkyl, haloalkyl, alkoxy, haloalkoxy, hydroxyalkyl, alkenyl, alkynyl, cycloalkyl, heterocyclyl, aryl and heteroaryl; wherein -L₄- is alkenylene;
R^{b} and R^{c} are each independently selected from the group consisting of hydrogen and C₁₋₁₀ alkyl, wherein the C₁₋₁₀ alkyl is optionally further substituted by carboxy.

21. The compound or the pharmaceutically acceptable salt thereof according to claim 1, wherein R⁷ is SR^{a}; R^{a} is C₁₋₆ alkyl, and the C₁₋₆ alkyl is optionally substituted by carboxy.

22. The compound or the pharmaceutically acceptable salt thereof according to claim 1, wherein R⁸ and R⁹ are each independently selected from the group consisting of hydrogen, C₁₋₆ alkyl and NR^{b}R^{c}; R^{b} and R^{c} are each independently selected from the group consisting of hydrogen and C₁₋₆ alkyl.

23. The compound or the pharmaceutically acceptable salt thereof according to claim 1, wherein R^{8a} and R^{9a} are each independently hydrogen.

24. The compound or the pharmaceutically acceptable salt thereof according to claim 1, wherein R^{8b} and R^{9b} are each independently hydrogen.

25. The compound or the pharmaceutically acceptable salt thereof according to claim 1, wherein R³ and R⁴ are each independently selected from the group consisting of hydrogen and C₁₋₆ alkyl.

26. The compound or the pharmaceutically acceptable salt thereof according to claim 1, wherein R⁵ and R⁶ are each independently selected from the group consisting of hydrogen and C₁₋₆ alkyl.

27. The compound or the pharmaceutically acceptable salt thereof according to claim 1, wherein the pharmaceutically acceptable salt is halide salt or carboxylate salt.

28. A compound selected from the group consisting of 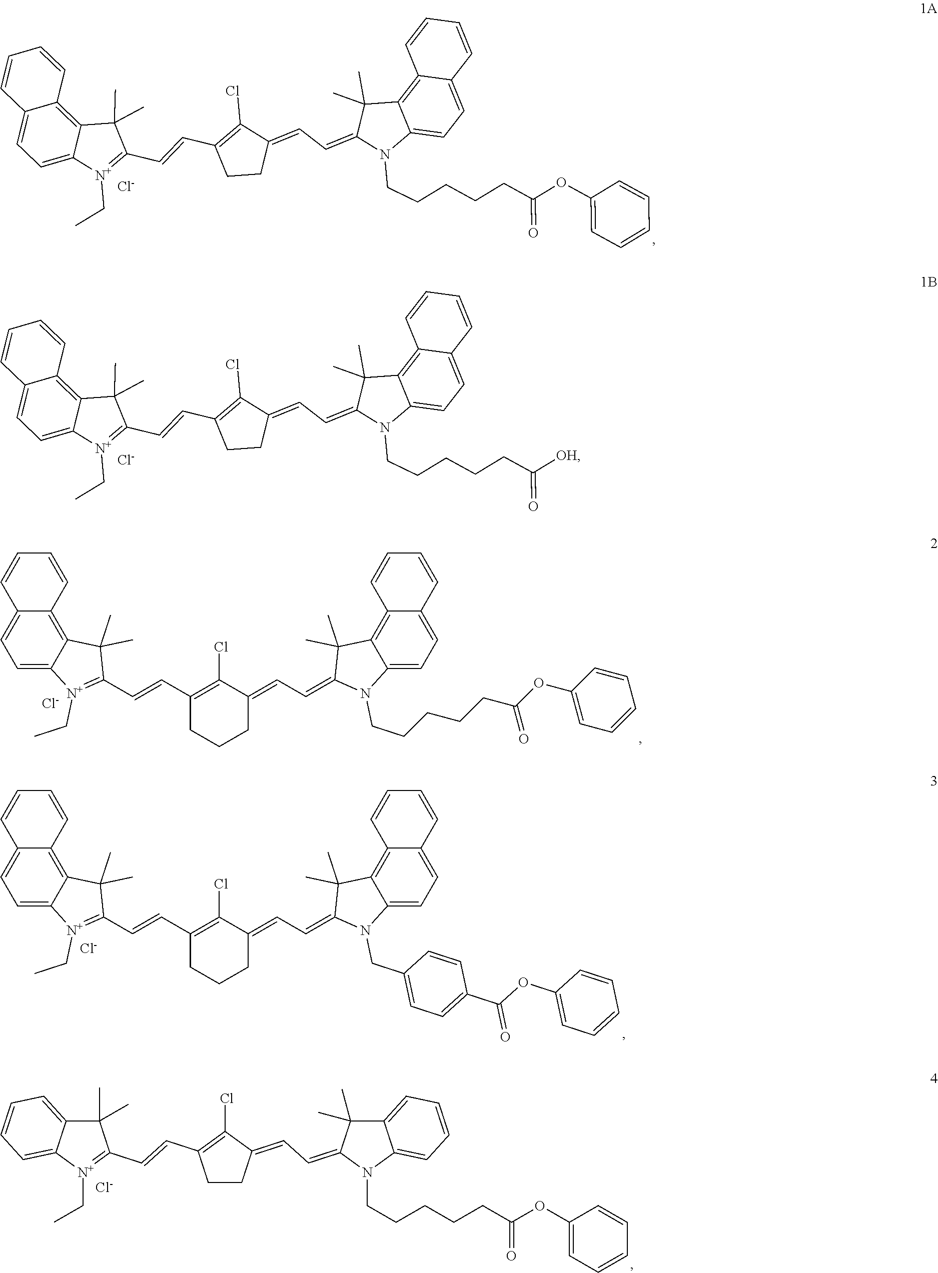 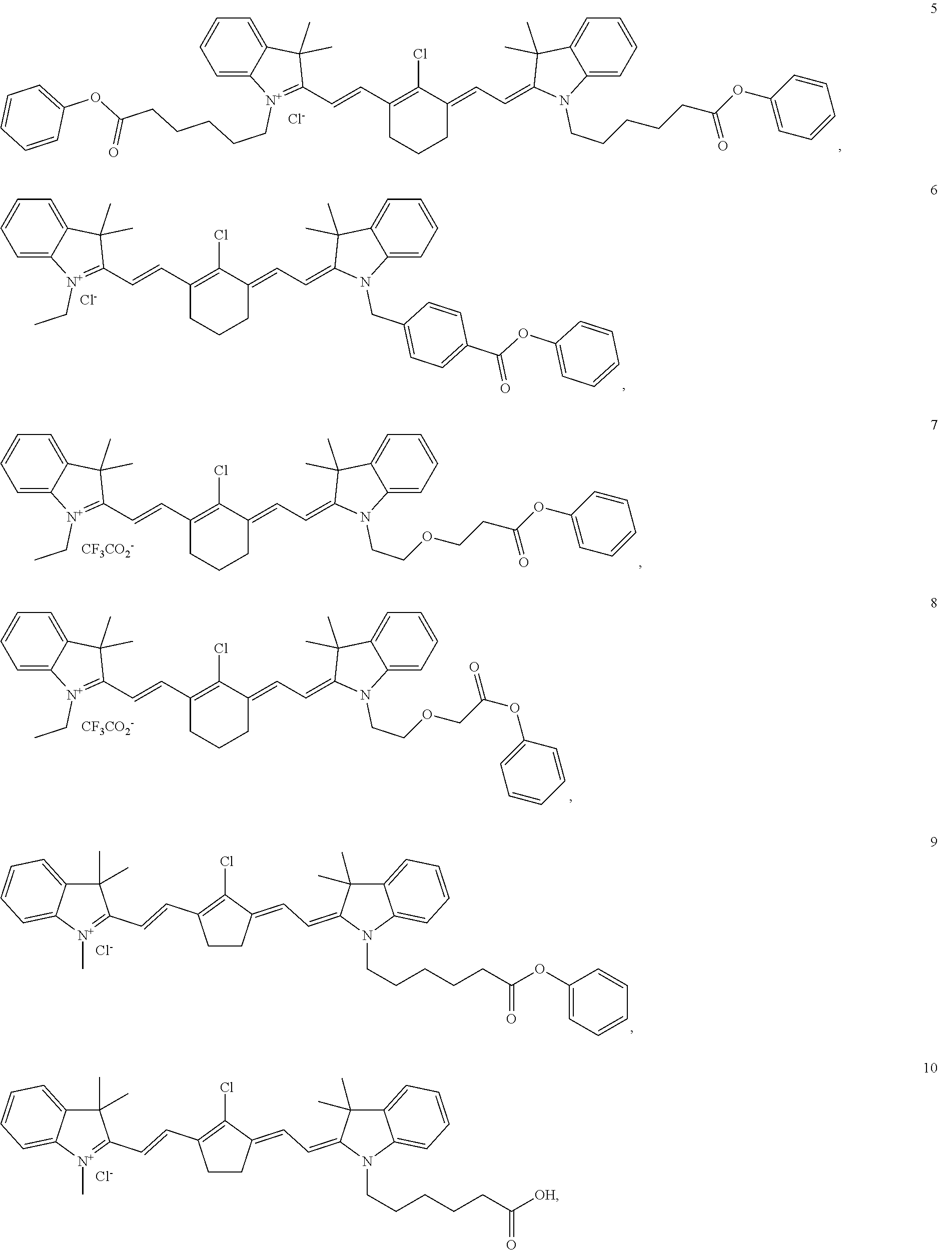 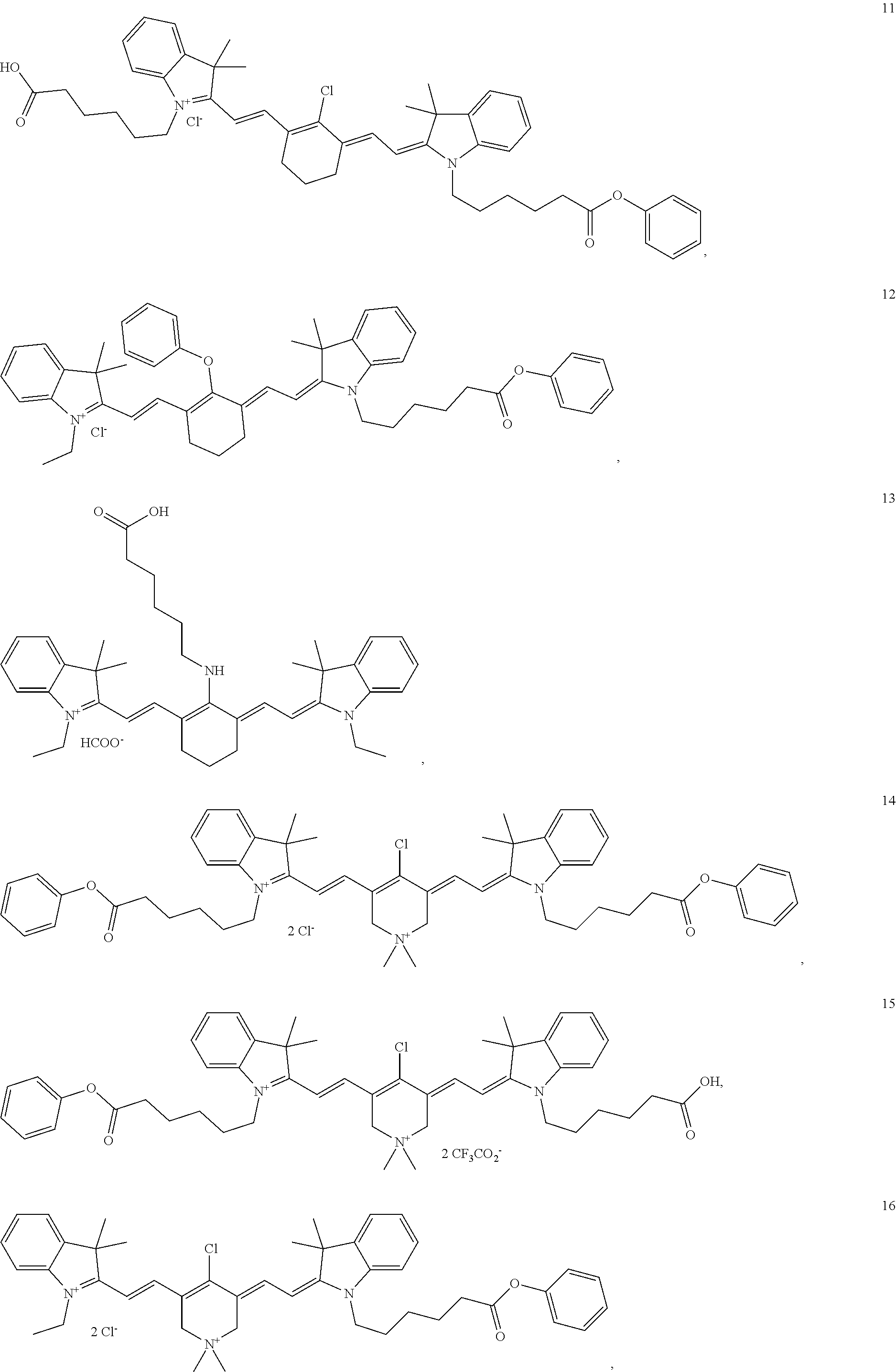 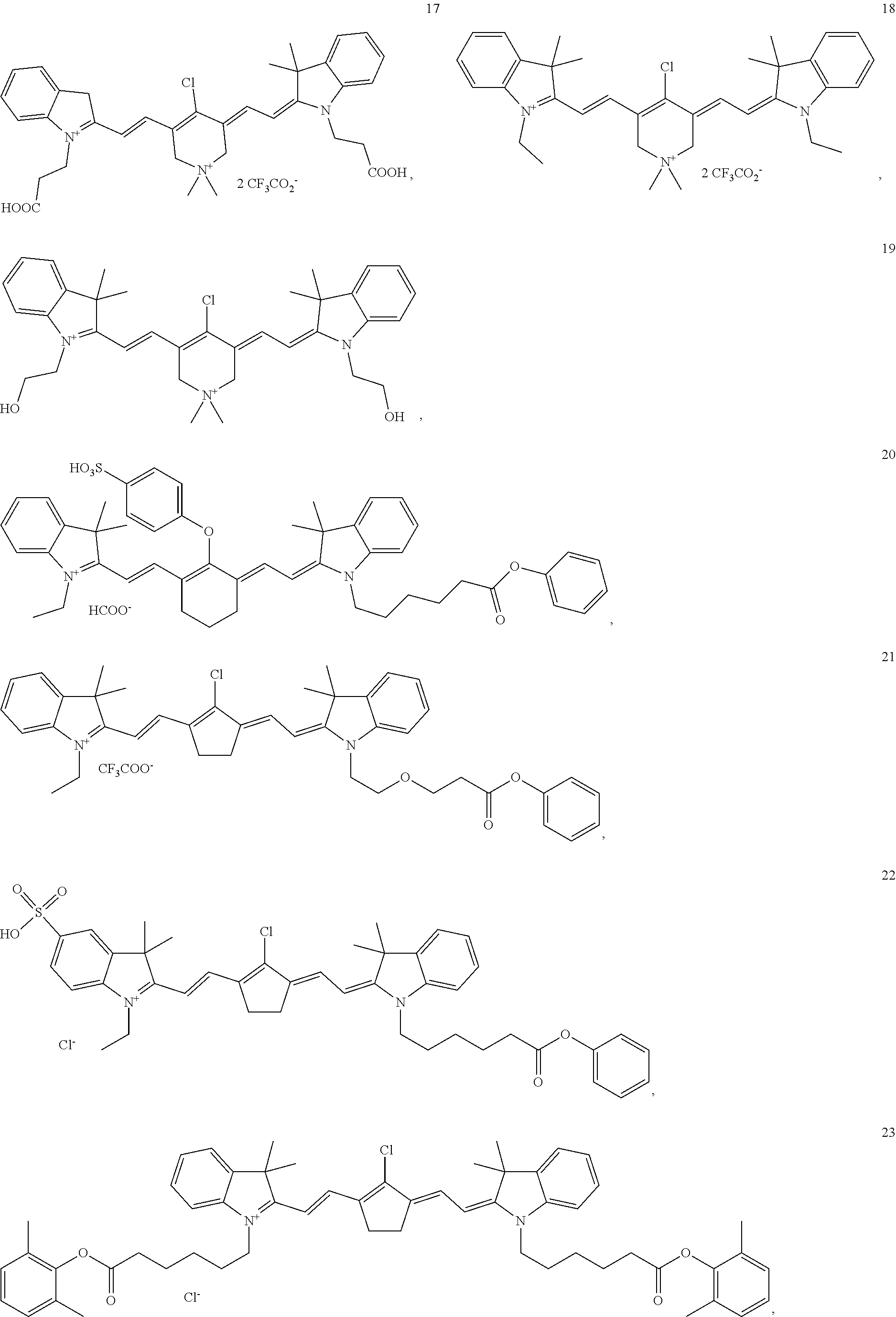 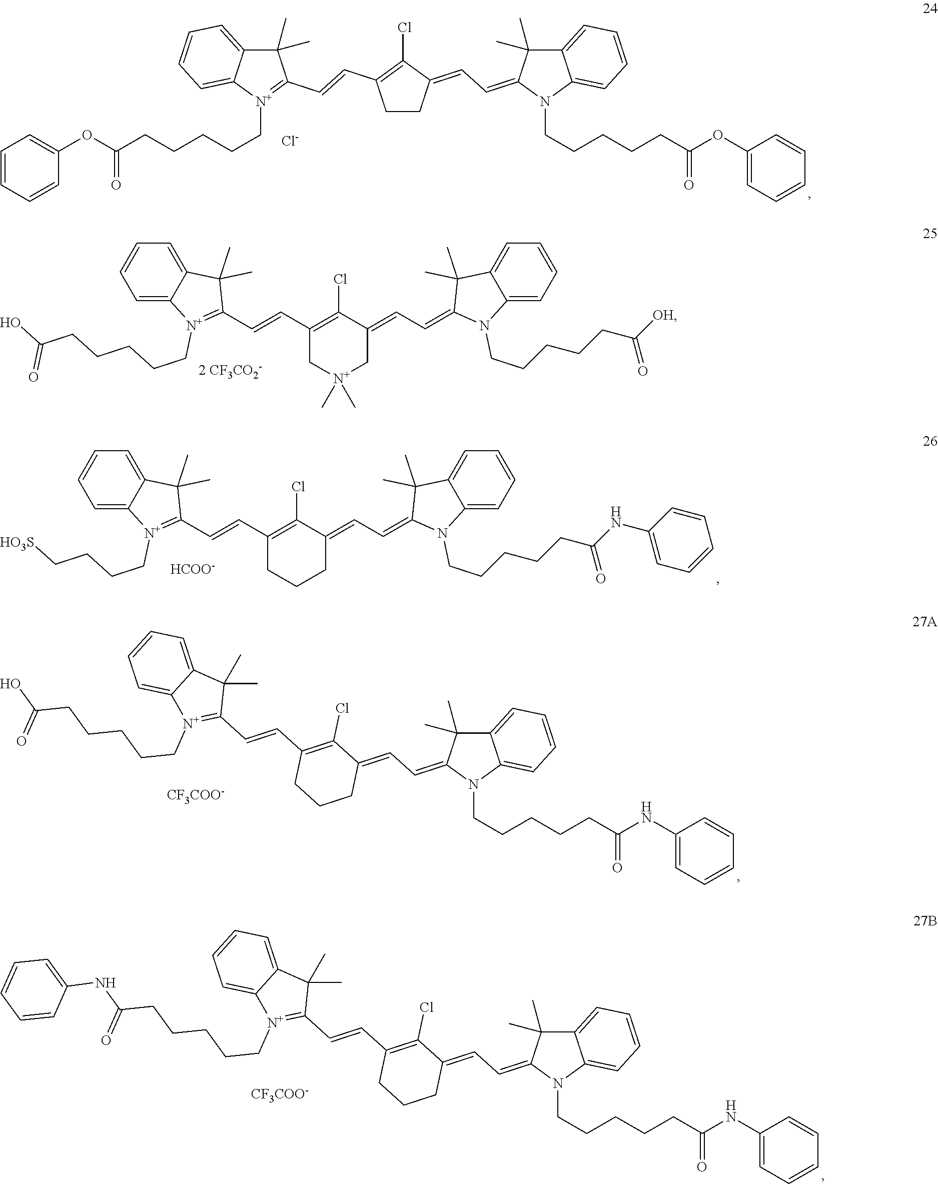 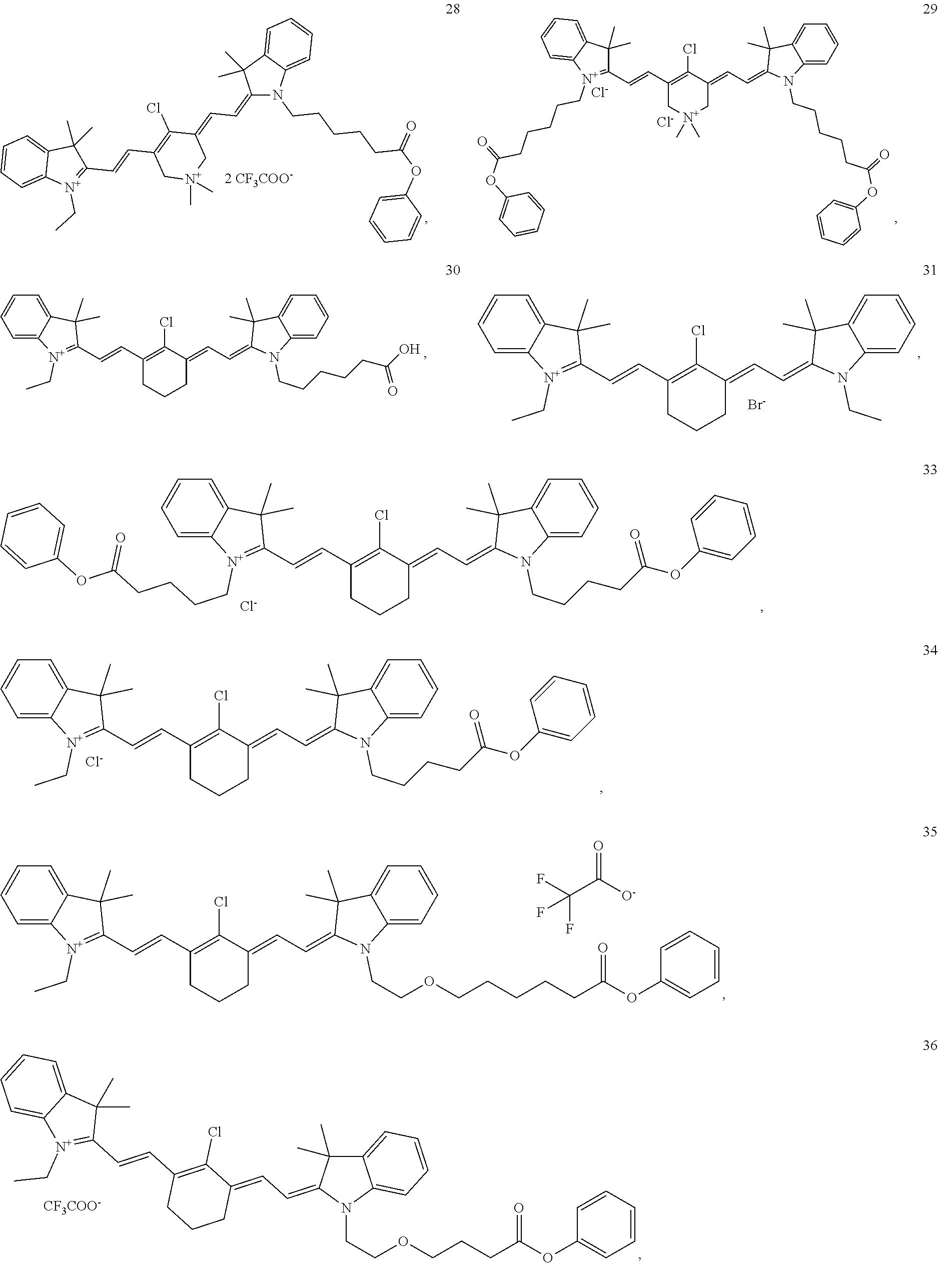 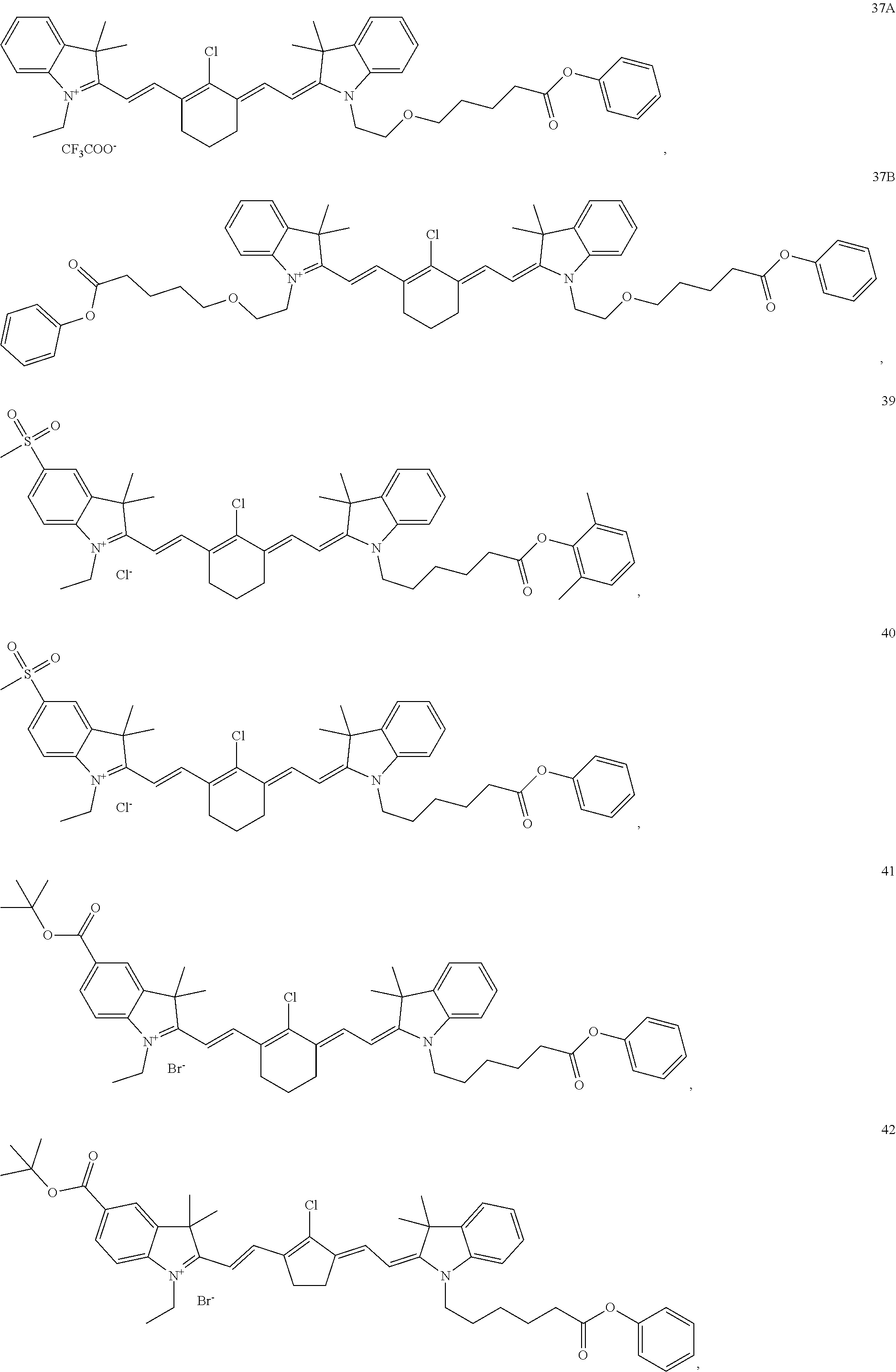 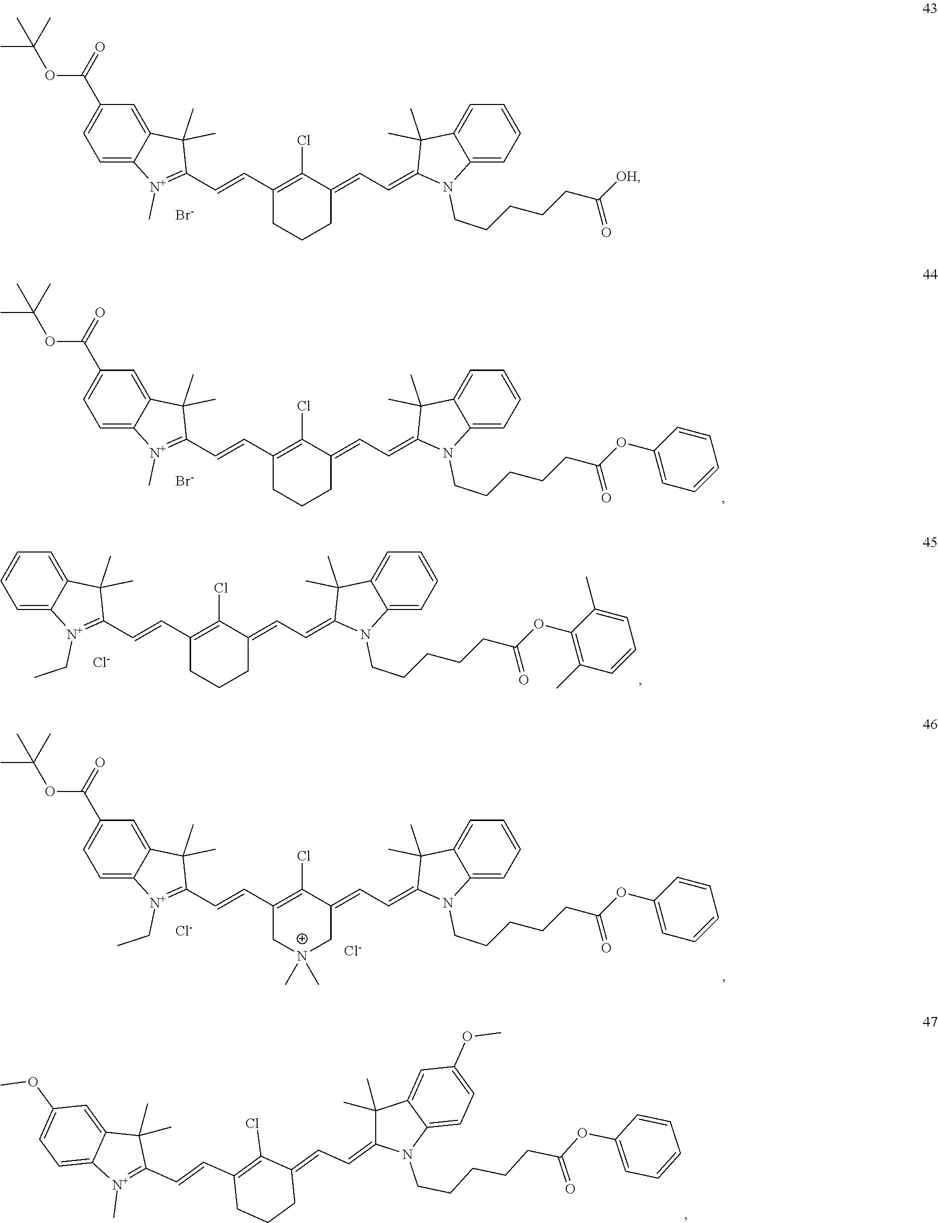 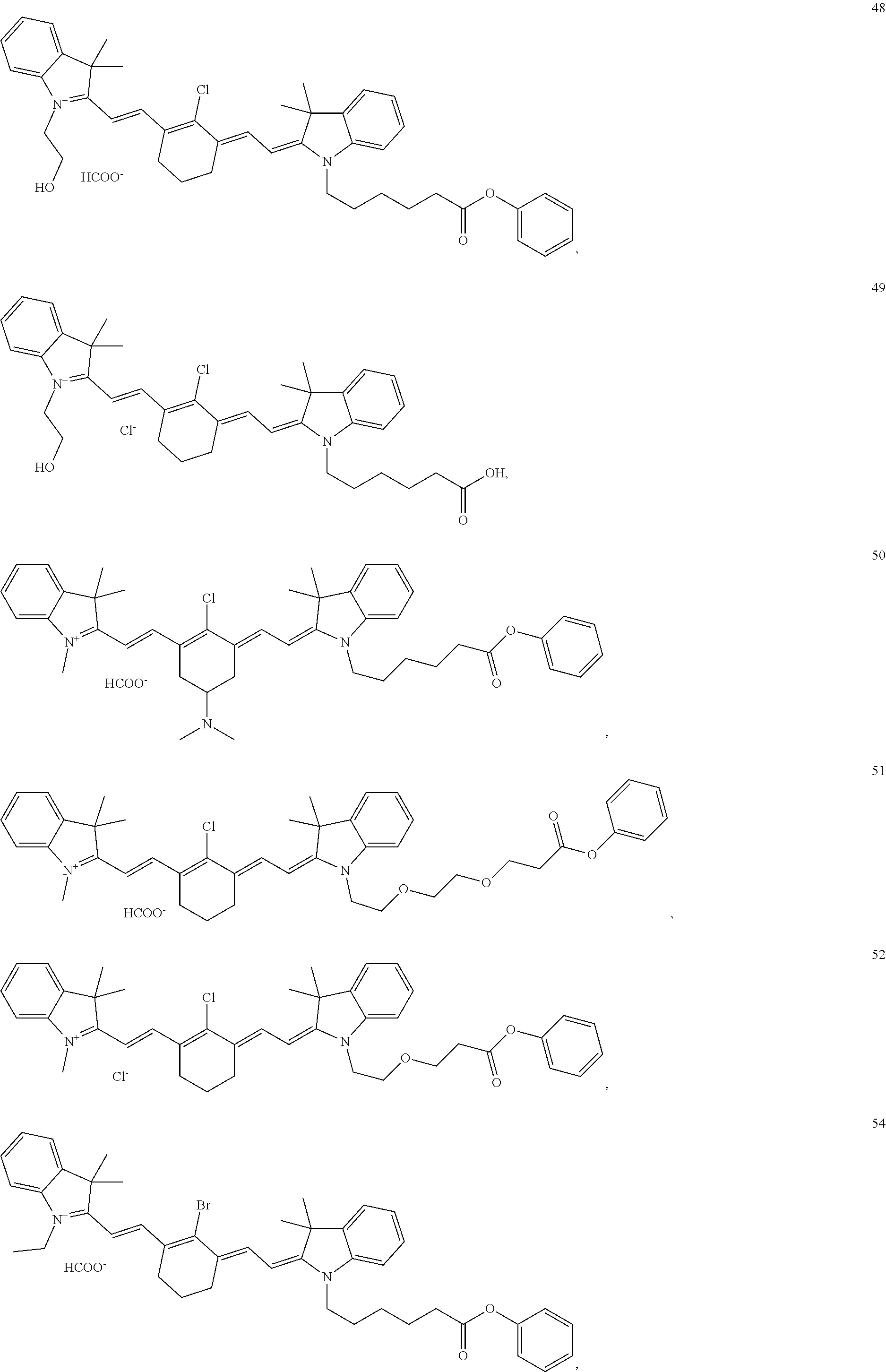 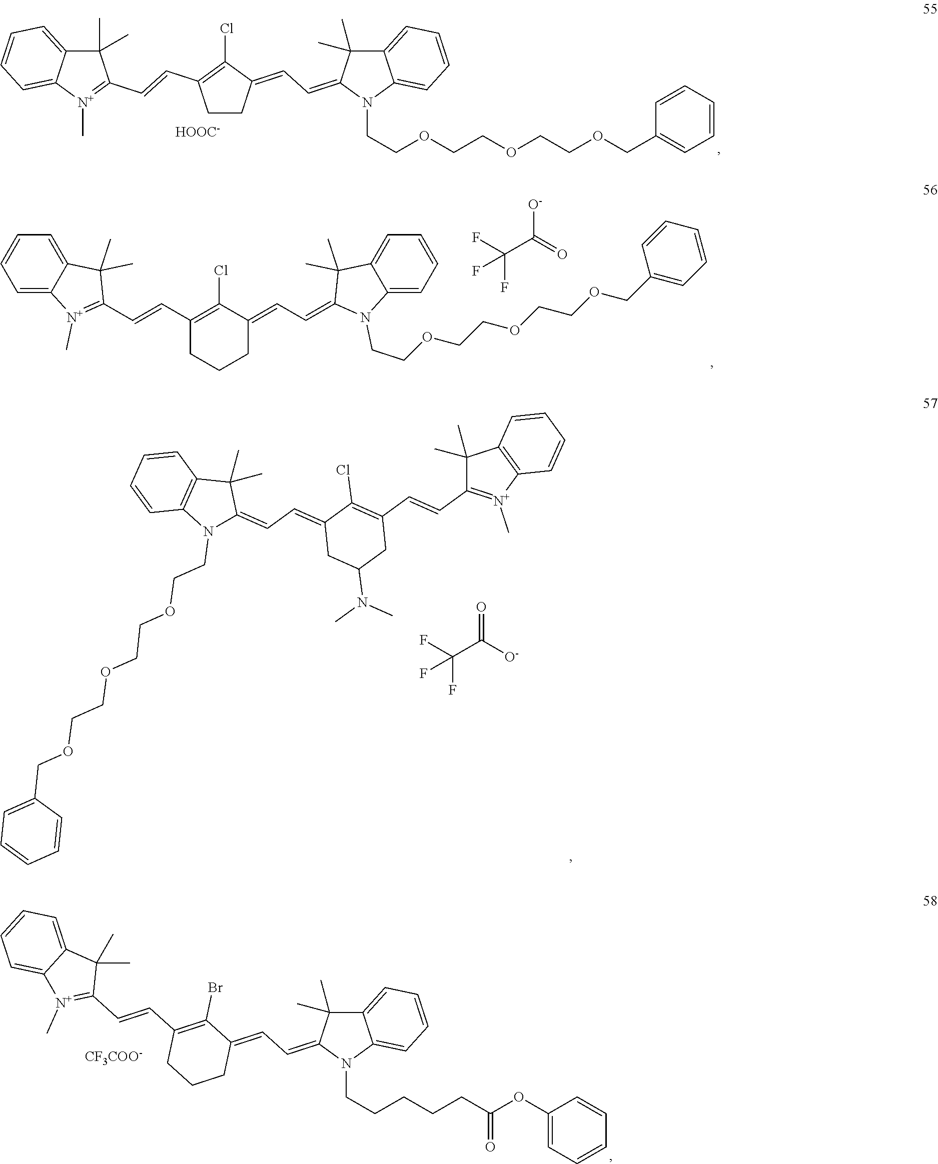 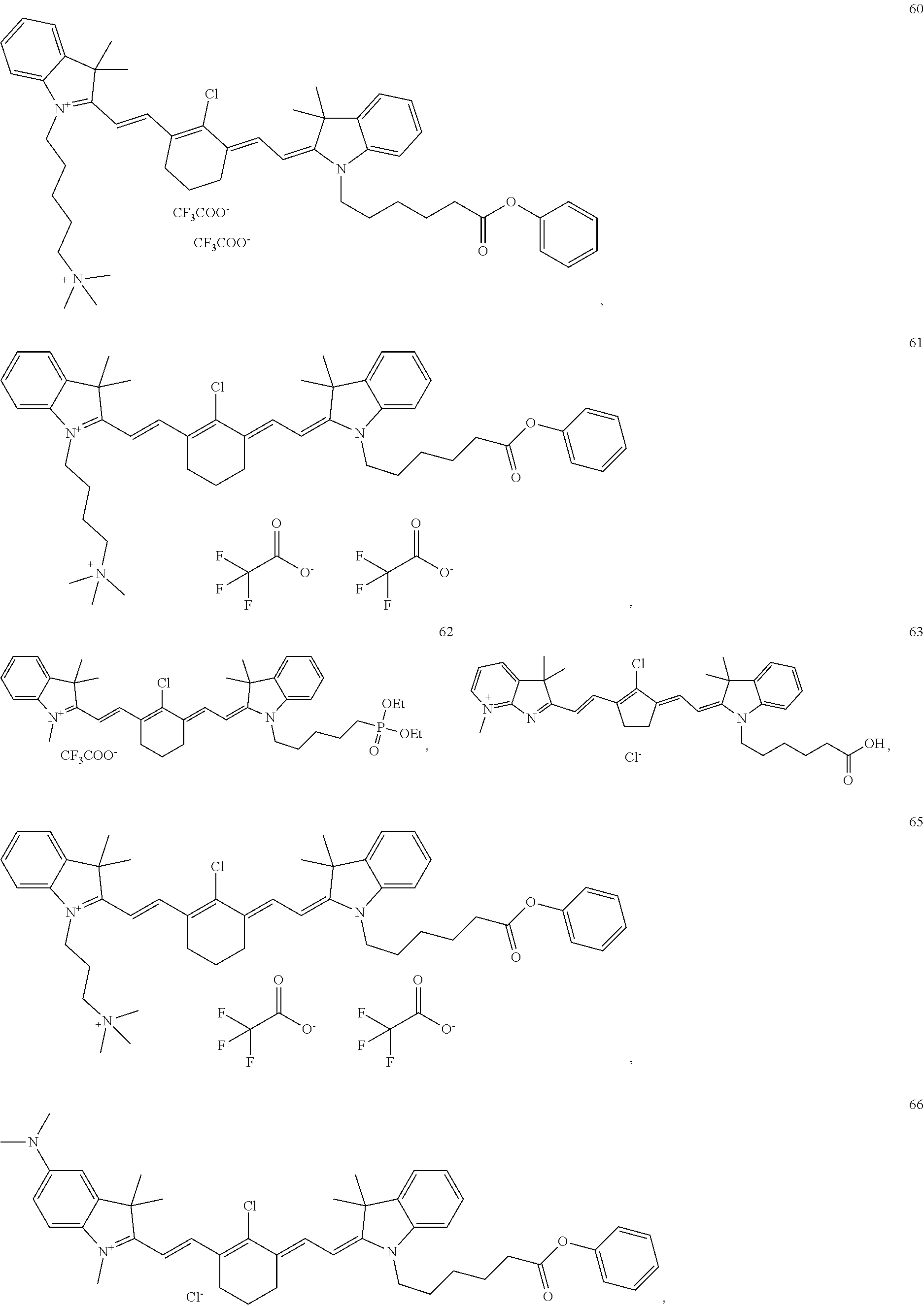 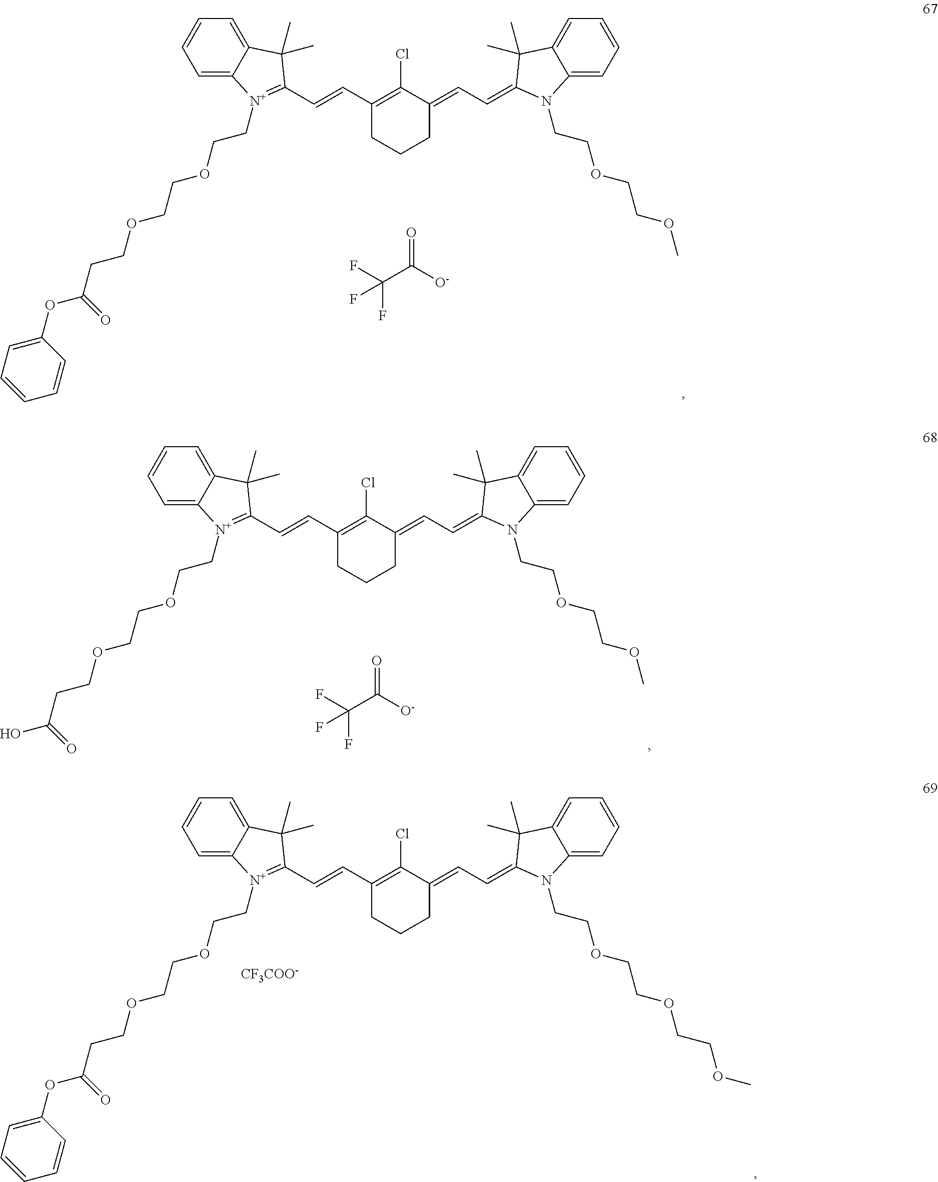 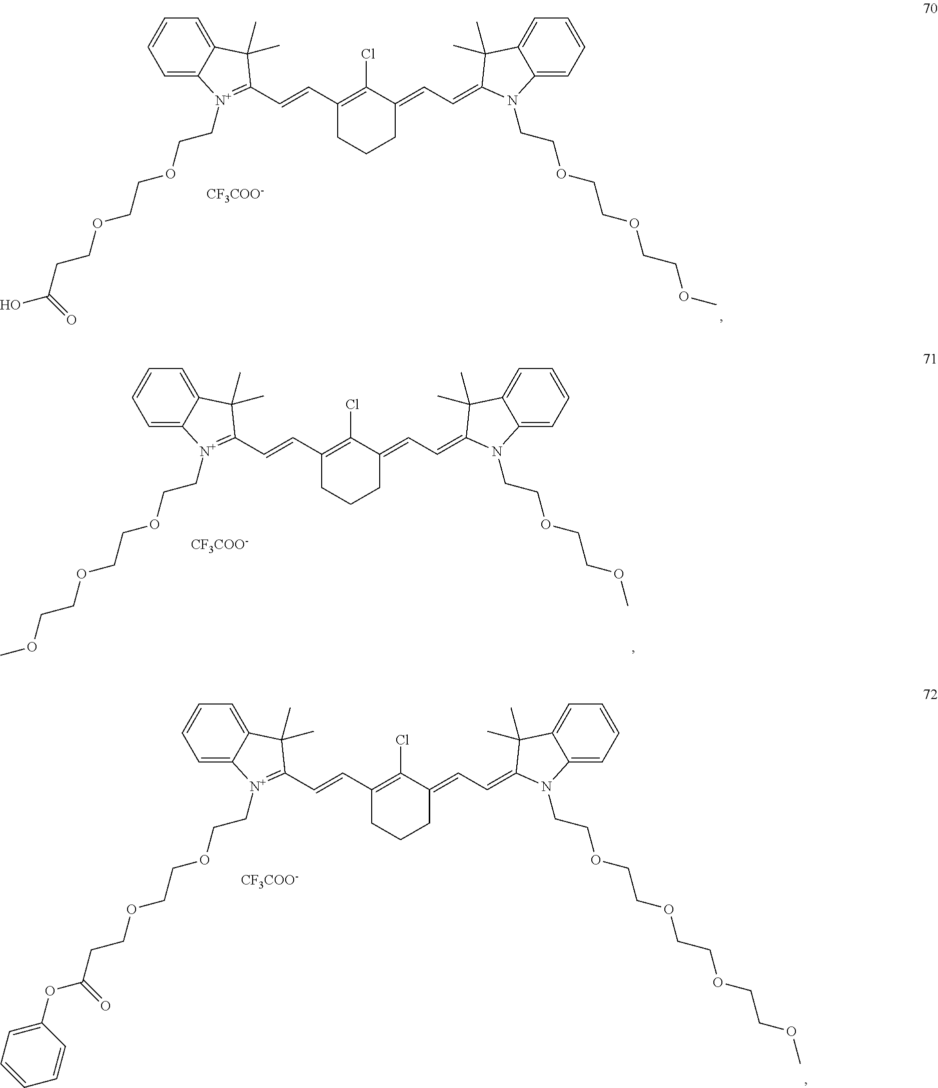 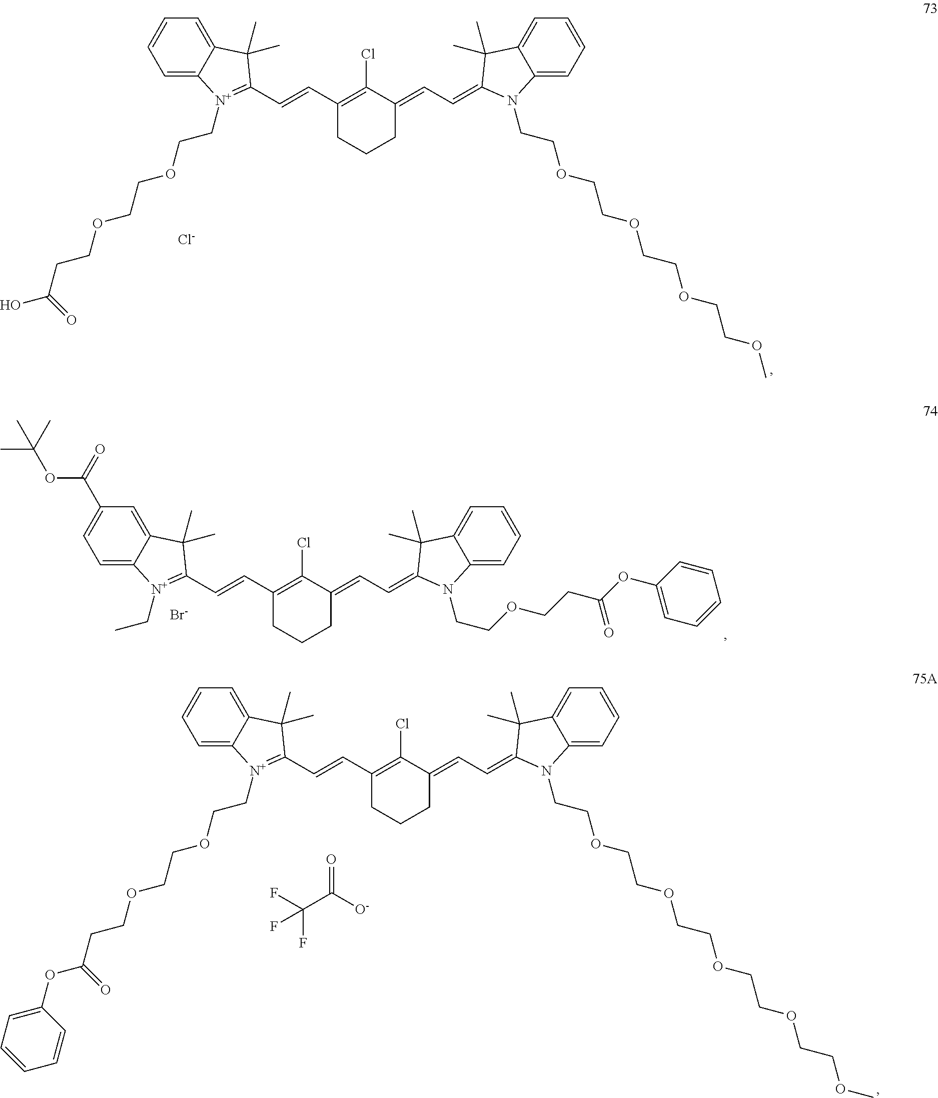 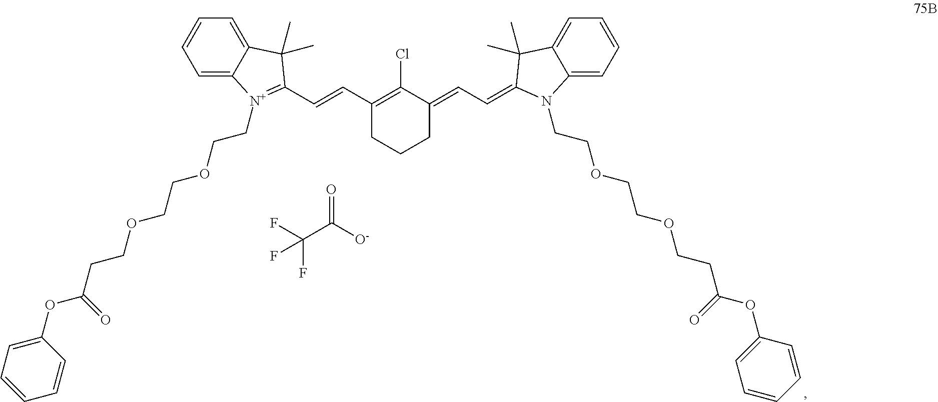 [SMILES: CC1(C)C(/C=C/C2=C(Cl)C(=C/C=C3/N(CCOCCOCCC(=O)Oc4ccccc4)c4ccccc4C3(C)C)/CCC2)=[N+](CCOCCOCCC(=O)Oc2ccccc2)c2ccccc21 ; O=C([O-])C(F)(F)F] 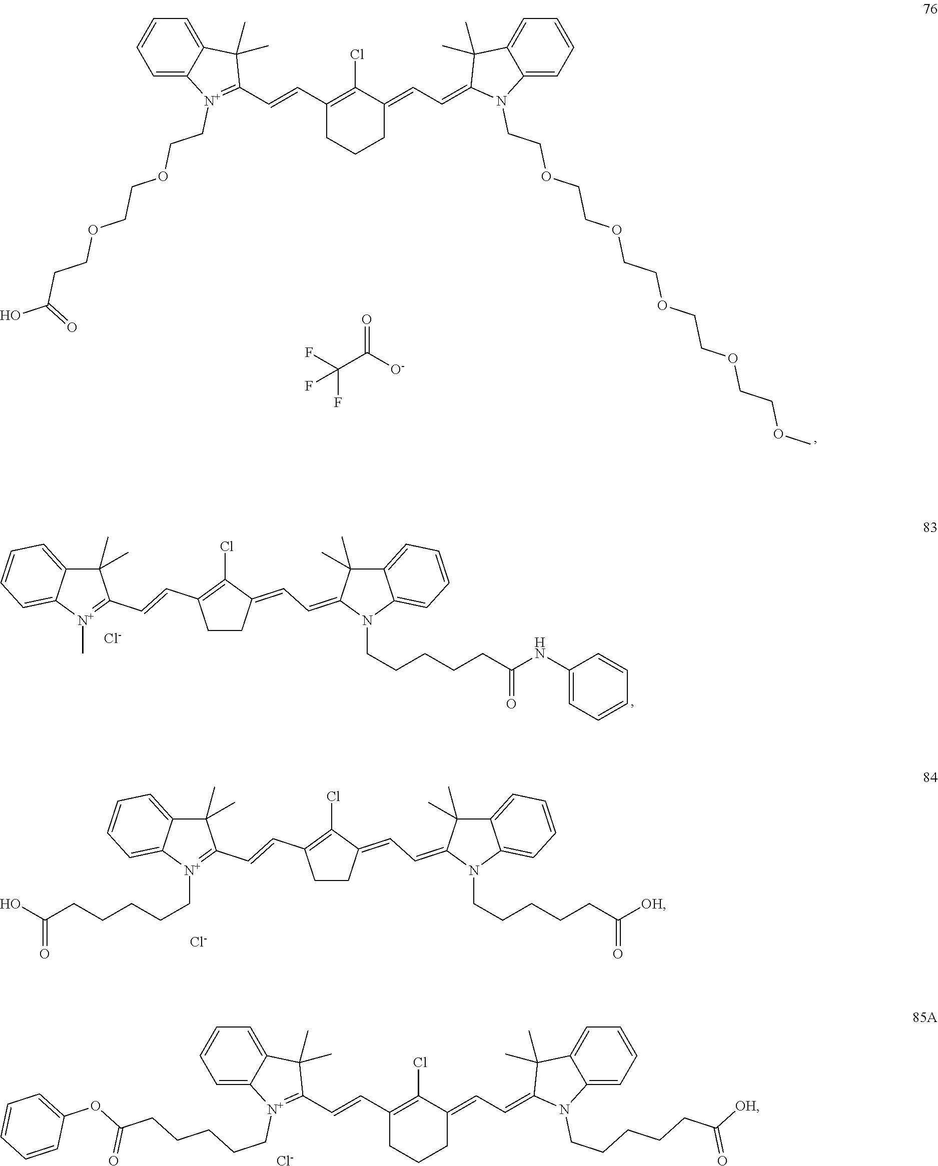 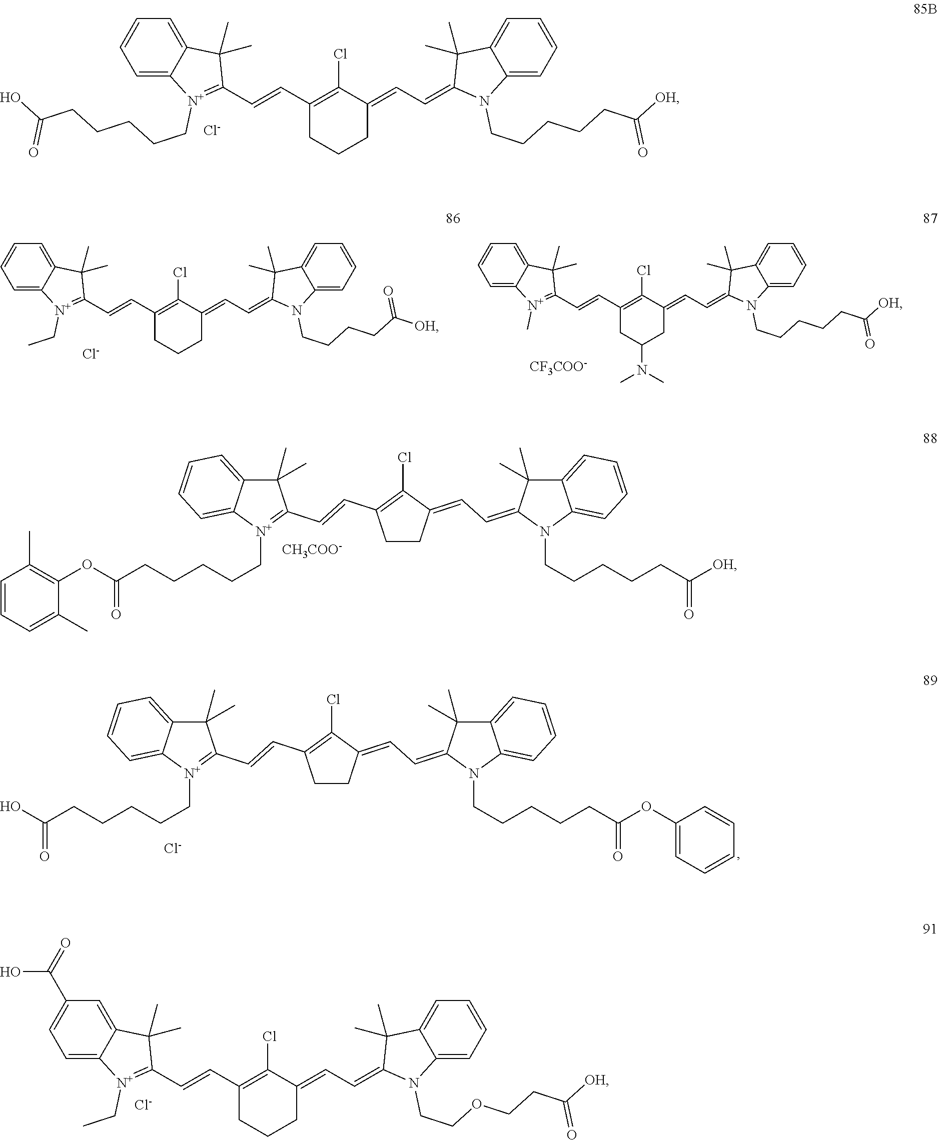 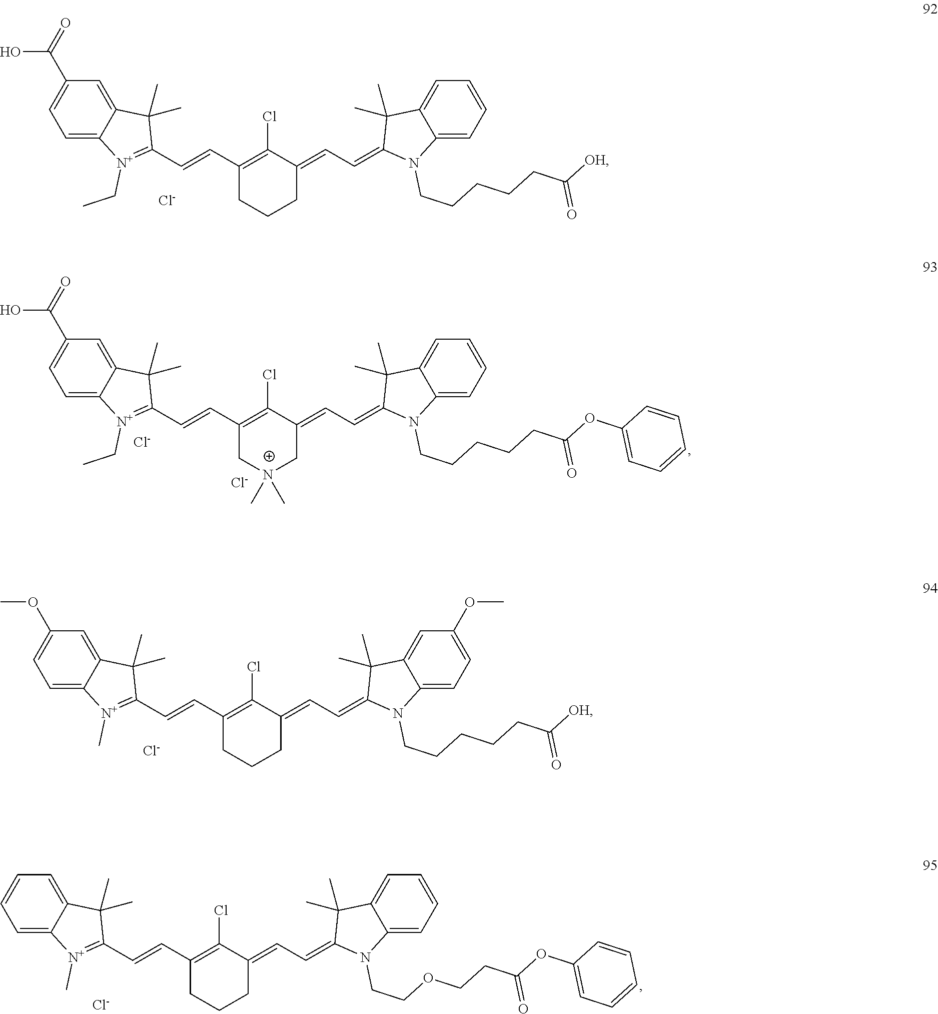 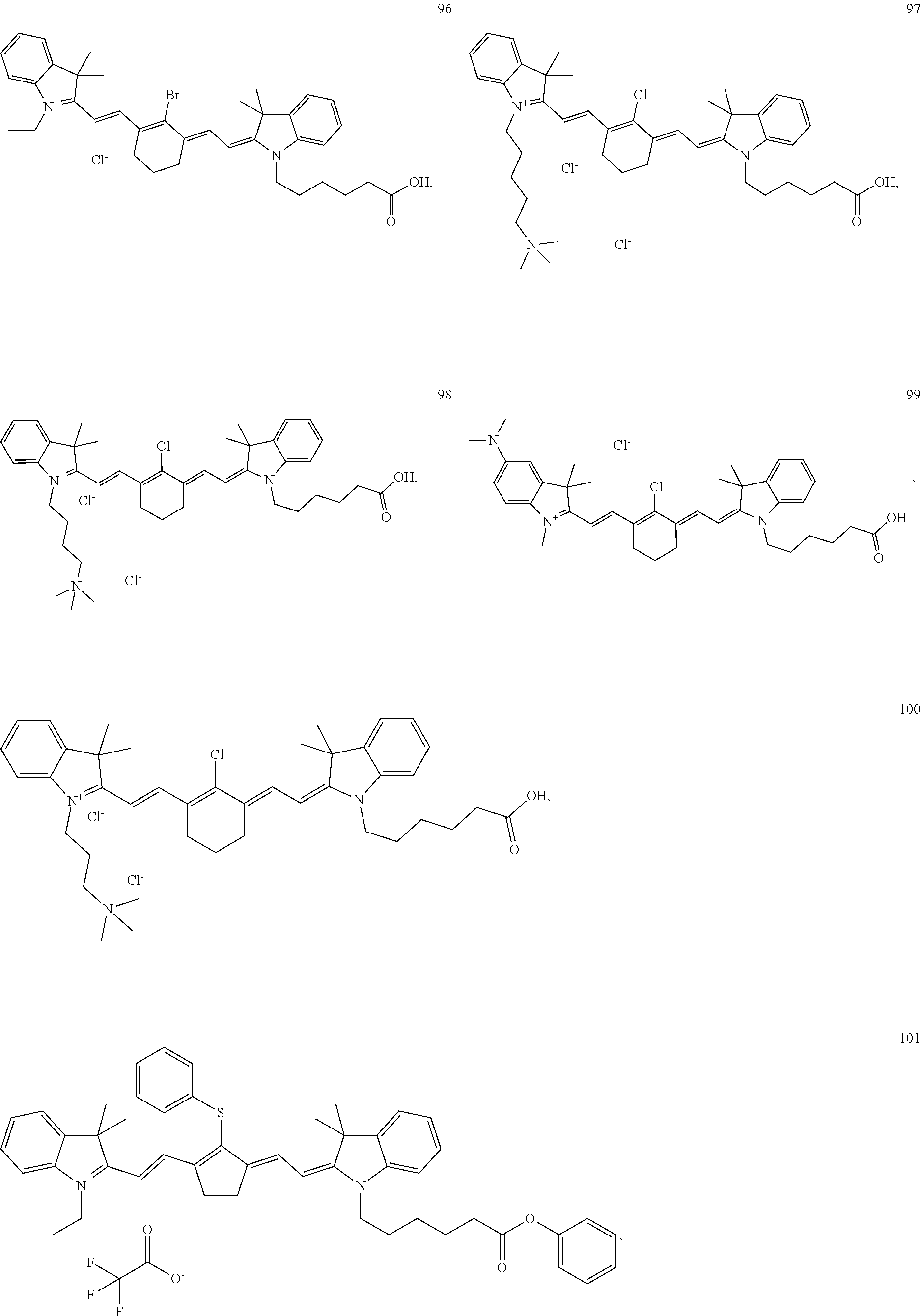 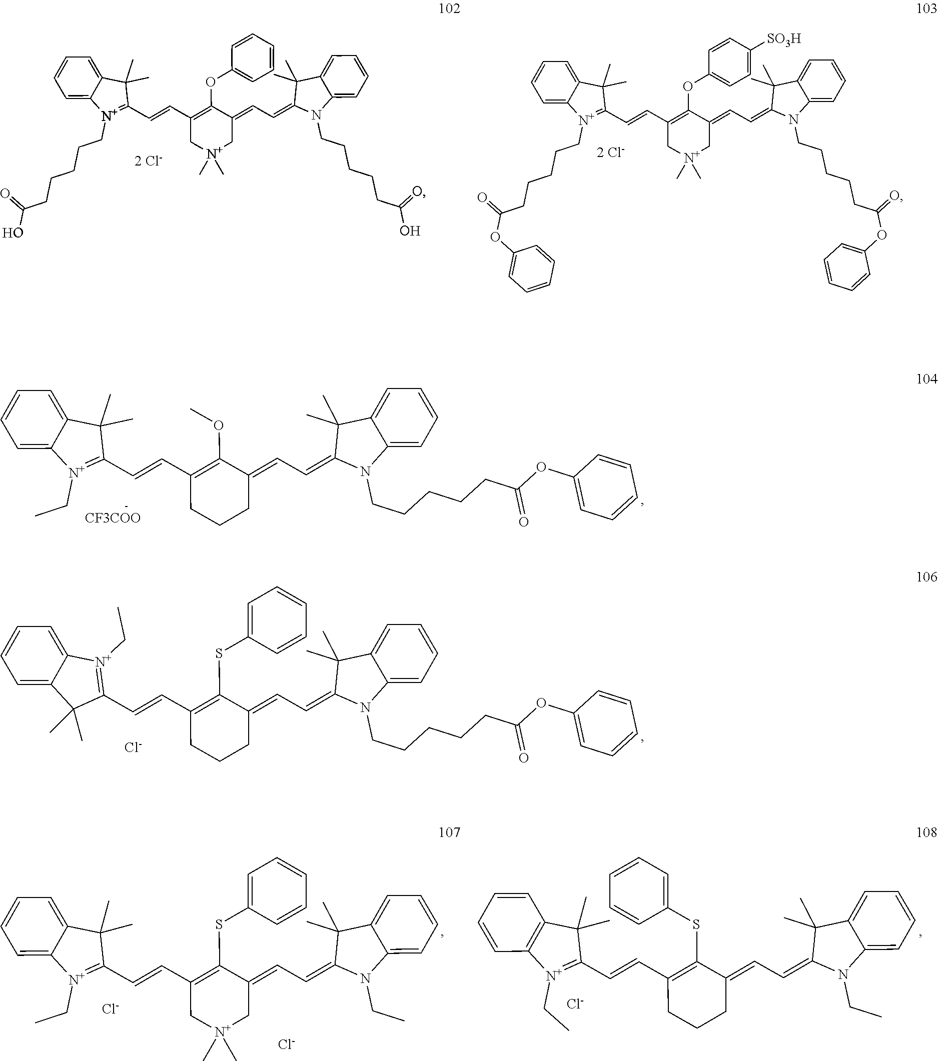 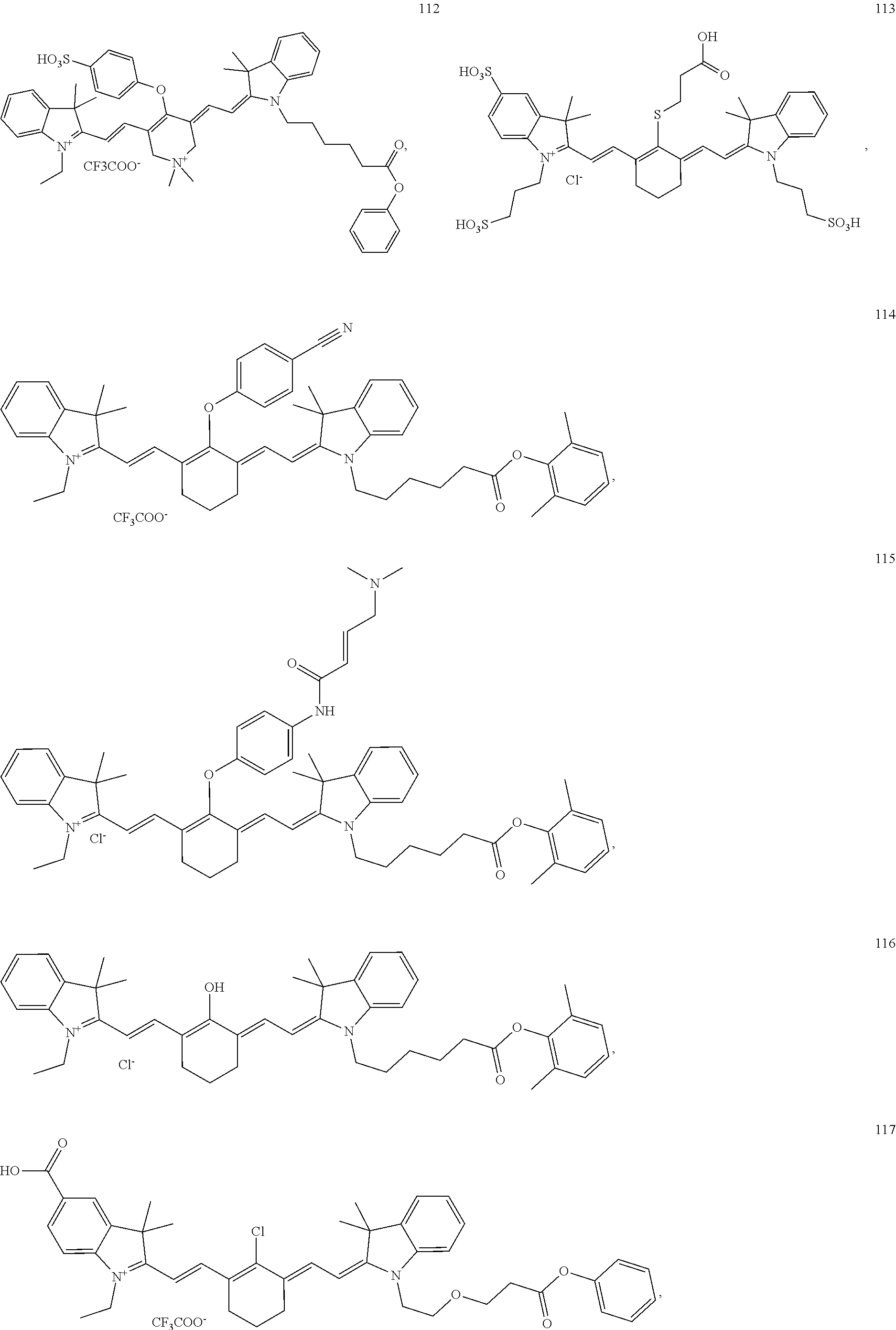 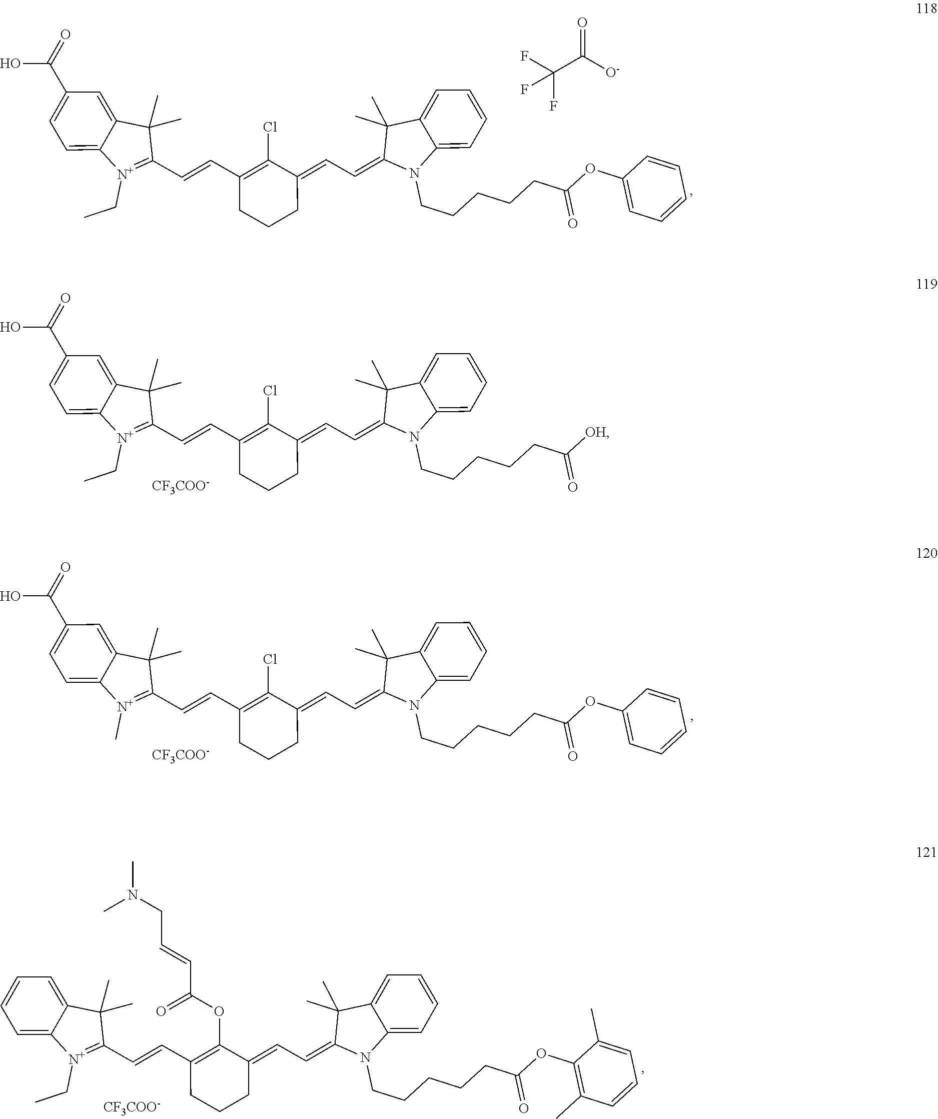 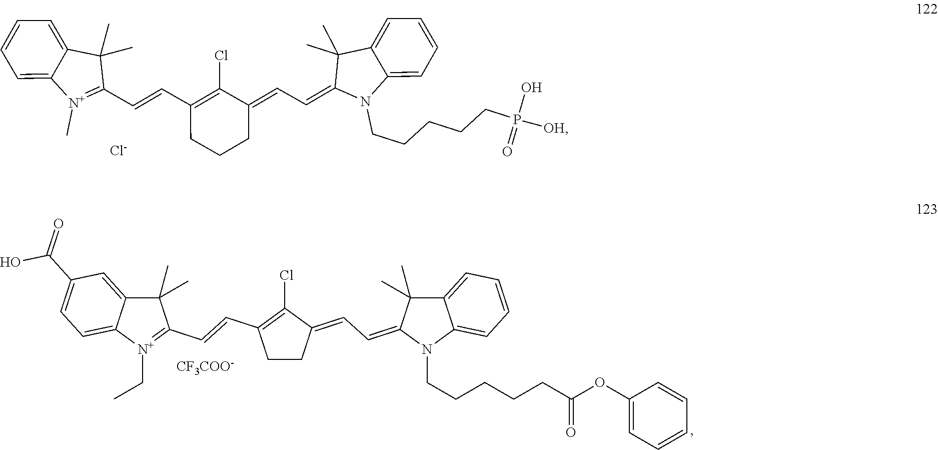
or a pharmaceutically acceptable salt thereof.

29.-34. (canceled)

35. A contrast agent comprising the compound or the pharmaceutically acceptable salt thereof according to claim 1 and an anionic surfactant.

36. The contrast agent according to claim 35, wherein, the anionic surfactant is selected from the group consisting of carboxylate salt, sulfonate salt, sulfate salt, phosphate salt and lipopeptide anionic surfactants; and/or
the molar ratio of the compound or the pharmaceutically acceptable salt thereof to the anionic surfactant ranges from 1:20 to 10:1; and/or
the compound or the pharmaceutically acceptable salt thereof in the contrast agent has a final concentration ranging from 1 to 100 μM; and/or
a solution of the contrast agent has a particle size ranging from 10 to 200 nm; and/or
the contrast agent is in a form of solution or lyophilization powder.

37. An assay kit comprising the compound or the pharmaceutically acceptable salt thereof according to claim 1 and an anionic surfactant.

38. A pharmaceutical composition comprising the compound or the pharmaceutically acceptable salt thereof according to claim 1, and one or more pharmaceutically acceptable carriers.

39. A method of imaging a tumor patient in need thereof, the method comprising administering to the patient the compound or the pharmaceutically acceptable salt thereof according to claim 1.
